SEQUENCE LISTING (152 sequences)

SEQ ID NO: 1 - DNA, 2059 bp, Artificial Sequence
  Synthetic
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggccgtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccaccccccaccccccacccccggaaaacgcgtcgtcccctgggctgggtggagacccccgtcccgcgaaacaccgggccccgcgcagcgtccgggcctgactccgctccggcggctcgcctcctgtgtgcccccgcgccaccgtcgcccgcccgcccgggcccctgcagcctcccagctgccagcgcggagctcctggcggtcaaaagcatacctctgtctgtctttgcccgcttcctggctagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccc

SEQ ID NO: 2 - protein, 424 aa, Artificial Sequence
  Synthetic
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 3 - DNA, 161 bp, Artificial Sequence
  Synthetic
gcgtttagtgaaccgtcagatggtaccgtttaaactcgagtgagcgacatgttgccactcgctttaatctgtaaagccacagatgggattaaagcgagtggcaacatggcgcctactagagcggccgccacagcggggagatccagacatgataagataca

SEQ ID NO: 4 - DNA, 161 bp, Artificial Sequence
  Synthetic
gcgtttagtgaaccgtcagatggtaccgtttaaactcgagtgagcgatccaggattcagatctggtttctgtaaagccacagatgggaaaccagatctgaatcctggactgcctactagagcggccgccacagcggggagatccagacatgataagataca

SEQ ID NO: 5 - DNA, 160 bp, Artificial Sequence
  Synthetic
gcgtttagtgaaccgtcagatggtaccgtttaaactcgagcccagggtctggtgcggagagggcccacagtggacttggtgacgctgtatgccctcaccgctcagcccctgggactagagcggccgccacagcggggagatccagacatgataagataca

SEQ ID NO: 6 - DNA, 92 bp, Artificial Sequence
  Synthetic
cccccagggtctggtgcggagagggcccacagtggacttggtgacgctgtatgccctcaccgctcagcccctggggaattcttcgattctgc

SEQ ID NO: 7 - DNA, 92 bp, Artificial Sequence
  Synthetic
gccccagggtctggtgcggagagggcccacagtggacttggtgacgctgtatgccctcaccgctcagcccctggggaattcttcgattctgc

SEQ ID NO: 8 - DNA, 76 bp, Artificial Sequence
  Synthetic
gccccagggtctggtgcggagagggcccacagtggacttggtgacgctgtatgccctcaccgctcagcccctgggg

SEQ ID NO: 9 - DNA, 92 bp, Artificial Sequence
  Synthetic
gccccagggtctggtgcggagagggcccacagtggacttggtgacgctgtatgccctcaccgctcagcccctggggtaactcctaatcacac

SEQ ID NO: 10 - DNA, 92 bp, Artificial Sequence
  Synthetic
cccccagggtctggtgcggagagggcccacagtggacttggtgacgctgtatgccctcaccgctcagcccctggggtaactcctaatcacac

SEQ ID NO: 11 - DNA, 102 bp, Artificial Sequence
  Synthetic
gaatcacactgccccagggtctggtgcggagagggcccacagtggacttggtgacgctgtatgccctcaccgctcagcccctggggatactcctaatcacac

SEQ ID NO: 12 - DNA, 102 bp, Artificial Sequence
  Synthetic
caatcacactgccccagggtctggtgcggagagggcccacagtggacttggtgacgctgtatgccctcaccgctcagcccctggggatactcctaatcacac

SEQ ID NO: 13 - DNA, 102 bp, Artificial Sequence; identical to SEQ ID NO: 11
  Synthetic

SEQ ID NO: 14 - DNA, 97 bp, Artificial Sequence
  Synthetic
aacgcgccccagggtctggtgcggagagggcccacagtggacttggtgacgctgtatgccctcaccgctcagcccctggggtaactcctaatcacac

SEQ ID NO: 15 - DNA, 92 bp, Artificial Sequence; identical to SEQ ID NO: 7
  Synthetic

SEQ ID NO: 16 - DNA, 93 bp, Artificial Sequence
  Synthetic
gccccagggtctggtgcggagagggcccacagtggacttggtgacgctgtatgccctcaccgctcagcccctggggataactcctaatcacac

SEQ ID NO: 17 - DNA, 103 bp, Artificial Sequence
  Synthetic
gaatcacactgccccagggtctggtgcggagagggcccacagtggacttggtgacgctgtatgccctcaccgctcagcccctggggataactcctaatcacac

SEQ ID NO: 18 - DNA, 96 bp, Artificial Sequence
  Synthetic
gaccgtttaaaccccagggtctggtgcggagagggcccacagtggacttggtgacgctgtatgccctcaccgctcagcccctggggcgcacgccag

SEQ ID NO: 19 - DNA, 103 bp, Artificial Sequence
  Synthetic
gctcgagtgagcgatccaggattcagatctggtttctgtaaagccacagatgggaaaccagatctgaatcctggactgcctactagtaattcttcgattctgc

SEQ ID NO: 20 - DNA, 87 bp, Artificial Sequence
  Synthetic
gctcgagtgagcgatccaggattcagatctggtttctgtaaagccacagatgggaaaccagatctgaatcctggactgcctactaga

SEQ ID NO: 21 - DNA, 108 bp, Artificial Sequence
  Synthetic
gaccgtttaaactcgagtgagcgatccaggattcagatctggtttctgtaaagccacagatgggaaaccagatctgaatcctggactgcctactagagcggccgccac

SEQ ID NO: 22 - DNA, 97 bp, Artificial Sequence
  Synthetic
gaccgtttaaactcgagtgagcgatccaggattcagatctggtttctgtaaagccacagatgggaaaccagatctgaatcctggactgcctactaga

SEQ ID NO: 23 - DNA, 98 bp, Artificial Sequence
  Synthetic
gctcgagtgagcgatccaggattcagatctggtttctgtaaagccacagatgggaaaccagatctgaatcctggactgcctactagagcggccgccac

SEQ ID NO: 24 - DNA, 99 bp, Artificial Sequence
  Synthetic
gactcgagtgagcgatccaggattcagatctggtttctgtaaagccacagatgggaaaccagatctgaatcctggactgcctactagagcggccgccac

SEQ ID NO: 25 - DNA, 99 bp, Artificial Sequence
  Synthetic
gactcgagtgagcgatccaggattcagatctggtttctgtaaagccacagatgggaaaccagatctgaatcctggactgcctactagagcgcacgccag

SEQ ID NO: 26 - DNA, 114 bp, Artificial Sequence
  Synthetic
gaccgtttaaactcgagtgagcgatccaggattcagatctggtttctgtaaagccacagatgggaaaccagatctgaatcctggactgcctactagagaattcttcgattctgc

SEQ ID NO: 27 - DNA, 108 bp, Artificial Sequence
  Synthetic
gaccgtttaaactcgagtgagcgatccaggattcagatctggtttctgtaaagccacagatgggaaaccagatctgaatcctggactgcctactagagcgcacgccag

SEQ ID NO: 28 - DNA, 161 bp, Artificial Sequence
  Synthetic
gcgtttagtgaaccgtcagatggtaccgtttaaactcgagtgagcgatcgtttggaccccgagccaaactgtaaagccacagatgggtttggctcggggtccaaacgagtgcctactagagcggccgccacagcggggagatccagacatgataagataca

SEQ ID NO: 29 - DNA, 103 bp, Artificial Sequence
  Synthetic
gctcgagtgagcgatcgtttggaccccgagccaaactgtaaagccacagatgggtttggctcggggtccaaacgagtgcctactagtaattcttcgattctgc

SEQ ID NO: 30 - DNA, 161 bp, Artificial Sequence
  Synthetic
gcgtttagtgaaccgtcagatggtaccgtttaaactcgagtgagcgaggtccagatttggtttcagaactgtaaagccacagatgggttctgaaaccaaatctggaccctgcctactagagcggccgccacagcggggagatccagacatgataagataca

SEQ ID NO: 31 - DNA, 103 bp, Artificial Sequence
  Synthetic
gctcgagtgagcgaggtccagatttggtttcagaactgtaaagccacagatgggttctgaaaccaaatctggaccctgcctactagtaattcttcgattctgc

SEQ ID NO: 32 - DNA, 161 bp, Artificial Sequence
  Synthetic
gcgtttagtgaaccgtcagatggtaccgtttaaactcgagtgagcgagtccagatttggtttcagaatctgtaaagccacagatgggattctgaaaccaaatctggacctgcctactagagcggccgccacagcggggagatccagacatgataagataca

SEQ ID NO: 33 - DNA, 103 bp, Artificial Sequence
  Synthetic
gctcgagtgagcgagtccagatttggtttcagaatctgtaaagccacagatgggattctgaaaccaaatctggacctgcctactagtaattcttcgattctgc

SEQ ID NO: 34 - DNA, 159 bp, Artificial Sequence
  Synthetic
gcgtttagtgaaccgtcagatggtaccgtttaaactcgagtgagcgagccctgctcctccgagcctttctgaaagccacagatgggaaaggctcggaggagcagggcgtgccactagagcggccgccacagcggggagatccagacatgataagataca

SEQ ID NO: 35 - DNA, 101 bp, Artificial Sequence
  Synthetic
gctcgagtgagcgagccctgctcctccgagcctttctgaaagccacagatgggaaaggctcggaggagcagggcgtgccactagtaattcttcgattctgc

SEQ ID NO: 36 - DNA, 161 bp, Artificial Sequence
  Synthetic
gcgtttagtgaaccgtcagatggtaccgtttaaactcgagtgagcgcgcctttgagaaggatcgctttctgtaaagccacagatgggaaagcgatccttctcaaaggcttgcctactagagcggccgccacagcggggagatccagacatgataagataca

SEQ ID NO: 37 - DNA, 103 bp, Artificial Sequence
  Synthetic
gctcgagtgagcgcgcctttgagaaggatcgctttctgtaaagccacagatgggaaagcgatccttctcaaaggcttgcctactagtaattcttcgattctgc

SEQ ID NO: 38 - DNA, 161 bp, Artificial Sequence
  Synthetic
gcgtttagtgaaccgtcagatggtaccgtttaaactcgagtgagcagaggctctcccacagggggctttctgaaagccacagatgggaaagccccctgtgggagagccctgcctactagagcggccgccacagcggggagatccagacatgataagataca

SEQ ID NO: 39 - DNA, 103 bp, Artificial Sequence
  Synthetic
gctcgagtgagcagaggctctcccacagggggctttctgaaagccacagatgggaaagccccctgtgggagagccctgcctactagtaattcttcgattctgc

SEQ ID NO: 40 - DNA, 161 bp, Artificial Sequence
  Synthetic
gcgtttagtgaaccgtcagatggtaccgtttaaactcgagtgagcgacaggcgcaacctctcctagaactgtaaagccacagatgggttctaggagaggttgcgcctgctgcctactagagcggccgccacagcggggagatccagacatgataagataca

SEQ ID NO: 41 - DNA, 103 bp, Artificial Sequence
  Synthetic
gctcgagtgagcgacaggcgcaacctctcctagaactgtaaagccacagatgggttctaggagaggttgcgcctgctgcctactagtaattcttcgattctgc

SEQ ID NO: 42 - DNA, 160 bp, Artificial Sequence
  Synthetic
gcgtttagtgaaccgtcagatggtaccgtttaaactcgagtgagcgaaggcgcaacctctcctagaaactgaaagccacagatgggtttctaggagaggttgcgcctgtgcctactagagcggccgccacagcggggagatccagacatgataagataca

SEQ ID NO: 43 - DNA, 102 bp, Artificial Sequence
  Synthetic
gctcgagtgagcgaaggcgcaacctctcctagaaactgaaagccacagatgggtttctaggagaggttgcgcctgtgcctactagtaattcttcgattctgc

SEQ ID NO: 44 - DNA, 161 bp, Artificial Sequence
  Synthetic
gcgtttagtgaaccgtcagatggtaccgtttaaactcgagtgagcgccccctcagcgaggaagaatacctgtaaagccacagatggggtattcttcctcgctgaggggttgcctactagagcggccgccacagcggggagatccagacatgataagataca

SEQ ID NO: 45 - DNA, 103 bp, Artificial Sequence
  Synthetic
gctcgagtgagcgccccctcagcgaggaagaatacctgtaaagccacagatggggtattcttcctcgctgaggggttgcctactagtaattcttcgattctgc

SEQ ID NO: 46 - DNA, 161 bp, Artificial Sequence
  Synthetic
gcgtttagtgaaccgtcagatggtaccgtttaaactcgagtgagcgagcggagaactgccattctttcctgtaaagccacagatggggaaagaatggcagttctccgcgtgcctactagagcggccgccacagcggggagatccagacatgataagataca

SEQ ID NO: 47 - DNA, 103 bp, Artificial Sequence
  Synthetic
gctcgagtgagcgagcggagaactgccattctttcctgtaaagccacagatggggaaagaatggcagttctccgcgtgcctactagtaattcttcgattctgc

SEQ ID NO: 48 - DNA, 476 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgacatgttgccactcgctttaatctgtaaagccacagatgggattaaagcgagtggcaacatggcgcctactagagcggccgccacagcggggagatccagacatgataagatacatttttt

SEQ ID NO: 49 - DNA, 476 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgatccaggattcagatctggtttctgtaaagccacagatgggaaaccagatctgaatcctggactgcctactagagcggccgccacagcggggagatccagacatgataagatacatttttt

SEQ ID NO: 50 - DNA, 476 bp, Artificial Sequence
  Synthetic
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagcccagggtctggtgcggagagggcccacagtggacttggtgacgctgtatgccctcaccgctcagcccctgggactagagcggccgccacagcggggagatccagacatgataagatacatttttt

SEQ ID NO: 51 - DNA, 313 bp, Artificial Sequence
  Synthetic
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccaccccccagggtctggtgcggagagggcccacagtggacttggtgacgctgtatgccctcaccgctcagcccctggggaattcttcgattctgctttttt

SEQ ID NO: 52 - DNA, 408 bp, Artificial Sequence
  Synthetic
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgccccagggtctggtgcggagagggcccacagtggacttggtgacgctgtatgccctcaccgctcagcccctggggaattcttcgattctgctttttt

SEQ ID NO: 53 - DNA, 403 bp, Artificial Sequence
  Synthetic
tctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgccccagggtctggtgcggagagggcccacagtggacttggtgacgctgtatgccctcaccgctcagcccctggggtttttt

SEQ ID NO: 54 - DNA, 419 bp, Artificial Sequence
  Synthetic
tctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgccccagggtctggtgcggagagggcccacagtggacttggtgacgctgtatgccctcaccgctcagcccctggggtaactcctaatcacactttttt

SEQ ID NO: 55 - DNA, 319 bp, Artificial Sequence
  Synthetic
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacttggatcccccagggtctggtgcggagagggcccacagtggacttggtgacgctgtatgccctcaccgctcagcccctggggtaactcctaatcacactttttt

SEQ ID NO: 56 - DNA, 429 bp, Artificial Sequence
  Synthetic
tctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgaatcacactgccccagggtctggtgcggagagggcccacagtggacttggtgacgctgtatgccctcaccgctcagcccctggggatactcctaatcacactttttt

SEQ ID NO: 57 - DNA, 330 bp, Artificial Sequence
  Synthetic
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacttggatccaatcacactgccccagggtctggtgcggagagggcccacagtggacttggtgacgctgtatgccctcaccgctcagcccctggggatactcctaatcacactttttt

SEQ ID NO: 58 - DNA, 433 bp, Artificial Sequence
  Synthetic
tctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgttaacgcgaatcacactgccccagggtctggtgcggagagggcccacagtggacttggtgacgctgtatgccctcaccgctcagcccctggggatactcctaatcacactttttt

SEQ ID NO: 59 - DNA, 423 bp, Artificial Sequence
  Synthetic
tctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgttaacgcgccccagggtctggtgcggagagggcccacagtggacttggtgacgctgtatgccctcaccgctcagcccctggggtaactcctaatcacactttttt

SEQ ID NO: 60 - DNA, 537 bp, Artificial Sequence
  Synthetic
ttttaaaagaaaaggggggattggggggtacagtgcaggggaaagaatagtagacataatagcaacagacatacaaactaaagaattacaaaaacaaattacaaaaattcaaaatttttctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgccccagggtctggtgcggagagggcccacagtggacttggtgacgctgtatgccctcaccgctcagcccctggggaattcttcgattctgctttttt

SEQ ID NO: 61 - DNA, 420 bp, Artificial Sequence
  Synthetic
tctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgccccagggtctggtgcggagagggcccacagtggacttggtgacgctgtatgccctcaccgctcagcccctggggataactcctaatcacactttttt

SEQ ID NO: 62 - DNA, 430 bp, Artificial Sequence
  Synthetic
tctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgaatcacactgccccagggtctggtgcggagagggcccacagtggacttggtgacgctgtatgccctcaccgctcagcccctggggataactcctaatcacactttttt

SEQ ID NO: 63 - DNA, 423 bp, Artificial Sequence
  Synthetic
tctagagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgaccgtttaaaccccagggtctggtgcggagagggcccacagtggacttggtgacgctgtatgccctcaccgctcagcccctggggcgcacgccagtttttt

SEQ ID NO: 64 - DNA, 418 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctcgagtgagcgatccaggattcagatctggtttctgtaaagccacagatgggaaaccagatctgaatcctggactgcctactagtaattcttcgattctgctttttt

SEQ ID NO: 65 - DNA, 402 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctcgagtgagcgatccaggattcagatctggtttctgtaaagccacagatgggaaaccagatctgaatcctggactgcctactagatttttt

SEQ ID NO: 66 - DNA, 423 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgaccgtttaaactcgagtgagcgatccaggattcagatctggtttctgtaaagccacagatgggaaaccagatctgaatcctggactgcctactagagcggccgccactttttt

SEQ ID NO: 67 - DNA, 412 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgaccgtttaaactcgagtgagcgatccaggattcagatctggtttctgtaaagccacagatgggaaaccagatctgaatcctggactgcctactagatttttt

SEQ ID NO: 68 - DNA, 413 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctcgagtgagcgatccaggattcagatctggtttctgtaaagccacagatgggaaaccagatctgaatcctggactgcctactagagcggccgccactttttt

SEQ ID NO: 69 - DNA, 414 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgactcgagtgagcgatccaggattcagatctggtttctgtaaagccacagatgggaaaccagatctgaatcctggactgcctactagagcggccgccactttttt

SEQ ID NO: 70 - DNA, 414 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgactcgagtgagcgatccaggattcagatctggtttctgtaaagccacagatgggaaaccagatctgaatcctggactgcctactagagcgcacgccagtttttt

SEQ ID NO: 71 - DNA, 429 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgaccgtttaaactcgagtgagcgatccaggattcagatctggtttctgtaaagccacagatgggaaaccagatctgaatcctggactgcctactagagaattcttcgattctgctttttt

SEQ ID NO: 72 - DNA, 423 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgaccgtttaaactcgagtgagcgatccaggattcagatctggtttctgtaaagccacagatgggaaaccagatctgaatcctggactgcctactagagcgcacgccagtttttt

SEQ ID NO: 73 - DNA, 476 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgatcgtttggaccccgagccaaactgtaaagccacagatgggtttggctcggggtccaaacgagtgcctactagagcggccgccacagcggggagatccagacatgataagatacatttttt

SEQ ID NO: 74 - DNA, 417 bp, Artificial Sequence
  Synthetic
cgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctcgagtgagcgatcgtttggaccccgagccaaactgtaaagccacagatgggtttggctcggggtccaaacgagtgcctactagtaattcttcgattctgctttttt

SEQ ID NO: 75 - DNA, 476 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgaggtccagatttggtttcagaactgtaaagccacagatgggttctgaaaccaaatctggaccctgcctactagagcggccgccacagcggggagatccagacatgataagatacatttttt

SEQ ID NO: 76 - DNA, 418 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctcgagtgagcgaggtccagatttggtttcagaactgtaaagccacagatgggttctgaaaccaaatctggaccctgcctactagtaattcttcgattctgctttttt

SEQ ID NO: 77 - DNA, 477 bp, Artificial Sequence
  Synthetic
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgagtccagatttggtttcagaatctgtaaagccacagatgggattctgaaaccaaatctggacctgcctactagagcggccgccacagcggggagatccagacatgataagatacatttttt

SEQ ID NO: 78 - DNA, 418 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctcgagtgagcgagtccagatttggtttcagaatctgtaaagccacagatgggattctgaaaccaaatctggacctgcctactagtaattcttcgattctgctttttt

SEQ ID NO: 79 - DNA, 474 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgagccctgctcctccgagcctttctgaaagccacagatgggaaaggctcggaggagcagggcgtgccactagagcggccgccacagcggggagatccagacatgataagatacatttttt

SEQ ID NO: 80 - DNA, 416 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctcgagtgagcgagccctgctcctccgagcctttctgaaagccacagatgggaaaggctcggaggagcagggcgtgccactagtaattcttcgattctgctttttt

SEQ ID NO: 81 - DNA, 477 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgcgcctttgagaaggatcgctttctgtaaagccacagatgggaaagcgatccttctcaaaggcttgcctactagagcggccgccacagcggggagatccagacatgataagatacattttttg

SEQ ID NO: 82 - DNA, 418 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctcgagtgagcgcgcctttgagaaggatcgctttctgtaaagccacagatgggaaagcgatccttctcaaaggcttgcctactagtaattcttcgattctgctttttt

SEQ ID NO: 83 - DNA, 476 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcagaggctctcccacagggggctttctgaaagccacagatgggaaagccccctgtgggagagccctgcctactagagcggccgccacagcggggagatccagacatgataagatacatttttt

SEQ ID NO: 84 - DNA, 418 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctcgagtgagcagaggctctcccacagggggctttctgaaagccacagatgggaaagccccctgtgggagagccctgcctactagtaattcttcgattctgctttttt

SEQ ID NO: 85 - DNA, 477 bp, Artificial Sequence
  Synthetic
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgacaggcgcaacctctcctagaactgtaaagccacagatgggttctaggagaggttgcgcctgctgcctactagagcggccgccacagcggggagatccagacatgataagatacatttttt

SEQ ID NO: 86 - DNA, 418 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctcgagtgagcgacaggcgcaacctctcctagaactgtaaagccacagatgggttctaggagaggttgcgcctgctgcctactagtaattcttcgattctgctttttt

SEQ ID NO: 87 - DNA, 475 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgaaggcgcaacctctcctagaaactgaaagccacagatgggtttctaggagaggttgcgcctgtgcctactagagcggccgccacagcggggagatccagacatgataagatacatttttt

SEQ ID NO: 88 - DNA, 417 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctcgagtgagcgaaggcgcaacctctcctagaaactgaaagccacagatgggtttctaggagaggttgcgcctgtgcctactagtaattcttcgattctgctttttt

SEQ ID NO: 89 - DNA, 477 bp, Artificial Sequence
  Synthetic
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgccccctcagcgaggaagaatacctgtaaagccacagatggggtattcttcctcgctgaggggttgcctactagagcggccgccacagcggggagatccagacatgataagatacatttttt

SEQ ID NO: 90 - DNA, 419 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctcgagtgagcgccccctcagcgaggaagaatacctgtaaagccacagatggggtattcttcctcgctgaggggttgcctactagtaattcttcgattctgcttttttc

SEQ ID NO: 91 - DNA, 477 bp, Artificial Sequence
  Synthetic
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgagcggagaactgccattctttcctgtaaagccacagatggggaaagaatggcagttctccgcgtgcctactagagcggccgccacagcggggagatccagacatgataagatacatttttt

SEQ ID NO: 92 - DNA, 418 bp, Artificial Sequence
  Synthetic
acgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgctcgagtgagcgagcggagaactgccattctttcctgtaaagccacagatggggaaagaatggcagttctccgcgtgcctactagtaattcttcgattctgctttttt

SEQ ID NO: 93 - DNA, 22 bp, Artificial Sequence
  Synthetic
auuaaagcgaguggcaacaugg

SEQ ID NO: 94 - DNA, 22 bp, Artificial Sequence
  Synthetic
aaaccagaucugaauccuggac

SEQ ID NO: 95 - DNA, 23 bp, Artificial Sequence
  Synthetic
uggugcggagagggcccacagug

SEQ ID NO: 96 - DNA, 22 bp, Artificial Sequence
  Synthetic
uuuggcucgggguccaaacgag

SEQ ID NO: 97 - DNA, 22 bp, Artificial Sequence
  Synthetic
uucugaaaccaaaucuggaccc

SEQ ID NO: 98 - DNA, 22 bp, Artificial Sequence
  Synthetic
auucugaaaccaaaucuggacc

SEQ ID NO: 99 - DNA, 22 bp, Artificial Sequence
  Synthetic
aaaggctcggaggagcagggcg

SEQ ID NO: 100 - DNA, 22 bp, Artificial Sequence
  Synthetic
aaagcgauccuucucaaaggcu

SEQ ID NO: 101 - DNA, 22 bp, Artificial Sequence
  Synthetic
aaagcccccugugggagagccc

SEQ ID NO: 102 - DNA, 22 bp, Artificial Sequence
  Synthetic
uucuaggagagguugcgccugc

SEQ ID NO: 103 - DNA, 22 bp, Artificial Sequence
  Synthetic
uuucuaggagagguugcgccug

SEQ ID NO: 104 - DNA, 22 bp, Artificial Sequence
  Synthetic
guauucuuccucgcugaggggu

SEQ ID NO: 105 - DNA, 22 bp, Artificial Sequence
  Synthetic
gaaagaauggcaguucuccgcg

SEQ ID NO: 106 - DNA, 22 bp, Artificial Sequence
  Synthetic
guccaggauucagaucugguuu

SEQ ID NO: 107 - DNA, 20 bp, Artificial Sequence
  Synthetic
ccaggguccagauuugguuu

SEQ ID NO: 108 - DNA, 20 bp, Artificial Sequence
  Synthetic
cgugggucgccuucgcccac

SEQ ID NO: 109 - DNA, 28 bp, Artificial Sequence
  Synthetic
gcgcugcagcccagccaggccgcgccgg

SEQ ID NO: 110 - DNA, 23 bp, Artificial Sequence
  Synthetic
ugcagcccagccaggccgcgccg

SEQ ID NO: 111 - DNA, 20 bp, Artificial Sequence
  Synthetic
uccucgcuggccuccgcacc

SEQ ID NO: 112 - DNA, 22 bp, Artificial Sequence
  Synthetic
cuccaccuccccagcccgcgcc

SEQ ID NO: 113 - DNA, 21 bp, Artificial Sequence
  Synthetic
ccggugagagacuccacaccg

SEQ ID NO: 114 - DNA, 21 bp, Artificial Sequence
  Synthetic
ccggugagagacuccacaccg

SEQ ID NO: 115 - DNA, 22 bp, Artificial Sequence
  Synthetic
cguuuggaccccgagccaaacu

SEQ ID NO: 116 - DNA, 22 bp, Artificial Sequence
  Synthetic
guccagauuugguuucagaacu

SEQ ID NO: 117 - DNA, 22 bp, Artificial Sequence
  Synthetic
uccagauuugguuucagaaucu

SEQ ID NO: 118 - DNA, 22 bp, Artificial Sequence
  Synthetic
cccugcuccuccgagccuuucu

SEQ ID NO: 119 - DNA, 22 bp, Artificial Sequence
  Synthetic
ccuuugagaaggaucgcuuucu

SEQ ID NO: 120 - DNA, 22 bp, Artificial Sequence
  Synthetic
gcucucccacagggggcuuucu

SEQ ID NO: 121 - DNA, 22 bp, Artificial Sequence
  Synthetic
aggcgcaaccucuccuagaacu

SEQ ID NO: 122 - DNA, 22 bp, Artificial Sequence
  Synthetic
ggcgcaaccucuccuagaaacu

SEQ ID NO: 123 - DNA, 22 bp, Artificial Sequence
  Synthetic
cccucagcgaggaagaauaccu

SEQ ID NO: 124 - DNA, 22 bp, Artificial Sequence
  Synthetic
cggagaacugccauucuuuccu

SEQ ID NO: 125 - DNA, 26 bp, Artificial Sequence
  Synthetic
ccggctcgagatggccctcccgacac

SEQ ID NO: 126 - DNA, 34 bp, Artificial Sequence
  Synthetic
acgactagtgggagggggcattttaatatatctc

SEQ ID NO: 127 - DNA, 26 bp, Artificial Sequence
  Synthetic
ccggctcgagatggccctcccgacac

SEQ ID NO: 128 - DNA, 34 bp, Artificial Sequence; identical to SEQ ID NO: 126
  Synthetic

SEQ ID NO: 129 - DNA, 7 bp, Artificial Sequence
  Synthetic
attaaaa

SEQ ID NO: 130 - DNA, 29 bp, Artificial Sequence
  Synthetic
ttactagtattaatagtaatcaattacgg

SEQ ID NO: 131 - DNA, 30 bp, Artificial Sequence
  Synthetic
caatgaattcgttaatgattaacccgccat

SEQ ID NO: 132 - DNA, 44 bp, Artificial Sequence
  Synthetic
ccgagaattcctcgacttattaatagtaatcaattacggggtca

SEQ ID NO: 133 - DNA, 39 bp, Artificial Sequence
  Synthetic
acccaagatctggggcaaggtgggcaaaagccgggagga

SEQ ID NO: 134 - DNA, 39 bp, Artificial Sequence
  Synthetic
caccttgccccagatcttgggtgcctgagggtgggagag

SEQ ID NO: 135 - DNA, 32 bp, Artificial Sequence
  Synthetic
cgggtaccctacgtagaatcgagcccgaggag

SEQ ID NO: 136 - DNA, 29 bp, Artificial Sequence
  Synthetic
ttactagtattaatagtaatcaattacgg

SEQ ID NO: 137 - DNA, 24 bp, Artificial Sequence
  Synthetic
attcgttaatgattaacccgccat

SEQ ID NO: 138 - DNA, 26 bp, Artificial Sequence
  Synthetic
ccggctcgagatggccctcccgacac

SEQ ID NO: 139 - DNA, 34 bp, Artificial Sequence; identical to SEQ ID NO: 126
  Synthetic

SEQ ID NO: 140 - DNA, 26 bp, Artificial Sequence
  Synthetic
ccggctcgagatggccctcccgacac

SEQ ID NO: 141 - DNA, 19 bp, Artificial Sequence
  Synthetic
cgggcaaaagccgggagga

SEQ ID NO: 142 - DNA, 35 bp, Artificial Sequence
  Synthetic
tcctcccggcttttgcccggcctgagggtgggaga

SEQ ID NO: 143 - DNA, 27 bp, Artificial Sequence
  Synthetic
agcggccgcaagctcctccagcagagc

SEQ ID NO: 144 - DNA, 50 bp, Artificial Sequence
  Synthetic
gtcgtatccagtgcagggtccgaggtattcgcactggatacgacgtccag

SEQ ID NO: 145 - DNA, 22 bp, Artificial Sequence
  Synthetic
cggcccaaaccagatctgaatc

SEQ ID NO: 146 - DNA, 16 bp, Artificial Sequence
  Synthetic
gtgcagggtccgaggt

SEQ ID NO: 147 - DNA, 16 bp, Artificial Sequence
  Synthetic
atacgacgtccaggat

SEQ ID NO: 148 - DNA, 23 bp, Artificial Sequence
  Synthetic
cactgtgggccctctccgcacca

SEQ ID NO: 149 - DNA, 4 bp, Artificial Sequence
  Synthetic
ugug

SEQ ID NO: 150 - DNA, 5 bp, Artificial Sequence
  Synthetic
uggug

SEQ ID NO: 151 - DNA, 4 bp, Artificial Sequence
  Synthetic
  misc_feature (2)..(3): n is a, c, g, t or u
cnnc

SEQ ID NO: 152 - DNA, 23 bp, Artificial Sequence
  Synthetic
uggugcggagagggcccacagug

(19) United States
(12) Patent Application Publication
(10) Pub. No.: US 2024/0093191 A1
(43) Pub. Date: Mar. 21, 2024

(54) COMPOSITIONS AND METHODS FOR TREATING DISEASE ASSOCIATED WITH DUX4 OVEREXPRESSION

(71) Applicants: Nizar Saad, Pickerington, OH (US); Scott Quenton Harper, Powell, OH (US)

(72) Inventors: Nizar Saad, Pickerington, OH (US); Scott Quenton Harper, Powell, OH (US)

(73) Assignee: RESEARCH INSTITUTE AT NATIONWIDE CHILDREN'S HOSPITAL, Columbus, OH (US)

(21) Appl. No.: 18/274,327

(22) PCT Filed: Feb. 3, 2022
(86) PCT No.: PCT/US22/15011

§ 371 Date: Jul. 26, 2023

Related U.S. Application Data
(60) Provisional application No. 63/145,255, filed on Feb. 3, 2021.

(51) Int. Cl. C12N 15/113; A61K 9/00; A61K 45/06; A61P 21/00; C12N 15/86

(52) U.S. Cl. CPC C12N 15/113; A61K 9/0019; A61K 45/06; A61P 21/00; C12N 15/86; C12N 2310/141; C12N 2750/14143

(57) ABSTRACT

Disclosed herein are products, methods, and uses for treating, ameliorating, delaying the progression of, and/or preventing a muscular dystrophy or a cancer including, but not limited to, facioscapulohumeral muscular dystrophy (FSHD) or a cancer associated with DUX4 expression or overexpression. More particularly, disclosed herein are RNA interference-based products, methods, and uses for inhibiting or downregulating the expression of double homeobox 4 (DUX4). Even more particularly, the disclosure provides microRNA (miRNA) for inhibiting or downregulating the expression of DUX4 and methods of using said miRNA to inhibit or downregulate DUX4 expression in cells and/or in cells of a subject having a muscular dystrophy or a cancer including, but not limited to, FSHD or a cancer associated with DUX4 expression or overexpression. Additionally, the disclosure provides an estrogen, synthetic estrogen, progesterone, progestin, melatonin, bleomycin, pyrazinamide, sorafenib, or a derivative thereof, or a combination of any thereof for upregulating expression of microRNA-675, inhibiting DUX4 expression, and for treating, ameliorating, delaying the progression of, and/or preventing a muscular dystrophy or a cancer including, but not limited to, FSHD or a cancer associated with DUX4 expression or overexpression.

### INCORPORATION BY REFERENCE OF THE SEQUENCE LISTING

This application contains, as a separate part of disclosure, a Sequence Listing in computer-readable form (Filename: 53445 Seqlisting.txt; Size: 61,557 bytes; Created: Feb. 1, 2022) which is incorporated by reference herein in its entirety.

### FIELD

This disclosure relates to the field of the treatment of disease associated with the overexpression of the double homeobox 4 (DUX4) gene. More particularly, the disclosure provides RNA interference-based products, methods, and uses for treating, ameliorating, delaying the progression of, and/or preventing a muscular dystrophy or cancer associated with DUX4 expression or overexpression of the DUX4 gene. Specifically, the disclosure provides products and methods for inhibiting or downregulating the expression of the DUX4 gene. More specifically, the disclosure provides microRNA (miRNA) for inhibiting or downregulating the expression of DUX4 and methods of using said miRNA to inhibit or downregulate DUX4 expression in cells and/or in a subject having a muscular dystrophy including, but not limited to facioscapulohumeral muscular dystrophy (FSHD), or a cancer associated with overexpressed DUX4. Additionally, the disclosure provides an estrogen, synthetic estrogen, progesterone, progestin, melatonin, bleomycin, pyrazinamide, sorafenib, or a derivative thereof, or a combination of any thereof for upregulating expression of microRNA-675, inhibiting DUX4 expression, and/or for treating, ameliorating, delaying the progression of, and/or preventing a muscular dystrophy or a cancer including, but not limited to, FSHD or a cancer associated with DUX4 expression or overexpression.

### BACKGROUND

Muscular dystrophies (MDs) are a group of genetic diseases. The group is characterized by progressive weakness and degeneration of the skeletal muscles that control movement. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

Facioscapulohumeral dystrophy (FSHD) is among the most commonly inherited muscular dystrophies, estimated to affect as many as 870,000 individuals. Classical descriptions of FSHD presentation include progressive muscle weakness in the face, shoulder-girdle and arms, but disease can manifest more broadly, including in muscles of the trunk and lower extremities. Variability is also commonly seen within individuals, as asymmetrical weakness is common. Age-at-onset can range from early childhood to adulthood, and is usually related to disease severity, where earlier onset is often associated with more severe muscle weakness. Although most patients with FSHD have a normal life span, respiratory insufficiency can occur, and the disease can be debilitating, as approximately 25% of affected individuals may become wheelchair dependent by their fifties, and even earlier in more severe forms of the disease, while others maintain lifelong ambulation.

FSHD is caused by aberrant expression of the double homeobox 4 gene (DUX4), which produces a transcription factor that is toxic to skeletal muscle. DUX4 is normally functional during the two-cell stage of human development but repressed thereafter in essentially all other tissues, except perhaps the testes. In skeletal muscles of people with FSHD, specific genetic and epigenetic factors conspire to permit DUX4 de-repression, where it then initiates several aberrant gene expression cascades, including those involved in differentiation abnormalities, oxidative stress, inflammatory infiltration, cell death and muscle atrophy.

Effective FSHD-targeted therapies would dramatically improve patient quality of life, but currently there are no approved treatments that slow FSHD progression or improve muscle weakness. Since FSHD arises from DUX4 de-repression, the most direct route to a therapy will involve inhibiting DUX4 in muscle. Gene silencing by RNA interference (RNAi) is one powerful approach to inhibit DUX4. Historically, RNAi-based therapies have relied upon two major strategies to silence dominant disease genes: (1) delivery of siRNA oligonucleotide drugs to permissive target cells or tissues; or (2) gene therapy in which designed microRNA or shRNA expression cassettes are packaged within a viral vector and expressed intracellularly following delivery.

RNA interference (RNAi) is a mechanism of gene regulation in eukaryotic cells that has been considered for the treatment of various diseases. RNAi refers to post-transcriptional control of gene expression mediated by microRNAs (miRNAs). The miRNAs are small (21-25 nucleotides), noncoding RNAs that share sequence homology and base-pair with 3′ untranslated regions of cognate messenger RNAs (mRNAs). The interaction between the miRNAs and mRNAs directs cellular gene silencing machinery to prevent the translation of the mRNAs. The RNAi pathway is summarized in Duan (Ed.), Section 7.3 of Chapter 7 in Muscle Gene Therapy, Springer Science+Business Media, LLC (2010).

As an understanding of natural RNAi pathways has developed, researchers have designed artificial miRNAs for use in regulating expression of target genes for treating disease. As described in Section 7.4 of Duan, supra, artificial miRNAs can be transcribed from DNA expression cassettes. The miRNA sequence specific for a target gene is transcribed along with sequences required to direct processing of the miRNA in a cell. Viral vectors, such as adeno-associated virus (AAV) have been used to deliver miRNAs to muscle [Fechner et al., J. Mol. Med., 86: 987-997 (2008)].

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is that it is an extremely stable and hardy virus. It easily withstands the conditions used to inactivate adenovirus (56° to 65° C. for several hours), making cold preservation of AAV less critical. AAV may even be lyophilized. Finally, AAV-infected cells are not resistant to superinfection.

There remains a need in the art for products and methods for treating diseases associated with overexpressed DUX4 including muscular dystrophies, such as FSHD, and cancer.

### SUMMARY

The disclosure provides products, methods, and uses for inhibiting DUX4 expression and for treating, ameliorating, delaying the progression of, and/or preventing a muscular dystrophy or cancer associated with the expression or overexpression of DUX4.

The disclosure provides nucleic acids designed to inhibit DUX4 expression, viral vectors comprising the nucleic acids, compositions comprising the nucleic acids and vectors, methods for using these products for inhibiting and/or interfering with expression of a DUX4 gene in a cell, and methods for treating or ameliorating disease in a subject suffering from a disease resulting from elevated expression of DUX4.

The disclosure provides a nucleic acid encoding a double homeobox 4 (DUX4)-targeting microRNA (miRNA) comprising: (a) a nucleotide sequence comprising at least 90% identity to the sequence set forth in any one of SEQ ID NOs: 5-47; (b) the nucleotide sequence set forth in any one of SEQ ID NOs: 5-47; (c) a nucleotide sequence that encodes the RNA sequence set forth in any one of SEQ ID NOs: 95-105; or (d) a nucleotide sequence that specifically hybridizes to the DUX4 sequence set forth in any one of SEQ ID NOs: 106-124. In some aspects, the nucleic acid further comprises a promoter sequence. In some aspects, the promoter is any of U6, U7, tRNA, H1, minimal CMV, T7, EF1-alpha, Minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, or CK1. In some aspects, the nucleic acid comprises (a) a nucleotide sequence comprising at least 90% identity to the sequence set forth in any one of SEQ ID NOs: 50-92; or (b) the nucleotide sequence set forth in any one of SEQ ID NOs: 50-92.

The disclosure provides an adeno-associated virus comprising the nucleic acid encoding a double homeobox 4 (DUX4)-targeting microRNA (miRNA) comprising: (a) a nucleotide sequence comprising at least 90% identity to the sequence set forth in any one of SEQ ID NOs: 5-47; (b) the nucleotide sequence set forth in any one of SEQ ID NOs: 5-47; (c) a nucleotide sequence that encodes the RNA sequence set forth in any one of SEQ ID NOs: 95-105; or (d) a nucleotide sequence that specifically hybridizes to the DUX4 sequence set forth in any one of SEQ ID NOs: 106-124. In some aspects, the nucleic acid further comprises a promoter sequence. In some aspects, the promoter is any of U6, U7, tRNA, H1, minimal CMV, T7, EF1-alpha, Minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, or CK1. In some aspects, the adeno-associated virus comprises a nucleic acid comprising (a) a nucleotide sequence comprising at least 90% identity to the sequence set forth in any one of SEQ ID NOs: 50-92; or (b) the nucleotide sequence set forth in any one of SEQ ID NOs: 50-92. In some aspects, the adeno-associated virus lacks rep and cap genes. In some aspects, the adeno-associated virus is a recombinant AAV (rAAV) or a self-complementary recombinant AAV (scAAV). In some aspects, the adeno-associated virus is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, or AAV-B1. In some aspects, the adeno-associated virus is AAV-9.

The disclosure provides a nanoparticle, extracellular vesicle, or exosome comprising the nucleic acid encoding a double homeobox 4 (DUX4)-targeting microRNA (miRNA) comprising: (a) a nucleotide sequence comprising at least 90% identity to the sequence set forth in any one of SEQ ID NOs: 5-47; (b) the nucleotide sequence set forth in any one of SEQ ID NOs: 5-47; (c) a nucleotide sequence that encodes the RNA sequence set forth in any one of SEQ ID NOs: 95-105; or (d) a nucleotide sequence that specifically hybridizes to the DUX4 sequence set forth in any one of SEQ ID NOs: 106-124. In some aspects, the nanoparticle, extracellular vesicle, or exosome comprises the nucleic acid comprising the RNA sequence set forth in any one of SEQ ID NOs: 94-105. In some aspects, the nucleic acid further comprises a promoter sequence. In some aspects, the promoter is any of U6, U7, tRNA, H1, minimal CMV, T7, EF1-alpha, Minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, or CK1. In some aspects, the nanoparticle, extracellular vesicle, or exosome comprises a nucleic acid comprising (a) a nucleotide sequence comprising at least 90% identity to the sequence set forth in any one of SEQ ID NOs: 50-92; or (b) the nucleotide sequence set forth in any one of SEQ ID NOs: 50-92. In some aspects, the nanoparticle, extracellular vesicle, or exosome comprises a nucleic acid comprising at least 90% identity to the sequence set forth in any one of SEQ ID NOs: 94-105.

The disclosure provides a composition comprising a nucleic acid, an adeno-associated virus, or nanoparticle, extracellular vesicle, or exosome, as described herein the disclosure, and a pharmaceutically acceptable carrier.

The disclosure provides a method of inhibiting and/or interfering with expression of a double homeobox 4 (DUX4) gene in a cell comprising contacting the cell with a nucleic acid encoding a double homeobox 4 (DUX4)-targeting microRNA (miRNA) comprising: (a) a nucleotide sequence comprising at least 90% identity to the sequence set forth in any one of SEQ ID NOs: 5-47; (b) the nucleotide sequence set forth in any one of SEQ ID NOs: 5-47; (c) a nucleotide sequence that encodes the RNA sequence set forth in any one of SEQ ID NOs: 95-105; or (d) a nucleotide sequence that specifically hybridizes to the DUX4 sequence set forth in any one of SEQ ID NOs: 106-124. In some aspects, the nucleic acid further comprises a promoter sequence. In some aspects, the promoter is any of U6, U7, tRNA, H1, minimal CMV, T7, EF1-alpha, Minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, or CK1. In some aspects, the method comprises a nucleic acid comprising (a) a nucleotide sequence comprising at least 90% identity to the sequence set forth in any one of SEQ ID NOs: 50-92; or (b) the nucleotide sequence set forth in any one of SEQ ID NOs: 50-92.

The disclosure provides a method of inhibiting and/or interfering with expression of a double homeobox 4 (DUX4) gene in a cell comprising contacting the cell with an adeno-associated virus comprising the nucleic acid encoding a double homeobox 4 (DUX4)-targeting microRNA (miRNA) comprising: (a) a nucleotide sequence comprising at least 90% identity to the sequence set forth in any one of SEQ ID NOs: 5-47; (b) the nucleotide sequence set forth in any one of SEQ ID NOs: 5-47; (c) a nucleotide sequence that encodes the RNA sequence set forth in any one of SEQ ID NOs: 95-105; or (d) a nucleotide sequence that specifically hybridizes to the DUX4 sequence set forth in any one of SEQ ID NOs: 106-124. In some aspects, the nucleic acid further comprises a promoter sequence. In some aspects, the promoter is any of U6, U7, tRNA, H1, minimal CMV, T7, EF1-alpha, Minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, or CK1. In some aspects, the adeno-associated virus comprises a nucleic acid comprising (a) a nucleotide sequence comprising at least 90% identity to the sequence set forth in any one of SEQ ID NOs: 50-92; or (b) the nucleotide sequence set forth in any one of SEQ ID NOs: 50-92. In some aspects, the adeno-associated virus lacks rep and cap genes. In some aspects, the adeno-associated virus is a recombinant AAV (rAAV) or a self-complementary recombinant AAV (scAAV). In some aspects, the adeno-associated virus is AAV-1, AAV-2, AAV-3, AAV-4, AAV-5, AAV-6, AAV-7, AAV-8, AAV-9, AAV-10, AAV-11, AAV-12, AAV-13, AAV-anc80, or AAV rh.74. In some aspects, the adeno-associated virus is AAV-9.

The disclosure provides a method of inhibiting and/or interfering with expression of a double homeobox 4 (DUX4) gene in a cell comprising contacting the cell with a nanoparticle, extracellular vesicle, or exosome comprising the nucleic acid encoding a double homeobox 4 (DUX4)-targeting microRNA (miRNA) comprising: (a) a nucleotide sequence comprising at least 90% identity to the sequence set forth in any one of SEQ ID NOs: 5-47; (b) the nucleotide sequence set forth in any one of SEQ ID NOs: 5-47; (c) a nucleotide sequence that encodes the RNA sequence set forth in any one of SEQ ID NOs: 95-105; or (d) a nucleotide sequence that specifically hybridizes to the DUX4 sequence set forth in any one of SEQ ID NOs: 106-124. In some aspects, the disclosure provides a method of inhibiting and/or interfering with expression of a double homeobox 4 (DUX4) gene in a cell comprising contacting the cell with a nanoparticle, extracellular vesicle, or exosome comprising the nucleic acid comprising the sequence set forth in any one of SEQ ID NOs: 94-105. In some aspects, the nucleic acid further comprises a promoter sequence. In some aspects, the promoter is any of U6, U7, tRNA, H1, minimal CMV, T7, EF1-alpha, Minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, or CK1. In some aspects, the nanoparticle, extracellular vesicle, or exosome comprises a nucleic acid comprising (a) a nucleotide sequence comprising at least 90% identity to the sequence set forth in any one of SEQ ID NOs: 50-92; or (b) the nucleotide sequence set forth in any one of SEQ ID NOs: 50-92. In some aspects, the nanoparticle, extracellular vesicle, or exosome comprises a nucleic acid comprising at least 90% identity or 100% identity to the sequence set forth in any one of SEQ ID NOs: 94-105.

The disclosure provides a method of inhibiting and/or interfering with expression of a double homeobox 4 (DUX4) gene in a cell comprising contacting the cell with a composition comprising a nucleic acid, an adeno-associated virus, or nanoparticle, extracellular vesicle, or exosome, as described herein the disclosure, and a pharmaceutically acceptable carrier.

The disclosure provides a method of treating a subject having a muscular dystrophy or a cancer comprising administering to the subject an effective amount of a nucleic acid encoding a double homeobox 4 (DUX4)-targeting microRNA (miRNA) comprising: (a) a nucleotide sequence comprising at least 90% identity to the sequence set forth in any one of SEQ ID NOs: 5-47; (b) the nucleotide sequence set forth in any one of SEQ ID NOs: 5-47; ( ) a nucleotide sequence that encodes the RNA sequence set forth in any one of SEQ ID NOs: 95-105; or (d) a nucleotide sequence that specifically hybridizes to the DUX4 sequence set forth in any one of SEQ ID NOs: 106-124. In some aspects, the nucleic acid further comprises a promoter sequence. In some aspects, the promoter is any of U6, U7, tRNA, H1, minimal CMV, T7, EF1-alpha, Minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, or CK1. In some aspects, the method comprises a nucleic acid comprising (a) a nucleotide sequence comprising at least 90% identity to the sequence set forth in any one of SEQ ID NOs: 50-92; or (b) the nucleotide sequence set forth in any one of SEQ ID NOs: 50-92.

The disclosure provides a method of treating a subject having a muscular dystrophy or a cancer comprising administering to the subject an effective amount of an adeno-associated virus comprising the nucleic acid encoding a double homeobox 4 (DUX4)-targeting microRNA (miRNA) comprising: (a) a nucleotide sequence comprising at least 90% identity to the sequence set forth in any one of SEQ ID NOs: 5-47; (b) the nucleotide sequence set forth in any one of SEQ ID NOs: 5-47; (c) a nucleotide sequence that encodes the RNA sequence set forth in any one of SEQ ID NOs: 95-105; or (d) a nucleotide sequence that specifically hybridizes to the DUX4 sequence set forth in any one of SEQ ID NOs: 106-124. In some aspects, the method of treating comprises administering a nanoparticle, extracellular vesicle, or exosome comprising a nucleic acid comprising at least 90% identity to the sequence set forth in any one of SEQ ID NOs: 94-105. In some aspects, the nucleic acid further comprises a promoter sequence. In some aspects, the promoter is any of U6, U7, tRNA, H1, minimal CMV, T7, EF1-alpha, Minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, or CK1. In some aspects, the adeno-associated virus comprises a nucleic acid comprising (a) a nucleotide sequence comprising at least 90% identity to the sequence set forth in any one of SEQ ID NOs: 50-92; or (b) the nucleotide sequence set forth in any one of SEQ ID NOs: 50-92. In some aspects, the adeno-associated virus lacks rep and cap genes. In some aspects, the adeno-associated virus is a recombinant AAV (rAAV) or a self-complementary recombinant AAV (scAAV). In some aspects, the adeno-associated virus is AAV-1, AAV-2, AAV-3, AAV-4, AAV-5, AAV-6, AAV-7, AAV-8, AAV-9, AAV-10, AAV-11, AAV-12, AAV-13, AAV-anc80, or AAV rh.74. In some aspects, the adeno-associated virus is AAV-9.

The disclosure provides a method of treating a subject having a muscular dystrophy or a cancer comprising administering to the subject an effective amount of a nanoparticle, extracellular vesicle, or exosome comprising the nucleic acid encoding a double homeobox 4 (DUX4)-targeting microRNA (miRNA) comprising: (a) a nucleotide sequence comprising at least 90% identity to the sequence set forth in any one of SEQ ID NOs: 5-47; (b) the nucleotide sequence set forth in any one of SEQ ID NOs: 5-47; (c) a nucleotide sequence that encodes the RNA sequence set forth in any one of SEQ ID NOs: 95-105; or (d) a nucleotide sequence that specifically hybridizes to the DUX4 sequence set forth in any one of SEQ ID NOs: 106-124. In some aspects, the nucleic acid further comprises a promoter sequence. In some aspects, the promoter is any of U6, U7, tRNA, H1, minimal CMV, T7, EF1-alpha, Minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, or CK1. In some aspects, the nanoparticle, extracellular vesicle, or exosome comprises a nucleic acid comprising (a) a nucleotide sequence comprising at least 90% identity to the sequence set forth in any one of SEQ ID NOs: 50-92; or (b) the nucleotide sequence set forth in any one of SEQ ID NOs: 50-92. In some aspects, the nanoparticle, extracellular vesicle, or exosome comprises a nucleic acid comprising at least 90% identity to the sequence set forth in any one of SEQ ID NOs: 94-105.

The disclosure provides a method of treating a subject having a muscular dystrophy or a cancer comprising administering to the subject an effective amount of a composition comprising a nucleic acid, an adeno-associated virus, or nanoparticle, extracellular vesicle, or exosome, as described herein the disclosure, and a pharmaceutically acceptable carrier.

In some aspects, the muscular dystrophy is facioscapulohumeral muscular dystrophy (FSHD). In some aspects, the cancer is a cancer associated with expression or overexpression of DUX4. In some aspects, the cancer is a sarcoma, a B-cell lymphoma, or a DUX4-expressing cancer of the adrenal, bile duct, bladder, breast, cervix, colon, endometrium, esophagus, head/neck, liver, brain, lung, mesothelium, neural crest, ovary, pancreas, prostate, kidney, skin, soft tissue, stomach, testicles, or thymus.

The disclosure provides uses of a nucleic acid, an adeno-associated virus, a nanoparticle, extracellular vesicle, or exosome, or a composition, as described herein the disclosure, for the preparation of a medicament for inhibiting expression of a double homeobox 4 (DUX4) gene in a cell, for treating or ameliorating a muscular dystrophy or a cancer, and/or for the preparation of a medicament for treating or ameliorating a muscular dystrophy or a cancer. In some aspects, the muscular dystrophy is facioscapulohumeral muscular dystrophy. In some aspects, the cancer is a cancer associated with expression or overexpression of DUX4. In some aspects, the cancer is a sarcoma, a B-cell lymphoma, or a DUX4-expressing cancer of the adrenal, bile duct, bladder, breast, cervix, colon, endometrium, esophagus, head/neck, liver, brain, lung, mesothelium, neural crest, ovary, pancreas, prostate, kidney, skin, soft tissue, stomach, testicles, or thymus.

The disclosure provides a nucleic acid, an adeno-associated virus, a nanoparticle, extracellular vesicle, or exosome, or a composition, as described herein the disclosure, wherein the nucleic acid, adeno-associated virus, nanoparticle, extracellular vesicle, exosome, composition, or medicament is formulated for intramuscular injection, subcutaneous injection, oral administration, subcutaneous, intradermal, or transdermal transport, injection into the blood stream, or for aerosol administration.

The disclosure provides a method of upregulating expression of microRNA-675 in a cell comprising contacting the cell with an effective amount of an estrogen, synthetic estrogen, progesterone, progestin, melatonin, bleomycin or a derivative thereof, pyrazinamide or a derivative thereof, sorafenib (4-[4-[[4-chloro-3-(trifluoromethyl)phenyl]carbamoylamino]phenoxy]-N-methylpyridine-2-carboxamide), or a derivative thereof, or combination of any thereof.

In some aspects, the derivative is a bleomycin derivative. Such bleomycin derivatives include, but are not limited to, bleomycin A2, deglyco-bleomycin A2, bleomycin A5, bleomycin A6, bleomycin B2, and also includes drugs which are synonyms of bleomycin, for example, Bleocin, Bleomicin, Bleomicina (in Spanish), Bleomycine (in French), and Bleomycinum (in Latin).

In some aspects, the derivative is a pyrazinamide derivative. Such pyrazinamide derivative includes, but is not limited to, pyrazine-2-carboxylic acid chloride, N-(1-bromine methyl) pyrazine formamide, N-(bromomethyl)pyrazine-2-carboxamide, N-(2-bromoethyl)pyrazine-2-carboxamide, N-(3-bromopropyl)pyrazine-2-carboxamide, N-(piperidin-1-ylmethyl)pyrazine-2-carboxamide, N-(piperazin-1-ylmethyl)pyrazine-2-carboxamide, N-(thiomorpholinomethyl)pyrazine-2-carboxamide, N-(2-(piperidin-1-yl)ethyl)pyrazine-2-carboxamide, N-(2-(piperazin-1-yl)ethyl)pyrazine-2-carboxamide, N-(2-morpholinoethyl)pyrazine-2-carboxamide, N-(2-thiomorpholinoethyl)pyrazine-2-carboxamide, N-(3-(piperidin-1-yl)propyl)pyrazine-2-carboxamide, N-(3-(piperazin-1-yl)propyl)pyrazine-2-carboxamide, N-(3-morpholinopropyl)pyrazine-2-carboxamide, N-(3-thiomorpholinopropyl)pyrazine-2-carboxamide, 3-chloropyrazine-2-carboxamide, 3-[(4-methylbenzyl)amino]pyrazine-2-carboxamide, N-Benzylpyrazine-2-carboxamides, pyrazine-1,2,3-triazoles, N-alkyl substituted 3-aminopyrazine-2-carboxamides, Pyrazinoic acidn-octyl ester, Pyrazine thiocarboxamide, N-Hydroxymethyl pyrazine, thiocarboxamide, Pyrazinoic acid pivaloyloxymethyl ester, 3-(Benzylamino)pyrazine-2-carboxamide, 3-[(3-Chlorobenzyl)amino]pyrazine-2-carboxamide, 3-[(3,4-Dichlorobenzyl)amino]pyrazine-2-carboxamide, 3-[(3-Trifluoromethylbenzyl)amino]pyrazine-2-carboxamide, 3-[(4-Chlorobenzyl)amino]pyrazine-2-carboxamide, 3-[(2-Methylbenzyl)amino]pyrazine-2-carboxamide, 3-[(4-Methoxybenzyl)amino]pyrazine-2-carboxamide, 3-[(4-Methylbenzyl)amino]pyrazine-2-carboxamide, 3-[(4-Aminobenzyl)amino]pyrazine-2-carboxamide, 3-[(2-Chlorobenzyl)amino]pyrazine-2-carboxamide, 3-[(2-Fluorobenzyl)amino]pyrazine-2-carboxamide, 3-[(4-Trifluoromethylbenzyl)amino]pyrazine-2-carboxamide, 3-[(2-Trifluoromethylbenzyl)amino]pyrazine-2-carboxamide, 3-[(2,4-Dimethoxybenzyl)amino]pyrazine-2-carboxamide, 3-[(3-Nitrobenzyl)amino]pyrazine-2-carboxamide, 3-(benzylamino)-5-cyanopyrazine-2-carboxamide, 3-(4-methylbenzylamino)-5-cyanopyrazine-2-carboxamide, 3-(4-methoxybenzylamino)-5-cyanopyrazine-2-carboxamide, 3-(4-aminobenzylamino)-5-cyanopyrazine-2-carboxamide, 3-(3-chlorobenzylamino)-5-cyanopyrazine-2-carboxamide, 3-(4-chlorobenzylamino)-5-cyanopyrazine-2-carboxamide, 3-(3,4-dichlorobenzylamino)-5-cyanopyrazine-2-carboxamide, 3-(3-nitrobenzylamino)-5-cyanopyrazine-2-carboxamide, 3-(3-trifluoromethylbenzylamino)-5-cyanopyrazine-2-carboxamide, 3-(benzylamino)pyrazine-2,5-dicarbonitrile, 3-(4-methylbenzylamino)pyrazine-2,5-dicarbonitrile, 3-(4-methoxybenzylamino)pyrazine-2,5-dicarbonitrile, 3-(4-aminobenzylamino)pyrazine-2,5-dicarbonitrile, 3-(3-chlorobenzylamino)pyrazine-2,5-dicarbonitrile, 3-(4-chlorobenzylamino)pyrazine-2,5-dicarbonitrile, 3-(3,4-dichlorobenzylamino)pyrazine-2,5-dicarbonitrile, 3-(3-nitrobenzylamino)pyrazine-2,5-dicarbonitrile, 3-(3-trifluoromethylbenzylamino)pyrazine-2,5-dicarbonitrile, 3-(2-methylbenzylamino)pyrazine-2,5-dicarbonitrile, and also includes drugs which are synonyms of pyrazinamide, such as 2-carbamylpyrazine, 2-pyrazinecarboxamide, Aldinamide, Pyrazine carboxamide, pyrazine-2-carboxamide, Pyrazineamide, Pyrazinecarboxamide, Pyrazinoic acid amide, Pyrizinamide, Pirazinamida or Pyrazinamida (in Spanish), Pyrazinamid (in German), and Pyrazinamidum (in Latin).

In some aspects, the derivative is a sorafenib derivative. Such sorafenib derivative includes, but is not limited to, 4-Chloropyridine-2-carbonyl chloride hydrochloride, 4-Chloro-N-cyclopentylpyridine-2-carboxamide, 4-Chloro-N-cyclohexylpyridine-2-carboxamide, 4-Chloro-N-cyclohexylmethylpyridine-2-carboxamide, 4-Chloro-N-benzylpyridine-2-carboxamide, 4-Chloro-N-phenylethylpyridine-2-carboxamide, 4-(4-Aminophenoxy)-N-cyclopentylpyridine-2-carboxamide, 4-(4-Aminophenoxy)-N-cyclohexylpyridine-2-carboxamide, 4-(4-Aminophenoxy)-N-cyclohexylmethylpyridine-2-carboxamide, 4-(4-Aminophenoxy)-N-benzylpyridine-2-carboxamide, 4-(4-Aminophenoxy)-N-phenylethylpyridine-2-carboxamide, 4-[4-[[4-Chloro-3-(trifluoromethyl)phenyl]carbamoylamino]phenoxy]-N-cyclopentyl-pyridine-2-carboxamide, 4-[4-[[4-Chloro-3-(trifluoromethyl)phenyl]carbamoylamino]phenoxy]-N-cyclohexyl-pyridine-2-carboxamide, 4-[4-[[4-Chloro-3-(trifluoromethyl)phenyl]carbamoylamino]phenoxy]-N-cyclohexylmethyl-pyridine-2-carboxamide, 4-[4-[[4-Chloro-3-(trifluoromethyl)phenyl]carbamoylamino]phenoxy]-N-benzyl-pyridine-2-carbox-amide, 4-[4-[[4-Chloro-3-(trifluoromethyl)phenyl]carbamoylamino]phenoxy]-N-phenylethyl-pyridine-2-carboxamide, Sorafenib derivatives containing a phenylcyano group, Sorafenib derivatives containing the nitrogen heterocyclic, sorafenib derivatives with a quinoxalinedione structure, sorafenib derivatives containing a chalcone moiety, sorafenib derivatives containing thioether and nicotinamide, class of diaryl thiourea derivatives of sorafenib, orafenib derivatives containing dithiocarbamate moiety, orafenib derivatives bearing a pyrazole scaffold, sorafenib derivatives containing a cyclohexyl moiety, sorafenib derivatives containing quinoline nucleus, sorafenib derivatives containing a dimer-based structure, a,b-unsaturated ketones derivatives of sorafenib, orafenib derivatives containing a 1,2,3-triazoles framework, orafenib derivatives containing a 1,3,4-triarylpyrazole framework, imidazo [2,1-b] thiazole derivatives of sorafenib, 4-(4-(5-(2,4-Dichlorophenyl)-4,5-dihydro-1H-pyrazol-3-yl)phenoxy)-N-methylpicolinamide, 4-(4-(5-(3-Bromophenyl)-4,5-dihydro-1H-pyrazol-3-yl)phenoxy)-N-methylpicolinamide, N-Methyl-4-(4-(5-(3,4,5-trimethoxyphenyl)-4,5-dihydro-1H-pyrazol-3-yl)phenoxy)picolinamide, 4-(4-(5-(4-Cyanophenyl)-4,5-dihydro-1H-pyrazol-3-yl)phenoxy)-N-methylpicolinamide, 4-(4-(5-(2-Chloro-4-fluorophenyl)-4,5-dihydro-1H-pyrazol-3-yl)phenoxy)-N-methylpicolinamide, N-Methyl-4-(4-(5-(4-nitrophenyl)-4,5-dihydro-1H-pyrazol-3-yl)phenoxy)picolinamide, N-Methyl-4-(4-(5-(3-nitrophenyl)-4,5-dihydro-1H-pyrazol-3-yl)phenoxy)picolinamide, 4-(4-(5-(4-Methoxyphenyl)-4,5-dihydro-1H-pyrazol-3-yl)phenoxy)-N-methylpicolinamide, N-Methyl-4-(4-(5-phenyl-4,5-dihydro-1H-pyrazol-3-yl)phenoxy)picolinamide, 4-(4-(5-(3,4-Dichlorophenyl)-4,5-dihydro-1H-pyrazol-3-yl)phenoxy)-N-methylpicolinamide, 4-(4-(5-(4-Fluorophenyl)-4,5-dihydro-1H-pyrazol-3-yl)phenoxy)-N-methylpicolinamide, 4-(4-(5-(4-Bromophenyl)-4,5-dihydro-1H-pyrazol-3-yl)phenoxy)-N-methylpicolinamide, N-Methyl-4-(4-(5-(2,3,4-trimethoxyphenyl)-4,5-dihydro-1H-pyrazol-3-yl)phenoxy)picolinamide, 4-(4-(5-(2,3-Dichlorophenyl)-4,5-dihydro-1H-pyrazol-3-yl)phenoxy)-N-methylpicolinamide, N-Methyl-4-(4-(3-(4-nitrophenyl)-4,5-dihydro-1H-pyrazol-5-yl)phenoxy) picolinamide, 4-(4-(3-(4-Bromophenyl)-4,5-dihydro-1H-pyrazol-5-yl)phenoxy)-N-methylpicolinamide, 4-(4-(3-(4-Chlorophenyl)-4,5-dihydro-1H-pyrazol-5-yl)phenoxy)-N-methylpicolinamide, 4-(4-(1-Carbamothioyl-5-(3-nitrophenyl)-4,5-dihydro-1H-pyrazol-3-yl)phenoxy)-N-methylpicolinamide, 4-(4-(1-Carbamothioyl-5-(4-fluorophenyl)-4,5-dihydro-1H-pyrazol-3-yl)phenoxy)-N-methylpicolinamide, 4-(4-(1-Carbamothioyl-5-(4-chlorophenyl)-4,5-dihydro-1H-pyrazol-3-yl)phenoxy)-N-methylpicolinamide, 4-(4-(1-Carbamothioyl-5-(2,3-dichlorophenyl)-4,5-dihydro-1H-pyrazol-3-yl)phenoxy)-N-methylpicolinamide, 4-(4-(1-Carbamothioyl-5-(4-cyanophenyl)-4,5-dihydro-1H-pyrazol-3-yl)phenoxy)-N-methylpicolinamide, 4-(4-(1-Carbamothioyl-3-(4-nitrophenyl)-4,5-dihydro-1H-pyrazol-5-yl)phenoxy)-N-methylpicolinamide, HLC-080, benzimidazole derivative bearing a pyrrolidine side chain, N-(4-chloro-3-(trifluoromethyl)phenyl)-2-(2-oxoindolin-3-ylidene)hydrazine-1-carboxamide, N-(3,4-difluorophenyl)-2-(2-oxoindolin-3-ylidene)hydrazine-1-carboxamide, N-(4-chloro-3-(trifluoromethyl)phenyl)-2-(5-methyl-2-oxoindolin-3-ylidene)hydrazine-1-carboxamide, 2-((1H-indol-3-yl)methylene)-N-(3-bromophenyl)hydrazine-1-carboxamide, 2-((1H-indol-3-yl)methylene)-N-(3,4-difluorophenyl)hydrazine-1-carboxamide, 2-((1H-indol-3-yl)methylene)-N-(4-chloro-3-(trifluoromethyl)phenyl)hydrazine-1-carboxamide, 2-((1H-indol-3-yl)methylene)-N-(p-tolyphydrazine-1-carboxamide, 2-((2-chloro-1H-indol-3-yl)methylene)-N-(3,4-difluorophenyl)hydrazine-1-carboxamide, 2-((2-chloro-1H-indol-3-yl)methylene)-N-(3-chlorophenyl)hydrazine-1-carboxamide, N-(3-bromophenyl)-2-((2-chloro-1H-indol-3-yl)methylene)hydrazine-1-carboxamide, 2-((2-chloro-1H-indol-3-yl)methylene)-N-(4-methoxyphenyl)hydrazine-1-carboxamide, 2-((2-chloro-1H-indol-3-yl)methylene)-N-(4-chloro-3-(trifluoromethyl)phenyl)hydrazine-1-carboxamide, 2-((2-chloro-1-ethyl-1H-indol-3-yl)methylene)-N-(4-chloro-3-(trifluoromethyl)phenyl)hydrazine-1-carboxamide, 2-((2-chloro-1-ethyl-1H-indol-3-yl)methylene)-N-(4-fluorophenyl)hydrazine-1-carboxamide, N-(3-bromophenyl)-2-((2-chloro-1-ethyl-1H-indol-3-yl)methylene)hydrazine-1-carboxamide, 2-((2-chloro-1-ethyl-1H-indol-3-yl)methylene)-N-(2-fluorophenyl)hydrazine-1-carboxamide, 2-((2-chloro-1-ethyl-1H-indol-3-yl)methylene)-N-(3-fluorophenyl)hydrazine-1-carboxamide, 2-((2-chloro-1-ethyl-1H-indol-3-yl)methylene)-N-(4-methoxyphenyl)hydrazine-1-carboxamide, 2-((2-chloro-1-ethyl-1H-indol-3-yl)methylene)-N-(3-chlorophenyl)hydrazine-1-carboxamide, N-(3-bromophenyl)-2-((2-chloro-1-propyl-1H-indol-3-yl)methylene)hydrazine-1-carboxamide, N-(4-(2-(methylcarbamoyl) pyridin-4-yloxy) phenyl)-4-phenylpicolinamide, 4-(4-fluorophenyl)-N-(4-(2-(methylcarbamoyl) pyridin-4-yloxy) phenyl) picolinamide, 4-(2,4-Difluorophenyl)-N-(4-(2-(methylcarbamoyl) pyridin-4-yloxy) phenyl) picolinamide, 4-(4-Chlorophenyl)-N-(4-(2-(methylcarbamoyl) pyridin-4-yloxy) phenyl) picolinamide, 4-(4-Methoxyphenyl)-N-(4-(2-(methylcarbamoyl) pyridin-4-yloxy) phenyl) picolinamide, N-(4-(2-(methylcarbamoyl) pyridin-4-yloxy) phenyl)-4-p-tolylpicolinamide, N-(4-(2-(methylcarbamoyl) pyridin-4-yloxy) phenyl)-4-m-tolylpicolinamide, 4-(3-Fluorophenyl)-N-(4-(2-(methylcarbamoyl) pyridin-4-yloxy)phenyl) picolinamide, N-(4-(2-(methylcarbamoyl) pyridin-4-yloxy) phenyl)-4-(4-(trifluoromethyl) phenyl) picolinamide, 4-(4-Ethylphenyl)-N-(4-(2-(methylcarbamoyl) pyridin-4-yloxy) phenyl) picolinamide, 4-(2, 4-dimethylphenyl)-N-(4-(2-(methylcarbamoyl) pyridin-4-yloxy) phenyl) picolinamide, N-(4-(2-(methylcarbamoyl) pyridin-4-yloxy) phenyl)-5-phenylpicolinamide, 5-(4-Fluorophenyl)-N-(4-(2-(methylcarbamoyl) pyridin-4-yloxy) phenyl) picolinamide, 5-(2, 4-Difluorophenyl)-N-(4-(2-(methylcarbamoyl) pyridin-4-yloxy) phenyl) picolinamide, 5-(4-Chlorophenyl)-N-(4-(2-(methylcarbamoyl) pyridin-4-yloxy) phenyl) picolinamide, 5-(4-Methoxyphenyl)-N-(4-(2-(methylcarbamoyl) pyridin-4-yloxy)phenyl) picolinamide, N-(4-(2-(methylcarbamoyl) pyridin-4-yloxy) phenyl)-5-p-Tolylpicolinamide, N-(4-(2-(methylcarbamoyl) pyridin-4-yloxy) phenyl)-5-m-tolylpicolinamide, 5-(3-Fluorophenyl)-N-(4-(2-(methylcarbamoyl) pyridin-4-yloxy) phenyl) picolinamide, N-(4-(2-(methylcarbamoyl) pyridin-4-yloxy) phenyl)-5-(4-(trifluoromethyl) phenyl)picolinamide, 5-(4-Ethylphenyl)-N-(4-(2-(methylcarbamoyl) pyridin-4-yloxy) phenyl) picolinamide, 5-(2, 4-Dimethylphenyl)-N-(4-(2-(methylcarbamoyl) pyridin-4-yloxy) phenyl) picolinamide, and also includes drugs which are synonyms of sorafenib, such as Sorafenib (in French) and Sorafenibum (in Latin).

The disclosure provides a method of inhibiting and/or interfering with expression of a DUX4 gene or protein in a cell comprising contacting the cell with an effective amount of an estrogen, synthetic estrogen, progesterone, progestin, melatonin, bleomycin, pyrazinamide, sorafenib, or a derivative thereof, or a combination of any thereof.

The disclosure provides a method of treating a subject having a muscular dystrophy or a cancer associated with DUX4 expression or overexpression comprising administering to the subject an effective amount of an estrogen, synthetic estrogen, progesterone, progestin, melatonin, bleomycin, pyrazinamide, sorafenib, or a derivative thereof, or a combination of any thereof. In some aspects, the muscular dystrophy is facioscapulohumeral muscular dystrophy (FSHD). In some aspects, the cancer is a sarcoma, a B-cell lymphoma, or a DUX4-expressing cancer of the adrenal, bile duct, bladder, breast, cervix, colon, endometrium, esophagus, head/neck, liver, brain, lung, mesothelium, neural crest, ovary, pancreas, prostate, kidney, skin, soft tissue, stomach, testicles, or thymus.

The disclosure provides use of an estrogen, synthetic estrogen, progesterone, progestin, melatonin, bleomycin, pyrazinamide, sorafenib, or a derivative thereof, or a combination of any thereof for upregulating expression of microRNA-675 in a cell.

The disclosure provides use of an estrogen, synthetic estrogen, progesterone, progestin, melatonin, bleomycin, pyrazinamide, sorafenib, or a derivative thereof, or a combination of any thereof for inhibiting and/or interfering with expression of a DUX4 gene and/or protein in a cell.

The disclosure provides use of an estrogen, synthetic estrogen, progesterone, progestin, melatonin, bleomycin, pyrazinamide, sorafenib, or a derivative thereof, or a combination of any thereof for treating a subject having a muscular dystrophy or a cancer associated with DUX4 expression or overexpression. In some aspects, the muscular dystrophy is facioscapulohumeral muscular dystrophy. In some aspects, the cancer is a sarcoma, a B-cell lymphoma, or a DUX4-expressing cancer of the adrenal, bile duct, bladder, breast, cervix, colon, endometrium, esophagus, head/neck, liver, brain, lung, mesothelium, neural crest, ovary, pancreas, prostate, kidney, skin, soft tissue, stomach, testicles, or thymus.

In some aspects of the disclosure, the estrogen or synthetic estrogen is estrone, estradiol, estriol, estetrol, 27-hydroxycholesterol, dehydroepiandrosterone (DHEA), 7-oxo-DHEA, 7α-hydroxy-DHEA, 16α-hydroxy-DHEA, 7β-hydroxyepiandrosterone, androstenedione (A4), androstenediol (A5), 3α-androstanediol, and 3β-androstanediol, 2-hydroxyestradiol, 2-hydroxyestrone, 4-hydroxyestradiol, 4-hydroxyestrone, 16α-hydroxyestrone, ethinyl estradiol, estradiol valerate, estropipate, conjugate esterified estrogen, and quinestrol.

In some aspects of the disclosure, the progesterone or progestin is medroxyprogesterone acetate (MPA), 17α-hydroxyprogesterone, chlormadinone acetate, cyproterone acetate, gestodene, or etonogestrel.

In some further aspects, the estrogen, synthetic estrogen, progesterone, progestin, a melatonin, bleomycin, pyrazinamide, sorafenib, or a derivative thereof, or a combination of any thereof is formulated for intramuscular injection, oral administration, subcutaneous, intradermal, or transdermal transport, injection into the blood stream, or for aerosol administration.

Further aspects and advantages of the disclosure will be apparent to those of ordinary skill in the art from a review of the following detailed description, taken in conjunction with the drawings. It should be understood, however, that the detailed description (including the drawings and the specific examples), while indicating embodiments of the disclosed subject matter, are given by way of illustration only, because various changes and modifications within the spirit and scope of the disclosure will become apparent to those skilled in the art from this detailed description.

### BRIEF DESCRIPTION OF THE DRAWINGS

FIG. 1A-B shows U6.mir-675 with long 5′ and 3′ flanking sequences reduces DUX4 protein levels with only forty percent inhibition efficiency. FIG. 1A. Dual-luciferase assay to test the ability of U6.mir-675 to target DUX4. Shown here is the mir-675 expression plasmid used in this study. In this construct, the RNA polymerase III U6 promoter (U6p) controls the expression of mir-675. A terminal signal formed of a stretch of six T nucleotides allows the termination of transcription. On the right, the secondary structure of the mir-675 along with its 5′ and 3′ end flanking sequences (Boxed) is shown. At the 5′ end, the flanking sequence is of 40 nucleotides long and at the 3′ end, the flanking sequence is of 47 nucleotides long starting from the nucleotide at position 114. U6.mir-675 was tested to target DUX4 using the dual-luciferase assay. To do this, DUX4 was cloned in the RenLuc-DUX4-FL expression plasmid (FIG. 1A), i.e., DUX4-FL (DUX4 ORF without V5 tag+3′UTR) was PCR amplified using CMV.DUX4-FLΔV5 as template with the following primers: forward: 5′ CCGGCTCGAGATGGCCCTCCCGACAC 3′ (SEQ ID NO: 125), reverse: 5′ ACGACTAGTGGGAGGGGGCATTTTAATATATCTC 3′ (SEQ ID NO: 126). The PCR product was then cloned into a previously designed RenLuc SD5 mutant plasmid using XhoI/SpeI restriction sites and the RenLuc.SD5 mutant-DUX4 3′UTR plasmid backbone. The Renilla luciferase gene has a splicing donor mutation (*SID5) that prevents the alternative splicing of the DUX4-FL mRNA (Ansseau et al. (2015) PLoS One, 10, e0118813). The dual-luciferase assay was performed by co-transfecting the RenLuc-DUX4-FL and U6.mir-675 expression plasmids into the human embryonic kidney HEK293 cells. 48 hours after transfection, both the Renilla and Firefly luciferase activities were measured. The latter was used to normalize for Renilla luciferase activity. The non-targeting RenLuc control backbone plasmid (RenLuc) was co-transfected with U6.mir-675 and the RenLuc-DUX4-FL co-transfected with U6.milacZ as negative control reactions. In the other reactions, mir-675 reduced the relative Renilla luciferase activity by 24±3% (P<0.0001, ANOVA, N=3), 28±2% (P<0.0001, ANOVA, N=3) and 33±3% (P<0.0001, ANOVA, N=3) at a molarity ratio U6.mir-675: RenLuc-DUX4-FL (n:n) of 10 to 1, 20 to 1 and 40 to 1, respectively. All readings were normalized to the U6.milacZ (negative control). Six replicate data were averaged per experiment and individual experiments were performed in triplicate (N=3). Results were reported as the average relative Renilla luciferase activity±SEM for all combined experiments. The U6.miDUX4.405 expression plasmid was used as a positive control [Wallace et al., Mol Ther Methods Clin Dev. 2018 Mar. 16; 8: 121-130]. The U6.miDUX4.405 reduced the relative Renilla luciferase activity by 80±2% (P<0.0001, ANOVA, N=3). FIG. 1B. U6.mir-675 reduced DUX4 protein levels when tested in a western blot. A representative western blot gel and the quantification results of three independent western blot replicates (N=3) performed on 15 μg of total protein extracts collected from HEK293 cells 24 hours after co-transfection with the AAV.CMV.DUX4-FL and the U6.mir-675 or the human long non-coding H19 expression plasmid (CMV.H19) is shown here. The full-length DUX4 is fused to a COOH-terminal V5 epitope. Therefore, to detect DUX4, an anti-V5 primary antibody was used. An antibody to detect the β-actin protein that was used as a normalizer was also used. As a result, U6.mir-675 and CMV.H19 reduced DUX4 protein levels by 46±11% (P<0.02, ANOVA, N=3) and 48±12% (P<0.02, ANOVA. N=3), respectively. All values were normalized to DUX4 protein levels from cells co-transfected with the AAV.CMV.DUX4-FL and U6.milacZ. The results are reported as the average percent DUX4 protein levels±SEM of three independent replicates.

FIG. 2A-B shows that U6.mir-675-2.1.1 and U6.mir-675H showed the highest inhibition efficiency of DUX4 protein levels when compared to the remaining mir-675 constructs in vitro. FIG. 2A, on the right, shows the secondary structures of mir-675 constructs that have different flanking sequences at the 5′ and the 3′ end of the stem-loop structure are shown. FIG. 2A, on the left, shows the expression plasmids for these constructs. The H1.mir-675 and the U6.mir-675F2 expression plasmid contain the cPPT/CTS sequence normally used to increase the nuclear import HIV lentivirus genome. In the designed mir-675 constructs, the 5′ end flanking sequences have a size range between 40 mer for U6.mir-675 and 1 mer for U6.mir-675NF (NF=no flanking) H1.mir-675, U6.mir-675F, U6.mir-675F2, U6.mir-675-2.1, U6.mir-675-2.2, and U6.mir-675-2.1.1. The 3′ end flanking sequence have a size range between 47 mer (U6.mir-675) and no mer (U6.mir-675NF). In U6.mir-675 stem-loop, the highlighted nucleotides correspond to restriction sites XhoI and SpeI/XbaI degenerate sites. The “CNNC” motif corresponds to the serine/arginine-rich splicing factor 3 (SRSF3) site required for efficient cleavage of the primary miRNA (pri-miRNA). N nucleotides represent all nucleotides. U6.mir-675 has five CNNC motifs. For U6.mir-675NF; H1.mir-675; U6.mir-675F; U6.mir-675F2, U6.mir-675-2.1; U6.mir-675-2.2 and U6.mir-675-2.1.1 the nucleotide at the 5′ end is a “C” when the H1 promoter is used and a “G” when the U6 promoter is used. The U6.mir-675 and U6.mir-675H have “UA” (boxed) dinucleotide as a potential Drosha recognition site. However, U6.mir-675-2.3; H1.mir-675-2.4; U6.mir-675-2.3.1 and U6.mir-675-2.5 have “UG” (boxed) as a Drosha recognition site that is normally found at the basal stem of the pri-miRNA. FIG. 2B. Western and northern blots using all fourteen mir-675 constructs. Shown here are two representative western blots and the quantification results of three to eight independent western blot replicates (N=3-8). Transfection and protein extraction were similar to FIG. 1A-B. The molar ratio of U6/H1.mir-675 to CMV.DUX4-FL/CMV.eGFP expression plasmids was 1 to 3 in all transfections. The graph on the right shows the average DUX4 protein levels following mir-675 transfection. All thirteen mir-675 constructs had better inhibition efficiency than U6.mir-675 (43±4%, N=6) (Table 2). U6.mir-675-2.1.1 and U6.mir-675H had an inhibition efficiency of 83±3 (N=3 independent replicates) and 89±6 (N=3 independent replicates), respectively. U6.mir-675-2.1.1 and U6.mir-675H had the highest inhibition efficiency of DUX4 protein levels, which was statistically significant when compared to all other mir-675 constructs (P<0.05, ANOVA, N=3-8 independent replicates). In the CMV.DUX4-FL/CMV.eGFP expression plasmid, the full-length DUX4 is fused to a COOH-terminal V5 epitope. Thus, DUX4 was detected using an anti-V5 primary antibody as in FIG. 1A-B. An antibody also was used to detect eGFP that was co-expressed from the same plasmid expressing DUX4 and was used as a normalizer. All values were normalized to DUX4 protein levels from cells co-transfected with the CMV.DUX4-FL/CMV.eGFP and U6-milacZ. On top of the column corresponding to each mir-675 construct quantifying percent DUX4 protein levels, northern blot results representing the full RNA profile for each construct are shown. Anti-mir-675-5p double biotinylated probe was used. 21-25 mer mature sequences are indicated to the right of the gel blot. Results are reported as the average percent DUX4 protein levels±SEM of three to eight independent replicates.

FIG. 3A-B shows that U6.mi405F showed higher inhibition efficiency of DUX4 expression when compared to the original U6.mi405 construct. FIG. 3A, on the left, shows the secondary structures of the three U6.mi405 constructs that have different flanking sequences at the 5′ and the 3′ end of the stem-loop structure are shown. U6.mi405 possesses a 34 mer and a 41 mer long flanking sequence at the 5′ and 3′ end, respectively. In this construct, the 3′ end flanking sequence has five “CNNC” motifs that could be recognized by the SRSF3 splicing factor. The U6.mi405F possesses one nucleotide at the 5′ end and a 16 mer long 3′ flanking sequence at the 3′ end. The latter has a single “CNNC” motif. The U6.mi405NF possesses only one nucleotide at the 5′ end and no “CNNC” motif at the 3′ end. The underlined sequence corresponds to the mi405 guide strand. On the right, the expression plasmids for these constructs as well as the expression plasmid of the RenLuc-DUX4 ORF dual-luciferase construct are shown. A dual-luciferase assay and western blot were used to assess the inhibition efficiency of the three mi405 constructs. In the dual-luciferase assay, U6.mi405 reduced the relative Renilla luciferase activity by 85±1% (P<0.0001, ANOVA, N=3), U6.mi405F by 89±1% (P<0.0001, ANOVA, N=3) and U6.mi405NF by 66±1% (P<0.0001, ANOVA, N=3) when tested against the RenLuc-DUX4 ORF construct. All readings were normalized to the miGFP negative control. U6.mi405F had a statistically significant higher inhibition efficiency than U6.mi405 when tested at the 1:4 DUX4:mi405 molar ratio (P<0.04 ANOVA, N=3 independent replicates). Western blot also was used to test the efficiency of the mi405 constructs to reduce DUX4 protein levels in HEK293 cells. The same 1:4 DUX4:mi405 molar ratio was used. 24 hours post-transfection, U6.mi405, U6.mi405F and U6.mi405NF reduced DUX4 protein levels by 79±1% (P<0.0001, ANOVA, N=2), 99±1% (P<0.0001, ANOVA, N=2) and 70±13% (P<0.0036, ANOVA, N=2), respectively. U6.mi405F had his inhibition efficiency increase by an average of 20% when compared to U6.mi405 (P<0.0079, ANOVA, N=2 independent replicates). All quantifications were normalized to the miGFP negative control. Results are reported as the average percent DUX4 protein levels±SEM of two independent replicates. FIG. 3B. Dual-luciferase assay to test the inhibition efficiency of the 10 miDUX4 candidates previously identified by [Wallace et al. Mol Ther Methods Clin Dev. 2018 Mar. 16; 8: 121-130] using the mi405F flanking sequences. The use of the latter did not enhance the inhibition efficiency of any of the 10 miDUX4 miRNAs tested. On the contrary, mi185F, mi186F, mi318F, mi599F, mi1156F and mi1311F had lower inhibition efficiency than their original counterparts as shown by the increase in the relative Renilla Luciferase activity by 160±8% (P<0.0001, ANOVA, N=3), 27±9% (P<0.026, ANOVA, N=3), 44±15% (P<0.018, ANOVA, N=3), 24±9%, (P<0.039, ANOVA, N=3), 34±8% (P<0.0071, ANOVA, N=3) and 27±9% (P<0.021, ANOVA, N=3), respectively. All readings were normalized to the miGFP negative control. Results are reported as the average relative Renilla luciferase activity±SEM of three independent replicates.

FIG. 4A-B shows that the decrease of DUX4 to miDUX4 molar ratio increased the inhibition efficiency of U6.mi405F but not of the other ten U6.miDUX4F constructs. FIG. 4A shows results from a dual-luciferase assay that was used to test the inhibition efficiency of ten miDUX4 miRNAs and their cognate miDUX4F constructs using the following DUX4 to miDUX4 molar ratios: 1:1, 1:2, 1:3 and 1:4. U6.mi405F showed enhanced inhibition efficiency at all ratios when compared to U6.mi405 (relative Renilla luciferase activity decreased by 33±2%; 32±2%; 34±1% and 32±1% at 1:1, 1:2, 1:3 and 1:4 ratio, respectively) (P<0.0001, ANOVA, N=3 independent replicates). FIG. 4B shows results of a dose de-escalation study of mi405, mi405NF and mi405F using the dual-luciferase assay in HEK293 cells 24 hours post-transfection. The DUX4:mi405 molar ratio ranged between 1:4 to 40:1. The silencing efficiency of U6.mi405 and U6.mi405F was dose dependent. However, the inhibition efficiency of U6.mi405F was always better than that of U6.mi405 at all molar ratios (P<0.0001, ANOVA, N=3). U6.mi405F was most efficient at the 8:1 molar ratio with a decrease in the relative Renilla luciferase activity by 48±6% when compared to U6.mi405 (P<0.0001, ANOVA, N=3). All readings were normalized to the miGFP negative control. Results are reported as the average relative Renilla luciferase activity±SEM of three independent replicates.

FIG. 5A-C shows that changing the 5′ and 3′ end sequences flanking the mi405 stem-loop structure impacts the silencing efficiency of the miRNA. FIG. 5A, on the right, shows the secondary structure of ten mi405 constructs that have different flanking sequences at the 5′ and the 3′ end of the stem-loop structure are shown. FIG. 5A, on the left, shows the expression plasmids for the mi405 constructs, the dual-luciferase assay (RenLuc-DUX4-ORF) and the western blot (CMV.DUX4-FL/CMV.eGFP). Annotations are similar as in FIG. 1A and FIG. 2A. FIG. 5B shows results of a dual-luciferase assay with the RenLuc-DUX4 ORF and U6.mi405 constructs with a DUX4:mi405 molar ratio of 2 to 1 and 12 to 1. 24 hours post-transfection; the relative Renilla luciferase activity was measured. In the case of the 2 to 1 ratio, all U6.mi405 constructs efficiently reduced the relative Renilla luciferase activity except U6.mi405NF and U6.mi405B, which reduced the Renilla luciferase activity by 3.6±1.8% (P>0.80, ANOVA, N=3) and 25±3 (P<0.0001, ANOVA, N=3), respectively. However, U6.mi405, U6.mi405A, U6.mi405C, U6.mi405D, U6.mi405E, U6.mi405F, U6.mi405G and U6.mi405H reduced the Renilla luciferase activity by 62±2%, 72±1%, 64±1%, 71±1%, 73±1%, 77±1%, 81±1%, and 80±1% (P<0.0001, ANOVA, N=3), respectively. When compared to U6.mi405F, none of the other U6.mi405 constructs had a statistically significant enhanced inhibition efficiency. When using the 12 to 1 DUX4:mi405 molar ratio, U6.mi405F, U6.mi405G and U6.mi405H reduced the Renilla luciferase activity by 45±4%, 59±2% and 58±2% (P<0.0001, ANOVA, N=3 independent replicates), respectively. When compared to U6.mi405F, U6.mi405G and U6.mi405H reduced the Renilla luciferase activity by an additional 26±5% (P<0.033, ANOVA, N=3) and 25±6% (0.042, ANOVA, N=3), respectively. All readings were normalized to the U6.miGFP negative control. Results are reported as the average relative Renilla luciferase activity±SEM of three independent replicates. FIG. 5C shows a western blot of total proteins extracted from HEK293 cells co-transfected 24 hours earlier with U6.miGFP, U6.mi405, U6.mi405F, U6.mi405G or U6.mi405H and CMV.DUX4-FL/CMV.eGFP expression plasmids at a molar ratio of U6.miRNA to CMV.DUX4-FL/CMV.eGFP of 12 to 1. The graph shows the average DUX4 protein levels of four independent experiments. When compared to U6.mi405, U6.mi405F, U6.mi405G and U6.mi405H induced a significant reduction of DUX4 protein levels by 40±5% (P<0.0209, ANOVA, N=4), 71±5% (P<0.0001, ANOVA, N=4) and 60±8% (P<0.0009, ANOVA, N=4), respectively. When compared to U6.mi405F, U6.mi405G and U6.mi405H induced a significant reduction of DUX4 protein levels by 52±9% (P<0.0009, ANOVA, N=4) and 33±14% (P<0.0498, ANOVA, N=4), respectively. Results are reported as the average percent DUX4 protein levels±SEM of four independent replicates.

FIG. 6A-B shows that differential expression of mature mi405 is detected following change in the 5′ and 3′ end flanking sequences. FIG. 6A shows QPCR used to assess expression of the mature mi405 microRNA sequence from all U6.mi405 expression plasmids using standard TaqMan cDNA synthesis reaction. The latter uses a reverse primer that detects the mature mi405 sequence following a stem-loop primer-based small RNA detection principle (ThermoFisher) [Jung et al., RNA (2013) 19: 1-10]. A standard TaqMan probe specific to mi405 was then used for amplification step. This probe base pairs at the junction between the 3′ end of the mi405 mature sequence and the 5′ end of the reverse primer sequence. QPCR analysis was carried out on all U6.mi405 constructs 24 hours after transfection in HEK293 cells. All values were normalized to U6.mi405. U6.mi405F, U6.mi405B and U6.mi405C had their mature mi405 sequence expression reduced by 85±5% (P<0.0019, ANOVA, N=3 independent replicates), 71±9% (P<0.0038, ANOVA, N=4) and 63±27% (P<0.0133, ANOVA, N=3), respectively. However, U6.mi405A, U6.mi405D, U6.mi405E, U6.mi405G and U6.mi405H expressed the mature mi405 sequence at levels that were minimally increased (not statistically significant) when compared to the levels expressed from U6.mi405. Gene expression was normalized to hsa-RPL13A. Results are reported as relative mi405 expression±SEM of three to four independent replicates. Q: Quencher. F: Fluorophore. FIG. 6B shows droplet digital PCR to quantify mi405, mi405F, mi405B, mi405C, mi405G and mi405H expression levels. cDNA was generated using the TaqMan advanced cDNA synthesis kit (ThermoFisher) (cDNA outcome illustrated above the ddPCR graph) and two TaqMan advanced custom made mi405 probes (embedded mi405 probe and overlapped mi405 probe). The embedded probe base pairs only within the mi405 sequence. The overlapped probe base pairs with mi405 3′ end sequence and with part of the adaptor region. In this assay, mi405 levels were normalized to mir-191-5p endogenous control miRNA levels. Results are reported as copies of mi405 relative to mir-191-5p±SEM of three independent replicates. R: Reporter dye. NFQ: Non-fluorescent quencher dye. MGB: Minor groove binder.

FIG. 7 shows uncropped western blot replicates supplementary to FIG. 1A-B.

FIG. 8 shows uncropped western blot replicates supplementary to FIG. 2A-B.

FIG. 9A-B shows quantification of mature mir-675 levels relative to U6.mir-675. FIG. 9A shows RT-qPCR used to quantify the 23 mer mature mir-675-5p levels after transfection of the fourteen mir-675 constructs in HEK293 cells (see FIG. 2A-B). 24 hours post-transfection, RNA was extracted using the mirVana total RNA isolation kit following the manufacturer protocol. The results show differences between mir-675 constructs regarding mature mir-675-5p and pri-mir-675 expression levels. H1.mir-675 (232±35%, P<0.0002, ANOVA, N=3 independent experiments), U6.mir-675F2 (882±108%, P<0.0001, ANOVA, N=3) and U6.mir-675H (774±93%, P<0.0001, ANOVA, N=3) produced the highest levels of mir-675-5p when compared to U6.mir-675. However, H1.mir-675 (320±43%, P<0.0001, ANOVA, N=3), U6.mir-675NF (213±28%, P<0.0087, ANOVA, N=3), U6.mir-675-2.3.1 (274±49%, P<0.0007, ANOVA, N=3) and U6.mir-675H (256±37%, P<0.0008, ANOVA, N=3) produced the highest levels of pri-mir-675 when compared to U6.mir-675. When looking at the ratio of mir-675-5p/pri-mir-675, U6.mir-675F2 (918±169%, N=3), U6.mir-675-2.1.1 (207±38%, N=3) and U6.mir-675H (303±57%, N=3) had the highest ratio when compared to U6.mir-675 (P<0.0001, ANOVA, N=3). FIG. 9B shows ddPCR used to quantify all mature mir-675-5p sequences using the TaqMan Advanced miRNA cDNA Synthesis method. All constructs were transfected in HEK293 cells as in FIG. 9A. As a result, U6.mir-675NF (176±60%, N=3), U6.mir-675F (133±38%, N=3), U6.mir-675F2 (181±34%, N=3), U6.mir-675-2.1 (142±45%, N=3), U6.mir-675-2.1.1 (213±51%, N=3), U6.mir-675-2.3 (114±8c)/0, N=3), U6.mir-675-2.3.1 (187±40%, N=3), U6.mir-675-2.5 (153±52%, N=3) and U6.mir-675H (201±38%, N=3) showed higher fold change levels when compared to U6.mir-675. Only U6.mir-675-2.1.1 (P<0.014, ANOVA, N=3), U6.mir-675-2.3.1 (P<0.024, ANOVA, N=3) and U6.mir-675H (P<0.007, ANOVA, N=3) have statistically significant higher fold change.

FIG. 10 shows western blot of DUX4 protein levels using U6.mi405, U6.mi405F and U6.mi405NF. HEK293s cells were co-transfected with CMV.DUX4-FL/CMV.eGFP and U6.mi405, U6.mi405F or U6.mi405NF expression plasmids at a molar ratio of 1 to 3. Total protein was extracted 24 hours after transfection.

FIG. 11 shows data testing the inhibition efficiency of U6.mi405F, U6.mi405G and U6.mi405H using western blot. DUX4:mi405 was used at a molar ratio of 2 to 1. HEK293 cells were co-transfected for 24 hours with U6.miGFP, U6.mi405F, U6.mi405G or U6.mi405H and CMV.DUX4-FL/CMV.eGFP expression plasmids. U6.mi405F, U6.mi405G and U6.mi405H reduced DUX4 protein levels by 81±9%, 88±2% and 79±6%, respectively when compared to the negative control (U6.miGFP) transfected sample (P<0.0001, ANOVA, N=3 independent replicates). No statistically significant difference was measured between the three tested mi405 constructs at the used DUX4:mi405 molar ratio. We reported the results as the average percent DUX4 protein levels±SEM of three independent replicates.

FIG. 12 shows four independent western blot replicates supplementary to FIG. 5C, which indicate that U6.mi405F, U6.mi405G and U6.mi405H induced a significant reduction of DUX4 protein levels by 40±5% (P<0.0209, ANOVA, N=4), 71±5% (P<0.0001, ANOVA, N=4) and 60±8% (P<0.0009, ANOVA, N=4) relative to U6.mi405. In addition, U6.mi405G and U6.mi405H induced a significant reduction of DUX4 protein levels by 52±9% (P<0.0009, ANOVA, N=4) and 33±14% (P<0.0498, ANOVA, N=4) when compared to U6.mi405F.

FIG. 13A-D shows results from a molecular beacon binding assay (MBB assay) which showed that mir-675 targets sites at DUX4 ORF and 3′UTR with high efficiency. FIG. 13A provides a schematic of DUX4 sequence showing predicted target site (TS) positions for mir-675-5p. FIG. 13B (left) provides a schematic explaining the fluorescence-based molecular beacon binding assay used to determine mir-675-5p binding to DUX4 sequence. Unbound, the molecular beacon (MB) folds into a stem loop structure that brings a quencher (zenBHQ) in close proximity to a fluorophore (6FAM), thereby quenching the fluorescence emission of 6FAM. The mature sequence of mir-675-5p was incorporated in the MB loop sequence. Hybridization of the MB to a complementary TS sequence separates the fluorophore and quencher, allowing fluorescence emission, which is then quantified as a measure of binding. FIG. 13B (right) provides a graph showing binding of the mature mir-675-5p molecular beacon to target sites shown in FIG. 13A. Each data point represents mean±SD of three separate experiments. mir-675-5p was able to bind eight target sites within the full length DUX4 sequence (TS527, TS649, TS668, TS754, TS780, TS1004, TS1340 and TS1471). The first six TS are in DUX4 ORF and the remaining two TS are found in the 3′UTR. Six predicted TS did not bind to mir-675-5p (see FIGS. 17A-B and FIG. 13D for TS position and sequence). The TS neg. ctrl is a random sequence. FIG. 13C shows the binding affinity (K d) of mir-675-5p molecular beacon to each target site was determined by subtracting background fluorescent signal from the molecular beacon signal (MBS), expressed in relative fluorescent units (RFU). The K_{d} corresponds to the TS concentration (μM) required to reach half of maximum fluorescence. Base-pairing between mir-675-5p and its TS (as predicted by the RNAhybrid algorithm) is also shown, as well as their corresponding Kd values. RNA “mimic” bases were generated in the mir-675-5p:TS pair, and replaced “G” nucleotides with “A” nucleotides (in grey) whenever the “G” is facing a “T”. FIG. 13D shows the molecular beacon for miRNA-5p w/5′ tag (6FAM dye) and 3′ tag (Zen black hole qTencher (ZenBHQ)) and the position, name, and sequence of each of the DUX 4 target sites.

FIG. 14 shows a northern blot on different mir-675 constructs to examine mir-675 processing. The northern blot was performed on RNA extracted from HEK293 cells transfected with U6.miGFP (negative control miRNA targeting gfp mRNA), U6.mir-675, H1.mir-675, U6.mir-675-3p and U6.mir-675-5p constructs. U6.miGFP and U6.mir-675-3p negative control constructs did not show any bands. The U6.mir-675 construct generated low levels of the mature mir-675-5p with a size between 21 and 25 mer. The H1.mir-675 construct generated abundant levels of the mature mir-675-5p with a size close to 25 mer. The U6.mir-675-5p construct also gave abundant levels of the mature mir-675-5p with a size close to 21 mer. H1.mir-675-generated mature mir-675-5p was 13-fold and 23-fold more abundant than the U6.mir-675-generated mature mir-675-5p at 24 and 48h post-transfection, respectively. In the case of H1.mir-675, 1.4-fold more mature mir-675-5p was produced at 48h versus 24h post-transfection.

FIG. 15A-B shows that mir-675 is capable of protecting mouse skeletal tibialis anterior (TA) muscles from DUX4-induced muscle damage. FIG. 15A shows H&E staining, central nuclei counts and gene expression (ddPCR) analysis of AAV-injected adult mouse (C57BL/6) TA muscles 2 weeks after intramuscular (IM) injection with the indicated doses of vectors. Images show 10 μm cryosections stained with H&E at high (20×) and low power (4×). To help visualize the breadth of potential lesions on low-power images, fibers with central nuclei (CN) or areas of active degeneration and inflammation were intentionally shaded with a purple digital overlay. DUX4-expressing muscles show histopathological evidence of degeneration, including myofibers with inflammatory infiltrates, central nuclei, and variable fiber size (top left). Co-injections of AAV.CMV.DUX4-FL and scAAV6.mir-675 vectors (top right, respectively) are histologically normal. High-power photos show representative images at indicated vector dosages of N=6 TA co-injected with scAAV6.mir-675 and AAV.CMV.DUX4-FL, n=3 TA co-injected with AAV.milacZ and AAV.CMV.DUX4-FL and n=3 TA co-injected with AAV.milacZ and scAAV6.mir-675. The latter TA muscles were histologically normal (bottom left), indicating that scAAV6.mir-675 is not toxic to muscle. Scale bars, 100 μm for high-power; 500 μm for low-power images. Central nuclei counts of 20×H&E stained TA muscle serial sections (10 μm). scAAV6.mir-675-treated muscles have 85±14% (N=6, two-tailed unpaired t-test, ****, P<0.0001) fewer myofibers with central nuclei when compared with TA muscles co-injected with AAV.milacZ and AAV.CMV.DUX4-FL (N=3). Droplet digital PCR (ddPCR) of DUX4-FL expression in scAAV6.mir-675-treated and untreated TA muscles. In treated TA muscles, DUX4-FL levels were reduced by 56±32% when compared to the untreated muscles (N=6, two-tailed unpaired t test, *, P<0.038). ddPCR of mir-675 and DUX4-responsive mouse biomarkers (Trim36 and Wfdc3) in scAAV6.mir-675 treated and un-treated TA muscles. Trim36 and Wfdc3 expression was reduced by 90±31% (N=6, ANOVA, ***, P<0.0003) and 54±20% (N=6, ANOVA, *, P<0.039), respectively. FIG. 15B provides images which show 10 μm cryosections immunofluorescently stained for DUX4 (V5 epitope, red) or nuclei (DAPI, blue). High-power photos show representative images at indicated vector dosages of N=6 TA co-injected with scAAV6.mir-675 and AAV.CMV.DUX4-FL, N=3 TA co-injected with AAV.milacZ and AAV.CMV.DUX4-FL. In the absence of mir-675, high levels of DUX4 proteins are detected. White arrows indicate representative fibers and myonuclei expressing DUX4 proteins.

FIG. 16A-C shows mir-675-5p is the mature miRNA strand targeting DUX4. FIG. 16A shows QPCR analysis of mir-675 expression in HEK293 cells transfected with hsa-H19 lncRNA (CMV.H19). miR-675-5p expression is relative to that of miR-675-3p. The QPCR was done using the Taqman probes designed to recognize the mature sequence of mir-675-5p and -3p. mir-675-3p expression was 2.20±0.15-fold higher than that of mir-675-5p (P<0.0001, ANOVA, N=3). Gene expression was normalized to the housekeeping gene RPL13A. Results were reported as the average relative gene expression±SEM of three replicates (N=3). FIG. 16B shows a dual-luciferase assay with U6.mir-675-3p, U6.mir-675-5p (see corresponding stem loop structures for mir-675-3p and mir-675-5p next to the graph) and RenLuc-DUX4-FL construct. The miRNA (pmoles) was used at 40-fold of the RenLuc-DUX4-FL (pmoles). When testing the U6.mir-675-3p construct, the relative Renilla luciferase kept on average 95% (P<0.2, ANOVA) of its activity, even though U6.mir-675-3p expressed high levels of mir-675-3p relative to the negative control mir-675-5p levels. On the other hand, U6.mir-675-5p was able to reduce the relative Renilla luciferase activity on average by 33±3% (P<0.0001, ANOVA, N=3). All readings were normalized to the milacZ negative control. Results were reported as the average relative Renilla luciferase activity±SEM of three replicates (N=3). FIG. 16C shows a dual-luciferase assay using the reverse complementary sequence of mir-675-5p as target sequence (mir-675R). This sequence was cloned as a 3′UTR downstream the Renilla luciferase gene. U6.mir-675 construct was tested for its targeting efficiency against a mir-675 perfect target site (PTS) mir-675R by measuring the inhibition efficiency of the corresponding relative Renilla luciferase activity. U6.mir-675 reduced the relative Renilla luciferase activity in a dose-dependent manner reaching a maximum inhibition of 41±12% (P<0.01, ANOVA, N=3), and CMV.H19 reduced the relative Renilla luciferase activity by 40±6% (P<0.0048, ANOVA, N=3). All readings were normalized to the milacZ negative control. Results were reported as the average relative Renilla luciferase activity±SEM of three replicates.

FIG. 17A-B shows mir-675 binding sites in the DUX4 sequence. FIG. 17A shows stem loop structures of mir-675, mir-675-5p and mir-675-3p. The mature sequences are highlighted in red. FIG. 17B shows the DUX4 sequence (DUX4 ORF+3′UTR without introns). The validated mir-675-5p binding sites are highlighted in red. Only mir-675-5p binding sites are shown here.

FIG. 18 shows that U6.mir-675, CMV.mir-675, H1.mir-675, U6.mir-675-5p, CMV.H19 and mir-675 mimic (mature double stranded mir-675 sequence) reduce DUX4 protein level.

FIG. 19 is a replicate of FIG. 18 and shows that U6.mir-675, H1.mir-675, and U6.mir-675-5p reduce DUX4 protein level. However, FIG. 19 also shows that these mir-675 constructs were not able to reduce DUX4 protein level when tested against the mir-675-resistant DUX4 construct (CMV.DUX4-mir-675Res: this expression plasmid encodes a DUX4 mutant sequence. This sequence is mutated in mir-675 target site 780 (TS780) found in ORF (see FIG. 17B) and has its 3′UTR deleted, rendering the expression of this DUX4 mutant resistant to mir-675-dependent inhibition).

FIG. 20 shows that a mir-675 construct under a CMV promoter elicited ˜50% inhibition of DUX4 expression, indicating a robust expression of mir-675 from a promoter mostly used to express CDS mRNAs. FIG. 20 is a replicate of FIG. 18 and shows that in a blinded western blot, U6.mir-675, CMV.mir-675, H1.mir-675, U6.mir-675-5p, CMV.H19 and mir-675 mimic reduce DUX4 protein level. FIG. 20 also shows that these mir-675 constructs were not able to reduce DUX4 protein level when tested against the mir-675-resistant DUX4 construct. Three repeated blinded western blots were performed on protein extracts from HEK293 cells co-transfected with various constructs expressing mir-675 and full length V5-tagged DUX4 constructs (DUX4-FL WT: CMV.DUX4-FL/CMV.eGFP and DUX4-mir-675Res: CMV.DUX4-mir-675Res). The latter co-expresses eGFP from the same plasmid backbone. eGFP was used as a transfection control and a reference gene for quantification purposes. DUX4 protein levels were quantified relative to milacZ samples as shown in the graphs.

FIG. 21 is another replicate of FIG. 18 and shows that in a blinded western blot, U6.mir-675, CMV.mir-675, H1.mir-675, U6.mir-675-5p and CMV.H19 reduce DUX4 protein level. FIG. 21 also shows that these mir-675 constructs were not able to reduce DUX4 protein level when tested against the mir-675-resistant DUX4 construct. Three repeated blinded western blots were performed on protein extracts from HEK293 cells co-transfected with various constructs expressing mir-675 and full length V5-tagged DUX4 constructs (DUX4-FL WT: CMV.DUX4-FL/CMV.eGFP and DUX4-mir-675Res:CMV.DUX4-mir-675Res). The latter co-expresses eGFP from the same plasmid backbone. eGFP was used as a transfection control and a reference gene for quantification purposes. DUX4 protein levels were quantified relative to milacZ samples as shown in the graphs.

FIG. 22 provides other replicates of FIG. 18 (see left and right panels (blots)) and shows that in a blinded western blot, H1.mir-675 and CMV.H19 reduce DUX4 protein level. In addition, FIG. 22 (right panel) shows that U6.mir-675, CMV.mir-675, H1.mir-675, U6.mir-675-5p, CMV.H19 and mir-675 mimic reduce DUX4 protein level. Three repeated blinded western blots were performed on protein extracts from HEK293 cells co-transfected with various constructs expressing mir-675 and full length V5-tagged DUX4 constructs (DUX4-FL WT: CMV.DUX4-FL/CMV.eGFP and DUX4-mir-675Res: CMV.DUX4-mir-675Res) (FIG. 20-22). The latter co-expresses eGFP from the same plasmid backbone. eGFP was used as a transfection control and a reference gene for quantification purposes. DUX4 protein levels were quantified relative to milacZ samples as shown in the graphs. In FIG. 20, a mir-675 construct under CMV promoter was tested and showed ˜50% inhibition of DUX4 expression, indicating a robust expression of mir-675 from a promoter mostly used to express CDS mRNAs. Three repeated blinded western blots were performed on protein extracts from HEK293 cells co-transfected with various constructs expressing mir-675 and full length V5-tagged DUX4 constructs (DUX4-FL WT: CMV.DUX4-FL/CMV.eGFP and DUX4-mir-675Res: CMV.DUX4-mir-675Res). The latter co-expresses eGFP from the same plasmid backbone. eGFP was used as a transfection control and a reference gene for quantification purposes. DUX4 protein levels were quantified relative to milacZ samples as shown in the graphs.

FIG. 23A-B shows DUX4 mRNA levels are reduced upon overexpression of H1.mir-675 in HEK293 cells co-transfected with CMV.DUX4-FL/CMV.eGFP expression plasmid. FIG. 23A shows QPCR measurement of DUX4 expression. H1.mir-675 construct was transfected in a 3 to 1 ratio with DUX4 construct in HEK293 cells, and collected RNA extracts 24 and 48h after transfection. Total RNA was prepared using the miRVANA isolation kit. DUX4 and egfp (used as the reference gene) mRNA levels were determined using the SYBR green master mix as described in materials and methods. All values were normalized to egfp, and were reported relative to milacZ treated samples. At 24 and 48h after transfection, DUX4 levels decreased by an average of 37±2% (P<0.0001, ANOVA, N=3) and 51±2% (P<0.0001, ANOVA, N=3), respectively. Results were reported as the average relative DUX4 expression±SEM of three replicates. FIG. 23B shows measurement of mir-675-5p expression after its overexpression in HEK293 cells by QPCR. When compared to the milacZ sample, mir-675-5p overexpressed levels were ˜2000-fold higher (P<0.0001, ANOVA, N=3). Results were reported as the average relative mir-675-5p expression (2^{-ΔCq})±SEM of three replicates.

FIG. 24 shows pri-mir-675 and mir-675-3p are expressed in human control (15V) and FSHD-affected (15A and 17A) myoblasts and myotubes. The expression of pri-mir-675 and mir-675-3p was measured in three different human skeletal muscle-derived myoblast cell lines 15V, 15A and 17A. RNA was prepared and gene expression of pri-mir-675 (the primary mir-675 transcript) and mir-675-3p was measured in myoblasts and in four days-differentiated (4DD) myotubes. As a result, pri-mir-675 and mir-675-3p were expressed in 15V, 15A and 17A myoblasts and differentiated myotubes but at various levels. In particular, both pri-mir-675 and mir-675-3p levels increased upon differentiation in all tested cell lines. More precisely, pri-mir-675 expression increased by 2.3±0.1-fold (P<0.0001, ANOVA, N=3) for 15V, 2.3±0.2-fold (P<0.0001, ANOVA, N=3) for 15A and 3.3±0.4-fold (P<0.0001, ANOVA, N=3) for 17A. On the other hand, mir-675-3p expression increased by 7.7±1.0-fold (P<0.0001, ANOVA, N=3) for 15V, 7.9±0.2-fold (P<0.0001, ANOVA, N=3) for 15A and 1.4±0.1-fold (P<0.0001, ANOVA, N=3) for 17A. Results were reported as relative gene expression (ΔΔCq)±SEM of three replicates (N=3) relative to gene expression in 15V myoblasts, with each QPCR assay performed in triplicate. All results were quantified using as reference gene the house keeping gene RPL13A.

FIG. 25 shows mir-675 targets SMAD1, SMAD5 and CDC6 in HEK293 cells. The expression of SMAD1, SMAD5 and CDC6 was measured by QPCR in HEK293 cells using TaqMan probes specific to each investigated gene. To do that, U6.milacZ (negative control), H1.mir-675, U6.mir-675-3p or U6.mir-675-5p expressing constructs were transfected into HEK293 cells, and total RNA was extracted 48h after transfection. As a result, U6.mir-675-3p reduced SMAD1 levels by an average of 32±6% (P<0.044, ANOVA, N=3) and SMAD5 levels by an average of 35±6% (P<0.0013, ANOVA, N=3). On the other hand, H1.mir-675 and U6.mir-675-5p repressed CDC6 levels by an average of 38±4% (P<0.0034, ANOVA, N=3) and 36±7% (P<0.0048, ANOVA, N=3), respectively. Results were reported as relative gene expression (ΔΔCq)±SEM of three replicates (N=3) relative to gene expression in cells transfected with U6.milacZ, with each QPCR assay performed in triplicate. All results were quantified using as reference gene the house keeping gene RPL13A.

FIG. 26 shows an uncropped western blot gel for the detection of Cdc6 protein in 15V Ctrl myotubes. Cdc6 is a natural target to mir-675. Thus, this figure shows that the inhibition of mir-675 using anti-mir-675 antagomir is working since transfection of 15V control (Ctrl) myotubes with anti-mir-675 led to induced expression of Cdc6 protein.

FIG. 27 shows three uncropped repeated western blots performed on protein extracts from 15A FSHD myotubes co-transfected with anti-mir-675-5p, DUX4-FL (WT) and DUX4-mir-675Res constructs. This figure shows that the transfection of anti-mir-675-5p (aka anti-mir-675) in 15A FSHD myotubes transfected with DUX4-expressing plasmid (DUX4-FL WT) leads to induced expression of DUX4, indicating that endogenously expressed mir-675 is capable of inhibiting the expression of DUX4. Myotubes were collected 4 days after differentiation. For Replicates 1 and 2, alpha-tubulin was used as a reference gene, and was detected using the alpha-tubulin rabbit polyclonal antibody (1:500 in 5% milk TBST buffer, ab15246; Abcam). DUX4 protein was detected using an anti-V5 antibody (HRP-coupled mouse monoclonal antibody used at 1:5,000 in 5% milk TBST buffer). For Rep. 3 the 15A myoblasts were transfected with CMV.DUX4-FL/CMV.eGFP or CMV.DUX4-mir-675Res plasmids, with both co-expressing eGFP. The latter was used as a transfection control and a reference gene for quantification purposes. In this replicated western blot, DUX4 protein was also detected using an anti-V5 antibody.

FIG. 28 shows that β-estradiol, β-estradiol+medroxyprogesterone acetate (MPA), or melatonin, significantly increased mir-675 levels when compared to the control, i.e., 100% ethanol treated DUX4-transfected cells. β-estradiol, MPA, and melatonin increased mir-675 expression and reduced the expression of DUX4 and DUX4-induced biomarker TRIM43 in HEK293 cells. Droplet Digital PCR (ddPCR) was carried out to measure mir-675-5p, DUX4 and TRIM43 levels. HEK293 cells were transfected with DUX4 and were treated with two drugs individually (i.e., β-estradiol and melatonin) or with a combination of β-estradiol and MPA at 10, 20 or 40 μM at the time of transfection. The effects of these drugs were evaluated by comparison to ethanol (vehicle)-treated cells. Numbers correspond to n=3 independent experiments (ANOVA, P<0.0001). The quantification (percent change) of gene expression from HEK293 cells treated with β-estradiol, β-estradiol+MPA, or melatonin was measured using droplet digital PCR (ddPCR) and is reported in Table 3 provided herein.

FIG. 29A-C shows the effects of the three treatment regimens, β-estradiol, β-estradiol+MPA, or melatonin, on the expression of endogenous mir-675-5p, DUX4 and TRIM43 in 15A (FIG. 29A), 17A (FIG. 29B), and 18A (FIG. 29C) FSHD differentiated muscle cell lines (myotubes). These FSHD cell lines were chosen because they exhibit low (15A), medium (18A) and high (17A) DUX4 expression [Jones et al., Hum. Mol. Genet. 21: 4419-30 (2012)]. Each of β-estradiol, β-estradiol+MPA, and melatonin increased mir-675 expression and reduced the expression of DUX4 and the DUX4-induced biomarker TRIM43 in three FSHD affected myotube lines. All treatments were compared to ethanol (vehicle)-treated control cells (n=6 independent experiments for 15A and N=3 for 17A and 18A. *, P<0.05. **, P<0.01. ***, P<0.001, ANOVA). The quantification of gene expression (mir-675-5p, DUX4 and TRIM43) in 5-day differentiated 15A, 17A and 18A myotubes is reported in Table 4 herein.

FIG. 30 shows that the endogenous mir-675 targets the CDC6 gene expression in control non-affected differentiated muscle cell lines (myotubes of 15V muscle cell lines) and prevents DUX4-induced toxicity in 15A FSHD-affected human myotubes. The targeting of CDC6 gene expression was tested by using a specific anti-mir-675 antagomir and by measuring Cdc6 protein levels in 4-days differentiated 15V control myotubes. Cdc6 was only detected in myotubes transfected with anti-mir-675 (also see FIG. 26 for uncropped gel). The housekeeping protein a-tubulin was used as reference.

FIG. 31A-C shows results from a molecular beacon binding assay (MBB assay) which showed that mir-675 targets sites at DUX4 ORF and 3′UTR with high efficiency (and provides an update to FIG. 13A-C). FIG. 31A provides a schematic of DUX4 sequence showing predicted target site (TS) positions for mir-675-5p. FIG. 31B (left panel) provides a schematic explaining the fluorescence-based molecular beacon binding assay used to determine mir-675-5p binding to DUX4 sequence. Unbound, the molecular beacon (MB) folds into a stem loop structure that brings a quencher (zenBHQ) in close proximity to a fluorophore (6FAM), thereby quenching the fluorescence emission of 6FAM. The mature sequence of mir-675-5p was incorporated in the MB loop sequence. Hybridization of the MB to a complementary TS sequence separates the fluorophore and quencher, allowing fluorescence emission, which was then quantified as a measure of binding. FIG. 31B (right panel) provides a graph showing binding of the mature mir-675-5p molecular beacon to target sites shown in FIG. 31A. Each data point represents mean±SD of three separate experiments. mir-675-5p was able to bind eight target sites within the full length DUX4 sequence (TS527, TS649, TS668, TS754, TS780, TS1004, TS1340 and TS1471). The first six TS are in DUX4 ORF and the remaining two TS are found in the 3′UTR. Six predicted TS did not bind to mir-675-5p (see FIGS. 17A-B and FIG. 13D for TS position and sequence; FIG. 13D shows the molecular beacon for miRNA-5p w/5′ tag (6FAM dye) and 3′ tag (Zen black hole qTencher (ZenBHQ)) and the position, name, and sequence of each of the DUX 4 target sites). The TS neg. ctrl is a random sequence. FIG. 31C shows the binding affinity (K d) of mir-675-5p molecular beacon to each target site was determined by subtracting background fluorescent signal from the molecular beacon signal (MBS), expressed in relative fluorescent units (RFU). The K_{d} corresponds to the TS concentration (μM) required to reach half of maximum fluorescence. Base-pairing between mir-675-5p and its TS (as predicted by the RNAhybrid algorithm) is also shown, as well as their corresponding Kd values. RNA “mimic” bases were generated in the mir-675-5p:TS pair, and replaced “G” nucleotides with “A” nucleotides (in grey) whenever the “G” is facing a “T”.

FIG. 32A-B shows that mir-675 is capable of protecting mouse skeletal tibialis anterior (TA) muscles from DUX4-induced muscle damage (and provides an update to FIG. 15A-B). FIG. 32A shows H&E staining, central nuclei counts and gene expression (ddPCR) analysis of AAV-injected adult mouse (C57BL/6) TA muscles 2 weeks after intramuscular (IM) injection with the indicated doses of vectors. Images show 10 μm cryosections stained with H&E at high (20×) and low power (4×). To help visualize the breadth of potential lesions on low-power images, fibers with central nuclei (CN) or areas of active degeneration and inflammation were intentionally shaded with a purple digital overlay. DUX4-expressing muscles show histopathological evidence of degeneration, including myofibers with inflammatory infiltrates, central nuclei, and variable fiber size (top left). After co-injections of AAV.CMV.DUX4-FL and scAAV6.mir-675 vectors (top right, respectively), muscles were histologically normal. High-power photos show representative images at indicated vector dosages of N=8 TA co-injected with scAAV6.mir-675 and AAV.CMV.DUX4-FL, N=5 TA co-injected with a negative control AAV (AAV.milacZ or AAV.eGFP) and AAV.CMV.DUX4-FL and N=3 TA co-injected with AAV.milacZ and scAAV6.mir-675. The latter TA muscles were histologically normal (bottom left), indicating that scAAV6.mir-675 is not toxic to muscle. Scale bars, 100 μm for high-power; 500 μm for low-power images. Central nuclei counts of 20×H&E stained TA muscle serial sections (10 μm). scAAV6.mir-675-treated muscles showed 81±6% (N=8, two-tailed unpaired t-test, ***, P=0.0004) fewer myofibers with central nuclei when compared with TA muscles co-injected with negative control AAV and AAV.CMV.DUX4-FL (N=5). Droplet digital PCR (ddPCR) of DUX4-FL expression in scAAV6.mir-675-treated and untreated TA muscles. In treated TA muscles, DUX4-FL levels were reduced by 56±12% when compared to the untreated muscles (N=8, two-tailed unpaired t test, *, P=0.01). ddPCR of mir-675 and DUX4-responsive mouse biomarkers (Trim36 and Wfdc3) in scAAV6.mir-675 treated and un-treated TA muscles. Trim36 and Wfdc3 expression were reduced by 88±4% (N=8, ANOVA, ***, P=0.0004) and 57±13% (N=8, ANOVA, *, P<0.011), respectively. FIG. 32A, middle right, Western blots on proteins collected from TA muscles co-injected with negative control AAV and AAV.CMV.DUX4-FL or AAV.CMV.DUX4-FL and scAAV6.mir-675. Anti-V5 epitope antibodies were used to detect V5-tagged DUX4. Alpha-tubulin (a-tubulin) was used as a loading control. FIG. 32B provides images which show 10 μm cryosections immunofluorescently stained for DUX4 (V5 epitope, red) or nuclei (DAPI, blue). High-power photos show representative images at indicated vector dosages of N=8 TA co-injected with scAAV6.mir-675 and AAV.CMV.DUX4-FL, N=5 TA co-injected with negative control AAV and AAV.CMV.DUX4-FL. In the absence of mir-675, high levels of DUX4 proteins were detected. White arrows indicate representative fibers and myonuclei expressing DUX4 proteins. These data show that target engagement by mir-675 reduced DUX4 protein levels in TA skeletal muscles co-injected with AAV.mir-675 and AAV.DUX4.

FIG. 33 shows three uncropped repeated western blots performed on protein extracts from 15A FSHD myotubes co-transfected with anti-mir-675-5p, DUX4-FL (WT) and DUX4-mir-675Res constructs (and provides an update to FIG. 27). Myoblasts were collected 24 and 48 hours after transfection. Myotubes were then collected 4 days after differentiation (5 days after transfection). Alpha-tubulin was used as a reference gene for Rep. 1 and 2. DUX4 protein was detected using an anti-V5 antibody (HRP-coupled mouse monoclonal antibody used at 1:5,000 in 5% milk TBST buffer). For Rep. 3, the 15A myoblasts were transfected with CMV.DUX4-FL/CMV.eGFP or CMV.DUX4-miR-675Res plasmids (both co-expressing eGFP from the same plasmid). For quantification, eGFP was used as a transfection control and as a reference gene. In this replicated western blot, DUX4 protein was also detected using an anti-V5 antibody. β-actin was used as an endogenously expressed protein reference. β-actin was detected using an anti-mouse monoclonal antibody (1:1000 in 5% milk TBST buffer, SIGMA). The graph shows quantification of DUX4 protein levels in all tested conditions. Source data are provided as a Source Data file. This figure shows that the transfection of anti-mir-675-5p (aka anti-mir-675) in 15A FSHD myotubes transfected with DUX4-expressing plasmid (DUX4-FL WT) leads to induced expression of DUX4, indicating that endogenously expressed mir-675 is capable of inhibiting the expression of DUX4.

FIG. 34 shows H&E staining of 10 μm muscle sections collected from C57BL/6 TA muscles injected with either 5×10⁹ DRP of AAV.CMV.DUX4-FL or AAV.U6.mi405 or AAV.U6.mi405F or AAV.U6.mi405G or AAV.U6.mi405H for 8 weeks. This figure also shows muscle sections from the TA muscles co-injected for 8 weeks with 5×10⁹ DRP of AAV.CMV.DUX4-FL and 5×10⁹ DRP of each of the four mi405 constructs (i.e., AAV.U6.mi405 or AAV.U6.mi405F or AAV.U6.mi405G or AAV.U6.mi405H). These data show that, at low doses, mi405G and mi405H are more efficient than mi405 in eliminating DUX4-induced muscle toxicity characterized by mononuclear cells infiltration and myofibers with central nuclei.

FIG. 35 shows ddPCR gene expression data on DUX4, TRIM43 and ZSCAN4 from 18A FSHD affected myotubes treated with increasing concentrations of Pyrazinamide or Sorafenib at the 4t h day of differentiation. Gene expression is shown as copies of each gene relative to the copies of the reference gene, RPL13A. These data show that with increasing concentrations of Pyrazinamide or Sorafenib, concentrations of DUX4 and DUX4-responsive biomarkers, e.g., TRIM43 and ZSCAN4, decreased in 18A FSHD affected myotubes.

### DETAILED DESCRIPTION

The disclosure provides a novel strategy to accomplish double homeobox protein 4 (DUX4) gene expression post-transcriptionally by repressing or inhibiting DUX4 protein production because the expression of DUX4 in muscle is known to cause cancer and muscular dystrophy including, but not limited to, facioscapulohumeral muscular dystrophy (FSHD). Thus, in some aspects, the products and methods described herein are used in treating, ameliorating, delaying the progression of, and/or preventing cancer and muscular dystrophy including, but not limited to sarcoma and FSHD.

The DUX4 gene encodes an approximately 45kDA protein; see UniProtKB-Q9UBX2 (DUX4_HUMAN). De-repression of the DUX4 gene is involved in disease pathogenesis of FSHD. De-repression can occur through two known mechanisms: D4Z4 repeat contraction, or mutation in chromatin modifier genes SMCHD1 or DNMT3B. For the former, in unaffected subjects, the D4Z4 array consists of 11-100 repeats, while in FSHD1 patients, the array is reduced to 1-10 repeats (Mostacciuolo et al., Clin. Genet. June; 75(6):550-5 (2009); PubMed:19320656). Either condition can cause DNA hypomethylation at chromosome 4q35, thereby creating a chromosomal environment permissive for DUX4 expression.

DUX4 is located in D4Z4 macrosatellite which is epigenetically repressed in somatic tissues. D4Z4 chromatin relaxation in FSHD1 results in inefficient epigenetic repression of DUX4 and a variegated pattern of DUX4 protein expression in a subset of skeletal muscle nuclei. Ectopic expression of DUX4 in skeletal muscle activates the expression of stem cell and germline genes, and, when overexpressed in somatic cells, DUX4 can ultimately lead to cell death.

Each D4Z4 repeat unit has an open reading frame (named DUX4) that encodes two homeoboxes; the repeat-array and ORF is conserved in other mammals. The encoded protein has been reported to function as a transcriptional activator of numerous genes, including some considered to be FSHD disease biomarkers, including ZSCAN4, PRAMEF12, TRIM43, and MBD3L2 (Yao et al., Hum Mol Genet. 2014 October 15; 23(20):5342-52; PMID: 24861551). Contraction of the macrosatellite repeat causes autosomal dominant FSHD. Alternative splicing results in multiple transcript variants.

In some aspects, the nucleic acid encoding human DUX4 is set forth in the nucleotide sequence set forth in SEQ ID NO: 1. In some aspects, the amino acid sequence of human DUX4 is set forth in the amino acid sequence set forth in SEQ ID NO: 2. In various aspects, the methods of the disclosure also target isoforms and variants of the nucleotide sequence set forth in SEQ ID NO: 1. In some aspects, the variants comprise 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to the nucleotide sequence set forth in SEQ ID NO: 1 In some aspects, the methods of the disclosure target isoforms and variants of nucleic acids comprising nucleotide sequences encoding the amino acid sequence set forth in SEQ ID NO: 2. In some aspects, the variants comprise 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, and 70% identity to a nucleotide sequence that encodes the amino acid sequence set forth in SEQ ID NO: 2.

| SEQ ID NO: | Sequence |
|---|---|
| 1 | DUX4 + 3′UTR NT: |
| | atggccctcccgacaccctcggacagcaccctccccgcggaagcccggggacgaggacggcgacgga |
| | gactcgtttggaccccgagccaaagcgaggccctgcgagcctgctttgagcggaacccgtacccgggcat |
| | cgccaccagagaacggctggcccaggccatcggcattccggagcccagggtccagatttggtttcagaat |
| | gagaggtcacgccagctgaggcagcaccggcgggaatctcggccctggcccgggagacgcggcccgc |
| | cagaaggccggcgaaagcggaccgccgtcaccggatcccagaccgccctgctcctccgagcctttgaga |
| | aggatcgctttccaggcatcgccgcccgggaggagctggccagagagacgggcctcccggagtccagg |
| | attcagatctggtttcagaatcgaagggccaggcacccgggacagggtggcagggcgcccgcgcaggc |
| | aggcggcctgtgcagcgcggcccccggcgggggtcaccctgctccctcgtgggtcgccttcgcccacacc |
| | ggcgcgtggggaacggggcttcccgcaccccacgtgccctgcgcgcctggggctctcccacagggggctt |
| | tcgtgagccaggcagcgagggccgcccccgcgctgcagcccagccaggccgcgccggcagaggggat |
| | ctcccaacctgccccggcgcgcggggatttcgcctacgccgccccggctcctccggacggggcgctctcc |
| | caccctcaggctcctcggtggcctccgcacccgggcaaaagccgggaggaccgggacccgcagcgcg |
| | acggcctgccgggcccctgcgcggtggcacagcctgggcccgctcaagcggggccgcagggccaagg |
| | ggtgcttgcgccacccacgtcccaggggagtccgtggtggggctggggccggggtccccaggtcgccgg |
| | ggcggcgtgggaaccccaagccggggcagctccacctccccagcccgcgcccccggacgcctccgcct |
| | ccgcgcggcaggggcagatgcaaggcatcccggcgccctcccaggcgctccaggagccggcgccctg |
| | gtctgcactcccctgcggcctgctgctggatgagctcctggcgagcccggagtttctgcagcaggcgcaacc |
| | tctcctagaaacggaggccccgggggagctggaggcctcggaagaggccgcctcgctggaagcacccc |
| | tcagcgaggaagaataccgggctctgctggaggagctttaggacgcggggttgggacggggtcgggtggt |
| | tcggggcagggccgtggcctctctttcgcggggaacacctggctggctacggaggggcgtgtctccgcccc |
| | gccccctccaccgggctgaccggcctgggattcctgccttctaggtctaggcccggtgagagactccacac |
| | cgcggagaactgccattctttcctgggcatcccggggatcccagagccggcccaggtaccagcaggtggg |
| | ccgcctactgcgcacgcgcgggtttgcgggcagccgcctgggctgtgggagcagcccgggcagagctct |
| | cctgcctctccaccagcccaccccgccgcctgaccgccccctccccaccccccaccccccacccccgga |
| | aaacgcgtcgtcccctgggctgggtggagacccccgtcccgcgaaacaccgggccccgcgcagcgtcc |
| | gggcctgactccgctccggcggctcgcctcctgtgtgcccccgcgccaccgtcgcccgcccgcccgggcc |
| | cctgcagcctcccagctgccagcgcggagctcctggcggtcaaaagcatacctctgtctgtctttgcccgctt |
| | cctggctagacctgcgcgcagtgcgcaccccggctgacgtgcaagggagctcgctggcctctctgtgccctt |
| | gttcttccgtgaaattctggctgaatgtctccccccaccttccgacgctgtctaggcaaacctggattagagtta |
| | catctcctggatgattagttcagagatatattaaaatgccccctccc |
| 2 | DUX4 AA: |
| | MALPTPSDSTLPAEARGRGRRRRLVWTPSQSEALRACFERNPYPGIATRER |
| | LAQAIGIPEPRVQIWFQNERSRQLRQHRRESRPWPGRRGPPEGRRKRTAV |
| | TGSQTALLLRAFEKDRFPGIAAREELARETGLPESRIQIWFQNRRARHPGQG |
| | GRAPAQAGGLCSAAPGGGHPAPSWVAFAHTGAWGTGLPAPHVPCAPGAL |
| | PQGAFVSQAARAAPALQPSQAAPAEGISQPAPARGDFAYAAPAPPDGALSH |
| | PQAPRWPPHPGKSREDRDPQRDGLPGPCAVAQPGPAQAGPQGQGVLAPP |
| | TSQGSPWWGWGRGPQVAGAAWEPQAGAAPPPQPAPPDASASARQGQM |
| | QGIPAPSQALQEPAPWSALPCGLLLDELLASPEFLQQAQPLLETEAPGELEA |
| | SEEAASLEAPLSEEEYRALLEEL |

There is currently no treatment for FSHD, and despite its relative abundance among the muscular dystrophies, very few FSHD-targeted translational studies have been published. Several FSHD candidate genes have been identified, but numerous recent studies support that the primary contributor to FSHD pathogenesis is the pro-apoptotic DUX4 gene, which encodes a transcription factor. Thus, in the simplest terms, DUX4-overexpression is a primary pathogenic insult underlying FSHD [Chen et al., (2016) Mol Ther 24: 1405-1411; Ansseau et al., (2017) Genes 8(3): 93; Lek et al., (2020) Sci Transl Med 12(536); Himeda et al., (2016) Mol Ther 24: 527-535; DeSimone et al., (2019) Sci Adv 5:12; Lim et al., (2020) Proc Natl Acad Sci USA 117: 16509-16515; Wallace et al., (2018), supra; Rojas et al., (2020) J Pharmacol Exp Ther. 374(3): 489-498].

The disclosure provides nucleic acids encoding microRNA (miRNA) targeting DUX4 and inhibiting the expression of DUX4. The disclosure provides nucleic acids encoding microRNA (miRNA) targeting DUX4 comprising and inhibiting the expression of DUX4 further comprising a promoter sequence. The disclosure provides nucleic acids comprising the RNA sequence targeted by the miRNA. The disclosure provides DUX4 sequences that the miRNA sequences are designed to target. The disclosure includes various nucleic acids comprising, consisting essentially of, or consisting of the various nucleotide sequences described herein. In some aspects, the nucleic acid comprises the nucleotide sequence. In some aspects, the nucleic acid consists essentially of the nucleotide sequence. In some aspects, the nucleic acid consists of the nucleotide sequence.

Exemplary nucleotide sequences used in miRNA targeting of DUX4 described herein include, but are not limited to, those identified in Table 1 below.

TABLE 1
Nucleic acids of the disclosure

| | | | | miRNA | | | | | | |
|---|---|---|---|---|---|---|---|---|---|---|
| | | | SEQ | name with | | SEQ | | SEQ | | SEQ |
| miRNA | miRNA | DNA | ID | the | DNA sequence with the | ID | RNA | ID | DUX4 target | ID |
| # | name | sequence | NO: | promoter | promoter | NO: | sequence | NO: | sequence | NO: |
| 1 | milacZ | GCGTTTAGT | 3 | U6.milacZ | ACGCCGCCATCTCTAGGCCCGCG | 48 | AUUAAAG | 93 | NONE | N/A |
| | | GAACCGTCA | | (control) | CCGGCCCCCTCGCACAGACTTGT | | CGAGUGG | | (control) | |
| | | GATGGTACC | | | GGGAGAAGCTCGGCTACTCCCCT | | CAACAUG | | | |
| | | GTTTAAACTC | | | GCCCCGGTTAATTTGCATATAATA | | G | | | |
| | | GAGTGAGCG | | | TTTCCTAGTAACTATAGAGGCTTA | | | | | |
| | | ACATGTTGC | | | ATGTGCGATAAAAGACAGATAATC | | | | | |
| | | CACTC | | | TGTTCTTTTTAATACTAGCTACATT | | | | | |
| | | GCTTTAATCT | | | TTACATGATAGGCTTGGATTTCTAT | | | | | |
| | | GTAAAGCCA | | | AAGAGATACAAATACTAAATTATTA | | | | | |
| | | CAGATGGGA | | | TTTTAAAAAACAGCACAAAAGGAA | | | | | |
| | | TTAAAGCGA | | | ACTCACCCTAACTGTAAAGTAATT | | | | | |
| | | GTGGCAACA | | | GTGTGTTTTGAGACTATAAATATC | | | | | |
| | | TGGCGCCTA | | | CCTTGGAGAAAAGCCTTGTTTGCG | | | | | |
| | | CTAGAGCGG | | | TTTAGTGAACCGTCAGATGGTACC | | | | | |
| | | CCGCCACAG | | | GTTTAAACTCGAGTGAGCGACATG | | | | | |
| | | CGGGGAGAT | | | TTGCCACTCGCTTTAATCTGTAAA | | | | | |
| | | CCAGACATG | | | GCCACAGATGGGATTAAAGCGAG | | | | | |
| | | ATAAGATACA | | | TGGCAACATGGCGCCTACTAGAG | | | | | |
| | | | | | CGGCCGCCACAGCGGGGAGATCC | | | | | |
| | | | | | AGACATGATAAGATACATTTTTT | | | | | |
| 2 | miDUX4. | GCGTTTAGT | 4 | U6.miDUX4. | ACGCCGCCATCTCTAGGCCCGCG | 49 | AAACCAG | 94 | 405TS: | 106 |
| | 405 | GAACCGTCA | | 405 | CCGGCCCCCTCGCACAGACTTGT | | AUCUGAA | | GUCCAGGAUU | |
| | | GATGGTACC | | (control) | GGGAGAAGCTCGGCTACTCCCCT | | UCCUGGA | | CAGAUCUGGU | |
| | | GTTTAAACTC | | | GCCCCGGTTAATTTGCATATAATA | | C | | UU | |
| | | GAGTGAGCG | | | TTTCCTAGTAACTATAGAGGCTTA | | | | 193TS: | 107 |
| | | ATCCAGGAT | | | ATGTGCGATAAAAGACAGATAATC | | | | CCAGGGUCCA | |
| | | TCAGATCTG | | | TGTTCTTTTTAATACTAGCTACATT | | | | GAUUUGGUU | |
| | | GTTTCTGTAA | | | TTACATGATAGGCTTGGATTTCTAT | | | | U | |
| | | AGCCACAGA | | | AAGAGATACAAATACTAAATTATTA | | | | | |
| | | TGGGAAACC | | | TTTTAAAAAACAGCACAAAAGGAA | | | | | |
| | | AGATCTGAAT | | | ACTCACCCTAACTGTAAAGTAATT | | | | | |
| | | CCTGGACTG | | | GTGTGTTTTGAGACTATAAATATC | | | | | |
| | | CCTACTAGA | | | CCTTGGAGAAAAGCCTTGTTTGCG | | | | | |
| | | GCGGCCGCC | | | TTTAGTGAACCGTCAGATGGTACC | | | | | |
| | | ACAGCGGGG | | | GTTTAAACTCGAGTGAGCGATCCA | | | | | |
| | | AGATCCAGA | | | GGATTCAGATCTGGTTTCTGTAAA | | | | | |
| | | CATGATAAG | | | GCCACAGATGGGAAACCAGATCT | | | | | |
| | | ATACA | | | GAATCCTGGACTGCCTACTAGAGC | | | | | |
| | | | | | GGCCGCCACAGCGGGGAGATCCA | | | | | |
| | | | | | GACATGATAAGATACATTTTTT | | | | | |
| 3 | mir- | GCGTTTAGT | 5 | U6.mir-675 | GACGCCGCCATCTCTAGGCCCGC | 50 | UGGUGCG | 95 | 527TS: | 108 |
| | 675U6 | GAACCGTCA | | | GCCGGCCCCCTCGCACAGACTTG | | GAGAGGG | | CGUGGGUCG | |
| | | GATGGTACC | | | TGGGAGAAGCTCGGCTACTCCCC | | CCCACAG | | CCUUCGCCCA | |
| | | GTTTAAACTC | | | TGCCCCGGTTAATTTGCATATAAT | | UG | | C | |
| | | GAGCCCAGG | | | ATTTCCTAGTAACTATAGAGGCTT | | | | 649TS: | 109 |
| | | GTCTGGTGC | | | AATGTGCGATAAAAGACAGATAAT | | | | GCGCUGCAG | |
| | | GGAGAGGGC | | | CTGTTCTTTTTAATACTAGCTACAT | | | | CCCAGCCAGG | |
| | | CCACAGTGG | | | TTTACATGATAGGCTTGGATTTCTA | | | | CCGCGCCGG | |
| | | ACTTGGTGA | | | TAAGAGATACAAATACTAAATTATT | | | | 668TS: | 110 |
| | | CGCTGTATG | | | ATTTTAAAAAACAGCACAAAAGGA | | | | UGCAGCCCAG | |
| | | CCCTCACCG | | | AACTCACCCTAACTGTAAAGTAAT | | | | CCAGGCCGC | |
| | | CTCAGCCCC | | | TGTGTGTTTTGAGACTATAAATATC | | | | GCCG | |
| | | TGGGACTAG | | | CCTTGGAGAAAAGCCTTGTTTGCG | | | | 780TS: | 111 |
| | | AGCGGCCGC | | | TTTAGTGAACCGTCAGATGGTACC | | | | UCCUCGCUG | |
| | | CACAGCGGG | | | GTTTAAACTCGAGCCCAGGGTCTG | | | | GCCUCCGCAC | |
| | | GAGATCCAG | | | GTGCGGAGAGGGCCCACAGTGGA | | | | C | |
| | | ACATGATAA | | | CTTGGTGACGCTGTATGCCCTCAC | | | | 1004TS: | 112 |
| | | GATACA | | | CGCTCAGCCCCTGGGACTAGAGC | | | | CUCCACCUCC | |
| | | | | | GGCCGCCACAGCGGGGAGATCCA | | | | CCAGCCCGC | |
| | | | | | GACATGATAAGATACATTTTTT | | | | GCC | |
| | | | | | | | | | 1340TS: | 113 |
| | | | | | | | | | CCGGUGAGA | |
| | | | | | | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 4 | mir- | CCCCCAGGG | 6 | H1.mir-675 | GAACGCTGACGTCATCAACCCGC | 51 | UGGUGCG | 95 | 527TS: | 108 |
| | 675H1 | TCTGGTGCG | | | TCCAAGGAATCGCGGGCCCAGTG | | GAGAGGG | | CGUGGGUCG | |
| | | GAGAGGGCC | | | TCACTAGGCGGGAACACCCAGCG | | CCCACAG | | CCUUCGCCCA | |
| | | CACAGTGGA | | | CGCGTGCGCCCTGGCAGGAAGAT | | UG | | C | |
| | | CTTGGTGAC | | | GGCTGTGAGGGACAGGGGAGTG | | | | 649TS: | 109 |
| | | GCTGTATGC | | | GCGCCCTGCAATATTTGCATGTCG | | | | GCGCUGCAG | |
| | | CCTCACCGC | | | CTATGTGTTCTGGGAAATCACCAT | | | | CCCAGCCAGG | |
| | | TCAGCCCCT | | | AAACGTGAAATGTCTTTGGATTTG | | | | CCGCGCCGG | |
| | | GGGGAATTC | | | GGAATCTTATAAGTTCTGTATGAG | | | | 668TS: | 110 |
| | | TTCGATTCTG | | | ACCACCCCCCAGGGTCTGGTGCG | | | | UGCAGCCCAG | |
| | | C | | | GAGAGGGCCCACAGTGGACTTGG | | | | CCAGGCCGC | |
| | | | | | TGACGCTGTATGCCCTCACCGCTC | | | | GCCG | |
| | | | | | AGCCCCTGGGGAATTCTTCGATTC | | | | 780TS: | 111 |
| | | | | | TGCTTTTTT | | | | UCCUCGCUG | |
| | | | | | | | | | GCCUCCGCAC | |
| | | | | | | | | | C | |
| | | | | | | | | | 1004TS: | 112 |
| | | | | | | | | | CUCCACCUCC | |
| | | | | | | | | | CCAGCCCGC | |
| | | | | | | | | | GCC | |
| | | | | | | | | | 1340TS: | 113 |
| | | | | | | | | | CCGGUGAGA | |
| | | | | | | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 5 | mir- | GCCCCAGGG | 7 | U6.mir-675F | GACGCCGCCATCTCTAGGCCCGC | 52 | UGGUGCG | 95 | 527TS: | 108 |
| | 675F | TCTGGTGCG | | | GCCGGCCCCCTCGCACAGACTTG | | GAGAGGG | | CGUGGGUCG | |
| | | GAGAGGGCC | | | TGGGAGAAGCTCGGCTACTCCCC | | CCCACAG | | CCUUCGCCCA | |
| | | CACAGTGGA | | | TGCCCCGGTTAATTTGCATATAAT | | UG | | C | |
| | | CTTGGTGAC | | | ATTTCCTAGTAACTATAGAGGCTT | | | | 649TS: | 109 |
| | | GCTGTATGC | | | AATGTGCGATAAAAGACAGATAAT | | | | GCGCUGCAG | |
| | | CCTCACCGC | | | CTGTTCTTTTTAATACTAGCTACAT | | | | CCCAGCCAGG | |
| | | TCAGCCCCT | | | TTTACATGATAGGCTTGGATTTCTA | | | | CCGCGCCGG | |
| | | GGGGAATTC | | | TAAGAGATACAAATACTAAATTATT | | | | 668TS: | 110 |
| | | TTCGATTCTG | | | ATTTTAAAAAACAGCACAAAAGGA | | | | UGCAGCCCAG | |
| | | | | | AACTCACCCTAACTGTAAAGTAAT | | | | CCAGGCCGC | |
| | | | | | TGTGTGTTTTGAGACTATAAATATC | | | | GCCG | |
| | | | | | CCTTGGAGAAAAGCCTTGTTTGCC | | | | 780TS: | 111 |
| | | | | | CCAGGGTCTGGTGCGGAGAGGGC | | | | UCCUCGCUG | |
| | | | | | CCACAGTGGACTTGGTGACGCTG | | | | GCCUCCGCAC | |
| | | | | | TATGCCCTCACCGCTCAGCCCCT | | | | C | |
| | | | | | GGGGAATTCTTCGATTCTGCTTTT | | | | 1004TS: | 112 |
| | | | | | TT | | | | CUCCACCUCC | |
| | | | | | | | | | CCAGCCCGC | |
| | | | | | | | | | GCC | |
| | | | | | | | | | 1340TS: | 113 |
| | | | | | | | | | CCGGUGAGA | |
| | | | | | | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 6 | mir- | GCCCCAGGG | 8 | U6.mir- | TCTAGAGATCCGACGCCGCCATCT | 53 | UGGUGCG | 95 | 527TS: | 108 |
| | 675NF | TCTGGTGCG | | 675NF | CTAGGCCCGCGCCGGCCCCCTCG | | GAGAGGG | | CGUGGGUCG | |
| | | GAGAGGGCC | | | CACAGACTTGTGGGAGAAGCTCG | | CCCACAG | | CCUUCGCCCA | |
| | | CACAGTGGA | | | GCTACTCCCCTGCCCCGGTTAATT | | UG | | C | |
| | | CTTGGTGAC | | | TGCATATAATATTTCCTAGTAACTA | | | | 649TS: | 109 |
| | | GCTGTATGC | | | TAGAGGCTTAATGTGCGATAAAAG | | | | GCGCUGCAG | |
| | | CCTCACCGC | | | ACAGATAATCTGTTCTTTTTAATAC | | | | CCCAGCCAGG | |
| | | TCAGCCCCT | | | TAGCTACATTTTACATGATAGGCTT | | | | CCGCGCCGG | |
| | | GGGG | | | GGATTTCTATAAGAGATACAAATA | | | | 668TS: | 110 |
| | | | | | CTAAATTATTATTTTAAAAAACAGC | | | | UGCAGCCCAG | |
| | | | | | ACAAAAGGAAACTCACCCTAACTG | | | | CCAGGCCGC | |
| | | | | | TAAAGTAATTGTGTGTTTTGAGACT | | | | GCCG | |
| | | | | | ATAAATATCCCTTGGAGAAAAGCC | | | | 780TS: | 111 |
| | | | | | TTGTTTGCCCCAGGGTCTGGTGC | | | | UCCUCGCUG | |
| | | | | | GGAGAGGGCCCACAGTGGACTTG | | | | GCCUCCGCAC | |
| | | | | | GTGACGCTGTATGCCCTCACCGC | | | | C | |
| | | | | | TCAGCCCCTGGGGTTTTTT | | | | 1004TS: | 112 |
| | | | | | | | | | CUCCACCUCC | |
| | | | | | | | | | CCAGCCCGC | |
| | | | | | | | | | GCC | |
| | | | | | | | | | 1340TS: | 113 |
| | | | | | | | | | CCGGUGAGA | |
| | | | | | | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 7 | mir-675- | GCCCCAGGG | 9 | U6.mir-675- | TCTAGAGATCCGACGCCGCCATCT | 54 | UGGUGCG | 95 | 527TS: | 108 |
| | 2.1 | TCTGGTGCG | | 2.1 | CTAGGCCCGCGCCGGCCCCCTCG | | GAGAGGG | | CGUGGGUCG | |
| | | GAGAGGGCC | | | CACAGACTTGTGGGAGAAGCTCG | | CCCACAG | | CCUUCGCCCA | |
| | | CACAGTGGA | | | GCTACTCCCCTGCCCCGGTTAATT | | UG | | C | |
| | | CTTGGTGAC | | | TGCATATAATATTTCCTAGTAACTA | | | | 649TS: | 109 |
| | | GCTGTATGC | | | TAGAGGCTTAATGTGCGATAAAAG | | | | GCGCUGCAG | |
| | | CCTCACCGC | | | ACAGATAATCTGTTCTTTTTAATAC | | | | CCCAGCCAGG | |
| | | TCAGCCCCT | | | TAGCTACATTTTACATGATAGGCTT | | | | CCGCGCCGG | |
| | | GGGGTAACT | | | GGATTTCTATAAGAGATACAAATA | | | | 668TS: | 110 |
| | | CCTAATCACA | | | CTAAATTATTATTTTAAAAAACAGC | | | | UGCAGCCCAG | |
| | | C | | | ACAAAAGGAAACTCACCCTAACTG | | | | CCAGGCCGC | |
| | | | | | TAAAGTAATTGTGTGTTTTGAGACT | | | | GCCG | |
| | | | | | ATAAATATCCCTTGGAGAAAAGCC | | | | 780TS: | 111 |
| | | | | | TTGTTTGCCCCAGGGTCTGGTGC | | | | UCCUCGCUG | |
| | | | | | GGAGAGGGCCCACAGTGGACTTG | | | | GCCUCCGCAC | |
| | | | | | GTGACGCTGTATGCCCTCACCGC | | | | C | |
| | | | | | TCAGCCCCTGGGGTAACTCCTAAT | | | | 1004TS: | 112 |
| | | | | | CACACTTTTTT | | | | CUCCACCUCC | |
| | | | | | | | | | CCAGCCCGC | |
| | | | | | | | | | GCC | |
| | | | | | | | | | 1340TS: | 113 |
| | | | | | | | | | CCGGUGAGA | |
| | | | | | | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 8 | mir-675- | CCCCCAGGG | 10 | U6.mir-675- | GAACGCTGACGTCATCAACCCGC | 55 | UGGUGCG | 95 | 527TS: | 108 |
| | 2.2 | TCTGGTGCG | | 2.2 | TCCAAGGAATCGCGGGCCCAGTG | | GAGAGGG | | CGUGGGUCG | |
| | | GAGAGGGCC | | | TCACTAGGGGGGAACACCCAGCG | | CCCACAG | | CCUUCGCCCA | |
| | | CACAGTGGA | | | CGCGTGCGCCCTGGCAGGAAGAT | | UG | | C | |
| | | CTTGGTGAC | | | GGCTGTGAGGGACAGGGGAGTG | | | | 649TS: | 109 |
| | | GCTGTATGC | | | GCGCCCTGCAATATTTGCATGTCG | | | | GCGCUGCAG | |
| | | CCTCACCGC | | | CTATGTGTTCTGGGAAATCACCAT | | | | CCCAGCCAGG | |
| | | TCAGCCCCT | | | AAACGTGAAATGTCTTTGGATTTG | | | | CCGCGCCGG | |
| | | GGGGTAACT | | | GGAATCTTATAAGTTCTGTATGAG | | | | 668TS: | 110 |
| | | CCTAATCACA | | | ACCACTTGGATCCCCCAGGGTCT | | | | UGCAGCCCAG | |
| | | C | | | GGTGCGGAGAGGGCCCACAGTG | | | | CCAGGCCGC | |
| | | | | | GACTTGGTGACGCTGTATGCCCTC | | | | GCCG | |
| | | | | | ACCGCTCAGCCCCTGGGGTAACT | | | | 780TS: | 111 |
| | | | | | CCTAATCACACTTTTTT | | | | UCCUCGCUG | |
| | | | | | | | | | GCCUCCGCAC | |
| | | | | | | | | | C | |
| | | | | | | | | | 1004TS: | 112 |
| | | | | | | | | | CUCCACCUCC | |
| | | | | | | | | | CCAGCCCGC | |
| | | | | | | | | | GCC | |
| | | | | | | | | | 1340TS: | 113 |
| | | | | | | | | | CCGGUGAGA | |
| | | | | | | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 9 | mir-675- | GAATCACAC | 11 | U6.mir-675- | TCTAGAGATCCGACGCCGCCATCT | 56 | UGGUGCG | 95 | 527TS: | 108 |
| | 2.3 | TGCCCCAGG | | 2.3 | CTAGGCCCGCGCCGGCCCCCTCG | | GAGAGGG | | CGUGGGUCG | |
| | | GTCTGGTGC | | | CACAGACTTGTGGGAGAAGCTCG | | CCCACAG | | CCUUCGCCCA | |
| | | GGAGAGGGC | | | GCTACTCCCCTGCCCCGGTTAATT | | UG | | C | |
| | | CCACAGTGG | | | TGCATATAATATTTCCTAGTAACTA | | | | 649TS: | 109 |
| | | ACTTGGTGA | | | TAGAGGCTTAATGTGCGATAAAAG | | | | GCGCUGCAG | |
| | | CGCTGTATG | | | ACAGATAATCTGTTCTTTTTAATAC | | | | CCCAGCCAGG | |
| | | CCCTCACCG | | | TAGCTACATTTTACATGATAGGCTT | | | | CCGCGCCGG | |
| | | CTCAGCCCC | | | GGATTTCTATAAGAGATACAAATA | | | | 668TS: | 110 |
| | | TGGGGATAC | | | CTAAATTATTATTTTAAAAAACAGC | | | | UGCAGCCCAG | |
| | | TCCTAATCAC | | | ACAAAAGGAAACTCACCCTAACTG | | | | CCAGGCCGC | |
| | | AC | | | TAAAGTAATTGTGTGTTTTGAGACT | | | | GCCG | |
| | | | | | ATAAATATCCCTTGGAGAAAAGCC | | | | 780TS: | 111 |
| | | | | | TTGTTTGAATCACACTGCCCCAGG | | | | UCCUCGCUG | |
| | | | | | GTCTGGTGCGGAGAGGGCCCACA | | | | GCCUCCGCAC | |
| | | | | | GTGGACTTGGTGACGCTGTATGC | | | | C | |
| | | | | | CCTCACCGCTCAGCCCCTGGGGA | | | | 1004TS: | 112 |
| | | | | | TACTCCTAATCACACTTTTTT | | | | CUCCACCUCC | |
| | | | | | | | | | CCAGCCCGC | |
| | | | | | | | | | GCC | |
| | | | | | | | | | 1340TS: | 113 |
| | | | | | | | | | CCGGUGAGA | |
| | | | | | | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 10 | mir-675- | CAATCACACT | 12 | U6.mir-675- | GAACGCTGACGTCATCAACCCGC | 57 | UGGUGCG | 95 | 527TS: | 108 |
| | 2.4 | GCCCCAGGG | | 2.4 | TCCAAGGAATCGCGGGCCCAGTG | | GAGAGGG | | CGUGGGUCG | |
| | | TCTGGTGCG | | | TCACTAGGCGGGAACACCCAGCG | | CCCACAG | | CCUUCGCCCA | |
| | | GAGAGGGCC | | | CGCGTGCGCCCTGGCAGGAAGAT | | UG | | C | |
| | | CACAGTGGA | | | GGCTGTGAGGGACAGGGGAGTG | | | | 649TS: | 109 |
| | | CTTGGTGAC | | | GCGCCCTGCAATATTTGCATGTCG | | | | GCGCUGCAG | |
| | | GCTGTATGC | | | CTATGTGTTCTGGGAAATCACCAT | | | | CCCAGCCAGG | |
| | | CCTCACCGC | | | AAACGTGAAATGTCTTTGGATTTG | | | | CCGCGCCGG | |
| | | TCAGCCCCT | | | GGAATCTTATAAGTTCTGTATGAG | | | | 668TS: | 110 |
| | | GGGGATACT | | | ACCACTTGGATCCAATCACACTGC | | | | UGCAGCCCAG | |
| | | CCTAATCACA | | | CCCAGGGTCTGGTGCGGAGAGGG | | | | CCAGGCCGC | |
| | | C | | | CCCACAGTGGACTTGGTGACGCT | | | | GCCG | |
| | | | | | GTATGCCCTCACCGCTCAGCCCC | | | | 780TS: | 111 |
| | | | | | TGGGGATACTCCTAATCACACTTT | | | | UCCUCGCUG | |
| | | | | | TTT | | | | GCCUCCGCAC | |
| | | | | | | | | | C | |
| | | | | | | | | | 1004TS: | 112 |
| | | | | | | | | | CUCCACCUCC | |
| | | | | | | | | | CCAGCCCGC | |
| | | | | | | | | | GCC | |
| | | | | | | | | | 1340TS: | 113 |
| | | | | | | | | | CCGGUGAGA | |
| | | | | | | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 11 | mir-675- | GAATCACAC | 13 | U6.mir-675- | TCTAGAGATCCGACGCCGCCATCT | 58 | UGGUGCG | 95 | 527TS: | 108 |
| | 2.5 | TGCCCCAGG | | 2.5 | CTAGGCCCGCGCCGGCCCCCTCG | | GAGAGGG | | CGUGGGUCG | |
| | | GTCTGGTGC | | | CACAGACTTGTGGGAGAAGCTCG | | CCCACAG | | CCUUCGCCCA | |
| | | GGAGAGGGC | | | GCTACTCCCCTGCCCCGGTTAATT | | UG | | C | |
| | | CCACAGTGG | | | TGCATATAATATTTCCTAGTAACTA | | | | 649TS: | 109 |
| | | ACTTGGTGA | | | TAGAGGCTTAATGTGCGATAAAAG | | | | GCGCUGCAG | |
| | | CGCTGTATG | | | ACAGATAATCTGTTCTTTTTAATAC | | | | CCCAGCCAGG | |
| | | CCCTCACCG | | | TAGCTACATTTTACATGATAGGCTT | | | | CCGCGCCGG | |
| | | CTCAGCCCC | | | GGATTTCTATAAGAGATACAAATA | | | | 668TS: | 110 |
| | | TGGGGATAC | | | CTAAATTATTATTTTAAAAAACAGC | | | | UGCAGCCCAG | |
| | | TCCTAATCAC | | | ACAAAAGGAAACTCACCCTAACTG | | | | CCAGGCCGC | |
| | | AC | | | TAAAGTAATTGTGTGTTTTGAGACT | | | | GCCG | |
| | | | | | ATAAATATCCCTTGGAGAAAAGCC | | | | 780TS: | 111 |
| | | | | | TTGTTAACGCGAATCACACTGCCC | | | | UCCUCGCUG | |
| | | | | | CAGGGTCTGGTGCGGAGAGGGCC | | | | GCCUCCGCAC | |
| | | | | | CACAGTGGACTTGGTGACGCTGT | | | | C | |
| | | | | | ATGCCCTCACCGCTCAGCCCCTG | | | | 1004TS: | 112 |
| | | | | | GGGATACTCCTAATCACACTTTTTT | | | | CUCCACCUCC | |
| | | | | | | | | | CCAGCCCGC | |
| | | | | | | | | | GCC | |
| | | | | | | | | | 1340TS: | 113 |
| | | | | | | | | | CCGGUGAGA | |
| | | | | | | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 12 | mir-675- | AACGCGCCC | 14 | U6.mir-675- | TCTAGAGATCCGACGCCGCCATCT | 59 | UGGUGCG | 95 | 527TS: | 108 |
| | 2.6 | CAGGGTCTG | | 2.6 | CTAGGCCCGCGCCGGCCCCCTCG | | GAGAGGG | | CGUGGGUCG | |
| | | GTGCGGAGA | | | CACAGACTTGTGGGAGAAGCTCG | | CCCACAG | | CCUUCGCCCA | |
| | | GGGCCCACA | | | GCTACTCCCCTGCCCCGGTTAATT | | UG | | C | |
| | | GTGGACTTG | | | TGCATATAATATTTCCTAGTAACTA | | | | 649TS: | 109 |
| | | GTGACGCTG | | | TAGAGGCTTAATGTGCGATAAAAG | | | | GCGCUGCAG | |
| | | TATGCCCTC | | | ACAGATAATCTGTTCTTTTTAATAC | | | | CCCAGCCAGG | |
| | | ACCGCTCAG | | | TAGCTACATTTTACATGATAGGCTT | | | | CCGCGCCGG | |
| | | CCCCTGGGG | | | GGATTTCTATAAGAGATACAAATA | | | | 668TS: | 110 |
| | | TAACTCCTAA | | | CTAAATTATTATTTTAAAAAACAGC | | | | UGCAGCCCAG | |
| | | TCACAC | | | ACAAAAGGAAACTCACCCTAACTG | | | | CCAGGCCGC | |
| | | | | | TAAAGTAATTGTGTGTTTTGAGACT | | | | GCCG | |
| | | | | | ATAAATATCCCTTGGAGAAAAGCC | | | | 780TS: | 111 |
| | | | | | TTGTTAACGCGCCCCAGGGTCTG | | | | UCCUCGCUG | |
| | | | | | GTGCGGAGAGGGCCCACAGTGGA | | | | GCCUCCGCAC | |
| | | | | | CTTGGTGACGCTGTATGCCCTCAC | | | | C | |
| | | | | | CGCTCAGCCCCTGGGGTAACTCC | | | | 1004TS: | 112 |
| | | | | | TAATCACACTTTTTT | | | | CUCCACCUCC | |
| | | | | | | | | | CCAGCCCGC | |
| | | | | | | | | | GCC | |
| | | | | | | | | | 1340TS: | 113 |
| | | | | | | | | | CCGGUGAGA | |
| | | | | | | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 13 | mir- | GCCCCAGGG | 15 | U6.mir- | TTTTAAAAGAAAAGGGGGGATTGG | 60 | UGGUGCG | 95 | 527TS: | 108 |
| | 675F2 | TCTGGTGCG | | 675F2 | GGGGTACAGTGCAGGGGAAAGAA | | GAGAGGG | | CGUGGGUCG | |
| | | GAGAGGGCC | | | TAGTAGACATAATAGCAACAGACA | | CCCACAG | | CCUUCGCCCA | |
| | | CACAGTGGA | | | TACAAACTAAAGAATTACAAAAACA | | UG | | C | |
| | | CTTGGTGAC | | | AATTACAAAAATTCAAAATTTTTCT | | | | 649TS: | 109 |
| | | GCTGTATGC | | | AGAGATCCGACGCCGCCATCTCT | | | | GCGCUGCAG | |
| | | CCTCACCGC | | | AGGCCCGCGCCGGCCCCCTCGCA | | | | CCCAGCCAGG | |
| | | TCAGCCCCT | | | CAGACTTGTGGGAGAAGCTCGGC | | | | CCGCGCCGG | |
| | | GGGGAATTC | | | TACTCCCCTGCCCCGGTTAATTTG | | | | 668TS: | 110 |
| | | TTCGATTCTG | | | CATATAATATTTCCTAGTAACTATA | | | | UGCAGCCCAG | |
| | | C | | | GAGGCTTAATGTGCGATAAAAGAC | | | | CCAGGCCGC | |
| | | | | | AGATAATCTGTTCTTTTTAATACTA | | | | GCCG | |
| | | | | | GCTACATTTTACATGATAGGCTTG | | | | 780TS: | 111 |
| | | | | | GATTTCTATAAGAGATACAAATACT | | | | UCCUCGCUG | |
| | | | | | AAATTATTATTTTAAAAAACAGCAC | | | | GCCUCCGCAC | |
| | | | | | AAAAGGAAACTCACCCTAACTGTA | | | | C | |
| | | | | | AAGTAATTGTGTGTTTTGAGACTAT | | | | 1004TS: | 112 |
| | | | | | AAATATCCCTTGGAGAAAAGCCTT | | | | CUCCACCUCC | |
| | | | | | GTTTGCCCCAGGGTCTGGTGCGG | | | | CCAGCCCGC | |
| | | | | | AGAGGGCCCACAGTGGACTTGGT | | | | GCC | |
| | | | | | GACGCTGTATGCCCTCACCGCTC | | | | 1340TS: | 113 |
| | | | | | AGCCCCTGGGGAATTCTTCGATTC | | | | CCGGUGAGA | |
| | | | | | TGCTTTTTT | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 14 | mir-675- | GCCCCAGGG | 16 | U6.mir-675- | TCTAGAGATCCGACGCCGCCATCT | 61 | UGGUGCG | 95 | 527TS: | 108 |
| | 2.1.1 | TCTGGTGCG | | 2.1.1 | CTAGGCCCGCGCCGGCCCCCTCG | | GAGAGGG | | CGUGGGUCG | |
| | | GAGAGGGCC | | | CACAGACTTGTGGGAGAAGCTCG | | CCCACAG | | CCUUCGCCCA | |
| | | CACAGTGGA | | | GCTACTCCCCTGCCCCGGTTAATT | | UG | | C | |
| | | CTTGGTGAC | | | TGCATATAATATTTCCTAGTAACTA | | | | 649TS: | 109 |
| | | GCTGTATGC | | | TAGAGGCTTAATGTGCGATAAAAG | | | | GCGCUGCAG | |
| | | CCTCACCGC | | | ACAGATAATCTGTTCTTTTTAATAC | | | | CCCAGCCAGG | |
| | | TCAGCCCCT | | | TAGCTACATTTTACATGATAGGCTT | | | | CCGCGCCGG | |
| | | GGGGATAAC | | | GGATTTCTATAAGAGATACAAATA | | | | 668TS: | 110 |
| | | TCCTAATCAC | | | CTAAATTATTATTTTAAAAAACAGC | | | | UGCAGCCCAG | |
| | | AC | | | ACAAAAGGAAACTCACCCTAACTG | | | | CCAGGCCGC | |
| | | | | | TAAAGTAATTGTGTGTTTTGAGACT | | | | GCCG | |
| | | | | | ATAAATATCCCTTGGAGAAAAGCC | | | | 780TS: | 111 |
| | | | | | TTGTTTGCCCCAGGGTCTGGTGC | | | | UCCUCGCUG | |
| | | | | | GGAGAGGGCCCACAGTGGACTTG | | | | GCCUCCGCAC | |
| | | | | | GTGACGCTGTATGCCCTCACCGC | | | | C | |
| | | | | | TCAGCCCCTGGGGATAACTCCTAA | | | | 1004TS: | 112 |
| | | | | | TCACACTTTTTT | | | | CUCCACCUCC | |
| | | | | | | | | | CCAGCCCGC | |
| | | | | | | | | | GCC | |
| | | | | | | | | | 1340TS: | 113 |
| | | | | | | | | | CCGGUGAGA | |
| | | | | | | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 15 | mir-675- | GAATCACAC | 17 | U6.mir-675- | TCTAGAGATCCGACGCCGCCATCT | 62 | UGGUGCG | 95 | 527TS: | 108 |
| | 2.3.1 | TGCCCCAGG | | 2.3.1 | CTAGGCCCGCGCCGGCCCCCTCG | | GAGAGGG | | CGUGGGUCG | |
| | | GTCTGGTGC | | | CACAGACTTGTGGGAGAAGCTCG | | CCCACAG | | CCUUCGCCCA | |
| | | GGAGAGGGC | | | GCTACTCCCCTGCCCCGGTTAATT | | UG | | C | |
| | | CCACAGTGG | | | TGCATATAATATTTCCTAGTAACTA | | | | 649TS: | 109 |
| | | ACTTGGTGA | | | TAGAGGCTTAATGTGCGATAAAAG | | | | GCGCUGCAG | |
| | | CGCTGTATG | | | ACAGATAATCTGTTCTTTTTAATAC | | | | CCCAGCCAGG | |
| | | CCCTCACCG | | | TAGCTACATTTTACATGATAGGCTT | | | | CCGCGCCGG | |
| | | CTCAGCCCC | | | GGATTTCTATAAGAGATACAAATA | | | | 668TS: | 110 |
| | | TGGGGATAA | | | CTAAATTATTATTTTAAAAAACAGC | | | | UGCAGCCCAG | |
| | | CTCCTAATCA | | | ACAAAAGGAAACTCACCCTAACTG | | | | CCAGGCCGC | |
| | | CAC | | | TAAAGTAATTGTGTGTTTTGAGACT | | | | GCCG | |
| | | | | | ATAAATATCCCTTGGAGAAAAGCC | | | | 780TS: | 111 |
| | | | | | TTGTTTGAATCACACTGCCCCAGG | | | | UCCUCGCUG | |
| | | | | | GTCTGGTGCGGAGAGGGCCCACA | | | | GCCUCCGCAC | |
| | | | | | GTGGACTTGGTGACGCTGTATGC | | | | C | |
| | | | | | CCTCACCGCTCAGCCCCTGGGGA | | | | 1004TS: | 112 |
| | | | | | TAACTCCTAATCACACTTTTTT | | | | CUCCACCUCC | |
| | | | | | | | | | CCAGCCCGC | |
| | | | | | | | | | GCC | |
| | | | | | | | | | 1340TS: | 113 |
| | | | | | | | | | CCGGUGAGA | |
| | | | | | | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 16 | mir- | GACCGTTTA | 18 | U6.mir-675H | TCTAGAGATCCGACGCCGCCATCT | 63 | UGGUGCG | 95 | 527TS: | 108 |
| | 675H | AACCCCAGG | | | CTAGGCCCGCGCCGGCCCCCTCG | | GAGAGGG | | CGUGGGUCG | |
| | | GTCTGGTGC | | | CACAGACTTGTGGGAGAAGCTCG | | CCCACAG | | CCUUCGCCCA | |
| | | GGAGAGGGC | | | GCTACTCCCCTGCCCCGGTTAATT | | UG | | C | |
| | | CCACAGTGG | | | TGCATATAATATTTCCTAGTAACTA | | | | 649TS: | 109 |
| | | ACTTGGTGA | | | TAGAGGCTTAATGTGCGATAAAAG | | | | GCGCUGCAG | |
| | | CGCTGTATG | | | ACAGATAATCTGTTCTTTTTAATAC | | | | CCCAGCCAGG | |
| | | CCCTCACCG | | | TAGCTACATTTTACATGATAGGCTT | | | | CCGCGCCGG | |
| | | CTCAGCCCC | | | GGATTTCTATAAGAGATACAAATA | | | | 668TS: | 110 |
| | | TGGGGCGCA | | | CTAAATTATTATTTTAAAAAACAGC | | | | UGCAGCCCAG | |
| | | CGCCAG | | | ACAAAAGGAAACTCACCCTAACTG | | | | CCAGGCCGC | |
| | | | | | TAAAGTAATTGTGTGTTTTGAGACT | | | | GCCG | |
| | | | | | ATAAATATCCCTTGGAGAAAAGCC | | | | 780TS: | 111 |
| | | | | | TTGTTTGACCGTTTAAACCCCAGG | | | | UCCUCGCUG | |
| | | | | | GTCTGGTGCGGAGAGGGCCCACA | | | | GCCUCCGCAC | |
| | | | | | GTGGACTTGGTGACGCTGTATGC | | | | C | |
| | | | | | CCTCACCGCTCAGCCCCTGGGGC | | | | 1004TS: | 112 |
| | | | | | GCACGCCAGTTTTTT | | | | CUCCACCUCC | |
| | | | | | | | | | CCAGCCCGC | |
| | | | | | | | | | GCC | |
| | | | | | | | | | 1340TS: | 113 |
| | | | | | | | | | CCGGUGAGA | |
| | | | | | | | | | GACUCCACAC | |
| | | | | | | | | | CG | |
| | | | | | | | | | 1471TS: | 114 |
| | | | | | | | | | CCGGUGAGA | |
| | | | | | | | | | GACUCCACAC | |
| | | | | | | | | | CG | |
| 17 | mi405F | GCTCGAGTG | 19 | U6.mi405F | ACGCCGCCATCTCTAGGCCCGCG | 64 | AAACCAG | 94 | 405TS: | 106 |
| | | AGCGATCCA | | | CCGGCCCCCTCGCACAGACTTGT | | AUCUGAA | | GUCCAGGAUU | |
| | | GGATTCAGA | | | GGGAGAAGCTCGGCTACTCCCCT | | UCCUGGA | | CAGAUCUGGU | |
| | | TCTGGTTTCT | | | GCCCCGGTTAATTTGCATATAATA | | C | | UU | |
| | | GTAAAGCCA | | | TTTCCTAGTAACTATAGAGGCTTA | | | | 193TS: | 107 |
| | | CAGATGGGA | | | ATGTGCGATAAAAGACAGATAATC | | | | CCAGGGUCCA | |
| | | AACCAGATC | | | TGTTCTTTTTAATACTAGCTACATT | | | | GAUUUGGUU | |
| | | TGAATCCTG | | | TTACATGATAGGCTTGGATTTCTAT | | | | U | |
| | | GACTGCCTA | | | AAGAGATACAAATACTAAATTATTA | | | | | |
| | | CTAGTAATTC | | | TTTTAAAAAACAGCACAAAAGGAA | | | | | |
| | | TTCGATTCTG | | | ACTCACCCTAACTGTAAAGTAATT | | | | | |
| | | C | | | GTGTGTTTTGAGACTATAAATATC | | | | | |
| | | | | | CCTTGGAGAAAAGCCTTGTTTGCT | | | | | |
| | | | | | CGAGTGAGCGATCCAGGATTCAG | | | | | |
| | | | | | ATCTGGTTTCTGTAAAGCCACAGA | | | | | |
| | | | | | TGGGAAACCAGATCTGAATCCTGG | | | | | |
| | | | | | ACTGCCTACTAGTAATTCTTCGATT | | | | | |
| | | | | | CTGCTTTTTT | | | | | |
| 18 | mi405NF | GCTCGAGTG | 20 | U6.mi405NF | ACGCCGCCATCTCTAGGCCCGCG | 65 | AAACCAG | 94 | 405TS: | 106 |
| | | AGCGATCCA | | | CCGGCCCCCTCGCACAGACTTGT | | AUCUGAA | | GUCCAGGAUU | |
| | | GGATTCAGA | | | GGGAGAAGCTCGGCTACTCCCCT | | UCCUGGA | | CAGAUCUGGU | |
| | | TCTGGTTTCT | | | GCCCCGGTTAATTTGCATATAATA | | C | | UU | |
| | | GTAAAGCCA | | | TTTCCTAGTAACTATAGAGGCTTA | | | | 193TS: | 107 |
| | | CAGATGGGA | | | ATGTGCGATAAAAGACAGATAATC | | | | CCAGGGUCCA | |
| | | AACCAGATC | | | TGTTCTTTTTAATACTAGCTACATT | | | | GAUUUGGUU | |
| | | TGAATCCTG | | | TTACATGATAGGCTTGGATTTCTAT | | | | U | |
| | | GACTGCCTA | | | AAGAGATACAAATACTAAATTATTA | | | | | |
| | | CTAGA | | | TTTTAAAAAACAGCACAAAAGGAA | | | | | |
| | | | | | ACTCACCCTAACTGTAAAGTAATT | | | | | |
| | | | | | GTGTGTTTTGAGACTATAAATATC | | | | | |
| | | | | | CCTTGGAGAAAAGCCTTGTTTGCT | | | | | |
| | | | | | CGAGTGAGCGATCCAGGATTCAG | | | | | |
| | | | | | ATCTGGTTTCTGTAAAGCCACAGA | | | | | |
| | | | | | TGGGAAACCAGATCTGAATCCTGG | | | | | |
| | | | | | ACTGCCTACTAGATTTTTT | | | | | |
| 19 | mi405A | GACCGTTTA | 21 | U6.mi405A | ACGCCGCCATCTCTAGGCCCGCG | 66 | AAACCAG | 94 | 405TS: | 106 |
| | | AACTCGAGT | | | CCGGCCCCCTCGCACAGACTTGT | | AUCUGAA | | GUCCAGGAUU | |
| | | GAGCGATCC | | | GGGAGAAGCTCGGCTACTCCCCT | | UCCUGGA | | CAGAUCUGGU | |
| | | AGGATTCAG | | | GCCCCGGTTAATTTGCATATAATA | | C | | UU | |
| | | ATCTGGTTTC | | | TTTCCTAGTAACTATAGAGGCTTA | | | | 193TS: | 107 |
| | | TGTAAAGCC | | | ATGTGCGATAAAAGACAGATAATC | | | | CCAGGGUCCA | |
| | | ACAGATGGG | | | TGTTCTTTTTAATACTAGCTACATT | | | | GAUUUGGUU | |
| | | AAACCAGAT | | | TTACATGATAGGCTTGGATTTCTAT | | | | U | |
| | | CTGAATCCT | | | AAGAGATACAAATACTAAATTATTA | | | | | |
| | | GGACTGCCT | | | TTTTAAAAAACAGCACAAAAGGAA | | | | | |
| | | ACTAGAGCG | | | ACTCACCCTAACTGTAAAGTAATT | | | | | |
| | | GCCGCCAC | | | GTGTGTTTTGAGACTATAAATATC | | | | | |
| | | | | | CCTTGGAGAAAAGCCTTGTTTGAC | | | | | |
| | | | | | CGTTTAAACTCGAGTGAGCGATCC | | | | | |
| | | | | | AGGATTCAGATCTGGTTTCTGTAA | | | | | |
| | | | | | AGCCACAGATGGGAAACCAGATC | | | | | |
| | | | | | TGAATCCTGGACTGCCTACTAGAG | | | | | |
| | | | | | CGGCCGCCACTTTTTT | | | | | |
| 20 | mi405B | GACCGTTTA | 22 | U6.mi405B | ACGCCGCCATCTCTAGGCCCGCG | 67 | AAACCAG | 94 | 405TS: | 106 |
| | | AACTCGAGT | | | CCGGCCCCCTCGCACAGACTTGT | | AUCUGAA | | GUCCAGGAUU | |
| | | GAGCGATCC | | | GGGAGAAGCTCGGCTACTCCCCT | | UCCUGGA | | CAGAUCUGGU | |
| | | AGGATTCAG | | | GCCCCGGTTAATTTGCATATAATA | | C | | UU | |
| | | ATCTGGTTTC | | | TTTCCTAGTAACTATAGAGGCTTA | | | | 193TS: | 107 |
| | | TGTAAAGCC | | | ATGTGCGATAAAAGACAGATAATC | | | | CCAGGGUCCA | |
| | | ACAGATGGG | | | TGTTCTTTTTAATACTAGCTACATT | | | | GAUUUGGUU | |
| | | AAACCAGAT | | | TTACATGATAGGCTTGGATTTCTAT | | | | U | |
| | | CTGAATCCT | | | AAGAGATACAAATACTAAATTATTA | | | | | |
| | | GGACTGCCT | | | TTTTAAAAAACAGCACAAAAGGAA | | | | | |
| | | ACTAGA | | | ACTCACCCTAACTGTAAAGTAATT | | | | | |
| | | | | | GTGTGTTTTGAGACTATAAATATC | | | | | |
| | | | | | CCTTGGAGAAAAGCCTTGTTTGAC | | | | | |
| | | | | | CGTTTAAACTCGAGTGAGCGATCC | | | | | |
| | | | | | AGGATTCAGATCTGGTTTCTGTAA | | | | | |
| | | | | | AGCCACAGATGGGAAACCAGATC | | | | | |
| | | | | | TGAATCCTGGACTGCCTACTAGAT | | | | | |
| | | | | | TTTTT | | | | | |
| 21 | mi405C | GCTCGAGTG | 23 | U6.mi405C | ACGCCGCCATCTCTAGGCCCGCG | 68 | AAACCAG | 94 | 405TS: | 106 |
| | | AGCGATCCA | | | CCGGCCCCCTCGCACAGACTTGT | | AUCUGAA | | GUCCAGGAUU | |
| | | GGATTCAGA | | | GGGAGAAGCTCGGCTACTCCCCT | | UCCUGGA | | CAGAUCUGGU | |
| | | TCTGGTTTCT | | | GCCCCGGTTAATTTGCATATAATA | | C | | UU | |
| | | GTAAAGCCA | | | TTTCCTAGTAACTATAGAGGCTTA | | | | 193TS: | 107 |
| | | CAGATGGGA | | | ATGTGCGATAAAAGACAGATAATC | | | | CCAGGGUCCA | |
| | | AACCAGATC | | | TGTTCTTTTTAATACTAGCTACATT | | | | GAUUUGGUU | |
| | | TGAATCCTG | | | TTACATGATAGGCTTGGATTTCTAT | | | | U | |
| | | GACTGCCTA | | | AAGAGATACAAATACTAAATTATTA | | | | | |
| | | CTAGAGCGG | | | TTTTAAAAAACAGCACAAAAGGAA | | | | | |
| | | CCGCCAC | | | ACTCACCCTAACTGTAAAGTAATT | | | | | |
| | | | | | GTGTGTTTTGAGACTATAAATATC | | | | | |
| | | | | | CCTTGGAGAAAAGCCTTGTTTGCT | | | | | |
| | | | | | CGAGTGAGCGATCCAGGATTCAG | | | | | |
| | | | | | ATCTGGTTTCTGTAAAGCCACAGA | | | | | |
| | | | | | TGGGAAACCAGATCTGAATCCTGG | | | | | |
| | | | | | ACTGCCTACTAGAGCGGCCGCCA | | | | | |
| | | | | | CTTTTTT | | | | | |
| 22 | mi405D | GACTCGAGT | 24 | U6.mi405D | ACGCCGCCATCTCTAGGCCCGCG | 69 | AAACCAG | 94 | 405TS: | 106 |
| | | GAGCGATCC | | | CCGGCCCCCTCGCACAGACTTGT | | AUCUGAA | | GUCCAGGAUU | |
| | | AGGATTCAG | | | GGGAGAAGCTCGGCTACTCCCCT | | UCCUGGA | | CAGAUCUGGU | |
| | | ATCTGGTTTC | | | GCCCCGGTTAATTTGCATATAATA | | C | | UU | |
| | | TGTAAAGCC | | | TTTCCTAGTAACTATAGAGGCTTA | | | | 193TS: | 107 |
| | | ACAGATGGG | | | ATGTGCGATAAAAGACAGATAATC | | | | CCAGGGUCCA | |
| | | AAACCAGAT | | | TGTTCTTTTTAATACTAGCTACATT | | | | GAUUUGGUU | |
| | | CTGAATCCT | | | TTACATGATAGGCTTGGATTTCTAT | | | | U | |
| | | GGACTGCCT | | | AAGAGATACAAATACTAAATTATTA | | | | | |
| | | ACTAGAGCG | | | TTTTAAAAAACAGCACAAAAGGAA | | | | | |
| | | GCCGCCAC | | | ACTCACCCTAACTGTAAAGTAATT | | | | | |
| | | | | | GTGTGTTTTGAGACTATAAATATC | | | | | |
| | | | | | CCTTGGAGAAAAGCCTTGTTTGAC | | | | | |
| | | | | | TCGAGTGAGCGATCCAGGATTCA | | | | | |
| | | | | | GATCTGGTTTCTGTAAAGCCACAG | | | | | |
| | | | | | ATGGGAAACCAGATCTGAATCCTG | | | | | |
| | | | | | GACTGCCTACTAGAGCGGCCGCC | | | | | |
| | | | | | ACTTTTTT | | | | | |
| 23 | mi405E | GACTCGAGT | 25 | U6.mi405E | ACGCCGCCATCTCTAGGCCCGCG | 70 | AAACCAG | 94 | 405TS: | 106 |
| | | GAGCGATCC | | | CCGGCCCCCTCGCACAGACTTGT | | AUCUGAA | | GUCCAGGAUU | |
| | | AGGATTCAG | | | GGGAGAAGCTCGGCTACTCCCCT | | UCCUGGA | | CAGAUCUGGU | |
| | | ATCTGGTTTC | | | GCCCCGGTTAATTTGCATATAATA | | C | | UU | |
| | | TGTAAAGCC | | | TTTCCTAGTAACTATAGAGGCTTA | | | | 193TS: | 107 |
| | | ACAGATGGG | | | ATGTGCGATAAAAGACAGATAATC | | | | CCAGGGUCCA | |
| | | AAACCAGAT | | | TGTTCTTTTTAATACTAGCTACATT | | | | GAUUUGGUU | |
| | | CTGAATCCT | | | TTACATGATAGGCTTGGATTTCTAT | | | | U | |
| | | GGACTGCCT | | | AAGAGATACAAATACTAAATTATTA | | | | | |
| | | ACTAGAGCG | | | TTTTAAAAAACAGCACAAAAGGAA | | | | | |
| | | CACGCCAG | | | ACTCACCCTAACTGTAAAGTAATT | | | | | |
| | | | | | GTGTGTTTTGAGACTATAAATATC | | | | | |
| | | | | | CCTTGGAGAAAAGCCTTGTTTGAC | | | | | |
| | | | | | TCGAGTGAGCGATCCAGGATTCA | | | | | |
| | | | | | GATCTGGTTTCTGTAAAGCCACAG | | | | | |
| | | | | | ATGGGAAACCAGATCTGAATCCTG | | | | | |
| | | | | | GACTGCCTACTAGAGCGCACGCC | | | | | |
| | | | | | AGTTTTTT | | | | | |
| 24 | mi405G | GACCGTTTA | 26 | U6.mi405G | ACGCCGCCATCTCTAGGCCCGCG | 71 | AAACCAG | 94 | 405TS: | 106 |
| | | AACTCGAGT | | | CCGGCCCCCTCGCACAGACTTGT | | AUCUGAA | | GUCCAGGAUU | |
| | | GAGCGATCC | | | GGGAGAAGCTCGGCTACTCCCCT | | UCCUGGA | | CAGAUCUGGU | |
| | | AGGATTCAG | | | GCCCCGGTTAATTTGCATATAATA | | C | | UU | |
| | | ATCTGGTTTC | | | TTTCCTAGTAACTATAGAGGCTTA | | | | 193TS: | 107 |
| | | TGTAAAGCC | | | ATGTGCGATAAAAGACAGATAATC | | | | CCAGGGUCCA | |
| | | ACAGATGGG | | | TGTTCTTTTTAATACTAGCTACATT | | | | GAUUUGGUU | |
| | | AAACCAGAT | | | TTACATGATAGGCTTGGATTTCTAT | | | | U | |
| | | CTGAATCCT | | | AAGAGATACAAATACTAAATTATTA | | | | | |
| | | GGACTGCCT | | | TTTTAAAAAACAGCACAAAAGGAA | | | | | |
| | | ACTAGAGAA | | | ACTCACCCTAACTGTAAAGTAATT | | | | | |
| | | TTCTTCGATT | | | GTGTGTTTTGAGACTATAAATATC | | | | | |
| | | CTGC | | | CCTTGGAGAAAAGCCTTGTTTGAC | | | | | |
| | | | | | CGTTTAAACTCGAGTGAGCGATCC | | | | | |
| | | | | | AGGATTCAGATCTGGTTTCTGTAA | | | | | |
| | | | | | AGCCACAGATGGGAAACCAGATC | | | | | |
| | | | | | TGAATCCTGGACTGCCTACTAGAG | | | | | |
| | | | | | AATTCTTCGATTCTGCTTTTTT | | | | | |
| 25 | mi405H | GACCGTTTA | 27 | U6.mi405H | ACGCCGCCATCTCTAGGCCCGCG | 72 | AAACCAG | 94 | 405TS: | 106 |
| | | AACTCGAGT | | | CCGGCCCCCTCGCACAGACTTGT | | AUCUGAA | | GUCCAGGAUU | |
| | | GAGCGATCC | | | GGGAGAAGCTCGGCTACTCCCCT | | UCCUGGA | | CAGAUCUGGU | |
| | | AGGATTCAG | | | GCCCCGGTTAATTTGCATATAATA | | C | | UU | |
| | | ATCTGGTTTC | | | TTTCCTAGTAACTATAGAGGCTTA | | | | 193TS: | 107 |
| | | TGTAAAGCC | | | ATGTGCGATAAAAGACAGATAATC | | | | CCAGGGUCCA | |
| | | ACAGATGGG | | | TGTTCTTTTTAATACTAGCTACATT | | | | GAUUUGGUU | |
| | | AAACCAGAT | | | TTACATGATAGGCTTGGATTTCTAT | | | | U | |
| | | CTGAATCCT | | | AAGAGATACAAATACTAAATTATTA | | | | | |
| | | GGACTGCCT | | | TTTTAAAAAACAGCACAAAAGGAA | | | | | |
| | | ACTAGAGCG | | | ACTCACCCTAACTGTAAAGTAATT | | | | | |
| | | CACGCCAG | | | GTGTGTTTTGAGACTATAAATATC | | | | | |
| | | | | | CCTTGGAGAAAAGCCTTGTTTGAC | | | | | |
| | | | | | CGTTTAAACTCGAGTGAGCGATCC | | | | | |
| | | | | | AGGATTCAGATCTGGTTTCTGTAA | | | | | |
| | | | | | AGCCACAGATGGGAAACCAGATC | | | | | |
| | | | | | TGAATCCTGGACTGCCTACTAGAG | | | | | |
| | | | | | CGCACGCCAGTTTTTT | | | | | |
| 26 | mi70 | GCGTTTAGT | 28 | U6.mi70 | ACGCCGCCATCTCTAGGCCCGCG | 73 | UUUGGCU | 96 | CGUUUGGAC | 115 |
| | | GAACCGTCA | | | CCGGCCCCCTCGCACAGACTTGT | | CGGGGUC | | CCCGAGCCAA | |
| | | GATGGTACC | | | GGGAGAAGCTCGGCTACTCCCCT | | CAAACGA | | ACU | |
| | | GTTTAAACTC | | | GCCCCGGTTAATTTGCATATAATA | | G | | | |
| | | GAGTGAGCG | | | TTTCCTAGTAACTATAGAGGCTTA | | | | | |
| | | ATCGTTTGG | | | ATGTGCGATAAAAGACAGATAATC | | | | | |
| | | ACCCCGAGC | | | TGTTCTTTTTAATACTAGCTACATT | | | | | |
| | | CAAACTGTAA | | | TTACATGATAGGCTTGGATTTCTAT | | | | | |
| | | AGCCACAGA | | | AAGAGATACAAATACTAAATTATTA | | | | | |
| | | TGGGTTTGG | | | TTTTAAAAAACAGCACAAAAGGAA | | | | | |
| | | CTCGGGGTC | | | ACTCACCCTAACTGTAAAGTAATT | | | | | |
| | | CAAACGAGT | | | GTGTGTTTTGAGACTATAAATATC | | | | | |
| | | GCCTACTAG | | | CCTTGGAGAAAAGCCTTGTTTGCG | | | | | |
| | | AGCGGCCGC | | | TTTAGTGAACCGTCAGATGGTACC | | | | | |
| | | CACAGCGGG | | | GTTTAAACTCGAGTGAGCGATCGT | | | | | |
| | | GAGATCCAG | | | TTGGACCCCGAGCCAAACTGTAAA | | | | | |
| | | ACATGATAA | | | GCCACAGATGGGTTTGGCTCGGG | | | | | |
| | | GATACA | | | GTCCAAACGAGTGCCTACTAGAG | | | | | |
| | | | | | CGGCCGCCACAGCGGGGAGATCC | | | | | |
| | | | | | AGACATGATAAGATACATTTTTT | | | | | |
| 27 | mi70F | GCTCGAGTG | 29 | U6.mi70F | CGCCGCCATCTCTAGGCCCGCGC | 74 | UUUGGCU | 96 | CGUUUGGAC | 115 |
| | | AGCGATCGT | | | CGGCCCCCTCGCACAGACTTGTG | | CGGGGUC | | CCCGAGCCAA | |
| | | TTGGACCCC | | | GGAGAAGCTCGGCTACTCCCCTG | | CAAACGA | | ACU | |
| | | GAGCCAAAC | | | CCCCGGTTAATTTGCATATAATATT | | G | | | |
| | | TGTAAAGCC | | | TCCTAGTAACTATAGAGGCTTAAT | | | | | |
| | | ACAGATGGG | | | GTGCGATAAAAGACAGATAATCTG | | | | | |
| | | TTTGGCTCG | | | TTCTTTTTAATACTAGCTACATTTT | | | | | |
| | | GGGTCCAAA | | | ACATGATAGGCTTGGATTTCTATA | | | | | |
| | | CGAGTGCCT | | | AGAGATACAAATACTAAATTATTAT | | | | | |
| | | ACTAGTAATT | | | TTTAAAAAACAGCACAAAAGGAAA | | | | | |
| | | CTTCGATTCT | | | CTCACCCTAACTGTAAAGTAATTG | | | | | |
| | | GC | | | TGTGTTTTGAGACTATAAATATCCC | | | | | |
| | | | | | TTGGAGAAAAGCCTTGTTTGCTCG | | | | | |
| | | | | | AGTGAGCGATCGTTTGGACCCCG | | | | | |
| | | | | | AGCCAAACTGTAAAGCCACAGATG | | | | | |
| | | | | | GGTTTGGCTCGGGGTCCAAACGA | | | | | |
| | | | | | GTGCCTACTAGTAATTCTTCGATT | | | | | |
| | | | | | CTGCTTTTTT | | | | | |
| 28 | mi185 | GCGTTTAGT | 30 | U6.mi185 | ACGCCGCCATCTCTAGGCCCGCG | 75 | UUCUGAA | 97 | GUCCAGAUUU | 116 |
| | | GAACCGTCA | | | CCGGCCCCCTCGCACAGACTTGT | | ACCAAAU | | GGUUUCAGAA | |
| | | GATGGTACC | | | GGGAGAAGCTCGGCTACTCCCCT | | CUGGACC | | CU | |
| | | GTTTAAACTC | | | GCCCCGGTTAATTTGCATATAATA | | C | | | |
| | | GAGTGAGCG | | | TTTCCTAGTAACTATAGAGGCTTA | | | | | |
| | | AGGTCCAGA | | | ATGTGCGATAAAAGACAGATAATC | | | | | |
| | | TTTGGTTTCA | | | TGTTCTTTTTAATACTAGCTACATT | | | | | |
| | | GAACTGTAA | | | TTACATGATAGGCTTGGATTTCTAT | | | | | |
| | | AGCCACAGA | | | AAGAGATACAAATACTAAATTATTA | | | | | |
| | | TGGGTTCTG | | | TTTTAAAAAACAGCACAAAAGGAA | | | | | |
| | | AAACCAAATC | | | ACTCACCCTAACTGTAAAGTAATT | | | | | |
| | | TGGACCCTG | | | GTGTGTTTTGAGACTATAAATATC | | | | | |
| | | CCTACTAGA | | | CCTTGGAGAAAAGCCTTGTTTGCG | | | | | |
| | | GCGGCCGCC | | | TTTAGTGAACCGTCAGATGGTACC | | | | | |
| | | ACAGCGGGG | | | GTTTAAACTCGAGTGAGCGAGGTC | | | | | |
| | | AGATCCAGA | | | CAGATTTGGTTTCAGAACTGTAAA | | | | | |
| | | CATGATAAG | | | GCCACAGATGGGTTCTGAAACCAA | | | | | |
| | | ATACA | | | ATCTGGACCCTGCCTACTAGAGC | | | | | |
| | | | | | GGCCGCCACAGCGGGGAGATCCA | | | | | |
| | | | | | GACATGATAAGATACATTTTTT | | | | | |
| 29 | mi185F | GCTCGAGTG | 31 | U6.mi185F | ACGCCGCCATCTCTAGGCCCGCG | 76 | UUCUGAA | 97 | GUCCAGAUUU | 116 |
| | | AGCGAGGTC | | | CCGGCCCCCTCGCACAGACTTGT | | ACCAAAU | | GGUUUCAGAA | |
| | | CAGATTTGG | | | GGGAGAAGCTCGGCTACTCCCCT | | CUGGACC | | CU | |
| | | TTTCAGAACT | | | GCCCCGGTTAATTTGCATATAATA | | C | | | |
| | | GTAAAGCCA | | | TTTCCTAGTAACTATAGAGGCTTA | | | | | |
| | | CAGATGGGT | | | ATGTGCGATAAAAGACAGATAATC | | | | | |
| | | TCTGAAACC | | | TGTTCTTTTTAATACTAGCTACATT | | | | | |
| | | AAATCTGGA | | | TTACATGATAGGCTTGGATTTCTAT | | | | | |
| | | CCCTGCCTA | | | AAGAGATACAAATACTAAATTATTA | | | | | |
| | | CTAGTAATTC | | | TTTTAAAAAACAGCACAAAAGGAA | | | | | |
| | | TTCGATTCTG | | | ACTCACCCTAACTGTAAAGTAATT | | | | | |
| | | C | | | GTGTGTTTTGAGACTATAAATATC | | | | | |
| | | | | | CCTTGGAGAAAAGCCTTGTTTGCT | | | | | |
| | | | | | CGAGTGAGCGAGGTCCAGATTTG | | | | | |
| | | | | | GTTTCAGAACTGTAAAGCCACAGA | | | | | |
| | | | | | TGGGTTCTGAAACCAAATCTGGAC | | | | | |
| | | | | | CCTGCCTACTAGTAATTCTTCGAT | | | | | |
| | | | | | TCTGCTTTTTT | | | | | |
| 30 | mi186 | GCGTTTAGT | 32 | U6.mi186 | GACGCCGCCATCTCTAGGCCCGC | 77 | AUUCUGA | 98 | UCCAGAUUUG | 117 |
| | | GAACCGTCA | | | GCCGGCCCCCTCGCACAGACTTG | | AACCAAA | | GUUUCAGAAU | |
| | | GATGGTACC | | | TGGGAGAAGCTCGGCTACTCCCC | | UCUGGAC | | CU | |
| | | GTTTAAACTC | | | TGCCCCGGTTAATTTGCATATAAT | | C | | | |
| | | GAGTGAGCG | | | ATTTCCTAGTAACTATAGAGGCTT | | | | | |
| | | AGTCCAGAT | | | AATGTGCGATAAAAGACAGATAAT | | | | | |
| | | TTGGTTTCAG | | | CTGTTCTTTTTAATACTAGCTACAT | | | | | |
| | | AATCTGTAAA | | | TTTACATGATAGGCTTGGATTTCTA | | | | | |
| | | GCCACAGAT | | | TAAGAGATACAAATACTAAATTATT | | | | | |
| | | GGGATTCTG | | | ATTTTAAAAAACAGCACAAAAGGA | | | | | |
| | | AAACCAAATC | | | AACTCACCCTAACTGTAAAGTAAT | | | | | |
| | | TGGACCTGC | | | TGTGTGTTTTGAGACTATAAATATC | | | | | |
| | | CTACTAGAG | | | CCTTGGAGAAAAGCCTTGTTTGCG | | | | | |
| | | CGGCCGCCA | | | TTTAGTGAACCGTCAGATGGTACC | | | | | |
| | | CAGCGGGGA | | | GTTTAAACTCGAGTGAGCGAGTCC | | | | | |
| | | GATCCAGAC | | | AGATTTGGTTTCAGAATCTGTAAA | | | | | |
| | | ATGATAAGAT | | | GCCACAGATGGGATTCTGAAACCA | | | | | |
| | | ACA | | | AATCTGGACCTGCCTACTAGAGCG | | | | | |
| | | | | | GCCGCCACAGCGGGGAGATCCAG | | | | | |
| | | | | | ACATGATAAGATACATTTTTT | | | | | |
| 31 | mi186F | GCTCGAGTG | 33 | U6.mi186F | ACGCCGCCATCTCTAGGCCCGCG | 78 | AUUCUGA | 98 | UCCAGAUUUG | 117 |
| | | AGCGAGTCC | | | CCGGCCCCCTCGCACAGACTTGT | | AACCAAA | | GUUUCAGAAU | |
| | | AGATTTGGTT | | | GGGAGAAGCTCGGCTACTCCCCT | | UCUGGAC | | CU | |
| | | TCAGAATCT | | | GCCCCGGTTAATTTGCATATAATA | | C | | | |
| | | GTAAAGCCA | | | TTTCCTAGTAACTATAGAGGCTTA | | | | | |
| | | CAGATGGGA | | | ATGTGCGATAAAAGACAGATAATC | | | | | |
| | | TTCTGAAACC | | | TGTTCTTTTTAATACTAGCTACATT | | | | | |
| | | AAATCTGGA | | | TTACATGATAGGCTTGGATTTCTAT | | | | | |
| | | CCTGCCTAC | | | AAGAGATACAAATACTAAATTATTA | | | | | |
| | | TAGTAATTCT | | | TTTTAAAAAACAGCACAAAAGGAA | | | | | |
| | | TCGATTCTG | | | ACTCACCCTAACTGTAAAGTAATT | | | | | |
| | | C | | | GTGTGTTTTGAGACTATAAATATC | | | | | |
| | | | | | CCTTGGAGAAAAGCCTTGTTTGCT | | | | | |
| | | | | | CGAGTGAGCGAGTCCAGATTTGG | | | | | |
| | | | | | TTTCAGAATCTGTAAAGCCACAGA | | | | | |
| | | | | | TGGGATTCTGAAACCAAATCTGGA | | | | | |
| | | | | | CCTGCCTACTAGTAATTCTTCGAT | | | | | |
| | | | | | TCTGCTTTTTT | | | | | |
| 32 | mi318 | GCGTTTAGT | 34 | U6.mi318 | ACGCCGCCATCTCTAGGCCCGCG | 79 | AAAGGCT | 99 | CCCUGCUCCU | 118 |
| | | GAACCGTCA | | | CCGGCCCCCTCGCACAGACTTGT | | CGGAGGA | | CCGAGCCUUU | |
| | | GATGGTACC | | | GGGAGAAGCTCGGCTACTCCCCT | | GCAGGGC | | CU | |
| | | GTTTAAACTC | | | GCCCCGGTTAATTTGCATATAATA | | G | | | |
| | | GAGTGAGCG | | | TTTCCTAGTAACTATAGAGGCTTA | | | | | |
| | | AGCCCTGCT | | | ATGTGCGATAAAAGACAGATAATC | | | | | |
| | | CCTCCGAGC | | | TGTTCTTTTTAATACTAGCTACATT | | | | | |
| | | CTTTCTGAAA | | | TTACATGATAGGCTTGGATTTCTAT | | | | | |
| | | GCCACAGAT | | | AAGAGATACAAATACTAAATTATTA | | | | | |
| | | GGGAAAGGC | | | TTTTAAAAAACAGCACAAAAGGAA | | | | | |
| | | TCGGAGGAG | | | ACTCACCCTAACTGTAAAGTAATT | | | | | |
| | | CAGGGCGTG | | | GTGTGTTTTGAGACTATAAATATC | | | | | |
| | | CCACTAGAG | | | CCTTGGAGAAAAGCCTTGTTTGCG | | | | | |
| | | CGGCCGCCA | | | TTTAGTGAACCGTCAGATGGTACC | | | | | |
| | | CAGCGGGGA | | | GTTTAAACTCGAGTGAGCGAGCC | | | | | |
| | | GATCCAGAC | | | CTGCTCCTCCGAGCCTTTCTGAAA | | | | | |
| | | ATGATAAGAT | | | GCCACAGATGGGAAAGGCTCGGA | | | | | |
| | | ACA | | | GGAGCAGGGCGTGCCACTAGAGC | | | | | |
| | | | | | GGCCGCCACAGCGGGGAGATCCA | | | | | |
| | | | | | GACATGATAAGATACATTTTTT | | | | | |
| 33 | mi318F | GCTCGAGTG | 35 | U6.mi318F | ACGCCGCCATCTCTAGGCCCGCG | 80 | AAAGGCT | 99 | CCCUGCUCCU | 118 |
| | | AGCGAGCCC | | | CCGGCCCCCTCGCACAGACTTGT | | CGGAGGA | | CCGAGCCUUU | |
| | | TGCTCCTCC | | | GGGAGAAGCTCGGCTACTCCCCT | | GCAGGGC | | CU | |
| | | GAGCCTTTC | | | GCCCCGGTTAATTTGCATATAATA | | G | | | |
| | | TGAAAGCCA | | | TTTCCTAGTAACTATAGAGGCTTA | | | | | |
| | | CAGATGGGA | | | ATGTGCGATAAAAGACAGATAATC | | | | | |
| | | AAGGCTCGG | | | TGTTCTTTTTAATACTAGCTACATT | | | | | |
| | | AGGAGCAGG | | | TTACATGATAGGCTTGGATTTCTAT | | | | | |
| | | GCGTGCCAC | | | AAGAGATACAAATACTAAATTATTA | | | | | |
| | | TAGTAATTCT | | | TTTTAAAAAACAGCACAAAAGGAA | | | | | |
| | | TCGATTCTG | | | ACTCACCCTAACTGTAAAGTAATT | | | | | |
| | | C | | | GTGTGTTTTGAGACTATAAATATC | | | | | |
| | | | | | CCTTGGAGAAAAGCCTTGTTTGCT | | | | | |
| | | | | | CGAGTGAGCGAGCCCTGCTCCTC | | | | | |
| | | | | | CGAGCCTTTCTGAAAGCCACAGAT | | | | | |
| | | | | | GGGAAAGGCTCGGAGGAGCAGG | | | | | |
| | | | | | GCGTGCCACTAGTAATTCTTCGAT | | | | | |
| | | | | | TCTGCTTTTTT | | | | | |
| 34 | mi333 | GCGTTTAGT | 36 | U6.mi333 | ACGCCGCCATCTCTAGGCCCGCG | 81 | AAAGCGA | 100 | CCUUUGAGAA | 119 |
| | | GAACCGTCA | | | CCGGCCCCCTCGCACAGACTTGT | | UCCUUCU | | GGAUCGCUU | |
| | | GATGGTACC | | | GGGAGAAGCTCGGCTACTCCCCT | | CAAAGGC | | UCU | |
| | | GTTTAAACTC | | | GCCCCGGTTAATTTGCATATAATA | | U | | | |
| | | GAGTGAGCG | | | TTTCCTAGTAACTATAGAGGCTTA | | | | | |
| | | CGCCTTTGA | | | ATGTGCGATAAAAGACAGATAATC | | | | | |
| | | GAAGGATCG | | | TGTTCTTTTTAATACTAGCTACATT | | | | | |
| | | CTTTCTGTAA | | | TTACATGATAGGCTTGGATTTCTAT | | | | | |
| | | AGCCACAGA | | | AAGAGATACAAATACTAAATTATTA | | | | | |
| | | TGGGAAAGC | | | TTTTAAAAAACAGCACAAAAGGAA | | | | | |
| | | GATCCTTCTC | | | ACTCACCCTAACTGTAAAGTAATT | | | | | |
| | | AAAGGCTTG | | | GTGTGTTTTGAGACTATAAATATC | | | | | |
| | | CCTACTAGA | | | CCTTGGAGAAAAGCCTTGTTTGCG | | | | | |
| | | GCGGCCGCC | | | TTTAGTGAACCGTCAGATGGTACC | | | | | |
| | | ACAGCGGGG | | | GTTTAAACTCGAGTGAGCGCGCCT | | | | | |
| | | AGATCCAGA | | | TTGAGAAGGATCGCTTTCTGTAAA | | | | | |
| | | CATGATAAG | | | GCCACAGATGGGAAAGCGATCCT | | | | | |
| | | ATACA | | | TCTCAAAGGCTTGCCTACTAGAGC | | | | | |
| | | | | | GGCCGCCACAGCGGGGAGATCCA | | | | | |
| | | | | | GACATGATAAGATACATTTTTTG | | | | | |
| 35 | mi333F | GCTCGAGTG | 37 | U6.mi333F | ACGCCGCCATCTCTAGGCCCGCG | 82 | AAAGCGA | 100 | CCUUUGAGAA | 119 |
| | | AGCGCGCCT | | | CCGGCCCCCTCGCACAGACTTGT | | UCCUUCU | | GGAUCGCUU | |
| | | TTGAGAAGG | | | GGGAGAAGCTCGGCTACTCCCCT | | CAAAGGC | | UCU | |
| | | ATCGCTTTCT | | | GCCCCGGTTAATTTGCATATAATA | | U | | | |
| | | GTAAAGCCA | | | TTTCCTAGTAACTATAGAGGCTTA | | | | | |
| | | CAGATGGGA | | | ATGTGCGATAAAAGACAGATAATC | | | | | |
| | | AAGCGATCC | | | TGTTCTTTTTAATACTAGCTACATT | | | | | |
| | | TTCTCAAAG | | | TTACATGATAGGCTTGGATTTCTAT | | | | | |
| | | GCTTGCCTA | | | AAGAGATACAAATACTAAATTATTA | | | | | |
| | | CTAGTAATTC | | | TTTTAAAAAACAGCACAAAAGGAA | | | | | |
| | | TTCGATTCTG | | | ACTCACCCTAACTGTAAAGTAATT | | | | | |
| | | C | | | GTGTGTTTTGAGACTATAAATATC | | | | | |
| | | | | | CCTTGGAGAAAAGCCTTGTTTGCT | | | | | |
| | | | | | CGAGTGAGCGCGCCTTTGAGAAG | | | | | |
| | | | | | GATCGCTTTCTGTAAAGCCACAGA | | | | | |
| | | | | | TGGGAAAGCGATCCTTCTCAAAGG | | | | | |
| | | | | | CTTGCCTACTAGTAATTCTTCGATT | | | | | |
| | | | | | CTGCTTTTTT | | | | | |
| 36 | mi599 | GCGTTTAGT | 38 | U6.mi599 | ACGCCGCCATCTCTAGGCCCGCG | 83 | AAAGCCC | 101 | GCUCUCCCAC | 120 |
| | | GAACCGTCA | | | CCGGCCCCCTCGCACAGACTTGT | | CCUGUGG | | AGGGGGCUU | |
| | | GATGGTACC | | | GGGAGAAGCTCGGCTACTCCCCT | | GAGAGCC | | UCU | |
| | | GTTTAAACTC | | | GCCCCGGTTAATTTGCATATAATA | | C | | | |
| | | GAGTGAGCA | | | TTTCCTAGTAACTATAGAGGCTTA | | | | | |
| | | GAGGCTCTC | | | ATGTGCGATAAAAGACAGATAATC | | | | | |
| | | CCACAGGGG | | | TGTTCTTTTTAATACTAGCTACATT | | | | | |
| | | GCTTTCTGAA | | | TTACATGATAGGCTTGGATTTCTAT | | | | | |
| | | AGCCACAGA | | | AAGAGATACAAATACTAAATTATTA | | | | | |
| | | TGGGAAAGC | | | TTTTAAAAAACAGCACAAAAGGAA | | | | | |
| | | CCCCTGTGG | | | ACTCACCCTAACTGTAAAGTAATT | | | | | |
| | | GAGAGCCCT | | | GTGTGTTTTGAGACTATAAATATC | | | | | |
| | | GCCTACTAG | | | CCTTGGAGAAAAGCCTTGTTTGCG | | | | | |
| | | AGCGGCCGC | | | TTTAGTGAACCGTCAGATGGTACC | | | | | |
| | | CACAGCGGG | | | GTTTAAACTCGAGTGAGCAGAGG | | | | | |
| | | GAGATCCAG | | | CTCTCCCACAGGGGGCTTTCTGAA | | | | | |
| | | ACATGATAA | | | AGCCACAGATGGGAAAGCCCCCT | | | | | |
| | | GATACA | | | GTGGGAGAGCCCTGCCTACTAGA | | | | | |
| | | | | | GCGGCCGCCACAGCGGGGAGAT | | | | | |
| | | | | | CCAGACATGATAAGATACATTTTTT | | | | | |
| 37 | mi599F | GCTCGAGTG | 39 | U6.mi599F | ACGCCGCCATCTCTAGGCCCGCG | 84 | AAAGCCC | 101 | GCUCUCCCAC | 120 |
| | | AGCAGAGGC | | | CCGGCCCCCTCGCACAGACTTGT | | CCUGUGG | | AGGGGGCUU | |
| | | TCTCCCACA | | | GGGAGAAGCTCGGCTACTCCCCT | | GAGAGCC | | UCU | |
| | | GGGGGCTTT | | | GCCCCGGTTAATTTGCATATAATA | | C | | | |
| | | CTGAAAGCC | | | TTTCCTAGTAACTATAGAGGCTTA | | | | | |
| | | ACAGATGGG | | | ATGTGCGATAAAAGACAGATAATC | | | | | |
| | | AAAGCCCCC | | | TGTTCTTTTTAATACTAGCTACATT | | | | | |
| | | TGTGGGAGA | | | TTACATGATAGGCTTGGATTTCTAT | | | | | |
| | | GCCCTGCCT | | | AAGAGATACAAATACTAAATTATTA | | | | | |
| | | ACTAGTAATT | | | TTTTAAAAAACAGCACAAAAGGAA | | | | | |
| | | CTTCGATTCT | | | ACTCACCCTAACTGTAAAGTAATT | | | | | |
| | | GC | | | GTGTGTTTTGAGACTATAAATATC | | | | | |
| | | | | | CCTTGGAGAAAAGCCTTGTTTGCT | | | | | |
| | | | | | CGAGTGAGCAGAGGCTCTCCCAC | | | | | |
| | | | | | AGGGGGCTTTCTGAAAGCCACAG | | | | | |
| | | | | | ATGGGAAAGCCCCCTGTGGGAGA | | | | | |
| | | | | | GCCCTGCCTACTAGTAATTCTTCG | | | | | |
| | | | | | ATTCTGCTTTTTT | | | | | |
| 38 | mi1155 | GCGTTTAGT | 40 | U6.mi1155 | GACGCCGCCATCTCTAGGCCCGC | 85 | UUCUAGG | 102 | AGGCGCAACC | 121 |
| | | GAACCGTCA | | | GCCGGCCCCCTCGCACAGACTTG | | AGAGGUU | | UCUCCUAGAA | |
| | | GATGGTACC | | | TGGGAGAAGCTCGGCTACTCCCC | | GCGCCUG | | CU | |
| | | GTTTAAACTC | | | TGCCCCGGTTAATTTGCATATAAT | | C | | | |
| | | GAGTGAGCG | | | ATTTCCTAGTAACTATAGAGGCTT | | | | | |
| | | ACAGGCGCA | | | AATGTGCGATAAAAGACAGATAAT | | | | | |
| | | ACCTCTCCTA | | | CTGTTCTTTTTAATACTAGCTACAT | | | | | |
| | | GAACTGTAA | | | TTTACATGATAGGCTTGGATTTCTA | | | | | |
| | | AGCCACAGA | | | TAAGAGATACAAATACTAAATTATT | | | | | |
| | | TGGGTTCTA | | | ATTTTAAAAAACAGCACAAAAGGA | | | | | |
| | | GGAGAGGTT | | | AACTCACCCTAACTGTAAAGTAAT | | | | | |
| | | GCGCCTGCT | | | TGTGTGTTTTGAGACTATAAATATC | | | | | |
| | | GCCTACTAG | | | CCTTGGAGAAAAGCCTTGTTTGCG | | | | | |
| | | AGCGGCCGC | | | TTTAGTGAACCGTCAGATGGTACC | | | | | |
| | | CACAGCGGG | | | GTTTAAACTCGAGTGAGCGACAG | | | | | |
| | | GAGATCCAG | | | GCGCAACCTCTCCTAGAACTGTAA | | | | | |
| | | ACATGATAA | | | AGCCACAGATGGGTTCTAGGAGA | | | | | |
| | | GATACA | | | GGTTGCGCCTGCTGCCTACTAGA | | | | | |
| | | | | | GCGGCCGCCACAGCGGGGAGAT | | | | | |
| | | | | | CCAGACATGATAAGATACATTTTTT | | | | | |
| 39 | mi1155F | GCTCGAGTG | 41 | U6.mi1155F | ACGCCGCCATCTCTAGGCCCGCG | 86 | UUCUAGG | 102 | AGGCGCAACC | 121 |
| | | AGCGACAGG | | | CCGGCCCCCTCGCACAGACTTGT | | AGAGGUU | | UCUCCUAGAA | |
| | | CGCAACCTC | | | GGGAGAAGCTCGGCTACTCCCCT | | GCGCCUG | | CU | |
| | | TCCTAGAACT | | | GCCCCGGTTAATTTGCATATAATA | | C | | | |
| | | GTAAAGCCA | | | TTTCCTAGTAACTATAGAGGCTTA | | | | | |
| | | CAGATGGGT | | | ATGTGCGATAAAAGACAGATAATC | | | | | |
| | | TCTAGGAGA | | | TGTTCTTTTTAATACTAGCTACATT | | | | | |
| | | GGTTGCGCC | | | TTACATGATAGGCTTGGATTTCTAT | | | | | |
| | | TGCTGCCTA | | | AAGAGATACAAATACTAAATTATTA | | | | | |
| | | CTAGTAATTC | | | TTTTAAAAAACAGCACAAAAGGAA | | | | | |
| | | TTCGATTCTG | | | ACTCACCCTAACTGTAAAGTAATT | | | | | |
| | | C | | | GTGTGTTTTGAGACTATAAATATC | | | | | |
| | | | | | CCTTGGAGAAAAGCCTTGTTTGCT | | | | | |
| | | | | | CGAGTGAGCGACAGGCGCAACCT | | | | | |
| | | | | | CTCCTAGAACTGTAAAGCCACAGA | | | | | |
| | | | | | TGGGTTCTAGGAGAGGTTGCGCC | | | | | |
| | | | | | TGCTGCCTACTAGTAATTCTTCGA | | | | | |
| | | | | | TTCTGCTTTTTT | | | | | |
| 40 | mi1156 | GCGTTTAGT | 42 | U6.mi1156 | ACGCCGCCATCTCTAGGCCCGCG | 87 | UUUCUAG | 103 | GGCGCAACCU | 122 |
| | | GAACCGTCA | | | CCGGCCCCCTCGCACAGACTTGT | | GAGAGGU | | CUCCUAGAAA | |
| | | GATGGTACC | | | GGGAGAAGCTCGGCTACTCCCCT | | UGCGCCU | | CU | |
| | | GTTTAAACTC | | | GCCCCGGTTAATTTGCATATAATA | | G | | | |
| | | GAGTGAGCG | | | TTTCCTAGTAACTATAGAGGCTTA | | | | | |
| | | AAGGCGCAA | | | ATGTGCGATAAAAGACAGATAATC | | | | | |
| | | CCTCTCCTA | | | TGTTCTTTTTAATACTAGCTACATT | | | | | |
| | | GAAACTGAA | | | TTACATGATAGGCTTGGATTTCTAT | | | | | |
| | | AGCCACAGA | | | AAGAGATACAAATACTAAATTATTA | | | | | |
| | | TGGGTTTCTA | | | TTTTAAAAAACAGCACAAAAGGAA | | | | | |
| | | GGAGAGGTT | | | ACTCACCCTAACTGTAAAGTAATT | | | | | |
| | | GCGCCTGTG | | | GTGTGTTTTGAGACTATAAATATC | | | | | |
| | | CCTACTAGA | | | CCTTGGAGAAAAGCCTTGTTTGCG | | | | | |
| | | GCGGCCGCC | | | TTTAGTGAACCGTCAGATGGTACC | | | | | |
| | | ACAGCGGGG | | | GTTTAAACTCGAGTGAGCGAAGG | | | | | |
| | | AGATCCAGA | | | CGCAACCTCTCCTAGAAACTGAAA | | | | | |
| | | CATGATAAG | | | GCCACAGATGGGTTTCTAGGAGA | | | | | |
| | | ATACA | | | GGTTGCGCCTGTGCCTACTAGAG | | | | | |
| | | | | | CGGCCGCCACAGCGGGGAGATCC | | | | | |
| | | | | | AGACATGATAAGATACATTTTTT | | | | | |
| 41 | mi1156F | GCTCGAGTG | 43 | U6.mi1156F | ACGCCGCCATCTCTAGGCCCGCG | 88 | UUUCUAG | 103 | GGCGCAACCU | 122 |
| | | AGCGAAGGC | | | CCGGCCCCCTCGCACAGACTTGT | | GAGAGGU | | CUCCUAGAAA | |
| | | GCAACCTCT | | | GGGAGAAGCTCGGCTACTCCCCT | | UGCGCCU | | CU | |
| | | CCTAGAAAC | | | GCCCCGGTTAATTTGCATATAATA | | G | | | |
| | | TGAAAGCCA | | | TTTCCTAGTAACTATAGAGGCTTA | | | | | |
| | | CAGATGGGT | | | ATGTGCGATAAAAGACAGATAATC | | | | | |
| | | TTCTAGGAG | | | TGTTCTTTTTAATACTAGCTACATT | | | | | |
| | | AGGTTGCGC | | | TTACATGATAGGCTTGGATTTCTAT | | | | | |
| | | CTGTGCCTA | | | AAGAGATACAAATACTAAATTATTA | | | | | |
| | | CTAGTAATTC | | | TTTTAAAAAACAGCACAAAAGGAA | | | | | |
| | | TTCGATTCTG | | | ACTCACCCTAACTGTAAAGTAATT | | | | | |
| | | C | | | GTGTGTTTTGAGACTATAAATATC | | | | | |
| | | | | | CCTTGGAGAAAAGCCTTGTTTGCT | | | | | |
| | | | | | CGAGTGAGCGAAGGCGCAACCTC | | | | | |
| | | | | | TCCTAGAAACTGAAAGCCACAGAT | | | | | |
| | | | | | GGGTTTCTAGGAGAGGTTGCGCC | | | | | |
| | | | | | TGTGCCTACTAGTAATTCTTCGATT | | | | | |
| | | | | | CTGCTTTTTT | | | | | |
| 42 | mi1230 | GCGTTTAGT | 44 | U6.mi1230 | GACGCCGCCATCTCTAGGCCCGC | 89 | GUAUUCU | 104 | CCCUCAGCGA | 123 |
| | | GAACCGTCA | | | GCCGGCCCCCTCGCACAGACTTG | | UCCUCGC | | GGAAGAAUAC | |
| | | GATGGTACC | | | TGGGAGAAGCTCGGCTACTCCCC | | UGAGGGG | | CU | |
| | | GTTTAAACTC | | | TGCCCCGGTTAATTTGCATATAAT | | U | | | |
| | | GAGTGAGCG | | | ATTTCCTAGTAACTATAGAGGCTT | | | | | |
| | | CCCCCTCAG | | | AATGTGCGATAAAAGACAGATAAT | | | | | |
| | | CGAGGAAGA | | | CTGTTCTTTTTAATACTAGCTACAT | | | | | |
| | | ATACCTGTAA | | | TTTACATGATAGGCTTGGATTTCTA | | | | | |
| | | AGCCACAGA | | | TAAGAGATACAAATACTAAATTATT | | | | | |
| | | TGGGGTATT | | | ATTTTAAAAAACAGCACAAAAGGA | | | | | |
| | | CTTCCTCGC | | | AACTCACCCTAACTGTAAAGTAAT | | | | | |
| | | TGAGGGGTT | | | TGTGTGTTTTGAGACTATAAATATC | | | | | |
| | | GCCTACTAG | | | CCTTGGAGAAAAGCCTTGTTTGCG | | | | | |
| | | AGCGGCCGC | | | TTTAGTGAACCGTCAGATGGTACC | | | | | |
| | | CACAGCGGG | | | GTTTAAACTCGAGTGAGCGCCCC | | | | | |
| | | GAGATCCAG | | | CTCAGCGAGGAAGAATACCTGTAA | | | | | |
| | | ACATGATAA | | | AGCCACAGATGGGGTATTCTTCCT | | | | | |
| | | GATACA | | | CGCTGAGGGGTTGCCTACTAGAG | | | | | |
| | | | | | CGGCCGCCACAGCGGGGAGATCC | | | | | |
| | | | | | AGACATGATAAGATACATTTTTT | | | | | |
| 43 | mi1230F | GCTCGAGTG | 45 | U6.mi1230F | ACGCCGCCATCTCTAGGCCCGCG | 90 | GUAUUCU | 104 | CCCUCAGCGA | 123 |
| | | AGCGCCCCC | | | CCGGCCCCCTCGCACAGACTTGT | | UCCUCGC | | GGAAGAAUAC | |
| | | TCAGCGAGG | | | GGGAGAAGCTCGGCTACTCCCCT | | UGAGGGG | | CU | |
| | | AAGAATACCT | | | GCCCCGGTTAATTTGCATATAATA | | U | | | |
| | | GTAAAGCCA | | | TTTCCTAGTAACTATAGAGGCTTA | | | | | |
| | | CAGATGGGG | | | ATGTGCGATAAAAGACAGATAATC | | | | | |
| | | TATTCTTCCT | | | TGTTCTTTTTAATACTAGCTACATT | | | | | |
| | | CGCTGAGGG | | | TTACATGATAGGCTTGGATTTCTAT | | | | | |
| | | GTTGCCTAC | | | AAGAGATACAAATACTAAATTATTA | | | | | |
| | | TAGTAATTCT | | | TTTTAAAAAACAGCACAAAAGGAA | | | | | |
| | | TCGATTCTG | | | ACTCACCCTAACTGTAAAGTAATT | | | | | |
| | | C | | | GTGTGTTTTGAGACTATAAATATC | | | | | |
| | | | | | CCTTGGAGAAAAGCCTTGTTTGCT | | | | | |
| | | | | | CGAGTGAGCGCCCCCTCAGCGAG | | | | | |
| | | | | | GAAGAATACCTGTAAAGCCACAGA | | | | | |
| | | | | | TGGGGTATTCTTCCTCGCTGAGG | | | | | |
| | | | | | GGTTGCCTACTAGTAATTCTTCGA | | | | | |
| | | | | | TTCTGCTTTTTTC | | | | | |
| 44 | mi1311 | GCGTTTAGT | 46 | U6.mi1311 | GACGCCGCCATCTCTAGGCCCGC | 91 | GAAAGAA | 105 | CGGAGAACUG | 124 |
| | | GAACCGTCA | | | GCCGGCCCCCTCGCACAGACTTG | | UGGCAGU | | CCAUUCUUUC | |
| | | GATGGTACC | | | TGGGAGAAGCTCGGCTACTCCCC | | UCUCCGC | | CU | |
| | | GTTTAAACTC | | | TGCCCCGGTTAATTTGCATATAAT | | G | | | |
| | | GAGTGAGCG | | | ATTTCCTAGTAACTATAGAGGCTT | | | | | |
| | | AGCGGAGAA | | | AATGTGCGATAAAAGACAGATAAT | | | | | |
| | | CTGCCATTCT | | | CTGTTCTTTTTAATACTAGCTACAT | | | | | |
| | | TTCCTGTAAA | | | TTTACATGATAGGCTTGGATTTCTA | | | | | |
| | | GCCACAGAT | | | TAAGAGATACAAATACTAAATTATT | | | | | |
| | | GGGGAAAGA | | | ATTTTAAAAAACAGCACAAAAGGA | | | | | |
| | | ATGGCAGTT | | | AACTCACCCTAACTGTAAAGTAAT | | | | | |
| | | CTCCGCGTG | | | TGTGTGTTTTGAGACTATAAATATC | | | | | |
| | | CCTACTAGA | | | CCTTGGAGAAAAGCCTTGTTTGCG | | | | | |
| | | GCGGCCGCC | | | TTTAGTGAACCGTCAGATGGTACC | | | | | |
| | | ACAGCGGGG | | | GTTTAAACTCGAGTGAGCGAGCG | | | | | |
| | | AGATCCAGA | | | GAGAACTGCCATTCTTTCCTGTAA | | | | | |
| | | CATGATAAG | | | AGCCACAGATGGGGAAAGAATGG | | | | | |
| | | ATACA | | | CAGTTCTCCGCGTGCCTACTAGAG | | | | | |
| | | | | | CGGCCGCCACAGCGGGGAGATCC | | | | | |
| | | | | | AGACATGATAAGATACATTTTTT | | | | | |
| 45 | mi1311F | GCTCGAGTG | 47 | U6.mi1311F | ACGCCGCCATCTCTAGGCCCGCG | 92 | GAAAGAA | 105 | CGGAGAACUG | 124 |
| | | AGCGAGCGG | | | CCGGCCCCCTCGCACAGACTTGT | | UGGCAGU | | CCAUUCUUUC | |
| | | AGAACTGCC | | | GGGAGAAGCTCGGCTACTCCCCT | | UCUCCGC | | CU | |
| | | ATTCTTTCCT | | | GCCCCGGTTAATTTGCATATAATA | | G | | | |
| | | GTAAAGCCA | | | TTTCCTAGTAACTATAGAGGCTTA | | | | | |
| | | CAGATGGGG | | | ATGTGCGATAAAAGACAGATAATC | | | | | |
| | | AAAGAATGG | | | TGTTCTTTTTAATACTAGCTACATT | | | | | |
| | | CAGTTCTCC | | | TTACATGATAGGCTTGGATTTCTAT | | | | | |
| | | GCGTGCCTA | | | AAGAGATACAAATACTAAATTATTA | | | | | |
| | | CTAGTAATTC | | | TTTTAAAAAACAGCACAAAAGGAA | | | | | |
| | | TTCGATTCTG | | | ACTCACCCTAACTGTAAAGTAATT | | | | | |
| | | C | | | GTGTGTTTTGAGACTATAAATATC | | | | | |
| | | | | | CCTTGGAGAAAAGCCTTGTTTGCT | | | | | |
| | | | | | CGAGTGAGCGAGCGGAGAACTGC | | | | | |
| | | | | | CATTCTTTCCTGTAAAGCCACAGA | | | | | |
| | | | | | TGGGGAAAGAATGGCAGTTCTCC | | | | | |
| | | | | | GCGTGCCTACTAGTAATTCTTCGA | | | | | |
| | | | | | TTCTGCTTTTTT | | | | | |

Exemplary nucleotide sequences are set out in Table 1 above. The various sequences have a different promoter and/or different flanking sequences. In some instances, the miRNA has one binding site on DUX4. In other instances, the miRNA has multiple binding sites on DUX4. For example, microRNA 675 (miR-675) is a natural microRNA that binds multiple binding sites on its target gene because it does not have 100% complementarity to the binding site, i.e., DUX4 target sequence.

In some aspects, a nucleic acid of the disclosure comprises a nucleotide sequence comprising at least or about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the sequence set forth in any one of SEQ ID NOs: 1-124. In some aspects, a nucleic acid of the disclosure comprises a nucleotide sequence comprising at least 90% identity to the sequence set forth in any one of SEQ ID NOs: 5-47; the nucleotide sequence set forth in any one of SEQ ID NOs: 5-47; a nucleotide sequence comprising at least 90% identity to the sequence set forth in any one of SEQ ID NOs: 50-92; the nucleotide sequence set forth in any one of SEQ ID NOs: 50-92; a nucleotide sequence that encodes the RNA sequence set forth in any one of SEQ ID NOs: 95-105; or a nucleotide sequence that specifically hybridizes to the DUX4 sequence set forth in any one of SEQ ID NOs:106-124.

In some aspects, the disclosure includes the use of RNA interference to downregulate or inhibit DUX4 expression. RNA interference (RNAi) is a mechanism of gene regulation in eukaryotic cells that has been considered for the treatment of various diseases. RNAi refers to post-transcriptional control of gene expression mediated by miRNAs. The miRNAs are small (about 21-25 nucleotides), noncoding RNAs that share sequence homology and base-pair with sequence target sites of cognate messenger RNAs (mRNAs). The interaction between the miRNAs and mRNAs directs cellular gene silencing machinery inducing mRNA decay and/or preventing mRNA translation into protein.

As an understanding of natural RNAi pathways has developed, researchers have designed artificial shRNAs and snRNAs for use in regulating expression of target genes for treating disease. Several classes of small RNAs are known to trigger RNAi processes in mammalian cells, including short (or small) interfering RNA (siRNA), and short (or small) hairpin RNA (shRNA) and microRNA (miRNA), which constitute a similar class of vector-expressed triggers [Davidson et al., Nat. Rev. Genet. 12:329-40, 2011; Harper, Arch. Neurol. 66:933-8, 2009]. shRNA and miRNA are expressed in vivo from plasmid- or virus-based vectors and may thus achieve long term gene silencing with a single administration, for as long as the vector is present within target cell nuclei and the driving promoter is active (Davidson et al., Methods Enzymol. 392:145-73, 2005). Importantly, this vector-expressed approach leverages the decades-long advancements already made in the muscle gene therapy field, but instead of expressing protein coding genes, the vector cargo in RNAi therapy strategies are artificial shRNA or miRNA cassettes targeting disease genes-of-interest. This strategy is used to express a natural miRNA. MicroRNA 675 has its own structure. Each other miRNA described herein is based on hsa-miR-30a sequences and structure. The natural mir-30a mature sequences are replaced by unique sense and antisense sequences derived from the target gene.

In some embodiments, the products and methods of the disclosure comprise microRNA (miRNA). MicroRNAs (miRNAs) are a class of non-coding RNAs that play important roles in RNA silencing and in regulating gene expression. The majority of miRNAs are transcribed from DNA sequences into primary miRNAs and processed into precursor miRNAs, and finally mature miRNAs. In most cases, miRNAs interact with the 3′ untranslated region (3′ UTR) of target mRNAs to induce mRNA degradation and translational repression. However, interaction of miRNAs with other regions, including the 5′ UTR, coding sequence, and gene promoters, have also been reported. Under certain conditions, miRNAs can also activate translation or regulate transcription. The interaction of miRNAs with their target genes is dynamic and dependent on many factors, such as subcellular location of miRNAs, the abundancy of miRNAs and target mRNAs, and the affinity of miRNA-mRNA interactions.

Most studies to date have shown that miRNAs bind to a specific sequence at the 3′ UTR of their target mRNAs to induce translational repression and mRNA deadenylation and decapping. miRNA binding sites have also been detected in other mRNA regions including the 5′ UTR and coding sequence, as well as within promoter regions. The binding of miRNAs to 5′ UTR and coding regions have silencing effects on gene expression while miRNA interaction with promoter region has been reported to induce transcription.

In various aspects, polymerase II promoters and polymerase III promoters, such as U6 and H1, are used. In some aspects, U6 miRNAs are used. In some aspects, H1 miRNAs are used. Thus, in some aspects, U6 miRNA or H1 miRNA are used to further inhibit, knockdown, or interfere with DUX4 gene expression. Traditional small/short hairpin RNA (shRNA) sequences are usually transcribed inside the cell nucleus from a vector containing a Pol III promoter, such as U6. The endogenous U6 promoter normally controls expression of the U6 RNA, a small nuclear RNA (snRNA) involved in splicing, and has been well-characterized [Kunkel et al., Nature. 322(6074):73-7 (1986); Kunkel et al., Genes Dev. 2(2):196-204 (1988); Paule et al., Nucleic Acids Res. 28(6):1283-98 (2000)]. In some aspects, the U6 or H1 promoter is used to control vector-based expression of shRNA molecules in mammalian cells [Paddison et al., Proc. Natl. Acad. Sci. USA 99(3):1443-8 (2002); Paul et al., Nat. Biotechnol. 20(5):505-8 (2002); Medina et al., Curr. Opin. Mol. Ther. 1:580-94 (1999)] because (1) the promoter is recognized by RNA polymerase III (poly III) and controls high-level, constitutive expression of shRNA; (2) the Pol III promoter possesses greater capacity than RNA polymerase II to synthesize shRNA of high yield [Boden et al., Nucleic Acids Res. 32:1154-8 (2004); Xia et al., Neurodegenerative Dis. 2:220-31 (2005)]; (3) the Pol III promoters are consistent of compact sequence and simple terminator that are easy to handle [Medina et al. (1999) supra]; and (2) the promoter is active in most mammalian cell types. In some aspects, the promoter is a type III Pol III promoter in that all elements required to control expression of the shRNA are located upstream of the transcription start site [Paule et al., Nucleic Acids Res. 28(6):1283-98 (2000)]. The disclosure includes both murine and human U6 promoters. The shRNA containing the sense and antisense sequences from a target gene connected by a loop is transported from the nucleus into the cytoplasm where Dicer processes it into small/short interfering RNAs (siRNAs).

The disclosure includes a composition comprising any of the nucleic acids described herein in combination with a diluent, excipient, or buffer. In some aspects, the disclosure includes a vector comprising any of the nucleic acids described herein.

In some embodiments, the disclosure includes a vector comprising any of the nucleic acids described herein. Thus, embodiments of the disclosure utilize vectors (for example, viral vectors, such as adeno-associated virus (AAV), adenovirus, retrovirus, lentivirus, equine-associated virus, alphavirus, pox virus, herpes virus, herpes simplex virus, polio virus, sindbis virus, vaccinia virus or a synthetic virus, e.g., a chimeric virus, mosaic virus, or pseudotyped virus, and/or a virus that contains a foreign protein, synthetic polymer, nanoparticle, or small molecule) to deliver the nucleic acids disclosed herein.

In some embodiments, the vectors are AAV vectors. In some aspects, the vectors are single stranded AAV vectors. In some aspects the AAV is recombinant AAV (rAAV). In some aspects, the rAAV lack rep and cap genes. In some aspects, rAAV are self-complementary (sc)AAV.

Thus, in some aspects, the viral vector is an adeno-associated virus (AAV), such as an AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), AAVrh.10 (i.e., an AAV containing AAVrh.10 ITRs and AAVrh.10 capsid proteins), AAV11 (i.e., an AAV containing AAV11 ITRs and AAV11 capsid proteins), AAV12 (i.e., an AAV containing AAV12 ITRs and AAV12 capsid proteins), AAV13 (i.e., an AAV containing AAV13 ITRs and AAV13 capsid proteins), AAV-anc80, AAV rh.74, AAV rh.8, AAVrh.10, or AAV-B1.

In some aspects, the disclosure utilizes adeno-associated virus (AAV) to deliver nucleic acids encoding the miRNA. AAV is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the complete genome of AAV1 is provided in GenBank Accession No. NC_002077; the complete genome of AAV2 is provided in GenBank Accession No. NC_001401 and Srivastava et al., J. Virol., 45: 555-564 {1983); the complete genome of AAV3 is provided in GenBank Accession No. NC_1829; the complete genome of AAV4 is provided in GenBank Accession No. NC_001829; the AAV5 genome is provided in GenBank Accession No. AF085716; the complete genome of AAV6 is provided in GenBank Accession No. NC_00 1862; at least portions of AAV7 and AAV8 genomes are provided in GenBank Accession Nos. AX753246 and AX753249, respectively (see also U.S. Pat. Nos. 7,282,199 and 7,790,449 relating to AAV8); the AAV9 genome is provided in Gao et al., J. Virol., 78: 6381-6388 (2004); the AAV10 genome is provided in Mol. Ther., 13(1): 67-76 (2006); the AAV11 genome is provided in Virology, 330(2): 375-383 (2004); the AAV12 genome is provided in J. Virol. 2008 February; 82(3): 1399-406; and the AAV13 genome is provided in J. Virol. 2008; 82: 8911. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka (Current Topics in Microbiology and Immunology, 158: 97-129 (1992)).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. In some aspects, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. AAV may be lyophilized and AAV-infected cells are not resistant to superinfection.

In some embodiments, DNA plasmids of the disclosure are provided which comprise rAAV genomes of the disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, AAV rh.74, AAV rh.8, AAVrh.10, and AAV-B1. In some aspects, AAV DNA in the rAAV genomes is from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, AAV rh.74, AAV rh.8, AAVrh.10, and AAV-B1. Other types of rAAV variants, for example rAAV with capsid mutations, are also included in the disclosure. See, for example, Marsic et al., Molecular Therapy 22(11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

Recombinant AAV genomes of the disclosure comprise one or more AAV ITRs flanking at least one DUX4-targeted polynucleotide or nucleotide sequence. In some embodiments, the polynucleotide is an miRNA or a polynucleotide encoding the miRNA. In some aspects, the miRNA is administered with other polynucleotide constructs targeting DUX4. In various aspects, the miRNA is expressed under various promoters including, but not limited to, such promoters as a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a CK8 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, or a desmin promoter AAV DNA in the rAAV genomes may be from any AAV serotype for which a recombinant virus can be derived including, but not limited to, AAV serotypes AAV1, AAV2, AAV3, AAV4, AAV5, AAV6, AAV7, AAV8, AAV9, AAV10, AAV11, AAV12, AAV13, AAVanc80, AAV rh.74, AAV rh.8, AAVrh.10, and AAV-B1. As set out herein above, the nucleotide sequences of the genomes of various AAV serotypes are known in the art.

DNA plasmids of the disclosure comprise rAAV genomes of the disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, or AAV-B1. In some aspects, AAV DNA in the rAAV genomes is from any AAV serotype for which a recombinant virus can be derived including, but not limited to, AAV serotypes AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, or AAV-B1. Other types of rAAV variants, for example rAAV with capsid mutations, are also included in the disclosure. See, for example, Marsic et al., Molecular Therapy 22(11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

In some embodiments, packaging cells are provided. Packaging cells are created in order to have a cell line that stably expresses all the necessary components for AAV particle production. Retroviral vectors are created by removal of the retroviral gag, pol, and env genes. These are replaced by the therapeutic gene. In order to produce vector particles, a packaging cell is essential. Packaging cell lines provide all the viral proteins required for capsid production and the virion maturation of the vector. Thus, packaging cell lines are made so that they contain the gag, pol and env genes. Following insertion of the desired gene into in the retroviral DNA vector, and maintenance of the proper packaging cell line, it is now a simple matter to prepare retroviral vectors

For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing [Samulski et al., 1982, Proc. Natl. Acad. S6. USA, 79:2077-2081], addition of synthetic linkers containing restriction endonuclease cleavage sites [Laughlin et al., 1983, Gene, 23:65-73] or by direct, blunt-end ligation [Senapathy & Carter, 1984, J. Biol. Chem., 259:4661-4666]. The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

In some embodiments, the disclosure includes a composition comprising any of the nucleic acids or any of the vectors described herein in combination with a diluent, excipient, or buffer.

In some embodiments, therefore, a method of generating a packaging cell to create a cell line that stably expresses all the necessary components for AAV particle production is provided. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing [Samulski et al., 1982, Proc. Natl. Acad. S6. USA, 79:2077-2081], addition of synthetic linkers containing restriction endonuclease cleavage sites (Laughlin et al., 1983, Gene, 23:65-73) or by direct, blunt-end ligation (Senapathy et al., 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbiol. and Immunol. 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine, 13:1244-1250 (1995); Paul et al., Human Gene Therapy, 4:609-615 (1993); Clark et al., Gene Therapy, 3:1124-1132 (1996); U.S. Pat. Nos. 5,786,211; 5,871,982; 6,258,595; and McCarty, Mol. Ther., 16(10): 1648-1656 (2008). The foregoing documents are hereby incorporated by reference in their entirety herein, with particular emphasis on those sections of the documents relating to rAAV production. The production and use of various types of rAAV are specifically contemplated and exemplified. Recombinant AAV (i.e., infectious encapsidated rAAV particles) are thus provided herein. In some aspects, genomes of the rAAV lack AAV rep and cap genes; that is, there is no AAV rep or cap DNA between the ITRs of the genomes of the rAAV. In some embodiments, the AAV is a recombinant linear AAV (rAAV), a single-stranded AAV (ssAAV), or a recombinant self-complementary AAV (scAAV).

The disclosure thus provides in some embodiments packaging cells that produce infectious rAAV. In one embodiment, packaging cells are stably transformed cancer cells, such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV, in some aspects, are purified by methods standard in the art, such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In some embodiments, the disclosure provides a composition or compositions comprising a nucleic acid or a vector, e.g., such as a viral vector, as described herein. Thus, compositions comprising delivery vehicles (such as rAAV) described herein are provided. In various aspects, such compositions also comprise a pharmaceutically acceptable carrier. In general, as used herein, “pharmaceutically acceptable carrier” means all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

The disclosure also provides various small molecule compounds and compositions comprising such small molecule compounds for downregulating DUX4 in the treatment of a muscular dystrophy or cancer associated with expression or overexpression of DUX4. Prior studies showed that mir-675 was induced with treatment of melatonin, and estrogen alone or in combination with progesterone [Cai et al., Journal Pineal Research 61: 82-95 (2016); Gaube et al., BMC Pharmacology 7:11 (2007); Hanifi-Moghaddam et al., Journal Molecular Medicine 85: 471-480 (2007)]. Estrogen or its derivative β-estradiol have been previously linked to FSHD pathogenesis, although the role of estrogen in FSHD is not definitive. Some previous reports suggested that estrogen might be protective in FSHD disease, as females were reported to be less severely affected than males and may have persistent worsening of symptoms after childbirth and menopause [Awater et al., European J. Obstetrics, Gynecology, and Reprod. Biol. 162: 153-9 (2012); Sacconi et al., Biochim. Biophys. Acta. 1852: 607-14 (2015); Zatz et al., Amer. J. Med. Genetics 77:155-61 (1998)]. One study suggested that the beneficial effects of estrogen were mediated by the estrogen receptor (ERR), which, when activated by estrogen, sequesters the DUX4 protein in the cytoplasm of cells and prevents its toxic effects in nuclei [Teveroni et al., J. Clin. Investigation 127: 1531-45 (2017)]. In contrast to these reports suggesting a protective effect of estrogen, a recent study of FLExDUX4 mice reported that females perform worse than males in some outcome measures [Jones et al., Skelet. Muscle 10:8 (2020). However, the ERT2 system that was used to generate these animals utilizes the anti-estrogen drug Tamoxifen to induce high levels of DUX4 expression, thereby complicating interpretation of the impacts of estrogen on phenotypes in these animals. In addition, a recent clinical study of 85 female FSHD patients did not find a significant correlation between differences in estrogen exposure and disease severity [Mul et al., Neuromuscul Disord 28, 508-11 (2018)]. Interestingly their clinical approach consisted of subtracting periods with high progesterone levels from the reproductive life span so a protective effect caused by interplay with other reproductive hormones, including progesterone, could not be ruled out. The data provided herein the disclosure suggest a new mechanism by which estrogen, and/or estrogen and progesterone, could at least partially protect cells from FSHD disease by counteracting DUX4 expression via mir-675 upregulation. Melatonin has been previously identified as a promising drug therapy for neuromuscular diseases due to its anti-inflammatory and antioxidant properties. For this purpose, it was tested in the mdx5Cv Duchenne muscular dystrophy (DMD) mouse model, where it improved muscle function and enhanced the redox status of the muscle [Hibaoui et al., J. Pineal Res. 51: 163-71 (2011)]. In another study, melatonin prevented the premature senescence of cardiac progenitor cells that occurs in heart diseases [Cai et al., J. Pineal Res. 61: 82-95 (2016)].

The disclosure shows that β-estradiol, β-estradiol plus medroxyprogesterone acetate (MPA), and melatonin can all downregulate DUX4 expression via mir-675 upregulation. Thus, the disclosure includes various compounds and combinations of compounds, such as p-estradiol+melatonin; melatonin+MPA; bleomycin; pyrazinamide; sorafenib; bleomycin+pyrazinamide; bleomycin+sorafenib; and pyrazinamide+sorafenib in the methods of treating a muscular dystrophy or a cancer associated with DUX4 expression or overexpression as described herein.

Gene expression studies showed that bleomycin [Liu et al., J. Immunol. 187: 450-61 (2011)], pyrazinamide [Manca et al., PloS One. 8:e74082 (2013);] and sorafenib (https_colon_forward_slash_forward slash_maayanlab.cloud/Harmonizome/gene_set/sorafenib_homo+sapiens_gpl6244_gse35907/GEO+Signatures+of+Differentially+Expressed+Genes+for+Small+Molecules) [Man et al., Blood. 119: 5133-43 (2012)] upregulated mir-675 expression. The disclosure therefore includes bleomycin, pyrazinamide, and sorafenib, or derivatives thereof, and/or combinations thereof for, in some aspects, a synergistic effect, in various methods of treating FSHD, as described herein.

The disclosure therefore includes bleomycin or a derivative thereof, pyrazinamide or a derivative thereof, sorafenib (4-[4-[[4-chloro-3-(trifluoromethyl)phenyl]carbamoylamino]phenoxy]-N-methylpyridine-2-carboxamide) or a derivative thereof, or a combination of any thereof.

In some aspects, the derivative is a bleomycin derivative. Such bleomycin derivatives include, but are not limited to, bleomycin A2, deglyco-bleomycin A2, bleomycin A5, bleomycin A6, bleomycin B2, and also includes drugs which are synonyms of bleomycin, for example, Bleocin, Bleomicin, Bleomicina (in Spanish), Bleomycine (in French), and Bleomycinum (in Latin).

In some aspects, the derivative is a pyrazinamide derivative. Such pyrazinamide derivative includes, but is not limited to, pyrazine-2-carboxylic acid chloride, N-(1-bromine methyl) pyrazine formamide, N-(bromomethyl)pyrazine-2-carboxamide, N-(2-bromoethyl)pyrazine-2-carboxamide, N-(3-bromopropyl)pyrazine-2-carboxamide, N-(piperidin-1-ylmethyl)pyrazine-2-carboxamide, N-(piperazin-1-ylmethyl)pyrazine-2-carboxamide, N-(thiomorpholinomethyl)pyrazine-2-carboxamide, N-(2-(piperidin-1-yl)ethyl)pyrazine-2-carboxamide, N-(2-(piperazin-1-yl)ethyl)pyrazine-2-carboxamide, N-(2-morpholinoethyl)pyrazine-2-carboxamide, N-(2-thiomorpholinoethyl)pyrazine-2-carboxamide, N-(3-(piperidin-1-yl)propyl)pyrazine-2-carboxamide, N-(3-(piperazin-1-yl)propyl)pyrazine-2-carboxamide, N-(3-morpholinopropyl)pyrazine-2-carboxamide, N-(3-thiomorpholinopropyl)pyrazine-2-carboxamide, 3-chloropyrazine-2-carboxamide, 3-[(4-methylbenzyl)amino]pyrazine-2-carboxamide, N-Benzylpyrazine-2-carboxamides, pyrazine-1,2,3-triazoles, N-alkyl substituted 3-aminopyrazine-2-carboxamides, Pyrazinoic acidn-octyl ester, Pyrazine thiocarboxamide, N-Hydroxymethyl pyrazine, thiocarboxamide, Pyrazinoic acid pivaloyloxymethyl ester, 3-(Benzylamino)pyrazine-2-carboxamide, 3-[(3-Chlorobenzyl)amino]pyrazine-2-carboxamide, 3-[(3,4-Dichlorobenzyl)amino]pyrazine-2-carboxamide, 3-[(3-Trifluoromethylbenzyl)amino]pyrazine-2-carboxamide, 3-[(4-Chlorobenzyl)amino]pyrazine-2-carboxamide, 3-[(2-Methylbenzyl)amino]pyrazine-2-carboxamide, 3-[(4-Methoxybenzyl)amino]pyrazine-2-carboxamide, 3-[(4-Methylbenzyl)amino]pyrazine-2-carboxamide, 3-[(4-Aminobenzyl)amino]pyrazine-2-carboxamide, 3-[(2-Chlorobenzyl)amino]pyrazine-2-carboxamide, 3-[(2-Fluorobenzyl)amino]pyrazine-2-carboxamide, 3-[(4-Trifluoromethylbenzyl)amino]pyrazine-2-carboxamide, 3-[(2-Trifluoromethylbenzyl)amino]pyrazine-2-carboxamide, 3-[(2,4-Dimethoxybenzyl)amino]pyrazine-2-carboxamide, 3-[(3-Nitrobenzyl)amino]pyrazine-2-carboxamide, 3-(benzylamino)-5-cyanopyrazine-2-carboxamide, 3-(4-methylbenzylamino)-5-cyanopyrazine-2-carboxamide, 3-(4-methoxybenzylamino)-5-cyanopyrazine-2-carboxamide, 3-(4-aminobenzylamino)-5-cyanopyrazine-2-carboxamide, 3-(3-chlorobenzylamino)-5-cyanopyrazine-2-carboxamide, 3-(4-chlorobenzylamino)-5-cyanopyrazine-2-carboxamide, 3-(3,4-dichlorobenzylamino)-5-cyanopyrazine-2-carboxamide, 3-(3-nitrobenzylamino)-5-cyanopyrazine-2-carboxamide, 3-(3-trifluoromethylbenzylamino)-5-cyanopyrazine-2-carboxamide, 3-(benzylamino)pyrazine-2,5-dicarbonitrile, 3-(4-methylbenzylamino)pyrazine-2,5-dicarbonitrile, 3-(4-methoxybenzylamino)pyrazine-2,5-dicarbonitrile, 3-(4-aminobenzylamino)pyrazine-2,5-dicarbonitrile, 3-(3-chlorobenzylamino)pyrazine-2,5-dicarbonitrile, 3-(4-chlorobenzylamino)pyrazine-2,5-dicarbonitrile, 3-(3,4-dichlorobenzylamino)pyrazine-2,5-dicarbonitrile, 3-(3-nitrobenzylamino)pyrazine-2,5-dicarbonitrile, 3-(3-trifluoromethylbenzylamino)pyrazine-2,5-dicarbonitrile, 3-(2-methylbenzylamino)pyrazine-2,5-dicarbonitrile, and also includes drugs which are synonyms of pyrazinamide, such as 2-carbamylpyrazine, 2-pyrazinecarboxamide, Aldinamide, Pyrazine carboxamide, pyrazine-2-carboxamide, Pyrazineamide, Pyrazinecarboxamide, Pyrazinoic acid amide, Pyrizinamide, Pirazinamida or Pyrazinamida (in Spanish), Pyrazinamid (in German), and Pyrazinamidum (in Latin).

In some aspects, the derivative is a sorafenib derivative. Such sorafenib derivative includes, but is not limited to, 4-Chloropyridine-2-carbonyl chloride hydrochloride, 4-Chloro-N-cyclopentylpyridine-2-carboxamide, 4-Chloro-N-cyclohexylpyridine-2-carboxamide, 4-Chloro-N-cyclohexylmethylpyridine-2-carboxamide, 4-Chloro-N-benzylpyridine-2-carboxamide, 4-Chloro-N-phenylethylpyridine-2-carboxamide, 4-(4-Aminophenoxy)-N-cyclopentylpyridine-2-carboxamide, 4-(4-Aminophenoxy)-N-cyclohexylpyridine-2-carboxamide, 4-(4-Aminophenoxy)-N-cyclohexylmethylpyridine-2-carboxamide, 4-(4-Aminophenoxy)-N-benzylpyridine-2-carboxamide, 4-(4-Aminophenoxy)-N-phenylethylpyridine-2-carboxamide, 4-[4-[[4-Chloro-3-(trifluoromethyl)phenyl]carbamoylamino]phenoxy]-N-cyclopentyl-pyridine-2-carboxamide, 4-[4-[[4-Chloro-3-(trifluoromethyl)phenyl]carbamoylamino]phenoxy]-N-cyclohexyl-pyridine-2-carboxamide, 4-[4-[[4-Chloro-3-(trifluoromethyl)phenyl]carbamoylamino]phenoxy]-N-cyclohexylmethyl-pyridine-2-carboxamide, 4-[4-[[4-Chloro-3-(trifluoromethyl)phenyl]carbamoylamino]phenoxy]-N-benzyl-pyridine-2-carbox-amide, 4-[4-[[4-Chloro-3-(trifluoromethyl)phenyl]carbamoylamino]phenoxy]-N-phenylethyl-pyridine-2-carboxamide, Sorafenib derivatives containing a phenylcyano group, Sorafenib derivatives containing the nitrogen heterocyclic, sorafenib derivatives with a quinoxalinedione structure, sorafenib derivatives containing a chalcone moiety, sorafenib derivatives containing thioether and nicotinamide, class of diaryl thiourea derivatives of sorafenib, orafenib derivatives containing dithiocarbamate moiety, orafenib derivatives bearing a pyrazole scaffold, sorafenib derivatives containing a cyclohexyl moiety, sorafenib derivatives containing quinoline nucleus, sorafenib derivatives containing a dimer-based structure, a,b-unsaturated ketones derivatives of sorafenib, orafenib derivatives containing a 1,2,3-triazoles framework, orafenib derivatives containing a 1,3,4-triarylpyrazole framework, imidazo [2,1-b] thiazole derivatives of sorafenib, 4-(4-(5-(2,4-Dichlorophenyl)-4,5-dihydro-1H-pyrazol-3-yl)phenoxy)-N-methylpicolinamide, 4-(4-(5-(3-Bromophenyl)-4,5-dihydro-1H-pyrazol-3-yl)phenoxy)-N-methylpicolinamide, N-Methyl-4-(4-(5-(3,4,5-trimethoxyphenyl)-4,5-dihydro-1H-pyrazol-3-yl)phenoxy)picolinamide, 4-(4-(5-(4-Cyanophenyl)-4,5-dihydro-1H-pyrazol-3-yl)phenoxy)-N-methylpicolinamide, 4-(4-(5-(2-Chloro-4-fluorophenyl)-4,5-dihydro-1H-pyrazol-3-yl)phenoxy)-N-methylpicolinamide, N-Methyl-4-(4-(5-(4-nitrophenyl)-4,5-dihydro-1H-pyrazol-3-yl)phenoxy)picolinamide, N-Methyl-4-(4-(5-(3-nitrophenyl)-4,5-dihydro-1H-pyrazol-3-yl)phenoxy)picolinamide, 4-(4-(5-(4-Methoxyphenyl)-4,5-dihydro-1H-pyrazol-3-yl)phenoxy)-N-methylpicolinamide, N-Methyl-4-(4-(5-phenyl-4,5-dihydro-1H-pyrazol-3-yl)phenoxy)picolinamide, 4-(4-(5-(3,4-Dichlorophenyl)-4,5-dihydro-1H-pyrazol-3-yl)phenoxy)-N-methylpicolinamide, 4-(4-(5-(4-Fluorophenyl)-4,5-dihydro-1H-pyrazol-3-yl)phenoxy)-N-methylpicolinamide, 4-(4-(5-(4-Bromophenyl)-4,5-dihydro-1H-pyrazol-3-yl)phenoxy)-N-methylpicolinamide, N-Methyl-4-(4-(5-(2,3,4-trimethoxyphenyl)-4,5-dihydro-1H-pyrazol-3-yl)phenoxy)picolinamide, 4-(4-(5-(2,3-Dichlorophenyl)-4,5-dihydro-1H-pyrazol-3-yl)phenoxy)-N-methylpicolinamide, N-Methyl-4-(4-(3-(4-nitrophenyl)-4,5-dihydro-1H-pyrazol-5-yl)phenoxy) picolinamide, 4-(4-(3-(4-Bromophenyl)-4,5-dihydro-1H-pyrazol-5-yl)phenoxy)-N-methylpicolinamide, 4-(4-(3-(4-Chlorophenyl)-4,5-dihydro-1H-pyrazol-5-yl)phenoxy)-N-methylpicolinamide, 4-(4-(1-Carbamothioyl-5-(3-nitrophenyl)-4,5-dihydro-1H-pyrazol-3-yl)phenoxy)-N-methylpicolinamide, 4-(4-(1-Carbamothioyl-5-(4-fluorophenyl)-4,5-dihydro-1H-pyrazol-3-yl)phenoxy)-N-methylpicolinamide, 4-(4-(1-Carbamothioyl-5-(4-chlorophenyl)-4,5-dihydro-1H-pyrazol-3-yl)phenoxy)-N-methylpicolinamide, 4-(4-(1-Carbamothioyl-5-(2,3-dichlorophenyl)-4,5-dihydro-1H-pyrazol-3-yl)phenoxy)-N-methylpicolinamide, 4-(4-(1-Carbamothioyl-5-(4-cyanophenyl)-4,5-dihydro-1H-pyrazol-3-yl)phenoxy)-N-methylpicolinamide, 4-(4-(1-Carbamothioyl-3-(4-nitrophenyl)-4,5-dihydro-1H-pyrazol-5-yl)phenoxy)-N-methylpicolinamide, HLC-080, benzimidazole derivative bearing a pyrrolidine side chain, N-(4-chloro-3-(trifluoromethyl)phenyl)-2-(2-oxoindolin-3-ylidene)hydrazine-1-carboxamide, N-(3,4-difluorophenyl)-2-(2-oxoindolin-3-ylidene)hydrazine-1-carboxamide, N-(4-chloro-3-(trifluoromethyl)phenyl)-2-(5-methyl-2-oxoindolin-3-ylidene)hydrazine-1-carboxamide, 2-((1H-indol-3-yl)methylene)-N-(3-bromophenyl)hydrazine-1-carboxamide, 2-((1H-indol-3-yl)methylene)-N-(3,4-difluorophenyl)hydrazine-1-carboxamide, 2-((1H-indol-3-yl)methylene)-N-(4-chloro-3-(trifluoromethyl)phenyl)hydrazine-1-carboxamide, 2-((1H-indol-3-yl)methylene)-N-(p-tolyphydrazine-1-carboxamide, 2-((2-chloro-1H-indol-3-yl)methylene)-N-(3,4-difluorophenyl)hydrazine-1-carboxamide, 2-((2-chloro-1H-indol-3-yl)methylene)-N-(3-chlorophenyl)hydrazine-1-carboxamide, N-(3-bromophenyl)-2-((2-chloro-1H-indol-3-yl)methylene)hydrazine-1-carboxamide, 2-((2-chloro-1H-indol-3-yl)methylene)-N-(4-methoxyphenyl)hydrazine-1-carboxamide, 2-((2-chloro-1H-indol-3-yl)methylene)-N-(4-chloro-3-(trifluoromethyl)phenyl)hydrazine-1-carboxamide, 2-((2-chloro-1-ethyl-1H-indol-3-yl)methylene)-N-(4-chloro-3-(trifluoromethyl)phenyl)hydrazine-1-carboxamide, 2-((2-chloro-1-ethyl-1H-indol-3-yl)methylene)-N-(4-fluorophenyl)hydrazine-1-carboxamide, N-(3-bromophenyl)-2-((2-chloro-1-ethyl-1H-indol-3-yl)methylene)hydrazine-1-carboxamide, 2-((2-chloro-1-ethyl-1H-indol-3-yl)methylene)-N-(2-fluorophenyl)hydrazine-1-carboxamide, 2-((2-chloro-1-ethyl-1H-indol-3-yl)methylene)-N-(3-fluorophenyl)hydrazine-1-carboxamide, 2-((2-chloro-1-ethyl-1H-indol-3-yl)methylene)-N-(4-methoxyphenyl)hydrazine-1-carboxamide, 2-((2-chloro-1-ethyl-1H-indol-3-yl)methylene)-N-(3-chlorophenyl)hydrazine-1-carboxamide, N-(3-bromophenyl)-2-((2-chloro-1-propyl-1H-indol-3-yl)methylene)hydrazine-1-carboxamide, N-(4-(2-(methylcarbamoyl) pyridin-4-yloxy) phenyl)-4-phenylpicolinamide, 4-(4-fluorophenyl)-N-(4-(2-(methylcarbamoyl) pyridin-4-yloxy) phenyl) picolinamide, 4-(2,4-Difluorophenyl)-N-(4-(2-(methylcarbamoyl) pyridin-4-yloxy) phenyl) picolinamide, 4-(4-Chlorophenyl)-N-(4-(2-(methylcarbamoyl) pyridin-4-yloxy) phenyl) picolinamide, 4-(4-Methoxyphenyl)-N-(4-(2-(methylcarbamoyl) pyridin-4-yloxy) phenyl) picolinamide, N-(4-(2-(methylcarbamoyl) pyridin-4-yloxy) phenyl)-4-p-tolylpicolinamide, N-(4-(2-(methylcarbamoyl) pyridin-4-yloxy) phenyl)-4-m-tolylpicolinamide, 4-(3-Fluorophenyl)-N-(4-(2-(methylcarbamoyl) pyridin-4-yloxy)phenyl) picolinamide, N-(4-(2-(methylcarbamoyl) pyridin-4-yloxy) phenyl)-4-(4-(trifluoromethyl) phenyl) picolinamide, 4-(4-Ethylphenyl)-N-(4-(2-(methylcarbamoyl) pyridin-4-yloxy) phenyl) picolinamide, 4-(2, 4-dimethylphenyl)-N-(4-(2-(methylcarbamoyl) pyridin-4-yloxy) phenyl) picolinamide, N-(4-(2-(methylcarbamoyl) pyridin-4-yloxy) phenyl)-5-phenylpicolinamide, 5-(4-Fluorophenyl)-N-(4-(2-(methylcarbamoyl) pyridin-4-yloxy) phenyl) picolinamide, 5-(2, 4-Difluorophenyl)-N-(4-(2-(methylcarbamoyl) pyridin-4-yloxy) phenyl) picolinamide, 5-(4-Chlorophenyl)-N-(4-(2-(methylcarbamoyl) pyridin-4-yloxy) phenyl) picolinamide, 5-(4-Methoxyphenyl)-N-(4-(2-(methylcarbamoyl) pyridin-4-yloxy)phenyl) picolinamide, N-(4-(2-(methylcarbamoyl) pyridin-4-yloxy) phenyl)-5-p-Tolylpicolinamide, N-(4-(2-(methylcarbamoyl) pyridin-4-yloxy) phenyl)-5-m-tolylpicolinamide, 5-(3-Fluorophenyl)-N-(4-(2-(methylcarbamoyl) pyridin-4-yloxy) phenyl) picolinamide, N-(4-(2-(methylcarbamoyl) pyridin-4-yloxy) phenyl)-5-(4-(trifluoromethyl) phenyl)picolinamide, 5-(4-Ethylphenyl)-N-(4-(2-(methylcarbamoyl) pyridin-4-yloxy) phenyl) picolinamide, 5-(2, 4-Dimethylphenyl)-N-(4-(2-(methylcarbamoyl) pyridin-4-yloxy) phenyl) picolinamide, and also includes drugs which are synonyms of sorafenib, such as Sorafenib (in French) and Sorafenibum (in Latin).

Thus, the disclosure provides a method of upregulating expression of microRNA-675 in a cell comprising contacting the cell with an effective amount of an estrogen, synthetic estrogen, progesterone, progestin, melatonin, bleomycin, pyrazinamide, sorafenib, or a derivative thereof, or a combination of any thereof. The disclosure also provides a method of inhibiting and/or interfering with expression of a DUX4 gene or protein in a cell comprising contacting the cell with an effective amount of an estrogen, synthetic estrogen, progesterone, progestin, melatonin, bleomycin, pyrazinamide, sorafenib, or a derivative thereof, or a combination of any thereof. The disclosure further provides a method of treating a subject having a muscular dystrophy or a cancer associated with DUX4 expression or overexpression comprising administering to the subject an effective amount of an estrogen, synthetic estrogen, progesterone, progestin, melatonin, bleomycin, pyrazinamide, sorafenib, or a derivative thereof, or a combination of any thereof. In some aspects, the muscular dystrophy is facioscapulohumeral muscular dystrophy (FSHD). In some aspects, the cancer is a sarcoma, a B-cell lymphoma, or a DUX4-expressing cancer of the adrenal, bile duct, bladder, breast, cervix, colon, endometrium, esophagus, head/neck, liver, brain, lung, mesothelium, neural crest, ovary, pancreas, prostate, kidney, skin, soft tissue, stomach, testicles, or thymus.

In some aspects, the estrogen or synthetic estrogen is estrone, estradiol, estriol, estetrol, 27-hydroxycholesterol, dehydroepiandrosterone (DHEA), 7-oxo-DHEA, 7α-hydroxy-DHEA, 16α-hydroxy-DHEA, 7β-hydroxyepiandrosterone, androstenedione (A4), androstenediol (A5), 3α-androstanediol, and 3β-androstanediol, 2-hydroxyestradiol, 2-hydroxyestrone, 4-hydroxyestradiol, 4-hydroxyestrone, 16α-hydroxyestrone, ethinyl estradiol, estradiol valerate, estropipate, conjugate esterified estrogen, and quinestrol.

In some aspects, the progesterone or progestin is medroxyprogesterone acetate (MPA), 17α-hydroxyprogesterone, chlormadinone acetate, cyproterone acetate, gestodene, or etonogestrel.

In some aspects, the estrogen, synthetic estrogen, progesterone, progestin, melatonin, bleomycin, pyrazinamide, sorafenib, or a derivative thereof, or a combination of any thereof is formulated for intramuscular injection, oral administration, subcutaneous, intradermal, or transdermal transport, injection into the blood stream, or for aerosol administration. In some aspects, the estrogen, synthetic estrogen, progesterone, progestin, a melatonin, bleomycin, pyrazinamide, sorafenib, or a derivative thereof, or a combination of any thereof is formulated in a composition.

In various aspects, any composition of the disclosure also comprises other ingredients, such as a diluent, excipients, and/or adjuvant. Acceptable carriers, diluents, excipients, and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

In some aspects, the nucleic acids are introduced into a vector for delivery. In some aspects, the vector for delivery is an AAV or an rAAV. Thus, embodiments of the disclosure include an rAAV genome comprising a nucleotide sequence comprising at least 90% identity to the sequence set forth in any one of SEQ ID NOs: 5-47; the nucleotide sequence set forth in any one of SEQ ID NOs: 5-47; a nucleotide sequence comprising at least 90% identity to the sequence set forth in any one of SEQ ID NOs: 50-92; the nucleotide sequence set forth in any one of SEQ ID NOs: 50-92; a nucleotide sequence that encodes the RNA sequence set forth in any one of SEQ ID NOs: 95-105; or a nucleotide sequence that specifically hybridizes to the DUX4 sequence set forth in any one of SEQ ID NOs:106-124.

In some other aspects, the nucleic acids are introduced into the cell via non-vectorized delivery. Thus, in an embodiment, the disclosure includes non-vectorized delivery of a nucleic acid encoding the DUX4-targeting miRNAs. In some aspects, in this context, synthetic carriers able to form complexes with nucleic acids, and protect them from extra- and intracellular nucleases, are an alternative to viral vectors. In some aspects, such non-vectorized delivery includes the use of nanoparticles, extracellular vesicles, or exosomes comprising the nucleic acids of the disclosure. The disclosure also includes compositions comprising any of the constructs described herein alone or in combination.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg) (e.g., 1×10⁷ vg, 1×10⁸ vg, 1×10⁹ vg, 1×10¹⁰ vg, 1×10¹¹ vg, 1×10¹² vg, 1×10¹³ vg, and 1×10¹⁴ vg, respectively).

In some aspects, therefore, the disclosure provides a method of delivering to a cell or to a subject any one or more nucleic acids comprising a nucleotide sequence comprising at least 90% identity to the sequence set forth in any one of SEQ ID NOs: 5-47; the nucleotide sequence set forth in any one of SEQ ID NOs: 5-47; a nucleotide sequence comprising at least 90% identity to the sequence set forth in any one of SEQ ID NOs: 50-92; the nucleotide sequence set forth in any one of SEQ ID NOs: 50-92; a nucleotide sequence that encodes the RNA sequence set forth in any one of SEQ ID NOs: 95-105; or a nucleotide sequence that specifically hybridizes to the DUX4 sequence set forth in any one of SEQ ID NOs:106-124.

In some aspects, the method comprises administering to a cell or to a subject an AAV comprising any one or more nucleic acids comprising a nucleotide sequence comprising at least 90% identity to the sequence set forth in any one of SEQ ID NOs: 5-47; the nucleotide sequence set forth in any one of SEQ ID NOs: 5-47; a nucleotide sequence comprising at least 90% identity to the sequence set forth in any one of SEQ ID NOs: 50-92; the nucleotide sequence set forth in any one of SEQ ID NOs: 50-92; a nucleotide sequence that encodes the RNA sequence set forth in any one of SEQ ID NOs: 95-105; or a nucleotide sequence that specifically hybridizes to the DUX4 sequence set forth in any one of SEQ ID NOs:106-124.

In yet another aspect, the disclosure provides a method of decreasing expression of the DUX4 gene or decreasing the expression of functional DUX4 in a cell or a subject, wherein the method comprises contacting the cell or the subject with any one or more nucleic acids comprising a nucleotide sequence comprising at least 90% identity to the sequence set forth in any one of SEQ ID NOs: 5-47; the nucleotide sequence set forth in any one of SEQ ID NOs: 5-47; a nucleotide sequence comprising at least 90% identity to the sequence set forth in any one of SEQ ID NOs: 50-92; the nucleotide sequence set forth in any one of SEQ ID NOs: 50-92; a nucleotide sequence that encodes the RNA sequence set forth in any one of SEQ ID NOs: 95-105; or a nucleotide sequence that specifically hybridizes to the DUX4 sequence set forth in any one of SEQ ID NOs:106-124.

In some aspects, the method comprises delivering the nucleic acids in one or more AAV vectors. In some aspects, the method comprises delivering the nucleic acids to the cell in non-vectorized delivery.

In some aspects, expression of DUX4 or the expression of functional DUX4 is decreased in a cell or in a subject by the methods provided herein by at least or about 5, about 10, about 15, about 20, about 25, about 30, about 35, about 40, about 45, about 50, about 55, about 60, about 65, about 70, about 75, about 80, about 85, about 90, about 95, about 96, about 97, about 98, about 99, or 100 percent.

In some aspects, the disclosure provides AAV transducing cells for the delivery of nucleic acids encoding the DUX4 miRNA as described herein. Methods of transducing a target cell with rAAV, in vivo or in vitro, are included in the disclosure. The methods comprise the step of administering an effective dose, or effective multiple doses, of a composition comprising a rAAV of the disclosure to a subject, including an animal (such as a human being) in need thereof. If the dose is administered prior to development of the muscular dystrophy, the administration is prophylactic. If the dose is administered after the development of the muscular dystrophy, the administration is therapeutic. In embodiments of the disclosure, an effective dose is a dose that alleviates (eliminates or reduces) at least one symptom associated with the muscular dystrophy being treated, that slows or prevents progression of the muscular dystrophy, that slows or prevents progression of the muscular dystrophy, that diminishes the extent of disease, that results in remission (partial or total) of the muscular dystrophy, and/or that prolongs survival. In some aspects, the muscular dystrophy is FSHD.

In some aspects, the disclosure provided non-vectorized delivery of nucleic acids encoding the DUX4 miRNA as described herein. In some aspects, the nucleic acids or compositions comprising the nucleic acids are delivered in nanoparticles, extracellular vesicles, or exosomes.

Combination therapies are also contemplated by the disclosure. Combination as used herein includes simultaneous treatment or sequential treatments. Combinations of methods of the disclosure with standard medical treatments (e.g., corticosteroids and/or immunosuppressive drugs) or with other inhibitory RNA constructs are specifically contemplated, as are combinations with other therapies such as those disclosed in International Publication No. WO 2013/016352, which is incorporated by reference herein in its entirety.

Administration of an effective dose of the compositions, including AAV, nanoparticles, extracellular vesicles, and exosomes comprising the compositions and nucleic acids of the disclosure, may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. Route(s) of administration and serotype(s) of AAV components of rAAV (in particular, the AAV ITRs and capsid protein) of the disclosure may be chosen and/or matched by those skilled in the art taking into account the disease state being treated and the target cells/tissue(s), such as cells that express DUX4. In some embodiments, the composition or medicament is formulated for intramuscular injection, oral administration, subcutaneous, intradermal, or transdermal transport, injection into the blood stream, or for aerosol administration. In some embodiments, the route of administration is intramuscular. In some embodiments, the route of administration is intravenous.

In some aspects, actual administration of rAAV of the present disclosure may be accomplished by using any physical method that will transport the rAAV recombinant vector into the target tissue of an animal. Administration according to the disclosure includes, but is not limited to, injection into muscle, the bloodstream, the central nervous system, and/or directly into the brain or other organ. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared for oral administration, as injectable formulations, or as topical formulations to be delivered to the muscles by subcutaneous, intradermal, and/or transdermal transport. Numerous formulations for both intramuscular injection and transdermal transport have been previously developed and can be used in the practice of the disclosure. The rAAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. In some aspects, proper fluidity is maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

In some aspects, the formulation comprises a stabilizer. The term “stabilizer” refers to a substance or excipient which protects the formulation from adverse conditions, such as those which occur during heating or freezing, and/or prolongs the stability or shelf-life of the formulation in a stable state. Examples of stabilizers include, but are not limited to, sugars, such as sucrose, lactose and mannose; sugar alcohols, such as mannitol; amino acids, such as glycine or glutamic acid; and proteins, such as human serum albumin or gelatin.

In some aspects, the formulation comprises an antimicrobial preservative. The term “antimicrobial preservative” refers to any substance which is added to the composition that inhibits the growth of microorganisms that may be introduced upon repeated puncture of the vial or container being used. Examples of antimicrobial preservatives include, but are not limited to, substances such as thimerosal, 2-phenoxyethanol, benzethonium chloride, and phenol.

The term “transduction” is used to refer to the administration/delivery of one or more of the DUX4 targeting constructs, e.g., DUX4 miRNA or nucleic acid encoding DUX miRNA, described herein to a recipient cell either in vivo or in vitro, via a replication-deficient rAAV of the disclosure resulting in decreased expression of DUX4 by the recipient cell.

In one aspect, transduction with rAAV is carried out in vitro. In one embodiment, desired target cells are removed from the subject, transduced with rAAV and reintroduced into the subject. Alternatively, syngeneic or xenogeneic cells can be used where those cells will not generate an inappropriate immune response in the subject.

Suitable methods for the transduction and reintroduction of transduced cells into a subject are known in the art. In one embodiment, cells are transduced in vitro by combining rAAV with cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

The disclosure provides methods of administering an effective dose (or doses, administered essentially simultaneously or doses given at intervals) of rAAV that comprise DNA that encodes microRNA designed to downregulate or inhibit the expression of DUX4 to a cell or to a subject in need thereof. In some aspects, the effective dose is therefore a therapeutically effective dose.

In some embodiments, the dose or effective dose of rAAV administered is about 1.0×10¹⁰ vg/kg to about 1.0×10¹⁶ vg/kg. In some aspects, 1.0×10¹⁰ vg/kg is also designated 1.0 E10 vg/kg, which is simply an alternative way of indicating the scientific notation. Likewise, 10¹¹ is equivalent to E11, and the like. In some aspects, the dose of rAAV administered is about 1.0×10¹¹ vg/kg to about 1.0×10¹⁵ vg/kg. In some aspects the dose of rAAV is about 1.0×10¹⁰ vg/kg, about 2.0×10¹⁰ vg/kg, about 3.0×10¹⁰ vg/kg, about 4.0×10¹⁰ vg/kg, about 5.0×10¹⁰ vg/kg, about 6.0×10¹⁰ vg/kg, about 7.0×10¹⁰ vg/kg, about 8.0×10¹⁰ vg/kg, about 9.0×10¹⁰ about 1.0×10¹¹ vg/kg, about 2.0×10¹¹ vg/kg, about 3.0×10¹¹ vg/kg, about 4.0×10¹¹ vg/kg, about 5.0×10¹¹ vg/kg, about 6.0×10¹¹ vg/kg, about 7.0×10¹¹ vg/kg, about 8.0×10¹¹ vg/kg, about 9.0×10¹¹ vg/kg, about 1.0×10¹² vg/kg, about 2.0×10¹² vg/kg, about 3.0×10¹² vg/kg, about 4.0×10¹² vg/kg, about 5.0×10¹² vg/kg, about 6.0×10¹² vg/kg, about 7.0×10¹² vg/kg, about 8.0×10¹² vg/kg, about 9.0×10¹² vg/kg, about 1.0×10¹³ vg/kg, about 2.0×10¹³ vg/kg, about 3.0×10¹³ vg/kg, about 4.0×10¹³ vg/kg, about 5.0×10¹³ vg/kg, about 6.0×10¹³ vg/kg, about 7.0×10¹³ vg/kg, about 8.0×10¹³ vg/kg, about 9.0×10¹³ vg/kg, about 1.0×10¹⁴ vg/kg, about 2.0×10¹⁴ vg/kg, about 3.0×10¹⁴ vg/kg, about 4.0×10¹⁴ vg/kg, about 5.0×10¹⁴ vg/kg, about 6.0×10¹⁴ vg/kg, about 7.0×10¹⁴ vg/kg, about 8.0×10¹⁴ vg/kg, about 9.0×10¹⁴ vg/kg, about 1.0×10¹⁵ vg/kg, about 2.0×10¹⁵ vg/kg, about 3.0×10¹⁵ vg/kg, about 4.0×10¹⁵ vg/kg, about 5.0×10¹⁵ vg/kg, about 6.0×10¹⁵ vg/kg, about 7.0×10¹⁵ vg/kg, about 8.0×10¹⁵ vg/kg, about 9.0×10¹⁵ vg/kg, or about 1.0×10¹⁶ vg/kg.

In some aspects, the dose is about 1.0×10¹¹ vg/kg to about 1.0×10¹⁵ vg/kg. In some aspects, the dose is about 1.0×10¹³ vg/kg to about 5.0×10¹³ vg/kg. In some aspects, the dose is about 2.0×10¹³ vg/kg to about 4.0×10¹³ vg/kg. In some aspects, the dose is about 3.0×10¹³ vg/kg.

In some aspects, an initial dose is followed by a second greater dose. In some aspects, an initial dose is followed by a second same dose. In some aspects, an initial dose is followed by one or more lesser doses. In some aspects, an initial dose is followed by multiple doses which are the same or greater doses.

Methods of transducing a target cell with a delivery vehicle (such as rAAV), in vivo or in vitro, are contemplated. Transduction of cells with an rAAV of the disclosure results in sustained expression of DUX4 miRNA sequence. The disclosure thus provides rAAV and methods of administering/delivering rAAV which express DUX4 miRNA sequence in the cell(s) in vitro or in vivo in a subject. In some aspects, the subject is a mammal. In some aspects, the mammal is a human. These methods include transducing cells and tissues (including, but not limited to, tissues such as muscle) with one or more rAAV described herein. Transduction may be carried out with gene cassettes comprising cell-specific control elements. The term “transduction” is used to refer to, as an example, the administration/delivery of a nucleic acid comprising a nucleotide sequence encoding a DUX4 miRNA sequence, e.g., DUX4 miRNA, to a target cell either in vivo or in vitro, via a replication-deficient rAAV described herein resulting in the decreased expression or inhibition of expression of DUX4 by the target cell.

The in vivo methods comprise the step of administering an effective dose, or effective multiple doses, of a composition comprising a delivery vehicle (such as rAAV) to a subject (including a human subject) in need thereof. Thus, methods are provided of administering an effective dose (or doses, administered essentially simultaneously or doses given at intervals) of rAAV described herein to a subject in need thereof. If the dose or doses is administered prior to development of a disorder/disease, the administration is prophylactic. If the dose or doses is administered after the development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival.

In some embodiments, compositions and methods of the disclosure are used in treating, ameliorating, or preventing a disease, such as a muscular dystrophy (MD). In various aspects, such MD is FSHD. FSHD is among the most commonly inherited muscular dystrophies, estimated to affect as many as 870,000 individuals. Classical descriptions of FSHD presentation include progressive muscle weakness in the face, shoulder-girdle and arms, but disease can manifest more broadly, including in muscles of the trunk and lower extremities. Variability is also commonly seen within individuals, as asymmetrical weakness is common. Age-at-onset can range from early childhood to adulthood, and is usually related to disease severity, where earlier onset is often associated with more severe muscle weakness. Although most patients with FSHD have a normal life span, respiratory insufficiency can occur, and the disease can be debilitating, as approximately 25% of affected individuals may become wheelchair dependent by their fifties, and even earlier in more severe forms of the disease, while others maintain lifelong ambulation.

FSHD is caused by aberrant expression of the double homeobox 4 gene (DUX4), which produces a transcription factor that is toxic to skeletal muscle. DUX4 is normally functional during the two-cell stage of human development but repressed thereafter in essentially all other tissues, except perhaps the testes. In skeletal muscles of people with FSHD, specific genetic and epigenetic factors conspire to permit DUX4 de-repression, where it then initiates several aberrant gene expression cascades, including those involved in differentiation abnormalities, oxidative stress, inflammatory infiltration, cell death and muscle atrophy.

In families known to carry pathological FSHD, the methods of the disclosure, in various aspects, are methods of preventing disease and they are carried out before the onset of disease. In other various aspects, the methods of the disclosure are carried out after diagnosis and, therefore, are methods of treating or ameliorating disease.

In some embodiments, compositions and methods of the disclosure are used in treating, ameliorating, or preventing a disease, such as a cancer. DUX4 has been shown to be activated in some cancer types, where it functions to mask tumor cells from the immune system [Chew et al., Dev. Cell 50(5):658-71 (2019)]. For example, DUX4 protein fusions are known to cause cancer, such as rhabdomyosarcoma and Ewing's sarcoma. A CIC-DUX4 gene fusion induces sarcomas and drives sarcoma metastasis [Yoshimoto et al., Cancer Res. 2017 Jun. 1; 77(11): 2927-2937; Okimoto et al., J Clin Invest. 2019; 129(8):3401-3406)]. Other cancer tissues, such as those tissues from the adrenal, B-cell lymphoma, bile duct, bladder, breast, cervix, colon, endometrium, esophagus, head/neck, liver, brain (e.g., lower grade glioma), lung, mesothelium, neural crest, ovary, pancreas, prostate, kidney, skin, soft tissue, stomach, testicles, and thymus, also were shown to express DUX4 [Chew et al., Dev. Cell 50(5):658-71 (2019)]. Thus, the nucleic acids, rAAV and compositions described herein are used in inhibiting DUX4 expression in the treatment, amelioration, or prevention of cancer.

Molecular, biochemical, histological, and functional outcome measures demonstrate the therapeutic efficacy of the products and methods disclosed herein for decreasing the expression of the DUX4 gene and protein and treating muscular dystrophies, such as FSHD. Outcome measures are described, for example, in Chapters 32, 35 and 43 of Dyck and Thomas, Peripheral Neuropathy, Elsevier Saunders, Philadelphia, PA, 4th Edition, Volume 1 (2005) and in Burgess et al., Methods Mol. Biol., 602: 347-393 (2010). Outcome measures include, but are not limited to, reduction or elimination of DUX4 mRNA or protein in affected tissues. The lack of expression of DUX4 and/or the downregulation of expression of DUX4 in the cell is detected by measuring the level of DUX4 protein by methods known in the art including, but not limited to, RT-PCR, QRT-PCR, RNAscope, Western blot, immunofluorescence, or immunohistochemistry in muscle biopsied before and after administration of the rAAV to determine the improvement.

In some embodiments, the level of DUX4 gene expression or protein expression in a cell of the subject is decreased after administration of the nucleic acid encoding the DUX4 miRNA or the vector, e.g., rAAV, comprising the nucleic acid encoding the DUX4 miRNA as compared to the level of DUX4 gene expression or protein expression before administration of the nucleic acid encoding the DUX4 miRNA or the vector, e.g. rAAV. In some aspects, expression of a DUX4 is decreased by at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%. In various aspects, improved muscle strength, improved muscle function, and/or improved mobility and stamina show an improvement by at least about 2%, at least about 5%, at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%.

Other outcome measures include measuring the level of serum creatinine kinase (CK) in the subject before and after treatment. Increased CK levels are a hallmark of muscle damage. In muscular dystrophy patients, CK levels are significantly increased above the normal range (10 to 100 times the normal level since birth). When elevated CK levels are found in a blood sample, it usually means muscle is being disintegrated by some abnormal process, such as a muscular dystrophy or inflammation. Thus, a positive therapeutic outcome for treatment with the methods of the disclosure is a reduction in the level of serum creatinine kinase after administration of the rAAV as compared to the level of serum creatinine kinase before administration of the rAAV.

Other outcome measure include measuring to determine if there is improved muscle strength, improved muscle function, improved mobility, improved stamina, or a combination of two or more thereof in the subject after treatment. Such outcome measures are important in determining muscular dystrophy progression in the subject and are measured by various tests known in the art. Some of these tests include, but are not limited to, the six minute walk test, time to rise test, ascend 4 steps test, ascend and descend 4 steps test, North Star Ambulatory Assessment (NSAA) test, 10 meter timed test, 100 meter timed test, hand held dynamometry (HHD) test, Timed Up and Go test, Gross Motor Subtest Scaled (Bayley-Ill) score, maximum isometric voluntary contraction test (MVICT), or a combination of two or more thereof.

Combination therapies are also contemplated by the disclosure. Combination as used herein includes both simultaneous treatment and sequential treatments. Combinations of methods described herein with standard medical treatments and supportive care are specifically contemplated, as are combinations with therapies, such as glucocorticoids. All types of glucocorticoids are included for use in the combination therapies disclosed herein. Such glucocorticoids include, but are not limited to, prednisone, prednisolone, dexamethasone, deflazacort, beclomethasone, betamethasone, budesonide, cortisone, hydrocortisone, methylprednisolone, and triamcinolone.

Other combination therapies included in the disclosure are the DUX4 miRNAs, as described herein, in combination with other miRNAs, or in combination with U7-snRNA-based gene therapy, a small molecule inhibitor of DUX4 expression, oligonucleotides to inhibit DUX4 through RNAi or RNAse H or exon skipping mechanisms, U7-snRNA plus a theoretical CRISPR-based gene therapy approach.

Administration of an effective dose of a nucleic acid, viral vector, or composition of the disclosure may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. In some aspects, an effective dose is delivered by a systemic route of administration, i.e., systemic administration. Systemic administration is a route of administration into the circulatory system so that the entire body is affected. Such systemic administration, in various aspects, takes place via enteral administration (absorption of the drug through the gastrointestinal tract) or parenteral administration (generally via injection, infusion, or implantation). In various aspects, an effective dose is delivered by a combination of routes. For example, in various aspects, an effective dose is delivered intravenously and/or intramuscularly, or intravenously and intracerebroventricularly, and the like. In some aspects, an effective dose is delivered in sequence or sequentially. In some aspects, an effective dose is delivered simultaneously. Route(s) of administration and serotype(s) of AAV components of the rAAV (in particular, the AAV ITRs and capsid protein) of the disclosure, in various aspects, are chosen and/or matched by those skilled in the art taking into account the condition or state of the disease or disorder being treated, the condition, state, or age of the subject, and the target cells/tissue(s) that are to express the nucleic acid or protein.

In particular, actual administration of delivery vehicle (such as rAAV) may be accomplished by using any physical method that will transport the delivery vehicle (such as rAAV) into a target cell of an animal. Administration includes, but is not limited to, injection into muscle, the bloodstream and/or directly into the nervous system or liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as neurons. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The delivery vehicle (such as rAAV) can be used with any pharmaceutically acceptable carrier for ease of administration and handling.

A dispersion of delivery vehicle (such as rAAV) can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

“Treating” includes ameliorating or inhibiting one or more symptoms of a muscular dystrophy including, but not limited to, muscle wasting, muscle weakness, myotonia, skeletal muscle problems, abnormalities of the retina, hip weakness, facial weakness, abdominal muscle weakness, joint and spinal abnormalities, lower leg weakness, shoulder weakness, hearing loss, muscle inflammation, and nonsymmetrical weakness.

Administration of an effective dose of a nucleic acid, viral vector, or composition of the disclosure may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. In some aspects, an effective dose is delivered by a systemic route of administration, i.e., systemic administration. Systemic administration is a route of administration into the circulatory system so that the entire body is affected. Such systemic administration, in various aspects, takes place via enteral administration (absorption of the drug through the gastrointestinal tract) or parenteral administration (generally via injection, infusion, or implantation). In various aspects, an effective dose is delivered by a combination of routes. For example, in various aspects, an effective dose is delivered intravenously and/or intramuscularly, or intravenously and intracerebroventricularly, and the like. In some aspects, an effective dose is delivered in sequence or sequentially. In some aspects, an effective dose is delivered simultaneously. Route(s) of administration and serotype(s) of AAV components of the rAAV (in particular, the AAV ITRs and capsid protein) of the disclosure, in various aspects, are chosen and/or matched by those skilled in the art taking into account the condition or state of the disease or disorder being treated, the condition, state, or age of the subject, and the target cells/tissue(s) that are to express the nucleic acid or protein.

In particular, actual administration of delivery vehicle (such as rAAV) may be accomplished by using any physical method that will transport the delivery vehicle (such as rAAV) into a target cell of an animal. Administration includes, but is not limited to, injection into muscle, the bloodstream and/or directly into the nervous system or liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as neurons. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The delivery vehicle (such as rAAV) can be used with any pharmaceutically acceptable carrier for ease of administration and handling.

A dispersion of delivery vehicle (such as rAAV) can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The disclosure also provides a kit comprising a nucleic acid, vector, or composition of the disclosure or produced according to a process of the disclosure. In the context of the disclosure, the term “kit” means two or more components, one of which corresponds to a nucleic acid, vector, or composition of the disclosure, and the other which corresponds to a container, recipient, instructions, or otherwise. A kit, therefore, in various aspects, is a set of products that are sufficient to achieve a certain goal, which can be marketed as a single unit.

The kit may comprise one or more recipients (such as vials, ampoules, containers, syringes, bottles, bags) of any appropriate shape, size and material containing the nucleic acid, vector, or composition of the disclosure in an appropriate dosage for administration (see above). The kit may additionally contain directions or instructions for use (e.g. in the form of a leaflet or instruction manual), means for administering the nucleic acid, vector, or composition, such as a syringe, pump, infuser or the like, means for reconstituting the nucleic acid, vector, or composition and/or means for diluting the nucleic acid, vector, or composition.

In some aspects, the kit comprises a label and/or instructions that describes use of the reagents provided in the kit. The kits also optionally comprise catheters, syringes or other delivering devices for the delivery of one or more of the compositions used in the methods described herein.

The disclosure also provides kits for a single dose of administration unit or for multiple doses. In some embodiments, the disclosure provides kits containing single-chambered and multi-chambered pre-filled syringes.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning.

Unless otherwise indicated, the term “at least” preceding a series of elements is to be understood to refer to every element in the series. Those skilled in the art will recognize, or be able to ascertain using no more than routine experimentation, many equivalents to the specific embodiments of the disclosure described herein. Such equivalents are intended to be encompassed by the disclosure.

The term “and/or” wherever used herein includes the meaning of “and”, “or” and “all or any other combination of the elements connected by said term.”

The term “about” or “approximately” as used herein means within 20%, preferably within 10%, and more preferably within 5% of a given value or range. It includes, however, also the concrete number, e.g., about 10 includes 10.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein, “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this disclosure is not limited to the particular methodology, protocols, material, reagents, and substances, etc., described herein and as such can vary. The terminology used herein is for the purpose of describing particular embodiments only, and is not intended to limit the scope of the subject matter of the disclosure, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material.

A better understanding of the disclosure and of its advantages will be obtained from the following examples, offered for illustrative purposes only. The examples are not intended to limit the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional aspects and details of the disclosure will be apparent from the following examples, which are intended to be illustrative rather than limiting.

### Example 1

### Materials and Methods

Study Design. The objective of the study was to explore new strategies for the treatment of a muscular dystrophy, such as FSHD, or a cancer resulting from expression or an overexpression of DUX4. FSHD is caused by de-repression of the DUX4 gene, which is toxic to muscle. FSHD therapies are thus focused on inhibiting DUX4, which was a main goal of this study. In this study, a novel strategy was developed to direct RNAi against DUX4. Specifically, drugs to up-regulate endogenous human microRNAs that naturally direct RNAi against DUX4 were tested, with the theory that this approach would offer a novel strategy to inhibit the DUX4 gene with RNAi. In this study, it was shown that mir-675 inhibits DUX4 efficiently and reduces DUX4-associated phenotypes in human HEK293 cells and FSHD muscle cell lines. It was also shown that mir-675 functions within a gene therapy vector to inhibit DUX4-associated pathologies in vivo—in an AAV.DUX4 mouse model previously developed and published [Wallace et al, Ann. Neurol. 69: 540-552 (2011)]. For the in vitro study, between n=3 to n=6 independent experiments were carried out, depending on the assay. Six independent blinded western blots were performed when testing mir-675 specific inhibition of DUX4 expression. In the small molecule treatment assay, three different FSHD cell lines, i.e., 15A, 17A, and 18A, were used. For the 15A FSHD cell line, 6 independent experiments were performed. For the 17A and 18A FSHD cell lines, 3 independent experiments were performed. For the in vivo study, sample size was chosen based on previously published studies [Wallace et al., Mol. Ther. 20: 1417-23 (2012); Wallace et al., Mol. Ther. Methods Clin. Dev. 8: 121-130 (2018)].

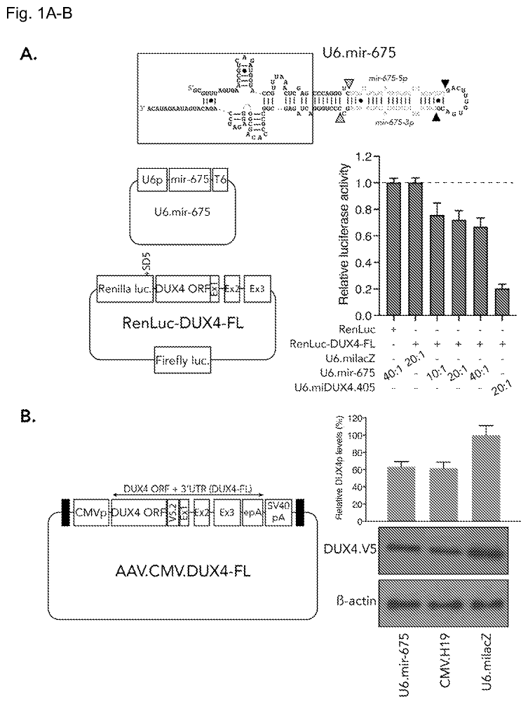

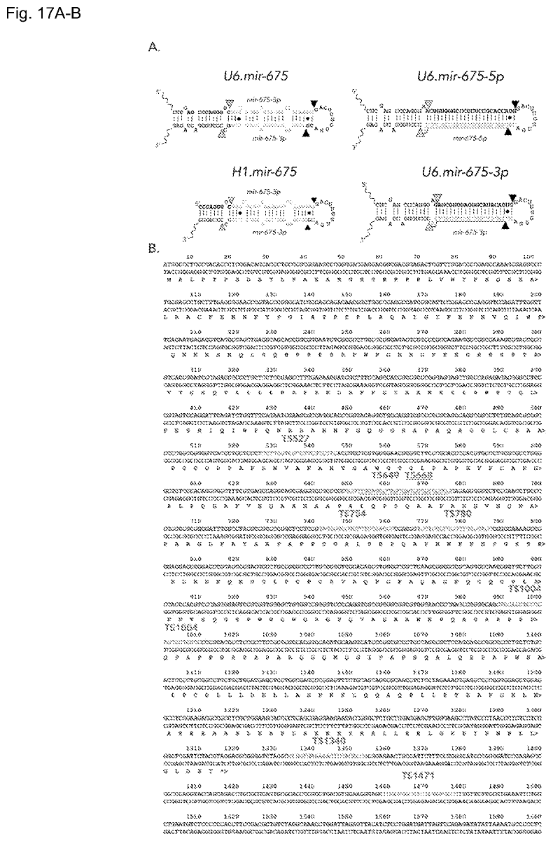

Sequence Generation and Cloning. All miRNAs used in this study were designed as previously reported in [Wallace et al., Mol. Ther. Methods Clin. Dev. 8:121-130 (2018)]. All miDUX4s were cloned into the mir-30 based/U6 construct as previously described [Wallace et al., Mol. Ther. 20:1417-23 (2012)]. MicroRNA-675 sequence was obtained from the miRBase database (www.mirbase.org), and all mir-675 constructs used in this study were cloned into an expression plasmid downstream of an RNA polymerase III class of promoters (U6 or H1 promoters). The sequence of the stem loop structure of mir-675, mir-675-5p and mir-675-3p constructs is shown in FIG. 17A-B. The CMV.H19 construct was purchased from OriGene. The psi2-DUX4FI was PCR amplified using the AAV.CMV.DUX44V5 construct as template, and the following primers: forward primer: CCGGCTCGAGATGGCCCTCCCGACAC (SEQ ID NO: 127), and reverse primer: ACGACTAGTGGGAGGGGGCATTTTAATATATCTC (SEQ ID NO: 128). The PCR product was then cloned into a previously designed psicheck2 (psi2) SD5 mutant (Renilla luciferase have the SD5 mutant) plasmid [Ansseau et al., Plos One 10: e0118813 (2015)] using XhoI/SpeI restriction sites and the psi2.SD5 mutant-DUX4 3′UTR plasmid backbone. The AAV.CMV.DUX4-FL construct expressing the full length DUX4 (DUX4-FL) encompassing DUX4 ORF and 3′UTR, as well as the V5 tag at its 3′ end, was PCR amplified and cloned into the AAV6.CMV pro-viral backbone plasmid as previously described [Wallace et al., Ann. Neurol. 69: 540-52(2011)]. The original V5 peptide tag was mutated (V5.2) to prevent mis-splicing of the V5 and DUX4 stop codon via recombinant PCR as previously described [Ansseau et al., Plos One 10: e0118813 (2015)]. In addition, this plasmid contains a cytomegalovirus promoter (CMVp)-driven DUX4 full-length sequence encompassing the DUX4 open reading frame (DUX4 ORF), the DUX4 3′UTR sequence (pLAM sequence). The latter is formed of exon 1 (Ex1), exon 2 (Ex2) and exon 3 (Ex3) and the endogenous DUX4 unconventional polyA sequence (ATTAAAA (SEQ ID NO: 129)) (epA). To clone CMV.eGFP into the AAV.CMV.DUX4-FL plasmid, CMV.eGFP was amplified by PCR using AAV.CMV.eGFP as a template and the following primers: forward primer: TTACTAGTATTAATAGTAATCAATTACGG (SEQ ID NO: 130), and reverse primer: CAATGAATTCGTTAATGATTAACCCGCCAT (SEQ ID NO: 131). The PCR product was then cloned in the plasmid backbone using the SpeI/EcoRI restriction sites. The CMV.DUX4 mir-675Res construct expressing the DUX4 ORF mutant resistant to mir-675 inhibition was cloned by recombinant PCR using EcoRI/KpnI restriction sites, using wild-type the DUX4 as template, and the following primers: forward: 5′CCGAGAATTCCTCGACTTATTAATAGTAATCAATTACGGGGTCA 3′ (SEQ ID NO: 132), forward middle: 5′ ACCCAAGATCTGGGGCAAGGTGGGCAAAAGCCGGGAGGA 3′ (SEQ ID NO: 133), reverse middle: 5′ CACCTTGCCCCAGATCTTGGGTGCCTGAGGGTGGGAGAG 3′ (SEQ ID NO: 134), reverse: 5′ CGGGTACCCTACGTAGAATCGAGCCCGAGGAG 3′ (SEQ ID NO: 135). The CMV.DUX4-mir-675Res contains a CMVp-driven DUX4 ORF with point mutations in the high affinity, ORF-located mir-675 binding site (see TS780M vs TS780WT sequence alignment), and has no DUX4 3′UTR. The absence of the latter eliminated the other mir-675 target sites on the DUX4 transcript. This construct also contains the mutated V5 epitope sequence (V5.2) and the SV40 polyadenylation signal (SV40 pA). To clone CMV.eGFP into the CMV.DUX4-FL/CMV.eGFP and CMV.DUX4 mir-675Res plasmids, CMV.eGFP was amplified by PCR using CMV.eGFP as template and the following primers: forward: TTACTAGTATTAATAGTAATCAATTACGG (SEQ ID NO: 136), reverse: CAATGA ATTCGTTAATGATTAACCCGCCAT (SEQ ID NO: 137). The PCR product was then cloned in the plasmid backbone using SpeI/EcoRI restriction sites. To make RenLuc-PTS (reverse complement of every mature miRNA sequence), an oligonucleotide containing target sites of every miRBase-predicted miRNA was commercially made and used in this study, and recombinant PCR was used to fuse it as the 3′UTR of Renilla luciferase in the psiCheck2 (RenLuc) dual luciferase plasmid (Promega). A similar strategy was used to clone the perfect target site for mir-675 at the 3′UTR of Renilla luciferase in RenLuc-mir-675R. To make the dual luciferase plasmids in which the DUX4 ORF, DUX4 3′ UTR, or full-length DUX4 (DUX4-FL) were inserted as the Renilla luciferase 3′UTR (RenLuc-DUX4 ORF, RenLuc-DUX4 3′UTR, and DUX4-FL), sequences were amplified by PCR using the CMV.DUX4-FLAV5 plasmid as a template, and cloned into the RenLuc. To make RenLuc-DUX4-FL expression plasmid (FIG. 1A), DUX4-FL (DUX4 ORF without V5 tag+3′UTR) was PCR amplified using CMV.DUX4-FLAV5 as template with the following primers: forward: 5′ CCGGCTCGAGATGGCCCTCCCGACAC 3′ (SEQ ID NO: 138), reverse: 5′ ACGACTAGTGGGAGGGGGCATTTTAATATATCTC 3′ (SEQ ID NO: 139). The PCR product was then cloned into a previously designed RenLuc SD5 mutant plasmid using XhoI/SpeI restriction sites and the RenLuc.SD5 mutant-DUX4 3′UTR plasmid backbone. The Renilla luciferase gene has a splicing donor mutation (*SID5) that prevents the alternative splicing of the DUX4-FL mRNA [Ansseau et al., Plos One 10: e0118813 (2015)]. To make the RenLuc-DUX4 ORF-mir-675Res expression plasmid, recombinant PCR was carried out to delete one of the strongest mir-675 target sites (TS780) in DUX4 ORF and eliminated the DUX4 3′UTR using the following primers: forward: 5′ CCGGCTCGAGATGGCCCTCCCGACAC 3′ (SEQ ID NO: 140), forward middle: 5′ CGGGCAAAAGCCGGGAGGA 3′ (SEQ ID NO: 141), reverse middle: 5′ TCCTCCCGGCTTTTGCCCGGCCTGAGGGTGGGAGA 3′ (SEQ ID NO: 142), and reverse: 5′ AGCGGCCGCAAGCTCCTCCAGCAGAGC 3′ (SEQ ID NO: 143). This construct was then cloned into the RenLuc-backbone using XhoI/NotI restriction sites.

Cell Culture.

HEK293 Cell Culture. HEK293 cells were grown using DMEM (Gibco) medium supplemented with 20% FBS (Corning), 1% L-glutamine (Gibco) and 1% Penicillin-Streptomycin (Gibco). Transfected cells were grown in the same DMEM medium but lacking Penicillin-Streptomycin.

Primary Cell Culture. 15A, 17A sand 18A FSHD human myoblasts and 15V control human myoblasts were provided by the UMMS Wellstone Center for FSHD and have been previously characterized [Jones et al., Hum. Mol. Genet. 21: 4419-4430 (2012)]. Previously immortalized 15A cell lines have a single 4qA permissive allele. Using immunocytochemistry (ICC), Jones (supra) showed that 15A cell lines have 1:104 DUX4+nuclei, which is low when compared to other FSHD affected cell lines (i.e. 17A and 18A). 15V cell lines have two 4qB non-permissive alleles. Cells were propagated by feeding them every two days with new LHCN medium [4:1 DMEM:Medium 199 (Gibco) supplemented with 15% characterized FBS (Corning), 0.02 M HEPES (Thermo Fisher), 0.03 μg/mL ZnSO4 (Honeywell Fluka), 1.4 μg/mL Vitamin B12 (Sigma-Aldrich), 0.055 μg/mL dexamethasone (Sigma-Aldrich), 1% antibiotics/antimycotics (Gibco), 2.5 ng/mL hepatocyte growth factor (Millipore) and 10 ng/mL basic fibroblast growth factor (Millipore)]. For differentiation, myoblasts were switched to a differentiation medium [4:1 DMEM: Medium 199 (Gibco) supplemented with 15% KnockOut Serum Replacement (ThermoFisher Scientific), 2 mM L-glutamine (Gibco), 1% antibiotics/antimycotics (Gibco), 1 mM sodium pyruvate (Gibco) and 20 mM HEPES (ThermoFisher Scientific)] when cells were at >90% confluency. Before adding differentiation medium, cells were washed with PBS (Gibco). Cells were seeded with new differentiation medium every three days for up to 7 days. To detach cells, TrypLE™ Express, phenol red (ThermoFisher Scientific) was used.

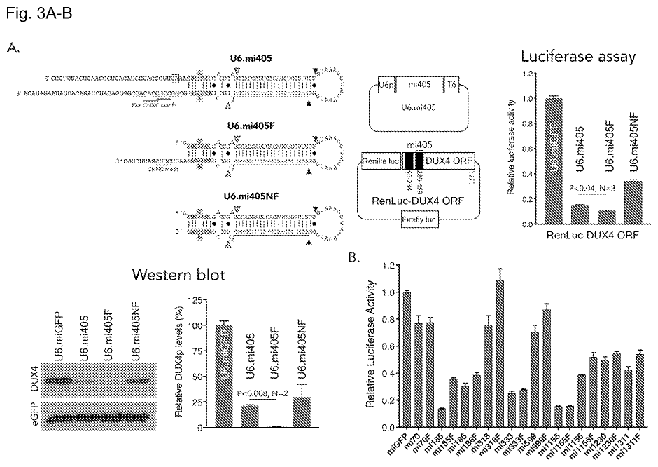

Dual Luciferase Assay. (See also FIGS. 1A, 3A-B, 4A-B, 5B, and 16B-C.) This assay was performed as previously described by Wallace et al. (Mol. Ther. Methods Clin. Dev. 8: 121-30 (2018)) and following the dual-luciferase reporter assay system (Promega) protocol with some modifications. All plasmid constructs had the psiCheck2 dual luciferase reporter plasmid (Promega) as backbone that contains separate Renilla and Firefly luciferase genes, where the former contains the various target sequences used in the experiments of the disclosure, and the latter serves as a transfection normalizer (control). All DUX4 and control sequences were cloned downstream of the Renilla luciferase stop codon, serving as a 3′UTR. HEK293 cells were pre-plated 24h before transfection. Cells were then co-transfected with the luciferase DUX4 reporter and individual microRNA expression plasmids in an increasing luciferase DUX4 reporter:miRNA molar ratio using Lipofectamine 2000 (Invitrogen). Luciferase activity was measured 24h or 48h after transfection. DUX4 gene silencing was determined as previously described [Wallace et al., Mol. Ther. Methods Clin. Dev. 8: 121-30 (2018)]. Triplicate data were averaged per experiment, and individual experiments were performed 3 times. Results were reported as the average ratio of Renilla to Firefly luciferase activity±SEM for all combined experiments.

RNA Extraction. RNA from HEK293 cells was extracted for Northern blot assay and QPCR. The miRVANA miRNA isolation kit (ThermoFisher Scientific) was used according to manufacturer's directions to extract total RNA encompassing small RNAs, such as miRNAs. To extract RNA from C57BL/6 skeletal muscles, cryopreserved muscles were crushed under suboptimal temperatures using liquid nitrogen and using mortar and pestle. Crushed muscles were then lysed using 6004 of miRVANA miRNA isolation kit lysis buffer, a TissueLyser and 1.0 mm zirconia beads (Biospec). Muscle was homogenized at 30 Hz for 30 sec with 10 sec rest. This was repeated 3 times.

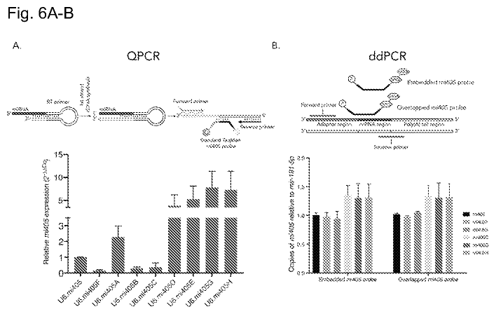

Quantitative RT-PCR Assays. (See also FIGS. 6A-B, 9A-B, 15A, 16A-B, 23A-B, 24, and 25.)

TaqMan Gene Expression Assay. QRT-PCR was carried out to quantify the expression of pri-mir-675, mir-675-5p and mi405 using TaqMan probes (ThermoFisher Scientific). Experiments were started by eliminating genomic DNA from the RNA preparations, and then cDNA was generated using the High-Capacity cDNA Reverse Transcription Kit (Applied Biosystems) following manufacturer's instructions. Within the same cDNA reaction, 1×RT random hexamer primers were used to generate the cDNA for RPL13A, used as a reference gene, and for pri-mir-675. To generate the cDNA for mir-675, the RT mir-675 reverse primer provided by ThermoFisher Scientific was used. For mi405, the mi405 qPCR protocol was done as it was previously described [Wallace et al., Mol. Ther. Methods Clin. Dev. 8: 121-30 (2018)]. To quantify mir-675 and mi405 levels, RNA was extracted using the total RNA protocol for the mirVana miRNA Isolation Kit (Ambion) from HEK293 cells. cDNA was generated using the High-Capacity cDNA Reverse Transcription Kit (Applied Biosystems) using a mix of random hexamer primers and specific reverse primer for pri-mir-675 and mir-675-5p. For mi405, 200 nM of the stem-loop forming primer (5′-GTCGTATCCAGTGCAGGGTCCGAGGTATTCGCACTGGATACGACGTCCAG-3′) (SEQ ID NO: 144) was also used for the RT step. A custom TaqMan assay (Applied Biosystems) including 1.5 μM of Forward primer (5′-CGGCCCAAACCAGATCTGAATC-3′) (SEQ ID NO: 145), 0.7 μM of Reverse primer (5′-GTGCAGGGTCCGAGGT-3′) (SEQ ID NO: 146), and 0.2 μM of mi405 probe (5′-6FAM-ATACGACGTCCAGGAT-3′) (SEQ ID NO: 147) was then run using the CFX Connect Real Time system apparatus (Bio-Rad). RPL13A (Mm02526700_g1; Applied Biosystems) served as the reference gene. The TaqMan gene expression assay consisted of using the TaqMan Gene Expression Master mix and TaqMan probes purchased from ThermoFisher Scientific. 1× of probe was mixed with 1× of the TaqMan Gene Expression Master mix (ThermoFisher Scientific), and with 20 ng of cDNA. The mir-675- and mi405-specific primers and probes were designed to quantify only the mir-675-5p and mi405 mature sequence (see TaqMan Gene Expression Master mix protocol).

Digital Droplet PCR (ddPCR). For mir-675 and mi405 constructs, RNA extraction was carried out as described for QPCR above. For cDNA synthesis, the TaqMan advanced cDNA synthesis kit (Thermo Fisher) was used and cDNA was prepared by following manufacturer's instructions. ddPCR was carried out using 1× ddPCR Supermix for probes (No dUTP) (Bio-Rad), 1× commercially available mir-675 advanced TaqMan probe, or a custom made mi405 advanced TaqMan probe (ThermoFisher) and 50 ng of cDNA. For DUX4 cDNA synthesis, the High-Capacity cDNA Reverse Transcription Kit (Applied Biosystems) was used according to manufacturer's instructions. Within the same cDNA reaction, 1×RT random hexamer primers and 25 pmoles of the Oligo(dT) primer were used to generate cDNA for DUX4 and DUX4-responsive biomarkers and for mouse Rpl13A, used as a reference gene. To quantify DUX4, the ddPCR reaction mixture (20 μL) contained 1× ddPCR Evagreen Supermix (Bio-Rad), 1 μM of forward and reverse DUX4 primers (Sharma et al., J Genet Syndr Gene Ther 2016 August; 7(4):303. doi: 10.4172/2157-7412.1000303. Epub 2016 Aug. 8), and 50 ng of cDNA. To quantify Rpl13A and DUX4-responsive biomarkers (Trim36 and Wfdc3), the 1× ddPCR Supermix for probes (No dUTP) and TaqMan specific probes were used for each of the genes. Droplets were generated using the Automatic Droplet generator QX200 AutoDG (Bio-Rad), the reactions were amplified in a C1000 Touch™ Thermal Cycler with 96-Deep Well Reaction Module (Bio-Rad). Cycling conditions were set up following the QX200 ddPCR Evagreen Supermix protocol. All assays are compatible with an annealing temperature of 58° C. Droplets were then read using the QX200 droplet reader (Bio-Rad). Finally, data were analyzed using the QuantaSoft analysis software (Bio-Rad). For quantification, each reaction of 20 μL was estimated to give at least 10,000 acceptable droplets.

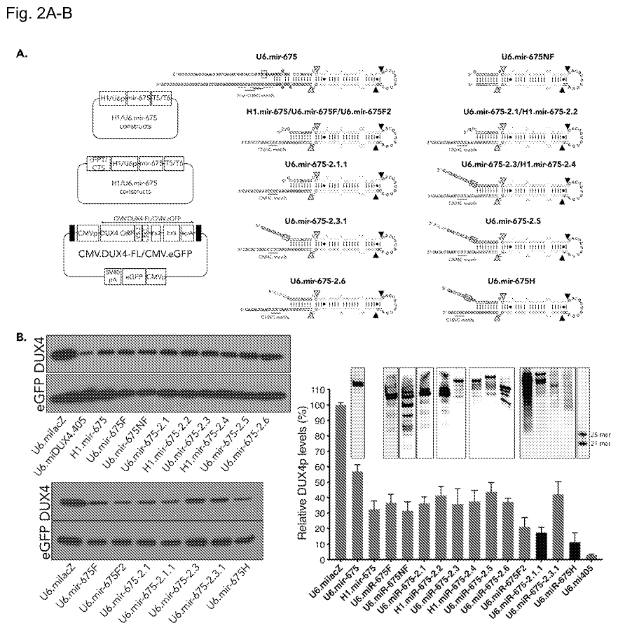

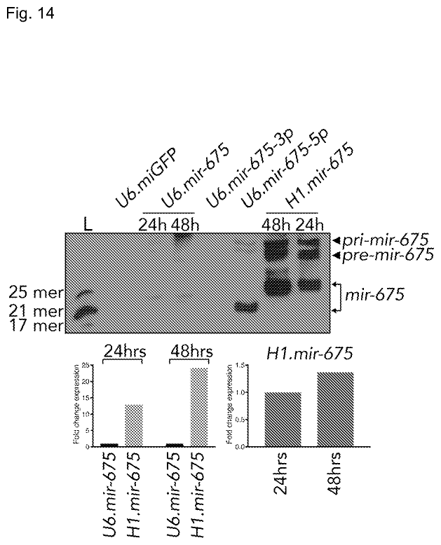

Small-Transcript Northern Blots. (See also FIGS. 2B and 14.) This blotting was performed as previously described in [McBride et al., Proc Natl Acad Sci USA (2008) 105(15):5868-73] with some modifications. Human embryonic kidney 293 (HEK293) cells were used to overexpress miR-675 and H19 transcripts. The expression plasmids of the latter were transfected into HEK293 using Lipofectamine 2000 (ThermoFisher Scientific). Cells were harvested 48 hrs post-transfection and lysed using the miRVANA miRNA isolation kit (Thermo Fisher Scientific) according to manufacturer's directions for use. No enrichment for miRNAs was done. 20 μg were loaded on a 15% acrylamide-bisacrylamide (19:1) gel containing 8M Urea (48%, wt/vol) and 1× Tris-Borate-EDTA (TBE) RNase free solution (Invitrogen). The miRNA Marker from New England BioLabs (NEB) was used as miRNA ladder and diluted 1:10, and 1.5 μL were loaded on the gel as a size reference. The DNA oligonucleotide probe specific to the miR-675 guide strand (miR-675-5p) was dual labeled with two biotin tags at the 5′ and 3′ ends, and used at 0.3 pmol (miR-675-5p probe: 5′biotin-CACTGTGGGCCCTCTCCGCACCA-3′biotin; (SEQ ID NO: 148)). The blot was then revealed using the Chemiluminescent Nucleic Acid Detection Module Kit (Thermo Fisher) according to manufacturer's directions for use, and exposed to the Hyblot CL Autoradiography film optimized for chemilluminescence.

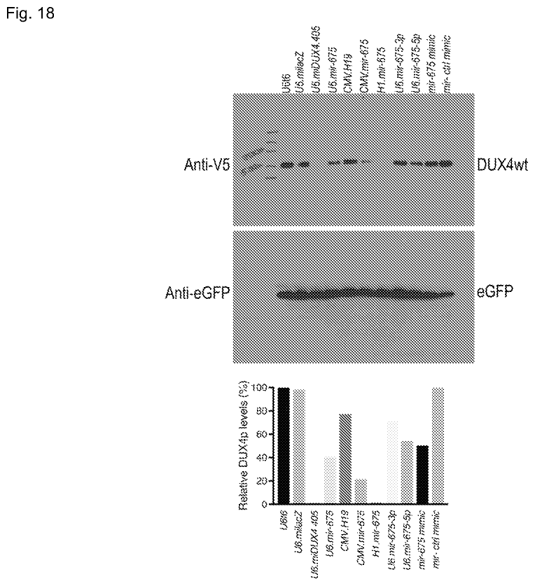

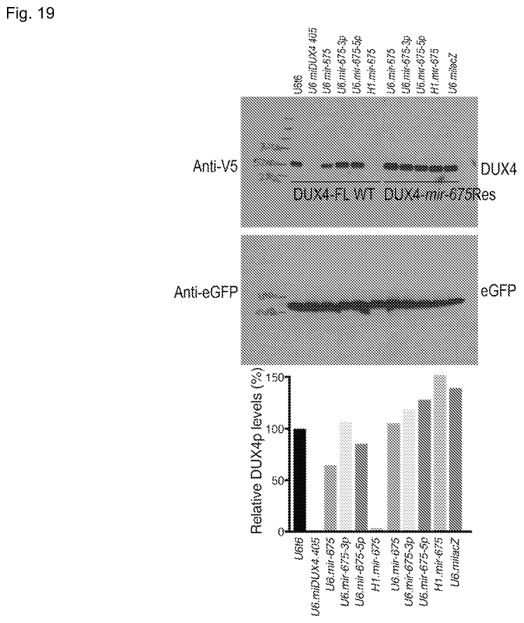

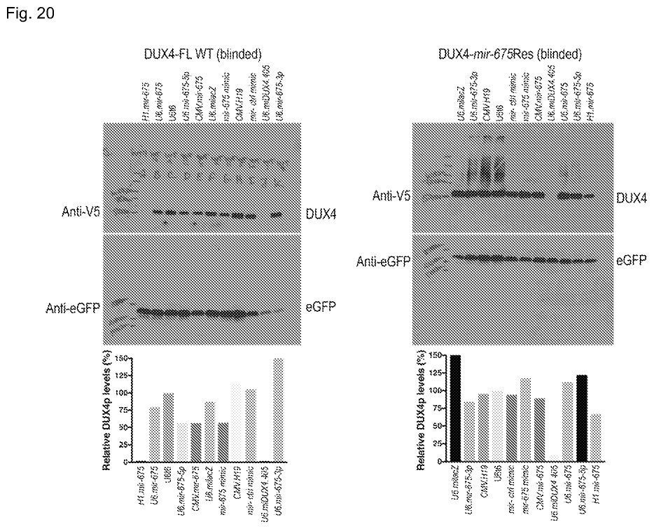

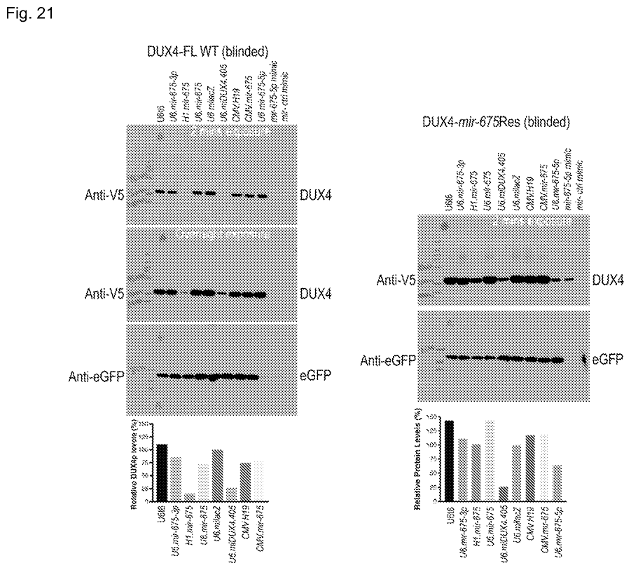

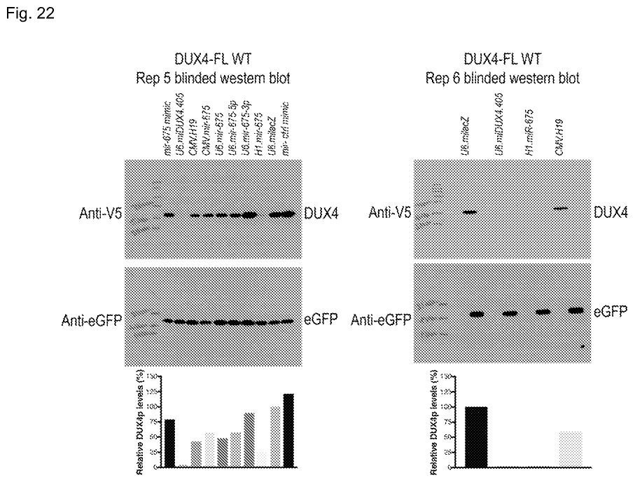

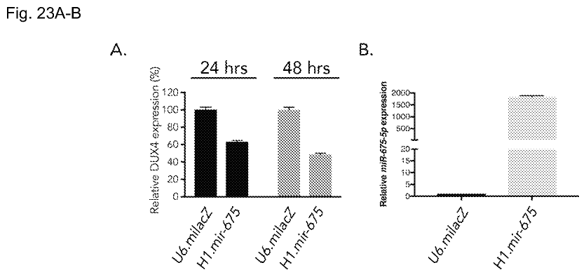

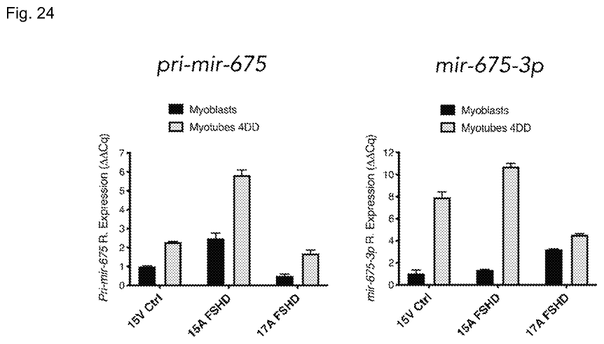

Western Blots. (See also FIGS. 1B, 2B, 3A, 5C, 7, 8, 10-12, 18-22, 26, 27, and 33.) To assess the inhibition of DUX4 protein expression by mir-675 and miDUX4.405, HEK293 cells were co-transfected using Lipofectamine 2000 (ThermoFisher Scientific) with AAV.CMV.DUX4-FL and mir-675 expression plasmids in various molar ratios. Total protein was extracted 48 hours after transfection using the RIPA buffer containing 50 mM Tris (pH 7.5-8.0), 150 mM NaCl, 0.1% (v/v) SDS, 0.5% (v/v) deoxycholate, 1% (v/v) triton X-100 (Fisher Scientific) and 1 tablet of protease inhibitor (ThermoFisher Scientific) per 10 mL of buffer. The total protein extract was quantified using the DC Protein Assay (Bio-Rad). Twenty microgram samples were separated on 12% SDS-PAGE, transferred to nitrocellulose membrane, using the wet transfer system, and incubated with the following antibodies: mouse monoclonal antibody to V5 (horseradish peroxidase [HRP]-coupled) [1:5,000 in 5% milk TBST buffer (150 mM NaCl, 50 mM Tris-HCl pH 7.4 and 0.1% Tween 20 (Fisher Scientific)), R961-25; Invitrogen); rabbit polyclonal eGFP antibody (1:50,000 in 3% BSA PBS, ab290; Abcam); mouse monoclonal β-actin antibodies (1:60,000; Sigma, St Louis, MO); overnight at 4° C. When using the eGFP antibody, membranes were blocked 30 mins with 3% BSA PBS prior to blotting. eGFP-probed blots were washed five times, 5 min each with 0.5% Tween 20 (Fisher Scientific) in TBST buffer, and then incubated with HRP-coupled goat anti-rabbit secondary antibody (1:250,000 in 3% BSA PBS, 115-035-144; Jackson ImmunoResearch) for 2 hours at room temperature. V5-probed blots were washed five times, 5 min each with 0.1% Tween 20 in TBST buffer. Following washes, blots were developed using Immobilon Western HRP substrate (Millipore) and exposed to film. DUX4.V5, and eGFP quantification was assessed using ImageJ.

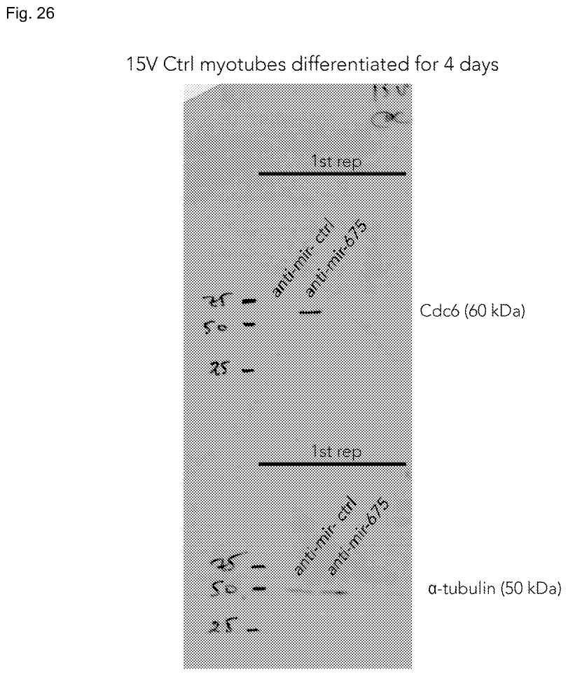

Western Blot to Detect Cdc6 Protein. (See also FIG. 26.) To assess the inhibition of Cdc6 protein expression by mir-675 and H19, we transfected 15A FSHD myoblasts were transfected with either mir-675 or H19 expression plasmids and myoblasts were differentiated for 7 days. Cdc6 was detected using the Cdc6 rabbit monoclonal antibody (C42F7; Cell Signaling Technology) at 1:500 dilution in 5% BSA TBST buffer. Cdc6-probed blots were washed 3 times, for 10 mins each with TBST buffer supplemented with 0.1% Tween 20 (ThermoFisher Scientific), and then incubated with HRP-coupled goat anti-rabbit secondary antibody (1:100,000 in 5% BSA TBST buffer) for 2 h at room temperature. Protein levels were quantified using ImageJ.

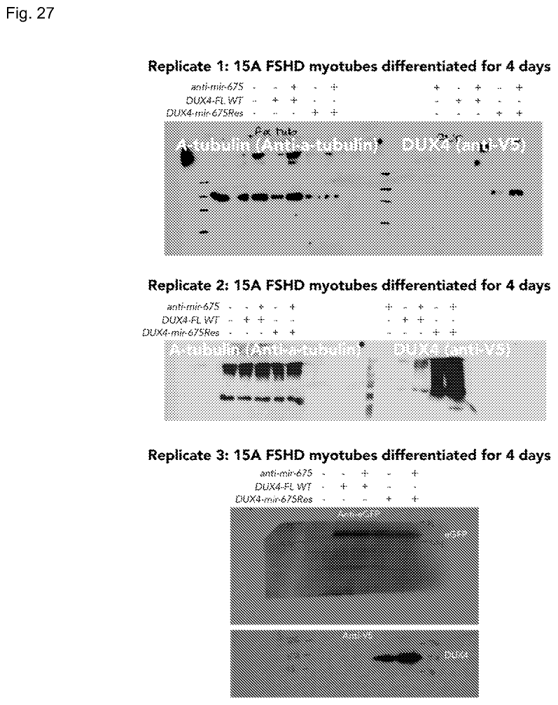

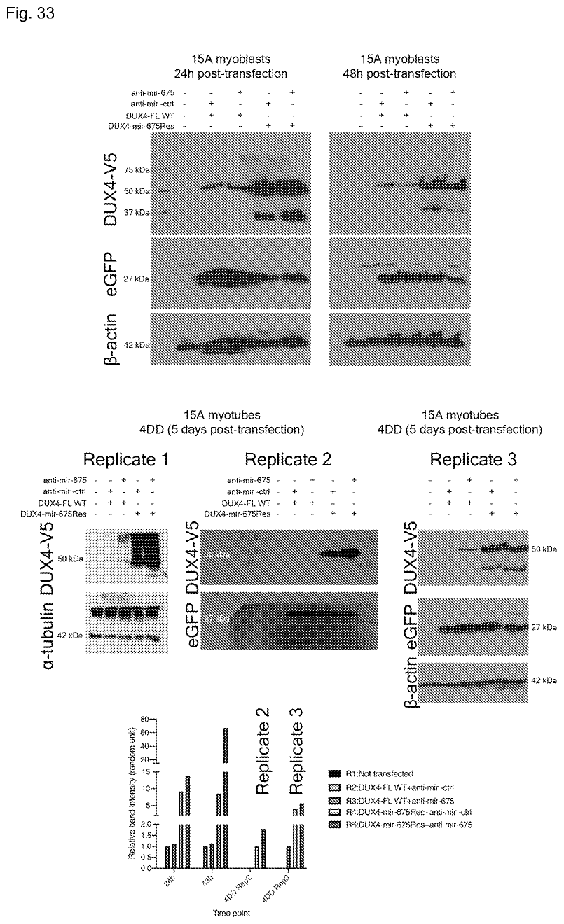

Western Blot to Detect Alpha-Tubulin (a-tubulin) Protein. (See also FIGS. 27 and 33.) Alpha-tubulin (a-tubulin) protein was detected using the a-tubulin rabbit polyclonal antibody (1:500 in 5% milk TBST buffer, ab15246; Abcam). Alpha-tubulin-probed blots were washed five times, for 5 mins each with TBST buffer supplemented with 0′0.1% Tween 20 (ThermoFisher Scientific), and then incubated with HRP-coupled goat anti-rabbit secondary antibody (1:100,000 in 5% milk TBST buffer) for 2 hrs at room temperature. All blots were developed by exposing them to X-ray films following treatment with Immobilon Western HRP substrate (Millipore). Protein levels were quantified using ImageJ.

Western Blot to Detect β-actin protein. (See also FIGS. 27 and 33.) β-actin protein was detected using a monoclonal antibody produced in mouse (1:1000 in 5% milk TBST buffer, SIGMA). Protein levels were quantified using ImageJ.

Apoptosis Assays.

Apoptosis in HEK293 Cells. To measure Caspase 3/7 activity in HEK293 cells, cells were co-transfected with mir-675 or H19 expression plasmids and CMV.DUX4-FL WT expression plasmid (not encompassing CMV.eGFP) using Lipofectamine 2000. 48h after transfection, cells were treated with the Apo-ONE® Homogeneous Caspase-3/7 Assay (Promega) following manufacturer's instructions. Caspase 3/7 activity (RFU) was measured using a fluorescence microplate reader.

Apoptosis in Human Myoblasts. To measure Caspase 3/7 activity in human myoblasts, mir-675, H19 expression plasmids or mir-675 antagomir (anti-mir-675, ThermoFisher Scientific) were electroporated into these cells using the high efficiency electroporation protocol, and were let to recover for 24h in their growth medium (LHCN medium) before starting differentiation using the KOSR (induces DUX4 expression (79)) supplemented differentiation medium. Cells were then allowed to differentiate for 7 days before reading the Caspase 3/7 activity.

Alkaline Comet Assay. HEK293 cells were co-transfected as in the apoptosis assay, described herein above, and collected 24 or 48h after transfection. The alkaline comet assay was carried out as previously described in Wang et al. (Cell Reports 9:90-103 (2014)) and Dmitriev et al. (Free Radic. Biol. Med. 99: 244-258 (2016)) without formamidopyrimidine DNA-glycosylase (FPG) enzyme treatment. For quantification of DNA damage, we used Tritek CometScore software was used to analyze 10 images of randomly selected non-overlapping cells. To evaluate the extent of DNA damage, the comet tail moment (the measure of tail length multiplied by tail intensity) was measured for each nucleus, and represented values as the average comet tail moments of 30 to 60 nuclei.

Flow Cytometry. For viability, cells were suspended in PBS containing 1:100 dilution of LIVE/DEAD® Fixable Near-IR stain. Cells were incubated for 30 minutes at room temperature and fixed with 1% formaldehyde for 10 minutes at room temperature. Cells were then washed one time and re-suspended in FACS buffer (2% FBS with 0.1% sodium azide in PBS) before flow cytometry analysis. Viable cells were gated by staining them with the LIVE/DEAD® Fixable Near-IR stain at the 633 nm excitation wavelength. Viable cells expressing GFP fluorescent signal using the 488 nm excitation wavelength were gated. All samples were analyzed using the Behemoth BD LSR II Flow Cytometer (BD Biosciences). The percent of GFP-positive cells was calculated based on the number of total live cells using the flow cytometer software FlowJo.

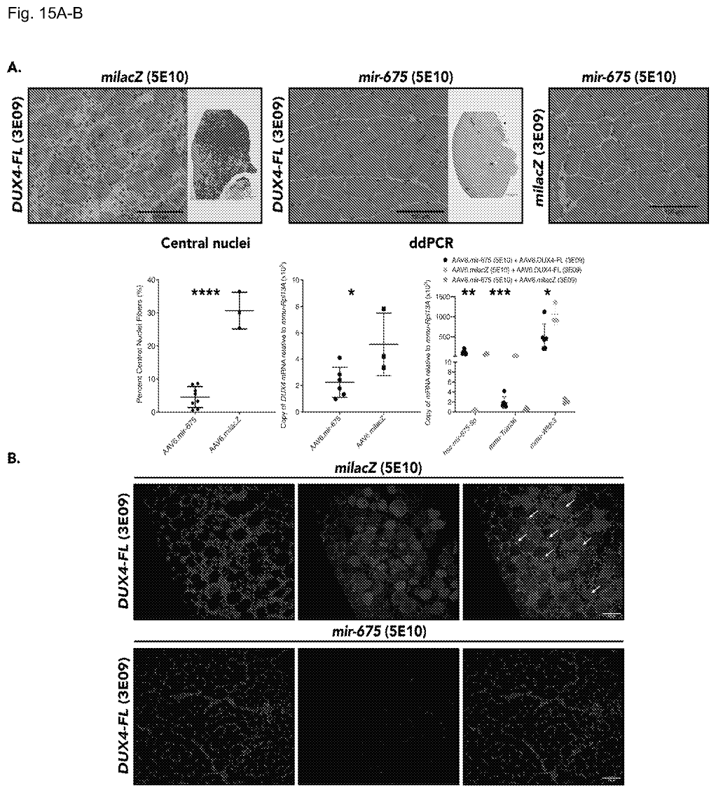

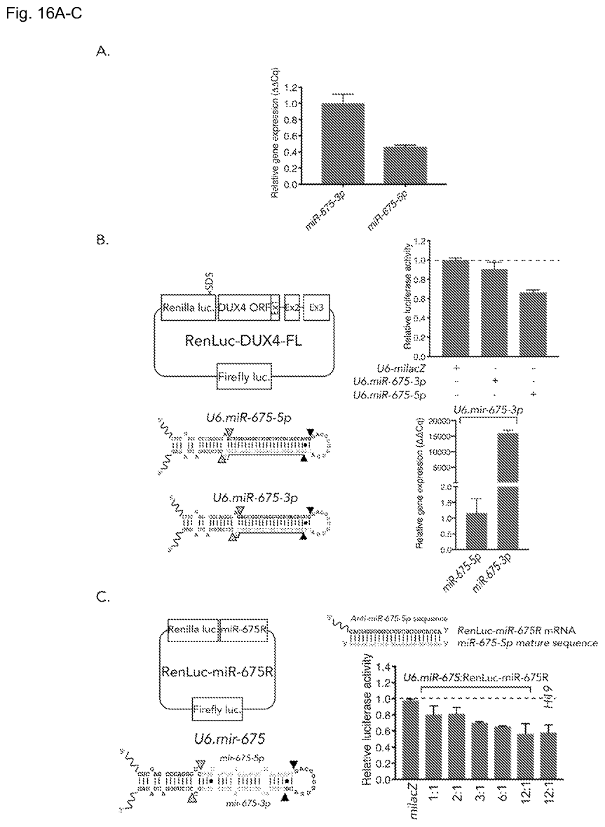

AAV Vector Delivery to Mice. (See also FIGS. 15A-B and 32A-B.) For determining bioactivity, 6- to 9-week-old C57BL/6 male and female mice received direct 35 μL IM injections into the TA. Mice received adeno-associated virus scAAV6.CMV.DUX4-FL at 5×10⁹ DNase-resistant particles (DRP) co-injected with scAAV6.CMV.eGFP at 1×10¹⁰ DRP, and a contralateral co-injection of scAAV6.CMV.DUX4-FL at 1×10⁹ DRP or 5×10⁹ DRP and scAAV6.U6.mir-675 at 5×10¹⁰ DRP. To test toxicity of mir-675, the tibialis anterior (TA) was intramuscularly injected with increasing doses of scAAV6.U6.mir-675 at 5×10¹⁰ DRP and 1×10¹¹ DRP, and injected contralateral TA with saline. To test mi405 toxicity, the TA was intramuscularly injected with 5×10⁸ DRP, 5×10⁹ DRP and 5×10¹⁰ DRP of scAAV6.U6.mi405F, scAAV6.U6.mi405G or scAAV6.U6.mi405H and injected contralateral TA with saline. To test mi405 inhibition efficiency, the TA was co-injected with scAAV6.CMV.DUX4-FL at 5×10⁹ DRP and scAAV6.U6.mi405F, G or H at 5×10⁸ DRP, 5×10⁹ DRP or 5×10¹⁰ DRP. For this, a contralateral injection of either scAAV6.U6.mi405F, G or H or of scAAV6.CMV.DUX4-FL at 5×10⁹ DRP was carried out. For mi405, all injections were compared to scAAV6.U6.mi405. Muscles were harvested at 2, 4 or 8 weeks post-injection. All animal studies were performed according to the NIH Guide for the Care and Use of Laboratory Animals.

Histology. (See also FIGS. 15A-B and 32A-B.) Dissected TA muscles were placed in O.C.T. Compound (Tissue-Tek) and frozen on liquid nitrogen-cooled isopentane. Cryosections were cut at 10 μm and then stained with H&E following standard protocols [Harper et al., Nat Med (2002) 8(3):253-61].

In Situ Immunofluorescence. (See also FIGS. 15A-B and 32A-B.) Gene expression and subcellular localization of DUX4 protein was visualized using V5 immunofluorescence as previously described in [Giesige et al., JCI Insight (2018) 3(22):e123538].

### Example 2

### The U6 Mir-675 Construct (U6.Mir-675) Targets DUX4 and Inhibits DUX4 Expression with Reduced Efficiency

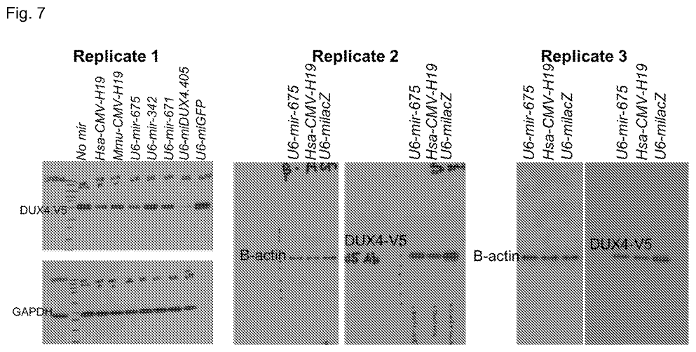

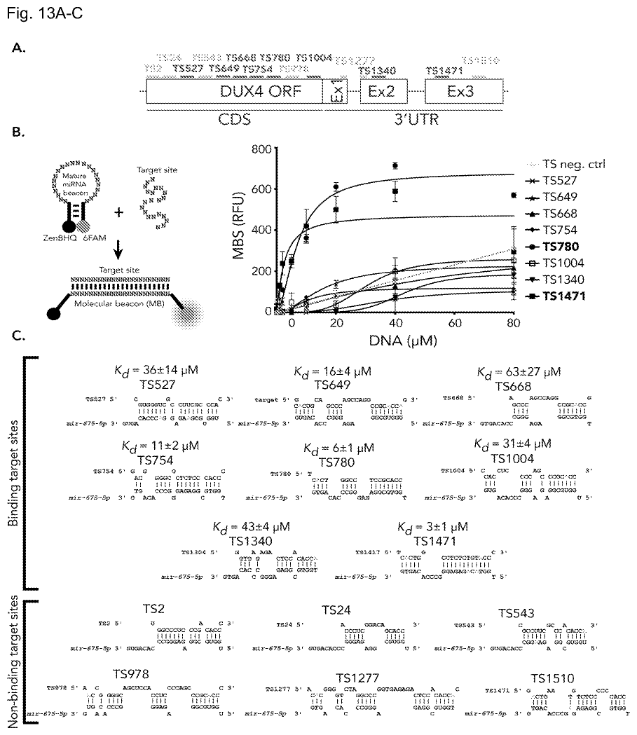

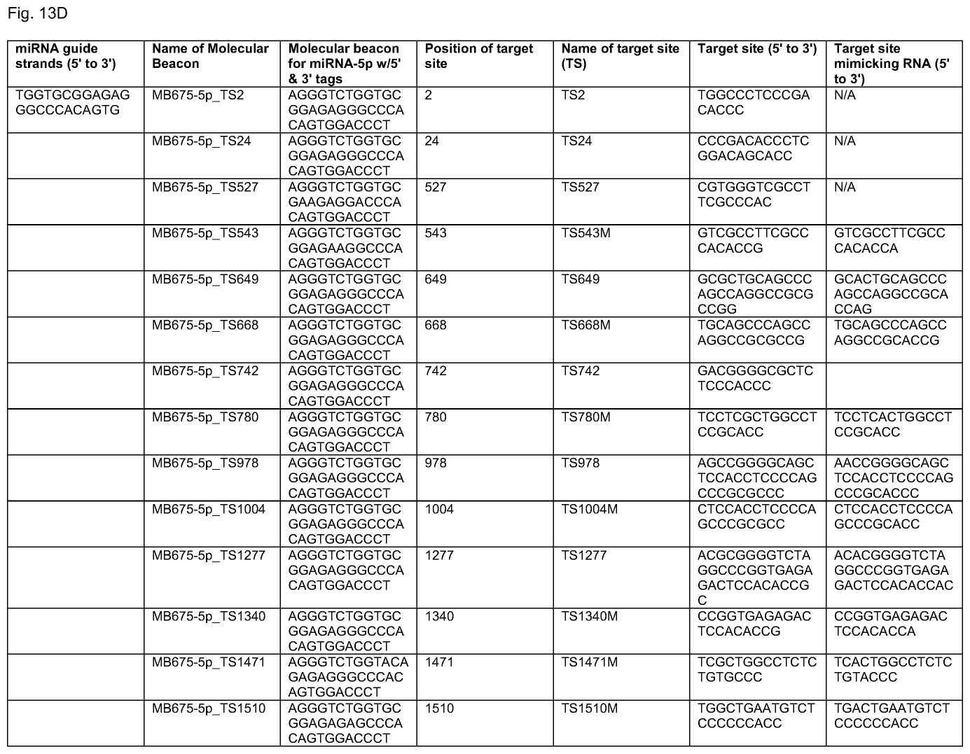

mir-675 has the ability to target DUX4 and inhibit its expression, as shown by using the dual-luciferase assay and western blot (FIGS. 1A-B, 2A-B, 7, 8, 15A-B, 16A-C, 18-23A, and 27). For both assays, the DUX4 construct containing all DUX4 pre-mRNA sequences (ORF and 3′ UTR), including potentially retained introns (RenLuc-DUX4-FL, where FL=full-length), was used. The ability of mir-675 to silence DUX4 in co-transfected HEK293 cells was then tested. mir-675 was delivered to cells by using a U6 promoter-driven mir-675 expression plasmid (U6.mir-675) (FIG. 1A). This construct was cloned using the same U6-based expression cassette, as was previously used to clone artificially designed miDUX4 miRNAs [Wallace et al., Mol Ther. 2012 July; 20(7): 1417-1423]. Thus, it has at its 5′ end flanking sequence 40 nucleotides and at the 3′ end flanking sequence 47 nucleotides. As seen in FIG. 1A, these sequences are able to base pair and form stem-loop structures. 48 hours post-transfection, both Renilla and Firefly luciferase activities were measured. First, the U6.mir-675 was not able to reduce the relative Renilla luciferase activity from the non-targeting RenLuc control backbone plasmid (RenLuc). However, when co-transfected with RenLuc-DUX4-FL, U6.mir-675 was able to reduce the relative Renilla luciferase activity in a dose-dependent manner. As a result, mir-675 reduced the relative Renilla luciferase activity by 24±3% (P<0.0001, ANOVA, N=3), 28±2% (P<0.0001, ANOVA, N=3) and 33±3% (P<0.0001, ANOVA, N=3) at a molarity ratio U6.mir-675: RenLuc-DUX4-FL (n:n) of 10 to 1, 20 to 1, and 40 to 1, respectively (FIG. 1A). Following the Luciferase assay, mir-675-mediated silencing of DUX4 expression was confirmed using western blot on total protein extracted from co-transfected HEK293 cells over-expressing mir-675 or H19 (mir-675 precursor) and DUX4-FL wild-type mRNA sequence and DUX4 protein (FIGS. 2A-B, 7, 8, 15A-B, and 18-22). As a result, 24 hours following co-transfection with U6.mir-675 or CMV.H19 and AAV.CMV.DUX4-FL expression plasmid encompassing a modified V5 epitope sequence [Ansseau et al., Plos One, published Jan. 27, 2016; https-colon-slash-slash-doi.org/10.1371/journal.pone.0146893] cloned downstream the DUX4 ORF (fused to DUX4 protein COOH-terminal) and the DUX4 3′UTR sequence, U6.mir-675 and CMV.H19 reduced DUX4 protein levels by 46±11% (P<0.02, ANOVA, N=3) and 48±12% (P<0.02, ANOVA. N=3), respectively (FIG. 1B and FIG. 7). These results were significant for a natural miRNA, which has normally weak base pairing to its target sites, because usually an inhibition efficiency between about 20-60% is achieved with a natural miRNA [Miyamoto-Mikami et al., Int. J. Sports Med. 37: 411-417 (2020); Dusl et al., Hum. Mol. Genet. 24: 3418-26 (2015); Saetrom et al., Cancer Res. 69: 7459-65 (2009); Clop et al., Nat. Genet. 38:813-8 (2006); Mencia et al., Nat. Genet. 41: 609-13 (2009)]. A molecular beacon binding assay (MBB assay) showed that mir-675 targets sites at DUX4 ORF and 3′UTR with high efficiency (FIGS. 13A-C and 31A-C), which would explain the relatively exceptionally high inhibition efficiency of DUX4 expression. However, even with a 50% inhibition efficiency, the ability to translate mir-675 into therapy for FSHD may be minimal. Therefore, it was reasoned that the U6.mir-675 expression plasmid might not be rationally designed to efficiently express and to allow optimal processing of both mir-675-5p and mir-675-3p mature sequences. Accordingly, commercially available mir-675 expression plasmids that might more efficiently express and allow better processing of mir-675 with the aim to reach higher inhibition efficiency of DUX4 expression were sought. Accordingly, H1.mir-675 (SBI Biosciences) was identified and tested; it showed higher inhibition efficiency of DUX4 expression and was better processed as was shown using northern blot (FIG. 14). However, when tested in vivo using intramuscular injection of C57BL/6 tibialis anterior (TA) muscles, scAAV6.mir-675 expressing H1.mir-675 construct showed muscle toxicity (data not shown). Accordingly, for the purpose of translating mir-675 as a viable miRNA-based gene therapy for FSHD, additional mir-675 expression cassettes were designed and tested by changing 5′ and 3′ end flanking sequences for better processing and to increase mir-675 potency in inhibiting DUX4 expression and reducing DUX4-induced toxicity.

### Example 3

### Inhibition of DUX4 Protein Levels In Vitro

Many previous publications have identified and validated the structures and motifs related to good miRNA processing and expression (e.g., see Treiber et al., Nat. Rev. Mol. Cell. Biol. 20:5-20 (2019)). Some of the important structures and motifs reside in the 5′ and 3′ end flanking sequences branching out from the stem-loop structure of the miRNA. An example, the “UG” dinucleotide motif, is usually found at the basal stem, ˜11 base pairs from the Drosha cut site at the 5′ miRNA strand. At the 3′ end, there is a single “CNNC” SRSF3 motif thought to be necessary to promote cleavage by the microprocessor. Even though these structures and motifs are highly conserved, many exceptions exist, which allow miRNA processing and expression in the absence of some of these structures and motifs. For example, mir-675 could be processed and expressed as a functional miRNA in the absence of these motifs, such as the “UG” motif. In addition, mir-675 structure encompasses at the 3′ end of its loop a degenerated “UGUG” (SEQ ID NO: 149) DGCR8 binding motif that became “UGGUG” (SEQ ID NO: 150), and is formed by a smaller stem with 33 instead of the ideal 35 nucleotides. mir-675 also lacks the mismatched “GHG” motif and was expressed and capable of inhibition of DUX4 expression in the absence of any or presence of multiple “CNNC” (SEQ ID NO: 151) SRSF3 motifs (FIG. 2A). Therefore, it was hypothesized that by adding some of these motifs to the mir-675 structure, mir-675 processing, expression and inhibition potency would be enhanced.

Accordingly, 14 mir-675 constructs encompassing 9 different flanking sequences at the 5′ and the 3′ end of the stem-loop structure (FIG. 2A) were designed. All constructs, except U6.mir-675, encompassed single “CNNC” motif, 3 (U6.mir-675-2.1, H1.mir-675-2.2, U6.mir-675-2.3, H1.mir-675-2.4, U6.mir-675-2.5 and U6.mir-675-2.6) or 4 (U6.mir-675-2.1.1, U6.mir-675-2.3.1, H1.mir-675, U6.mir-675F, U6.mir-675F2 and U6.mir-675H) nucleotides downstream of the 3′ end of mir-675 basal stem. H1.mir-675, U6.mir-675F and U6.mir-675F2 have similar flanking sequences but vary in the polymerase III promoter or the presence of additional structures upstream of the promoter, i.e., H1.mir-675 and U6.mir-675F2 encompassing the central polypurine tract/central termination sequence (cPPT/CTS) that creates a “DNA flap” allowing nuclear import of the HIV lentiviral genome during target-cell infection. U6.mir-675-2.1 and H1.mir-675-2.2 also have similar flanking sequences but are expressed from two different promoters (U6 or H1). A similar case is seen with U6.mir-675-2.3 and H1.mir-675-2.4. U6.mir-675NF has no flanking sequences. U6.mir-675-2.1, H1.mir-675-2.2, U6.mir-675-2.5, and U6.mir-675-2.3.1 have the “UG” Drosha recognition motif at the base of their stem-loop structures. As for U6.mir-675, and U6.mir-675H, the “UA” (boxed) dinucleotide might represent a degenerate Drosha recognition site. For all constructs, when designing the flanking sequences, it was made sure not to create any base pairing structures between the sequences by adjusting the nucleotide sequences accordingly.

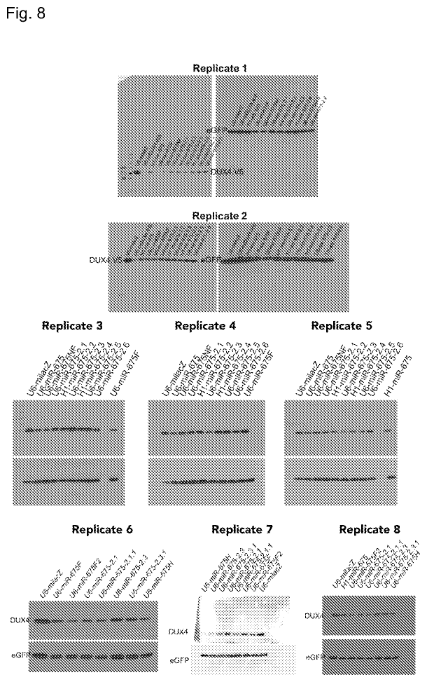

To assess the inhibition efficiency of these constructs, western blots using total proteins extracted from HEK293 cells co-transfected with H1/U6.mir-675 and CMV.DUX4-FL/CMV.eGFP (co-expressing the full length DUX4 (ORF+3′UTR) and eGFP) expression plasmids were carried out. All thirteen mir-675 constructs demonstrated better inhibition efficiency than U6.mir-675 (43±4%, N=6) (FIG. 2B, Table 2, and FIG. 8).

TABLE 2
Quantification of Western blots shown in
FIG. 2 and FIG. 8.

| | Average inhibition efficiency (%) |
|---|---|
| miRNA | (N = 3-8 independent replicates) |
| U6-milacZ | 0 ± 2 (N = 8) |
| U6-mir-675 | 43 ± 4 (N = 6) |
| H1-mir-675 | 68 ± 5 (N = 4) |
| U6-mir-675F | 64 ± 6 (N = 6) |
| U6-mir-675F2 | 79 ± 6 (N = 3) |
| U6-mir-675NF | 69 ± 6 (N = 5) |
| U6-mir-675-2.1 | 64 ± 4 (N = 8) |
| U6-mir-675-2.1.1 | 83 ± 3 (N = 3) |
| U6-mir-675-2.2 | 58 ± 6 (N = 5) |
| U6-mir-675-2.3 | 64 ± 10 (N = 8) |
| U6-mir-675-2.3.1 | 58 ± 8 (N = 3) |
| U6-mir-675-2.4 | 63 ± 7 (N = 5) |
| U6-mir-675-2.5 | 56 ± 6 (N = 5) |
| U6-mir-675-2.6 | 63 ± 2 (N = 5) |
| U6-mir-675H | 89 ± 6 (N = 3) |

In general, U6 controlled mir-675 showed better inhibition efficiency than H1 controlled mir-675 constructs. U6.mir-675-2.1.1 and U6.mir-675H had an inhibition efficiency of 83±3 (N=3 independent replicates) and 89±6 (N=3 independent replicates), respectively, and had the highest inhibition efficiency of DUX4 protein levels (P<0.05, ANOVA, N=3-8 independent replicates). Northern blot results showed that only U6.mir-675F, U6.mir-675NF, U6.mir-675-2.1, U6.mir-675-2.2, U6.mir-675F2, and U6.mir-675-2.1.1 have detectable mir-675 mature sequences ranging in size between 21 and 25 mer. Four out of these six constructs showed a typical miRNA processing profile [Nguyen et al., Cell 161:1374-87 (2015)]. Only U6.mir-675NF and U6.mir-675F2 showed additional processed bands: one band with a size smaller than 21 mer and one additional band with a size close to 25 mer. In addition, U6.mir-675NF, U6.mir-675F2, and U6.mir-675-2.1.1 showed a band with a size between 21 and 25 mer, which might correspond to the expected size of 23 mer of the mir-675-5p mature sequence (FIG. 2B).

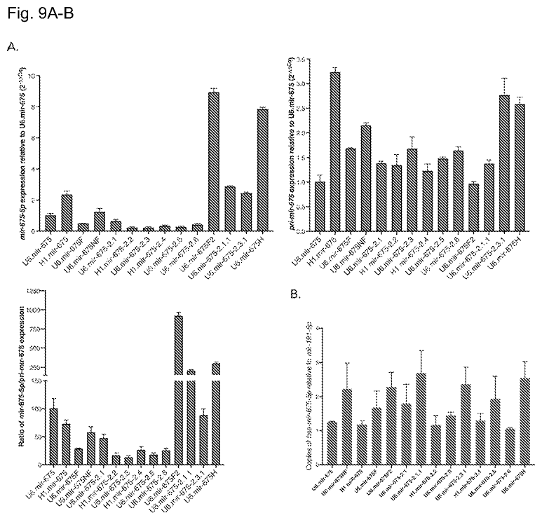

To quantify the expression of this mir-675 mature sequence (UGGUGCGGAGAGGGCCCACAGUG; (SEQ ID NO: 152)), QRT-PCR was carried out and nucleic acid levels were compared to the levels of primary mir-675 sequence (pri-mir-675) following transfection of H1/U6.mir-675 constructs into HEK293 cells. H1.mir-675, U6.mir-675F2, U6.mir-675-2.1.1, U6.mir-675-2.3.1, and U6.mir-675H expressed mir-675-5p levels with 232±35% (P<0.0002, ANOVA, N=3 independent experiments), 882±108% (P<0.0001, ANOVA, N=3), 282±34% (P<0.0001, ANOVA, N=3), 241±29% (P<0.0003, ANOVA, N=3) and 774±93% fold higher (P<0.0001, ANOVA, N=3) than the levels expressed from the U6.mir-675, respectively. When comparing ratios of mir-675-5p/pri-mir-675, U6.mir-675F2, U6.mir-675-2.1.1, and U6.mir-675H showed the highest relative mir-675-5p expression with a fold change of 918±169%, 207±38%, and 303±57% (P<0.0001, ANOVA, N=3), respectively, indicating that these constructs allow more processing of pri-mir-675 into mir-675-5p mature sequence (FIG. 9A-B). However, deep sequencing reads found in the miRbase database (http-colon-forward slash-forward slash-www.mirbase.org/cgi-bin/mirna_entry.pl?acc=Ml0005416), showed the presence of multiple processed mature mir-675-5p sequences ranging in size between 16 to 23 mer.

In order to quantify the level of all these mature sequences, the TaqMan advanced miRNA cDNA synthesis method that uses universal reverse transcription (RT) chemistry to synthesize cDNA templates that can be quantified using the TaqMan Advanced miRNA probes was used. In this method, most of the processed mature miRNA sequences were quantified. As a result, U6.mir-675-2.1.1, U6.mir-675-2.3.1, and U6.mir-675H showed the highest levels when compared to U6.mir-675 with a fold change of 213±51% (P<0.014, ANOVA, N=3), 187±40% (P<0.024, ANOVA, N=3) and 201±38% (P<0.0074, ANOVA, N=3), respectively (FIG. 9A-B). Two out of fourteen mir-675 constructs (U6.mir-675-2.1.1 and U6.mir-675H) showed the highest inhibition efficiency of DUX4 protein levels in vitro (FIGS. 2A-B and 8).

### Example 4

### The Inhibition Efficiency of Mi405 but not Other miDUX4 was Increased by Changing the 5′ and 3′ End Flanking Sequences

Following the success in increasing the inhibition efficiency of mir-675 through changes in the 5′ and 3′ end flanking sequences of the expression cassette, the same strategy was applied to the artificially designed miDUX4 (mi405) miRNA that is being developed as a miRNA-based gene therapy for FSHD [Wallace et al. Mol Ther Methods Clin Dev. 2018 Mar. 16; 8: 121-130]. This miRNA (U6.mi405) has the same flanking sequences found in U6.mir-675 expression plasmid.

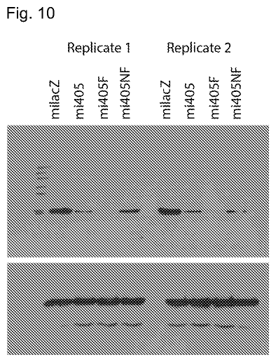

Accordingly, two new mi405 constructs, i.e., mi405F and mi405NF, were designed. The first construct, mi405F, lacks a flanking sequence at the 5′ end (only one “G” nucleotide for U6 transcription start site) of the stem-loop structure and possesses a 16 mer long 3′ end flanking sequence with a single “CNNC” (SEQ ID NO: 151) motif that is similar to that of H1.mir-675, U6.mir-675F, and U6.mir-675F2. The second construct, mi405NF, possesses only one “G” nucleotide at the 5′ end and no flanking sequence at the 3′ end (FIG. 3A). To assess the inhibition efficiency of the three mi405 constructs, a dual-luciferase assay and western blot analysis, similar to what was described for mir-675, were carried out. In the dual-luciferase assay, U6.mi405, U6.mi405F, or U6.mi405NF was co-transfected into HEK293 cells along with the RenLuc-DUX4 ORF (a construct expressing the open reading frame of DUX4 as a 3′UTR sequence of Renilla Luciferase). 24 h after transfection, U6.mi405, U6.mi405F, and U6.mi405NF reduced the relative Renilla luciferase activity by 85±1%, 89±1%, and 66±1% (P<0.0001, ANOVA, N=3), respectively, when tested against the RenLuc-DUX4 ORF construct. In addition, U6.mi405F demonstrated a significantly higher inhibition efficiency than U6.mi405 when tested at the 1:4 DUX4:mi405 molar ratio (P<0.04 ANOVA, N=3 independent experiments). Western blot analysis, under similar transfection conditions, showed that U6.mi405, U6.mi405F, and U6.mi405NF reduced DUX4 protein levels by 79±1% (P<0.0001, ANOVA, N=2 independent experiments), 99±1% (P<0.0001, ANOVA, N=2), and 70±13% (P<0.0036, ANOVA, N=2), respectively. Similarly, U6.mi405F was the most efficient and showed an inhibition efficiency increase by an average of 20% when compared to U6.mi405 (P<0.0079, ANOVA, N=2) (FIG. 3A and FIG. 10).

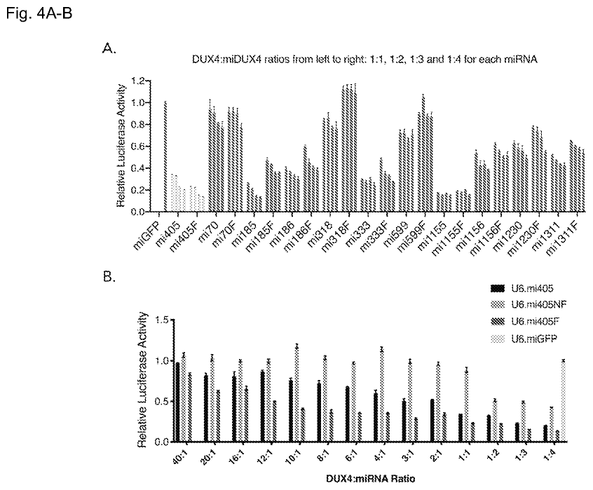

Following the success with U6.mi405F, the effect of the new flanking sequences were tested on the inhibition efficiency of other artificially designed miDUX4s that had been less efficient than mi405 in inhibiting DUX4 expression [Wallace et al., Mol. Ther. Methods Clin. Dev. 2018 Mar. 16; 8: 121-130]. With the expectation of obtaining an enhancement in their inhibition efficiency, ten miDUX4 were cloned using the same flanking sequences used for U6.mi405F. Their inhibition efficiency was tested using the dual-luciferase assay (FIG. 4A-B). Surprisingly, none of the ten miDUX4F constructs had its inhibition efficiency enhanced. On the contrary, the inhibition efficiency of mi185F, mi186F, mi318F, mi599F, mi1156F and mi1311F decreased when compared to their original counterparts, as shown by the increase in the relative Renilla Luciferase activity by 160±8% (P<0.0001, ANOVA, N=3), 27±9% (P<0.026, ANOVA, N=3), 44±15% (P<0.018, ANOVA, N=3), 24±9% (P<0.039, ANOVA, N=3), 34±8% (P<0.0071, ANOVA, N=3) and 27±9% (P<0.021, ANOVA, N=3), respectively (FIG. 9B).

The increase in miDUX4 levels in the co-transfected HEK293 cells showed a dose-dependent increase in the inhibition efficiency of most of the tested miDUX4 miRNAs, with the exception of mi70F, mi318, mi318F, mi333, mi599, mi599F, mi1155 and mi1155F (FIG. 4A). In addition, at molar ratios of DUX4 to miDUX4 greater than 1:4, none of the miDUX4F miRNAs performed better than its cognate miDUX4, with the exception of mi405F, which showed enhanced inhibition efficiency at all ratios. The latter was represented by a decrease of the relative Renilla luciferase activity by 33±2%; 32±2%; 34±1%, and 32±1% (P<0.0001, ANOVA, N=3 independent replicates) at ratios of 1:1, 1:2, 1:3, and 1:4, respectively.

Next, a dose de-escalation study was carried out for mi405F using the dual-luciferase assay in HEK293 cells (FIG. 4B). The increase in the DUX4:mi405 molar ratio from 1:4 to 40:1 led to a dose-dependent increase in the relative Renilla luciferase activity when the three miDUX4 miRNA constructs, i.e., U6.mi405, U6.mi405NF, and U6.mi405F, were tested. U6.mi405NF showed a significant increase in relative Renilla luciferase activity by 73±7% (P<0.0094, ANOVA, N=3), reaching 88±3% when switching from a molar ratio of 1:2 to 1:1 DUX4:miDUX4. Interestingly, U6.mi405F was still able to reduce relative Renilla luciferase activity, even at a molar ration of 40:1 DUX4:miDUX4, and was 13±2% (P<0.0001, ANOVA, N=3) more efficient than U6.mi405 in reducing relative Renilla luciferase activity. The biggest difference between U6.mi405 and U6.mi405F was observed at the 8:1 DUX4:miDUX4 molar ratio where U6.mi405F was 48±6% (P<0.0001, ANOVA, N=3) more efficient in reducing the relative Renilla luciferase activity reaching 38±2% (FIG. 4B).

### Example 5

### Changing the 5′ and 3′ End Sequences Flanking the Mi405 Stem-Loop Structure Affected the Silencing Efficiency and Expression of the miRNA

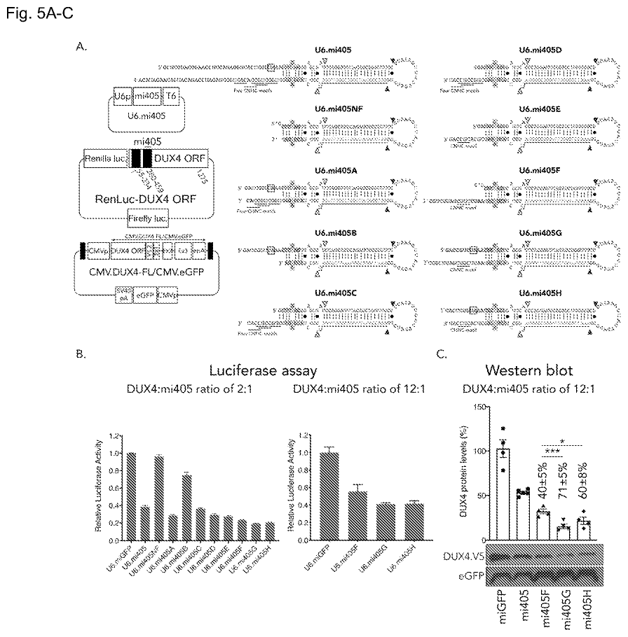

The discrepancy between the inhibition efficiency of mi405F and that of mi70F, mi185F, mi186F, mi318F, mi333F, mi599F, mi1155F, mi1156F, mi1230F and mi1311F suggested that enhancing the inhibition efficiency of a miRNA is not only related to its 5′ and 3′ end flanking sequences but also depends on the miRNA sequence (FIG. 3A-B). Since, with the exception of mi405, replacing the 5′ and 3′ end flanks of the other miDUX4s with those of mi405F did not enhance their inhibition efficiency, there was no reason to suggest that using other flanking sequences would change the actual outcome. Therefore, the lead miRNA mi405 was focused upon in order to identify the sequences responsible for enhancing its inhibition efficiency. In addition to U6.mi405, U6.mi405NF and U6.mi405F, seven additional constructs, i.e., U6.mi405A, U6.mi405B, U6.mi405C, U6.mi405D, U6.mi405E, U6.mi405G, and U6.mi405H (FIG. 5A) were designed.

In the flanking sequences branching out from the stem-loop structure, the “UA” motif was focused upon, as being a possible Drosha recognition site found in the 5′ end flanking sequence and on the “CNNC” (SEQ ID NO: 151) SRSF3 motif found in the 3′ end flanking sequence. Constructs, such as U6.mi405, U6.mi405A, U6.mi405B, U6.mi405G, and U6.mi405H possess the “UA” motif. U6.mi405, U6.mi405A, U6.mi405C, U6.mi405D, U6.mi405E, U6.mi405F, U6.mi405G and U6.mi405H possess one or multiple “CNNC” (SEQ ID NO: 151) motifs (FIG. 5A). Additionally, some constructs, such as the U6.mi405NF, lack flanking sequences. All these constructs were cloned in U6-expression plasmids and were tested for their inhibition efficiency using the dual luciferase assay.

Accordingly, the U6.mi405 and the RenLuc-DUX4 ORF expression plasmids were co-transfected into HEK293 cells with a DUX4:mi405 molar ratio of 2 to 1 and measured using the relative Renilla luciferase activity 24 hours post-transfection. All U6.mi405 constructs efficiently reduced the relative Renilla luciferase activity, except for U6.mi405NF and U6.mi405B, which reduced the Renilla luciferase activity by 4±2% (P>0.80, ANOVA, N=3) and 25±3 (P<0.0001, ANOVA, N=3), respectively. However, U6.mi405, U6.mi405A, U6.mi405C, U6.mi405D, U6.mi405E, U6.mi405F, U6.mi405G, and U6.mi405H reduced the Renilla luciferase activity by 62±2%, 72±1%, 64±1%, 71±1%, 73±1%, 77±1%, 81±1%, 80±1% (P<0.0001, ANOVA, N=3), respectively. Interestingly, at the DUX4:mi405 molar ratio of 2 to 1, none of the other U6.mi405 constructs was significantly more potent than U6.mi405F in inhibiting the relative Renilla luciferase activity (FIG. 5B). Only U6.mi405G and U6.mi405H have shown minimal enhancement in their inhibition efficiency when compared to U6.mi405F. Therefore, it was decided to further test their activity using western blot analysis using the same DUX4:mi405 molar ratio co-transfected for 24 hours in HEK293 cells. As expression plasmids, a miRNA negative control U6.miGFP was used. U6.mi405F, U6.mi405G or U6.mi405H also was used to express mi405. CMV.DUX4-FL/CMV.eGFP was used to express DUX4-FL and eGFP.

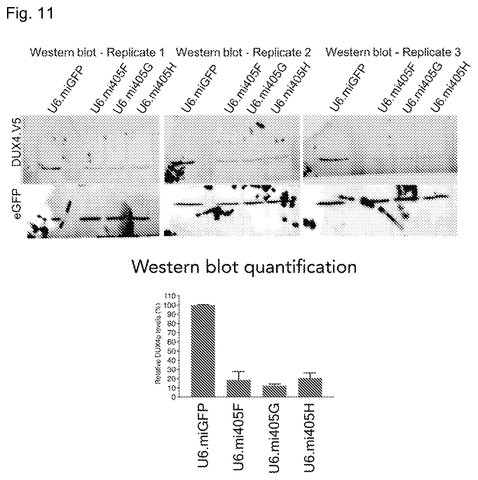

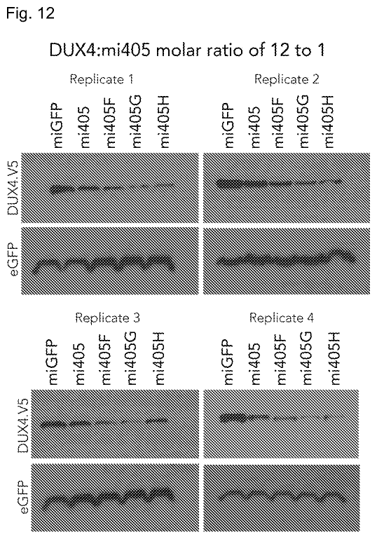

U6.mi405F, U6.mi405G, and U6.mi405H reduced DUX4 protein levels by 81±9%, 88±2%, and 79±6%, respectively when compared to U6.miGFP (P<0.0001, ANOVA, N=3 independent replicates) (FIG. 11). However, U6.mi405G and U6.mi405H were not significantly more efficient than U6.mi405F. Therefore, the DUX4:mi405 molar ratio was increased to 12 to 1 and re-tested with the three mi405 constructs using the dual luciferase assay and western blots (FIG. 5B-C). The dual luciferase assay was carried out as described herein above. As a result, when compared to U6.mi405F, U6.mi405G and U6.mi405H reduced the Renilla luciferase activity by an additional 26±5% (P<0.033, ANOVA, N=3) and 25±6% (P<0.042, ANOVA, N=3), respectively (FIG. 11). In conjunction with these results, the western blot results showed that U6.mi405F, U6.mi405G, and U6.mi405H induced a significant reduction of DUX4 protein levels by 40±5% (P<0.0209, ANOVA, N=4), 71±5% (P<0.0001, ANOVA, N=4), and 60±8% (P<0.0009, ANOVA, N=4), relative to U6.mi405. In addition, U6.mi405G and U6.mi405H induced a significant reduction of DUX4 protein levels by 52±9% (P<0.0009, ANOVA, N=4) and 33±14% (P<0.0498, ANOVA, N=4), when compared to U6.mi405F (FIG. 5C and FIG. 12).

Apart from the effect on the inhibition efficiency of mi405, the effect of the flanking sequences on the expression of the miRNA also was tested. Therefore, the expression of the processed mature mi405 sequences was quantified using the standard and advanced TaqMan cDNA synthesis reaction. In the former reaction, a reverse primer detects the mature mi405 sequence following a stem-loop primer-based small RNA detection principle (ThermoFisher Scientific) (Jung et al., RNA (2013) 19: 1-10). The amplification and quantification steps were then performed using a standard TaqMan probe specific to mi405 that base pairs at the junction between the mi405 mature sequence and the reverse primer sequence (FIG. 6A). As a result, QPCR analysis performed on cDNA from all U6.mi405 constructs generated using the standard TaqMan cDNA synthesis reaction showed that U6.mi405F, U6.mi405B and U6.mi405C had 85±5% (P<0.0019, ANOVA, N=3 independent replicates), 71±9% (P<0.0038, ANOVA, N=4) and 63±27% (P<0.0133, ANOVA, N=3) lesser or lower expression than their mature mi405 sequence in comparison to U6.mi405, respectively. When compared to U6.mi405, however, the levels of the mature mi405 sequence originating from U6.mi405A, U6.mi405D, U6.mi405E, U6.mi405G, and U6.mi405H were greater or higher with the difference not being statistically significant (P>0.05, ANOVA, N=3-4 independent experiments) (FIG. 6A). In the TaqMan advanced cDNA synthesis reaction, the mature sequence is extended through ligation of an adaptor sequence at the 5′ end and through the enzymatic addition of a polyA tail at the 3′ end of the mature mi405 sequence. The amplification and quantification steps were then performed using a TaqMan advanced probe specific to mi405 that normally base pairs with the 3′ end of the mature miRNA and with part of the adaptor sequence. Here, an additional TaqMan advanced probe (embedded probe) that only base pairs with the mature sequence of mi405 (FIG. 6B) was used. As a result, following TaqMan advanced cDNA synthesis reaction, droplet digital PCR (ddPCR) was performed to quantify mi405, mi405F, mi405B, mi405C, mi405G and mi405H expression levels. For this, TaqMan advanced embedded and overlapped mi405 probes (FIG. 6B) were used. As a result, all tested U6.mi405 constructs generated mature mi405 sequences with similar levels, although U6.mi405C, U6.mi405G and U6.mi405H showed higher levels that were not statistically significant.

### Example 6

### DUX4 miRNA Decrease DUX4-Activated Biomarker Expression in a Mouse Model of FSHD

AAV comprising the DUX4miRNA constructs of the disclosure are injected into a new FSHD mouse model (TIC-DUX4) or any other mouse model of FSHD mice intramuscularly (IM) or intravenously (IV). After 4, 8, 12, 16, 20, and 24 weeks, the expression level of a DUX4 biomarker, such as Wfdc3 or Trim36, are measured by qRT-PCR, RNAscope, or ddPCR.

Reduced levels of DUX4 biomarker expression are observed in muscles of mice treated with DUX4miRNA compared to the levels in muscles of untreated mice.

### Example 7

### DUX4 miRNA Decrease Endogenous DUX4 Expression in Muscle in a Mouse Model of FSHD

AAV comprising the DUX4miRNA constructs of the disclosure are injected into a new FSHD mouse model (TIC-DUX4) or any other mouse model of FSHD mice intramuscularly (IM) or intravenously (IV). After 4, 8, 12, 16, 20, and 24 weeks, the expression level of DUX4 mRNA is measured by qRT-PCR, RNAscope, or ddPCR.

Reduced levels of DUX4 mRNA are observed in muscles of mice treated with DUX4miRNA compared to the levels in muscles of untreated mice.

### Example 8

### DUX4 miRNA Decrease Endogenous DUX4 Expression in Muscle

AAV comprising the DUX4miRNA constructs of the disclosure are injected into patients suffering from FSHD intramuscularly (IM) or intravenously (IV). Prior to treatment and after 4, 8, 12, 16, 20, 24, 28, 32, 36 40, 44, 48, and 52 weeks, the expression level of DUX4 mRNA in muscle of the patients is measured in biopsied muscle by qRT-PCR, RNAscope, or ddPCR.

Reduced levels of DUX4 mRNA are observed in muscles of patients treated with AAV comprising the DUX4miRNA constructs of the disclosure compared to the levels of DUX4 mRNA in muscles of the same patients prior to treatment. Improvement in FSHD disease symptoms is also observed.

### Example 9

### Small Molecule Upregulation of Mir-675 Reduces DUX4 and DUX4-Responsive Biomarkers in FSHD Patient Myotubes

Mir-675, a microRNA that regulates DUX4, represents 0.05% of all known human miRNAs. Having identified mir-675 as a DUX4 regulator, experimental work was carried out to leverage this finding for a drug-based therapeutic approach in treating diseases associated with the expression or overexpression of DUX4, such as the muscular dystrophy, FSHD, and cancer. Such drug-based therapy is tunable and potentially stopped if untoward events arise. Having identified mir-675 as a strong endogenous regulator of DUX4, research was carried out to review previously published gene expression data for small molecule drugs that have been shown to increase mir-675 expression or its H19 precursor.

Three small molecule candidates (β-estradiol, a combination of β-estradiol+medroxyprogesterone acetate (MPA), and melatonin) were tested for their ability to upregulate mir-675-5p in HEK293 cells and in human myotubes. HEK293 cells normally express minimal amounts of mir-675. HEK293 cells were treated with (1) 20 μM β-estradiol alone; (2) 10 μM or 20 μM β-estradiol+MPA; or (3) 20 μM or 40 μM melatonin. 24 hours after treatment, mir-675-5p expression was measured by Droplet Digital PCR (ddPCR).

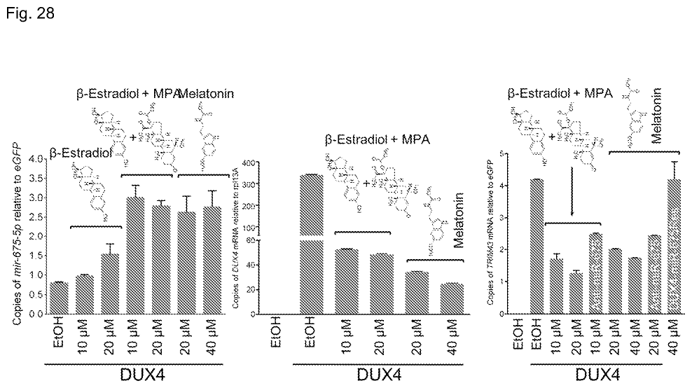

Each of the three treatment regimens, e.g., β-estradiol, β-estradiol+MPA, or melatonin, significantly increased mir-675 levels when compared to the control, i.e., 100% ethanol treated DUX4-transfected cells (FIG. 28 and Table 3). In FIG. 28, β-estradiol, medroxyprogesterone acetate (MPA) and melatonin increased mir-675 expression and reduced the expression of DUX4 and DUX4-induced biomarker TRIM43 in HEK293 cells. Droplet Digital PCR (ddPCR) was carried out to measure mir-675-5p, DUX4 and TRIM43 levels. HEK293 cells were transfected with DUX4 and were treated with two drugs individually (i.e., β-estradiol and melatonin) or with a combination of β-estradiol and MPA at 10, 20 or 40 μM at the time of transfection. The effects of these drugs were evaluated by comparison to ethanol (vehicle)-treated cells. Numbers correspond to n=3 independent experiments (ANOVA, P<0.0001). The quantification (percent change) of gene expression from HEK293 cells treated with β-estradiol, β-estradiol+MPA, or melatonin was measured using droplet digital PCR (ddPCR) and is reported in Table 3 set out below. Anti-mir-675 is an antagomiR targeting the mature sequence of mir-675-5p, inhibiting its function as inhibitor of DUX4 gene expression. CMV.DUX4-mir-675Res is an expression plasmid encoding a DUX4 mutant sequence. This sequence is mutated in mir-675 target site 780 (TS780) found in ORF (see FIG. 17B) and has its 3′UTR deleted, rendering the expression of this DUX4 mutant resistant to mir-675-dependent inhibition.

TABLE 3
Quantification of endogenous mir-675-5p, transfected DUX4, and endogenous TRIM43 gene expression
in HEK293 cells after treatment with β-estradiol, β-estradiol + MPA, or melatonin.

| | | | | | +anti-mir-675 | +CMV.DUX4- |
|---|---|---|---|---|---|---|
| | | | | | (300 nM) | mir-675Res |
| | Concentration | mir-675-5p | DUX4 | TRIM43 | TRIM43 | TRIM43 |
| | (μM) | (% increase) | (% decrease) | (% decrease) | (% increase) | (% decrease) |
| β-Estradiol | 10 | 22 ± 06 | | | | |
| | | (P > 0.72, | | | | |
| | | ANOVA, N = 3 | | | | |
| | | independent | | | | |
| | | experiments) | | | | |
| | 20 | 92 ± 34 | | | | |
| | | (P < 0.034, | | | | |
| | | ANOVA, N = 3 | | | | |
| | | independent | | | | |
| | | experiments) | | | | |
| β- | 10 each | 277 ± 41 | 85 ± 01 | 59 ± 04 | 45 ± 11 | |
| Estradiol + | | (P < 0.0017, | (P < 0.0001, | (P < 0.0001, | (P < 0.01, | |
| MPA | | ANOVA, N = 3 | ANOVA, N = 3 | ANOVA, N = 3 | ANOVA, N = 3 | |
| | | independent | independent | independent | independent | |
| | | experiments) | experiments) | experiments) | experiments) | |
| | 20 each | 250 ± 19 | 86 ± 01 | 70 ± 02 | | |
| | | (P < 0.0039, | (P < 0.0001, | (P < 0.0001, | | |
| | | ANOVA, N = 3 | ANOVA, N = 3 | ANOVA, N = 3 | | |
| | | independent | independent | independent | | |
| | | experiments) | experiments) | experiments) | | |
| Melatonin | 20 | 229 ± 53 | 90 ± 01 | 52 ± 01 | 21 ± 01 | |
| | | (P < 0.0032, | (P < 0.0001, | (P < 0.0001, | (P < 0.0001, | |
| | | ANOVA, N = 3 | ANOVA, N = 3 | ANOVA, N = 3 | ANOVA, N = 3 | |
| | | independent | independent | independent | independent | |
| | | experiments) | experiments) | experiments) | experiments) | |
| | 40 | 246 ± 53 | 93 ± 01 | 58 ± 01 | | 0.1 ± 16 |
| | | (P < 0.0018, | (P < 0.0001, | (P < 0.0001, | | (P > 0.99, |
| | | ANOVA, N = 3 | ANOVA, N = 3 | ANOVA, N = 3 | | ANOVA, N = 3 |
| | | independent | independent | independent | | independent |
| | | experiments) | experiments) | experiments) | | experiments) |

DUX4 levels were measured in the cells of each of the treatment groups. Control-treated cells transfected with DUX4 had an average of 339±2 copies/μL relative to the house keeping gene RPL13A. The addition of each of β-estradiol at 20 μM; β-estradiol+MPA at 20 μM each; and melatonin at 20 μM and 40 μM led to a significant decrease in the levels of DUX4 and the DUX4-responsive biomarker TRIM43 (FIG. 28 and Table 3).

The co-transfection with the anti-mir-675 antagomir increased TRIM43 levels when HEK293 cells were treated with 10 μM of the combination β-estradiol+MPA and 20 μM of melatonin, indicating that the drugs used exerted their effect on DUX4 and TRIM43 by directly inducing the expression of mir-675.

Treatment with 40 μM melatonin of HEK293 cells transfected with the mir-675-resistant DUX4 expressing plasmid (CMV.DUX4-mir-675Res) did not lead to a decrease in TRIM43 expression, confirming the mir-675-dependent effect of melatonin (FIG. 28 and Table 3).

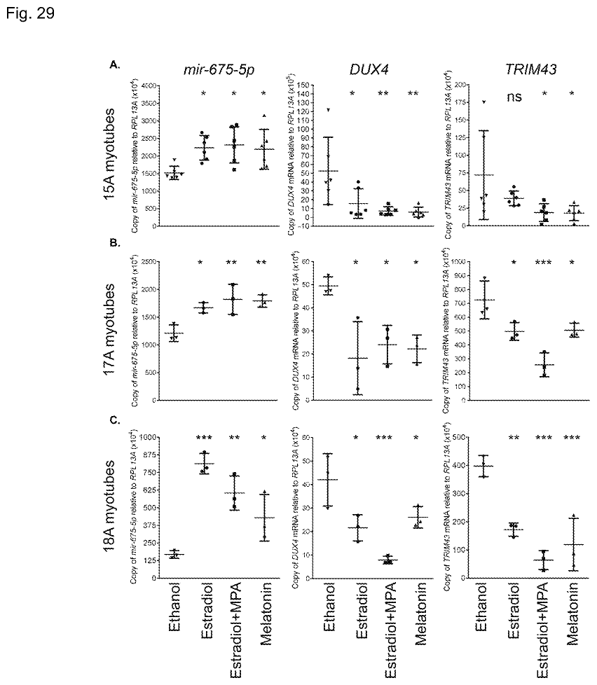

Next, the effects of the three treatment regimens were tested on the expression of endogenous mir-675-5p, DUX4 and TRIM43 in 15A, 17A and 18A FSHD differentiated muscle cell lines (myotubes) (FIG. 29 and Table 4). These FSHD cell lines were chosen because they exhibit low (15A), medium (18A) and high (17A) DUX4 expression [Jones et al., Hum. Mol. Genet. 21: 4419-30 (2012)]. In FIG. 29, β-estradiol, medroxyprogesterone acetate (MPA) and melatonin increased mir-675 expression and reduced the expression of DUX4 and the DUX4-induced biomarker TRIM43 in three FSHD affected myotube lines. Droplet digital PCR (ddPCR) was used to measure mir-675-5p, DUX4 and TRIM43 levels in 15A (A), 17A (B) and 18A (C) FSHD affected myotubes five days after differentiation. Two drugs (i.e. β-estradiol and melatonin) were added individually or as a combination (β-estradiol+MPA) at 20 μM at the 4th day of differentiation. All treatments were compared to ethanol (vehicle)-treated control cells (n=6 independent experiments for 15A and N=3 for 17A and 18A. *, P<0.05. **, P<0.01. ***, P<0.001, ANOVA). The quantification of gene expression (mir-675-5p, DUX4 and TRIM43) in 5-day differentiated 15A, 17A and 18A myotubes treated with β-estradiol, β-estradiol+MPA or melatonin is reported in Table 4 below and FIG. 29.

TABLE 4
Percent fold-change of gene expression in myotubes treated with
β-estradiol, β-estradiol + MPA or melatonin.

| | | Cell lines | mir-675-5p | DUX4 | TRIM43 | |
|---|---|---|---|---|---|---|
| | Concentration | 5DD | (% | (% | (% | |
| | (μM) | myotubes | increase) | decrease) | decrease) | N |
| β-estradiol | 20 | 15A | 47 ± 11 \| *, | 70 ± 38 \| *, | 46 ± 40 \| NS, | N = 6 I.E. |
| | | | P < 0.025 | P < 0.018 | P > 0.23 | |
| | | 17A | 38 ± 09 \| *, | 63 ± 19 \| *, | 31 ± 12 \| *, | N = 3 I.E. |
| | | | P < 0.0275 | P < 0.0103 | P < 0.0377 | |
| | | 18A | 382 ± 44 \| ***, | 49 ± 19 \| *, | 57 ± 07 \| ** , | N = 3 I.E. |
| | | | P < 0.0003 | P < 0.0143 | P < 0.0024 | |
| β-estradiol + | 20 each | 15A | 52 ± 15 \| *, | 86 ± 39 \| **, | 74 ± 45 \| *, | N = 6 I.E. |
| MPA | | | P < 0.012 | P < 0.0037 | P < 0.0296 | |
| | | 17A | 50 ± 15 \| **, | 51 ± 11 \| *, | 65 ± 15 \| ***, | N = 3 I.E. |
| | | | P < 0.0060 | P < 0.0298 | P < 0.0006 | |
| | | 18A | 258 ± 49 \| **, | 81 ± 20 \| ***, | 84 ± 09 \| ***, | N = 3 I.E. |
| | | | P < 0.0032 | P < 0.0007 | P < 0.0002 | |
| Melatonin | 20 | 15A | 44 ± 16 \| *, | 88 ± 40 \| **, | 75 ± 45 \| *, | N = 6 I.E. |
| | | | P < 0.035 | P < 0.0030 | P < 0.0271 | |
| | | 17A | 48 ± 09 \| **, | 55 ± 09 \| *, | 30 ± 12 \| *, | N = 3 I.E. |
| | | | P < 0.0079 | P < 0.0212 | P < 0.0446 | |
| | | 18A | 154 ± 59 \| *, | 38 ± 17 \| *, | 70 + 15 \| ***, | N = 3 I.E. |
| | | | P < 0.0476 | P < 0.0470 | P < 0.0006 | |

NS: not significant.
I.E : independent experiments.
5DD: 5-days differentiated. ANOVA statistical tests were performed on data from individual experiments.

The three treatment regimens were added to myotubes at their 4th day of differentiation. Cells were harvested 24 hours later. FSHD cells were treated at the differentiation stage because a boost in DUX4 expression occurs at the differentiation stage [Balog et al., Epigenetics 10: 1133-42 (2015)].

In 15A myotubes, all three treatment regimens triggered an increase in mir-675 expression (FIG. 29A and Table 4). Simultaneously, DUX4 and TRIM43 expression significantly decreased when 15A myotubes were treated with β-estradiol+MPA or melatonin. However, treatment with β-estradiol alone did not trigger a significant decrease in TRIM43 expression (FIG. 29A and Table 4). In 17A and 18A myotubes, β-estradiol, β-estradiol+MPA, or melatonin triggered a significant increase in mir-675 expression that was associated with a significant decrease in DUX4 and TRIM43 expression (FIGS. 29B-C and Table 4).

The therapeutic strategy disclosed herein shows that (1) endogenous microRNA gene expression can change in response to small molecule treatments; and (2) natural DUX4-targeted microRNAs can be upregulated to decrease DUX4 expression via the RNAi pathway. By combining these two principles, small molecules can be used to increase expression of natural microRNAs that target DUX4 for degradation within the cell, resulting in a new therapy for muscular dystrophies or cancers associated with DUX4 expression or an overexpression of DUX4.

Importantly, inhibiting mir-675 with the anti-mir-675 antagomir or preventing its binding to a mir-675-resistant DUX4 construct (CMV.DUX4 mir-675Res) supported that the three treatment regimens, i.e., β-estradiol, a combination of β-estradiol+MPA, and melatonin, exert their DUX4 inhibitory effect through mir-675 action (FIG. 28 and Table 3).

This study shows that estrogen, estrogen and progesterone, and melatonin can inhibit DUX4 expression and can be used in the treatment of diseases associated with the expression and/or overexpression of DUX4, such as FSHD or cancer.

### Example 10

### mir-675 Enhances Skeletal Muscle Regeneration and Differentiation

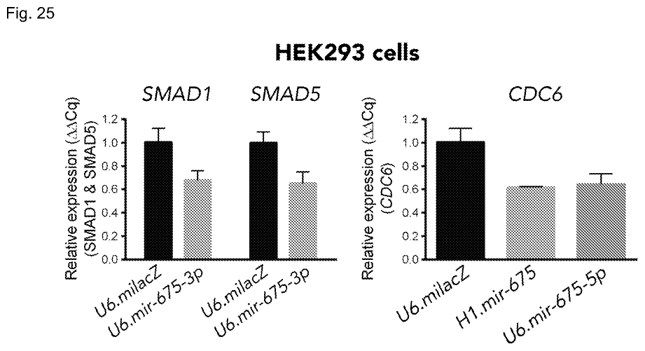

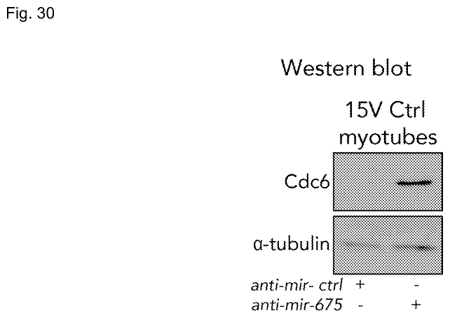

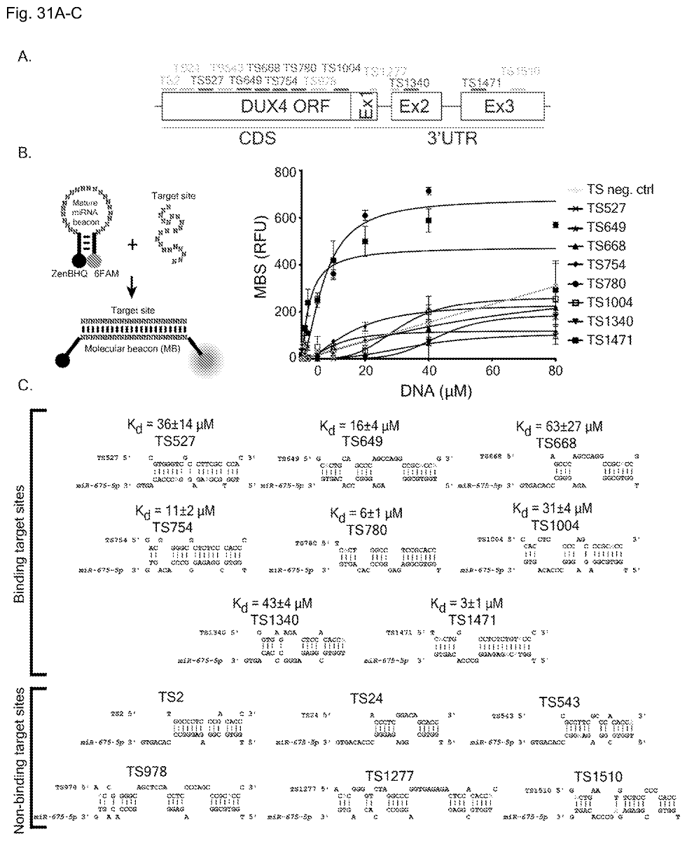

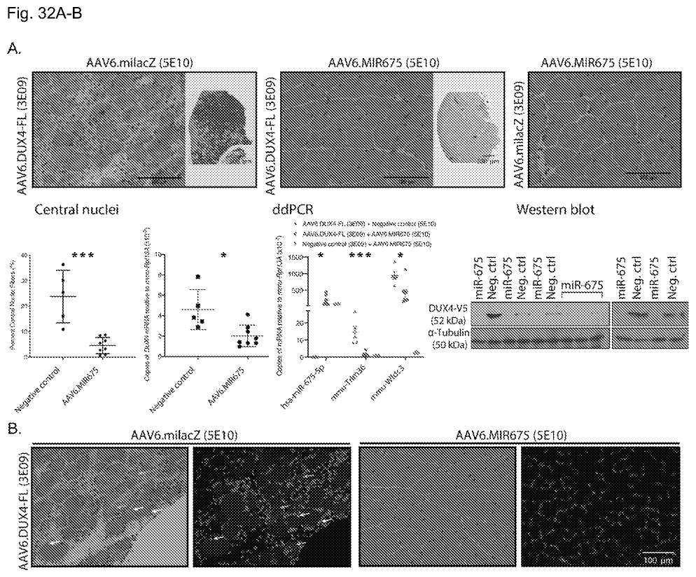

Along with a previous study showing the beneficial effect of mir-675 on regeneration and differentiation of injured mouse skeletal muscles [Dey et al., Genes Dev. 28: 491-501 (2014)], this study shows that mir-675 appears to enhance human skeletal muscle regeneration and differentiation since it was demonstrated that mir-675 can target and down-regulate the anti-differentiation Smad transcription factors (Smad 1 and 5), which are critical for the bone morphogenetic protein (BMP) pathway and the DNA replication initiation factor Cdc6 in human skeletal muscle and non-muscle cell lines (FIGS. 25, 26 and 30). FIG. 25 shows mir-675 targeting of SMAD1, SMAD5 and CDC6 in HEK293 cells. QPCR was used to measure the expression of SMAD1, SMAD5 and CDC6 in HEK293 cells using TaqMan probes specific to each investigated gene. To do that, U6.milacZ (negative control), H1.mir-675, U6.mir-675-3p or U6.mir-675-5p expressing constructs were transfected into HEK293 cells, and total RNA was extracted 48 h after transfection. U6.mir-675-3p reduced SMAD1 levels by an average of 32±6% (P<0.044, ANOVA, N=3) and SMAD5 levels by an average of 35±6% (P<0.0013, ANOVA, N=3). On the other hand, H1.mir-675 and U6.mir-675-5p repressed CDC6 levels by an average of 38±4% (P<0.0034, ANOVA, N=3) and 36±7% (P<0.0048, ANOVA, N=3), respectively. Results were reported as relative gene expression (ΔΔCq)±SEM of three replicates (N=3) relative to gene expression in cells transfected with U6.milacZ, with each QPCR assay performed in triplicate. All results were quantified using as reference gene the house keeping gene RPL13A. FIG. 26 shows the uncropped western blot gel of FIG. 30. In FIG. 30, the endogenous mir-675 targets the CDC6 gene expression in control non-affected differentiated muscle cell lines (myotubes of 15V muscle cell lines) and prevents DUX4-induced toxicity in 15A FSHD-affected human myotubes. The targeting of CDC6 gene expression was tested by using a specific anti-mir-675 antagomir and by measuring Cdc6 protein levels in 4-days differentiated 15V control myotubes. Cdc6 was only detected in myotubes transfected with anti-mir-675 (see FIG. 26 for uncropped gel). The housekeeping protein a-tubulin was used as reference.

Alongside the silencing of DUX4 expression, the involvement of mir-675 in regeneration is expected to be beneficial for FSHD affected skeletal muscles as it could help regenerate new muscle fibers in which DUX4 expression is then reduced.

In summary, mir-675 and the mir-675 analogs provided herein are useful as DUX4 inhibitors that have therapeutic applications for treating FSHD and other diseases associated with DUX4 expression or overexpression.

### Example 11

### mi405G and H are More Efficient than Mi405 in Reducing DUX4 Toxicity In Vivo at Low AAV Doses

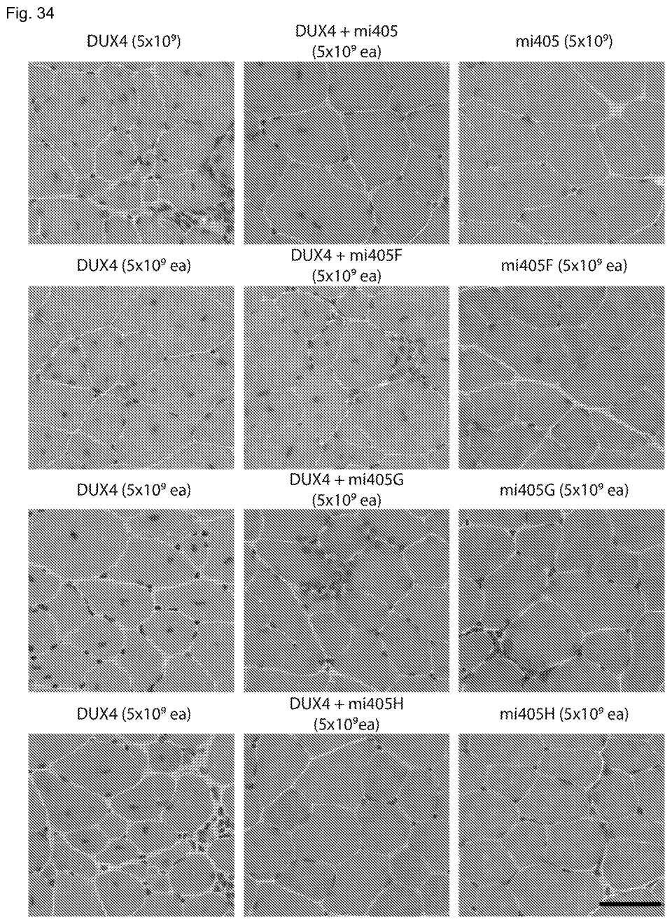

U6.mi405, U6.mi405F, U6.mi405G and U6.mi405H were co-injected using scAAV6 with AAV.CMV.DUX4-FL at equivalent doses (5e09 DNase Resistant Particles (DRP)) in the TA of C57BL/6 mice. This experiment was performed to investigate the efficiency of the four mi405 constructs to eliminate DUX4-induced muscle toxicity in vivo at doses equivalent to that of AAV.CMV.DUX4-FL. Previously, Wallace et al. [Wallace et al, Ann. Neurol. 69: 540-552 (2011); Wallace et al. Mol Ther Methods Clin Dev. 2018 Mar. 16; 8: 121-130] showed that AAV.U6.mi405 was highly efficient in counteracting DUX4-induced toxicity at one log higher dose than that of AAV.DUX4, but never tested AAV.U6.mi405 at lower doses. The data in FIG. 34 show that at lower doses, mi405G and mi405H, but not mi405F, were more efficient than mi405 in eliminating DUX4-induced muscle toxicity characterized by mononuclear cells infiltration and myofibers with central nuclei. This data is consistent with in vitro data (FIGS. 5C and 12) on the exception of mi405F that showed no enhanced inhibition efficiency in vivo when compared to mi405 (FIG. 34).

### Example 12

### Pyrazinamide and Sorafenib Reduced the Expression of DUX4 and DUX4-Responsive Biomarkers (TRIM43 and ZSCAN4) in FSHD Affected Muscle Cells

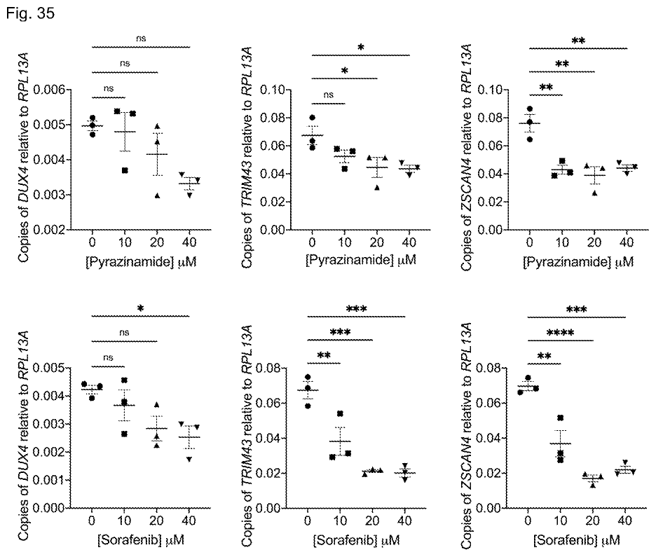

18A FSHD affected muscles cell lines were treated with increasing concentrations of Pyrazinamide or Sorafenib at the 4^{th} day of differentiation into myotubes. Cells were collected at the 5^{th} day of differentiation. RNA was extracted and ddPCR gene expression analysis on DUX4, TRIM43 and ZSCAN4 was carried out. As a result, 40 μM of Pyrazinamide reduced TRIM43 and ZSCAN4 expression by 35±7% (ANOVA, P=0.0378, N=3 independent experiments) and 42±6% (ANOVA, P=0.0043, N=3 independent experiments), respectively (FIG. 35). Sorafenib reduced DUX4, TRIM43 and ZSCAN4 expression in a dose dependent manner, where it reached maximum inhibition when used at a concentration of 40 μM. At the latter, Sorafenib reduced DUX4, TRIM43 and ZSCAN4 expression by 40±10% (ANOVA, P=0.0486, N=3 independent experiments), 70±4% (ANOVA, P=0.0003, N=3 independent experiments) and 68±3% (ANOVA, P=0.0001, N=3 independent experiments), respectively (FIG. 35).

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

We claim:

1. A nucleic acid encoding a double homeobox 4 (DUX4)-targeting microRNA (miRNA) comprising:
(a) a nucleotide sequence comprising at least 90% identity to the sequence set forth in any one of SEQ ID NOs: 5-47;
(b) the nucleotide sequence set forth in any one of SEQ ID NOs: 5-47;
(c) a nucleotide sequence that encodes the RNA sequence set forth in any one of SEQ ID NOs: 95-105; or
(d) a nucleotide sequence that specifically hybridizes to the DUX4 sequence set forth in any one of SEQ ID NOs: 106-124.

2. The nucleic acid of claim 1 further comprising a promoter sequence.

3. The nucleic acid of claim 2, wherein the promoter is any of U6, U7, tRNA, H1, minimal CMV, T7, EF1-alpha, Minimal EF1-alpha, or a muscle-specific promoter.

4. The nucleic acid of claim 3 or 4, wherein the promoter is U6 or H1.

5. The nucleic acid of any one of claims 3-5 comprising:
(a) a nucleotide sequence comprising at least 90% identity to the sequence set forth in any one of SEQ ID NOs: 50-92; or
(b) the nucleotide sequence set forth in any one of SEQ ID NOs: 50-92.

6. The nucleic acid of claim 3, wherein the muscle-specific promoter is unc45b, tMCK, minimal MCK, CK6, CK7, CK8, MHCK7, or CK1.

7. An adeno-associated virus comprising the nucleic acid of any one of claims 1-6.

8. The adeno-associated virus of claim 7, wherein the virus lacks rep and cap genes.

9. The adeno-associated virus of claim 7 or 8, wherein the virus is a recombinant AAV (rAAV) or a self-complementary recombinant AAV (scAAV).

10. The adeno-associated virus of any one of claims 7-9, wherein the virus is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, or AAV-B1.

11. The adeno-associated virus of any one of claims 7-10, wherein the virus is AAV9.

12. A nanoparticle, extracellular vesicle, or exosome comprising the nucleic acid of any one of claims 1-6.

13. A composition comprising
(a) the nucleic acid of any one of claims 1-6;
(b) the adeno-associated virus of any one of claims 7-11; or
(c) the nanoparticle, extracellular vesicle, or exosome of claim 12; and
a pharmaceutically acceptable carrier.

14. A method of inhibiting and/or interfering with expression of a double homeobox 4 (DUX4) gene in a cell comprising contacting the cell with
(a) the nucleic acid of any one of claims 1-6;
(b) the adeno-associated virus of any one of claims 7-11;
(c) the nanoparticle, extracellular vesicle, or exosome of claim 12; and/or
(d) the composition of claim 13.

15. A method of treating a subject having a muscular dystrophy or a cancer comprising administering to the subject an effective amount of
(a) the nucleic acid of any one of claims 1-6;
(b) the adeno-associated virus of any one of claims 7-11;
(c) the nanoparticle, extracellular vesicle, or exosome of claim 12; and/or
(d) the composition of claim 13.

16. The method of claim 14 or 15, wherein the muscular dystrophy is facioscapulohumeral muscular dystrophy (FSHD).

17. The method of claim 14 or 15, wherein the cancer is a sarcoma, a B-cell lymphoma, or a DUX4-expressing cancer of the adrenal, bile duct, bladder, breast, cervix, colon, endometrium, esophagus, head/neck, liver, brain, lung, mesothelium, neural crest, ovary, pancreas, prostate, kidney, skin, soft tissue, stomach, testicles, or thymus.

18. Use of
(a) the nucleic acid of any one of claims 1-6;
(b) the adeno-associated virus of any one of claims 7-11;
(c) the nanoparticle, extracellular vesicle, or exosome of claim 12; and/or
(d) the composition of claim 13
for the preparation of a medicament for inhibiting expression of a double homeobox 4 (DUX4) gene in a cell.

19. Use of
(a) the nucleic acid of any one of claims 1-6;
(b) the adeno-associated virus of any one of claims 7-11;
(c) the nanoparticle, extracellular vesicle, or exosome of claim 12; and/or
(d) the composition of claim 13
for treating or ameliorating a muscular dystrophy or a cancer.

20. Use of
(a) the nucleic acid of any one of claims 1-6;
(b) the adeno-associated virus of any one of claims 7-11;
(c) the nanoparticle, extracellular vesicle, or exosome of claim 12; and/or
(d) the composition of claim 13
for the preparation of a medicament for treating or ameliorating a muscular dystrophy or a cancer.

21. The use of any one of claims 18-20, wherein the muscular dystrophy is facioscapulohumeral muscular dystrophy.

22. The use of any one of claims 18-20, wherein the cancer is a sarcoma, a B-cell lymphoma, or a DUX4-expressing cancer of the adrenal, bile duct, bladder, breast, cervix, colon, endometrium, esophagus, head/neck, liver, brain, lung, mesothelium, neural crest, ovary, pancreas, prostate, kidney, skin, soft tissue, stomach, testicles, or thymus.

23. The
(a) nucleic acid of any one of claims 1-6;
(b) adeno-associated virus (AAV) of any one of claims 7-11;
(c) nanoparticle, extracellular vesicle, or exosome of claim 12;
(d) composition of claim 13;
(e) method of any one of claims 14-17; or
(f) use of any one of claims 18-22,
wherein the nucleic acid, AAV, nanoparticle, extracellular vesicle, exosome, or composition, or medicament is formulated for intramuscular injection, oral administration, subcutaneous, intradermal, or transdermal transport, injection into the blood stream, or for aerosol administration.

24. A method of upregulating expression of microRNA-675 in a cell comprising contacting the cell with an effective amount of an estrogen, synthetic estrogen, progesterone, progestin, melatonin, bleomycin, pyrazinamide, sorafenib, or a derivative thereof, or a combination of any thereof.

25. A method of inhibiting and/or interfering with expression of a double homeobox 4 (DUX4) gene in a cell comprising contacting the cell with an effective amount of an estrogen, synthetic estrogen, progesterone, progestin, melatonin, bleomycin, pyrazinamide, sorafenib, or a derivative thereof, or a combination of any thereof.

26. A method of treating a subject having a muscular dystrophy or a cancer associated with DUX4 expression or overexpression comprising administering to the subject an effective amount of an estrogen, synthetic estrogen, progesterone, progestin, melatonin, bleomycin, pyrazinamide, sorafenib, or a derivative thereof, or a combination of any thereof.

27. The method of claim 26, wherein the muscular dystrophy is facioscapulohumeral muscular dystrophy (FSHD).

28. The method of claim 26, wherein the cancer is a sarcoma, a B-cell lymphoma, or a DUX4-expressing cancer of the adrenal, bile duct, bladder, breast, cervix, colon, endometrium, esophagus, head/neck, liver, brain, lung, mesothelium, neural crest, ovary, pancreas, prostate, kidney, skin, soft tissue, stomach, testicles, or thymus.

29. The method of any one of claims 24-28, wherein the estrogen or synthetic estrogen is estrone, estradiol, estriol, estetrol, 27-hydroxycholesterol, dehydroepiandrosterone (DHEA), 7-oxo-DHEA, 7α-hydroxy-DHEA, 16α-hydroxy-DHEA, 7β-hydroxyepiandrosterone, androstenedione (A4), androstenediol (A5), 3α-androstanediol, and 3β-androstanediol, 2-hydroxyestradiol, 2-hydroxyestrone, 4-hydroxyestradiol, 4-hydroxyestrone, 16α-hydroxyestrone, ethinyl estradiol, estradiol valerate, estropipate, conjugate esterified estrogen, and quinestrol.

30. The method of any one of claims 24-28, wherein the progesterone or progestin is medroxyprogesterone acetate (MPA), 17α-hydroxyprogesterone, chlormadinone acetate, cyproterone acetate, gestodene, or etonogestrel.

31. Use of an estrogen, synthetic estrogen, progesterone, progestin, melatonin, bleomycin, pyrazinamide, sorafenib, or a derivative thereof, or a combination of any thereof for upregulating expression of microRNA-675 in a cell.

32. Use of an estrogen, synthetic estrogen, progesterone, progestin, melatonin, bleomycin, pyrazinamide, sorafenib, or a derivative thereof, or a combination of any thereof for inhibiting and/or interfering with expression of a double homeobox 4 (DUX4) gene in a cell.

33. Use of an estrogen, synthetic estrogen, progesterone, progestin, melatonin, bleomycin, pyrazinamide, sorafenib, or a derivative thereof, or a combination of any thereof for treating a subject having a muscular dystrophy or a cancer associated with DUX4 expression or overexpression.

34. The use of any one of claims 31-33, wherein the muscular dystrophy is facioscapulohumeral muscular dystrophy.

35. The use of any one of claims 31-33, wherein the cancer is a sarcoma, a B-cell lymphoma, or a DUX4-expressing cancer of the adrenal, bile duct, bladder, breast, cervix, colon, endometrium, esophagus, head/neck, liver, brain, lung, mesothelium, neural crest, ovary, pancreas, prostate, kidney, skin, soft tissue, stomach, testicles, or thymus.

36. The use of any one of claims 31-35, wherein the estrogen or synthetic estrogen is estrone, estradiol, estriol, estetrol, 27-hydroxycholesterol, dehydroepiandrosterone (DHEA), 7-oxo-DHEA, 7α-hydroxy-DHEA, 16α-hydroxy-DHEA, 7β-hydroxyepiandrosterone, androstenedione (A4), androstenediol (A5), 3α-androstanediol, and 3β-androstanediol, 2-hydroxyestradiol, 2-hydroxyestrone, 4-hydroxyestradiol, 4-hydroxyestrone, 16α-hydroxyestrone, ethinyl estradiol, estradiol valerate, estropipate, conjugate esterified estrogen, and quinestrol.

37. The use of any one of claims 31-36, wherein the progesterone or progestin is medroxyprogesterone acetate (MPA), 17α-hydroxyprogesterone, chlormadinone acetate, cyproterone acetate, gestodene, or etonogestrel.

38. The method of any one of claims 24-30 or the use of any one of claims 31-37, wherein the estrogen, synthetic estrogen, progesterone, progestin, melatonin, bleomycin, pyrazinamide, sorafenib, or the derivative thereof, or the combination of any thereof is formulated for intramuscular injection, oral administration, subcutaneous, intradermal, or transdermal transport, injection into the blood stream, or for aerosol administration.
